(19) United States
(12) Patent Application Publication
(10) Pub. No.: US 2024/0228459 A1
(43) Pub. Date: Jul. 11, 2024

(54) WEE1 INHIBITORS AND USES THEREOF

(71) Applicant: Boundless Bio, Inc., San Diego, CA (US)

(72) Inventors: Anthony B. PINKERTON, Rancho Santa Fe, CA (US); Huikai SUN, San Diego, CA (US); Ronghua LI, San Diego, CA (US); Srinivas KASIBHATLA, San Diego, CA (US)

(21) Appl. No.: 18/390,368

(22) Filed: Dec. 20, 2023

Related U.S. Application Data
(60) Provisional application No. 63/476,846, filed on Dec. 22, 2022.

(51) Int. Cl. C07D 401/12; A61K 45/06; C07D 401/14; C07D 413/14; C07D 471/04; C07D 471/18; C07D 498/04

(52) U.S. Cl. CPC C07D 401/12; A61K 45/06; C07D 401/14; C07D 413/14; C07D 471/04; C07D 471/18; C07D 498/04

(57) ABSTRACT

Provided herein are compounds and methods for the treatment of cancer. The methods include administering to a subject in need a therapeutically effective amount of a WEE1 inhibitor disclosed herein.

### CROSS-REFERENCE

This application claims the benefit of U.S. Provisional Application Ser. No. 63/476,846, filed Dec. 22, 2022, which is hereby incorporated by reference in its entirety.

### FIELD OF THE INVENTION

Described herein are compounds, methods of making such compounds, pharmaceutical compositions, and medicaments comprising such compounds, and methods of using such compounds for inhibiting WEE1.

### BACKGROUND OF THE INVENTION

Cells depend on cell cycle checkpoint enzymes to allow time for DNA repair and maintenance of genomic integrity before they begin cell division, a process known as mitosis. Damage to cellular DNA activates the intracellular DNA damage response mechanisms. Essential to this process is the activation of WEE1 tyrosine kinase. WEE1, a nuclear tyrosine kinase, regulates the G2-M cell-cycle checkpoint via phosphorylation and inactivation of Cyclin Dependent Kinase 1 (CDK1) at Tyrl5 in response to extrinsic DNA damage and errors in DNA synthesis, thereby preventing entry into mitosis. WEE1 is regarded as essential to the DNA repair process, and is viewed as a mitotic gatekeeper.

Many conventional anticancer treatments, including antimetabolites, ionizing radiation, alkylating agents, platinum compounds and DNA topoisomerase inhibitors exert their antitumor effects by damaging DNA in tumor cells. However, these treatments also cause activation of cell cycle checkpoints, including WEE1 and CDK1 and 2, which gives the tumor cell time to repair the damaged DNA before it begins mitosis. Thus, tumor cells can exploit the DNA repair process, rendering them somewhat refractory or even immune to the anticancer therapy.

Although certain WEE1 inhibitors have been identified, such as AZD1775, there are shortcomings such as dose-limiting toxicities, including neutropenia, thrombocytopenia, anemia, and nausea, and off-target activity against other kinases, for example, the Ser/Thr kinase, PLK1.

There is a need for an agent that more selectively eliminates WEE1 activity, as well as an agent that offers the potential for reducing off-target toxicities.

### BRIEF SUMMARY OF THE INVENTION

Described herein are WEE1 inhibitors that are useful in treating cancer.

Disclosed herein is a compound of Formula (I), or a pharmaceutically acceptable salt, solvate, or stereoisomer thereof:

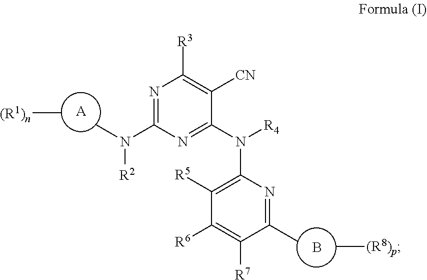

wherein:
Ring A is a monocyclic or a bicyclic ring.
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, hetercycloalkyl, aryl, and heteroaryl is optionally substituted with one or more R^{1a};
or two R¹ on the same atom form an oxo;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1b};
or two R^{1a} on the same atom form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R^{1b} on the same atom form an oxo;
n is 0, 1, 2, 3, 4, 5, or 6;
R² is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R³ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁷ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring B is a cycloalkyl, heterocycloalkyl, aryl, or heteroaryl;
each R⁸ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R⁸ on the same carbon form an oxo;
p is 0, 1, 2, 3, 4, 5, or 6;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom form an oxo.

In some embodiments of a compound of Formula (I), compound is of Formula (Ia):

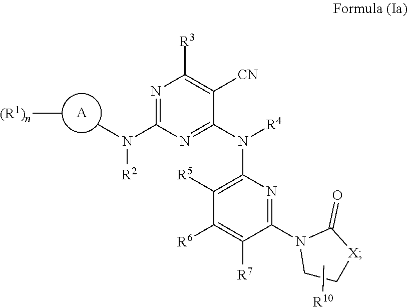

wherein:
X is —O—, —S—, or —C(R⁹)₂—;
each R⁹ is independently hydrogen deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R¹⁰ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R.

Also disclosed herein is a compound of Formula (II), or a pharmaceutically acceptable salt, solvate, or stereoisomer thereof:

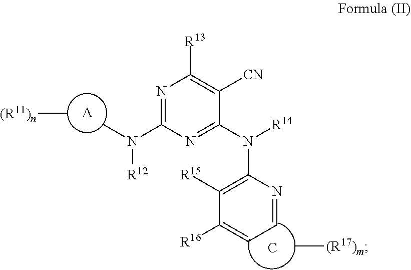

wherein:
Ring A is cycloalkyl, heterocycloalkyl, aryl, or heteroaryl;
each R¹¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{11a};
or two R¹¹ on the same atom form an oxo;
each R^{11a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{11b};
or two R^{11a} on the same atom form an oxo;
each R^{11b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R^{11b} on the same atom form an oxo;
n is 0, 1, 2, 3, 4, 5, or 6;
R¹² is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R₁₃ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R¹⁴ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R¹⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R¹⁶ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring C is cycloalkyl or heterocycloalkyl;
each R¹⁷ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R¹⁷ on the same carbon form an oxo;
m is 0, 1, 2, 3, 4, 5, or 6;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl). C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom form an oxo;
provided that the compound is not

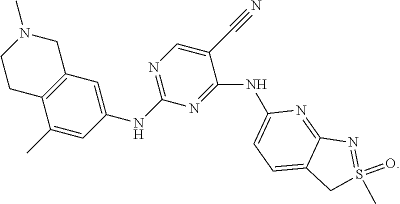 [SMILES: Cc1cc(Nc2ncc(C#N)c(Nc3ccc4c(n3)N=S(C)(=O)C4)n2)cc2c1CCN(C)C2]

In some embodiments of a compound of Formula (II), the compound is of Formula (IIa):

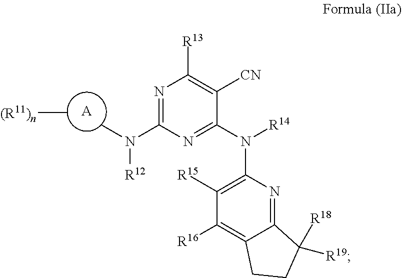

wherein:
R¹⁸ and R¹⁹ are independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R.

Also disclosed herein is a pharmaceutical composition comprising a compound disclosed herein, or a pharmaceutically acceptable salt, solvate, tautomer, or stereoisomer thereof, and a pharmaceutically acceptable excipient.

Also disclosed herein is a method of treating cancer in a subject, comprising administering to the subject a compound disclosed herein, or a pharmaceutically acceptable salt, solvate, tautomer, or stereoisomer thereof, or a pharmaceutical composition disclosed herein.

Also disclosed herein is a method of inhibiting WEE1 in a subject, comprising administering to the subject a compound disclosed herein, or a pharmaceutically acceptable salt, solvate, tautomer, or stereoisomer thereof, or a pharmaceutical composition disclosed herein.

Also disclosed herein is a method of treating a tumor or tumor cells, comprising administering the compound disclosed herein, or a pharmaceutically acceptable salt, solvate, tautomer, or stereoisomer thereof, and a cancer-targeted therapeutic agent to a subject identified as having the tumor or tumor cells, wherein the tumor or tumor cells comprise a gene amplification, whereby growth or size of the tumor or growth or number of tumor cells is reduced.

Also disclosed herein is a method of delaying resistance to a cancer-targeted therapeutic agent, the method comprising: administering the compound disclosed herein, or a pharmaceutically acceptable salt, solvate, tautomer, or stereoisomer thereof, to a subject in an amount sufficient to induce replication stress in tumor or tumor cells and administering a cancer-targeted therapeutic agent concurrently or subsequent to the administration of the compound.

In some embodiments, the subject is identified as having the tumor or tumor cells comprising a gene amplification.

In some embodiments, the gene amplification is a focal gene amplification.

In some embodiments, the gene amplification is an ecDNA-derived amplification.

In some embodiments, the gene amplification is comprised on ecDNA or an HSR (homogeneously staining region).

In some embodiments, the cancer-targeted therapeutic agent is directed against a protein encoded by a gene contained within the gene amplification.

In some embodiments, the tumor or tumor cells comprise an ecDNA signature.

In some embodiments, cells comprised within the tumor or the tumor cells are ecDNA competent.

Also disclosed herein is a method of treating a subject having a tumor or tumor cells that are non-responsive to a first cancer-targeted therapeutic agent comprising administering the compound disclosed herein, or a pharmaceutically acceptable salt, solvate, tautomer, or stereoisomer thereof, and a second cancer-targeted therapeutic agent to the subject.

In some embodiments, prior to treatment with the compound, or a pharmaceutically acceptable salt, solvate, tautomer, or stereoisomer thereof, the tumor or tumor cells comprise a gene amplification and wherein the first cancer-targeted therapeutic agent has an activity directed against a protein encoded by a gene contained within the gene amplification.

Also disclosed herein is a method of provoking a response in a subject having a tumor or tumor cells that are non-responsive or have decreased responsiveness to a first cancer-targeted therapeutic agent comprising administering the compound disclosed herein, or a pharmaceutically acceptable salt, solvate, tautomer, or stereoisomer thereof, and the first cancer-targeted therapeutic agent to the subject.

In some embodiments, prior to treatment with the compound, or a pharmaceutically acceptable salt, solvate, tautomer, or stereoisomer thereof, the tumor or tumor cells comprise a gene amplification and wherein the first cancer-targeted therapeutic agent has an activity directed against a protein encoded by a gene contained within the gene amplification.

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Oxo” refers to ═O.

“Alkyl” refers to an optionally substituted straight-chain, or optionally substituted branched-chain saturated hydrocarbon monoradical having from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen. In some embodiments, the alkyl is optionally substituted with —COOH, —COOMe, —CONH₂, —CONHMe, or —CONMe₂.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s) and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen. In some embodiments, the alkenyl is optionally substituted with —COOH, —COOMe, —CONH₂, —CONHMe, or —CONMe₂.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. In some embodiments, the alkynyl is optionally substituted with —COOH, —COOMe, —CONH₂, —CONHMe, or —CONMe₂.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —N₂. In some embodiments, an alkylene is optionally substituted with oxo, halogen, —CN, —CF₃, —OH, or —OMe. In some embodiments, the alkylene is optionally substituted with halogen. In some embodiments, the alkylene is optionally substituted with —COOH, —COOMe, —CONH₂, —CONHMe, or —CONMe₂.

“Alkoxy” refers to a radical of the formula —Oalkyl where alkyl is as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen. In some embodiments, the alkoxy is optionally substituted with —COOH, —COOMe, —CONH₂, —CONHMe, or —CONMe₂.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen. In some embodiments, the aryl is optionally substituted with —COOH, —COOMe, —CONH₂, —CONHMe, or —CONMe₂.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 6-membered cycloalkyl. In some embodiments, the cycloalkyl is a 5- to 6-membered cycloalkyl. In some embodiments, the cycloalkyl is a 3- to 10-membered monocyclic or bicyclic cycloalkyl. In some embodiments, the cycloalkyl is a 3- to 8-membered monocyclic or bicyclic cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen. In some embodiments, the cycloalkyl is optionally substituted with —COOH, —COOMe, —CONH₂, —CONHMe, or —CONMe₂.

“Deuteroalkyl” refers to an alkyl radical, as defined above, that is substituted by one or more deuterium atoms. In some embodiments, the alkyl is substituted with one deuterium atom. In some embodiments, the alkyl is substituted with one, two, or three deuterium atoms. In some embodiments, the alkyl is substituted with one, two, three, four, five, or six deuterium atoms. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogen atoms. In some embodiments, the alkyl is substituted with one, two, or three halogen atoms. In some embodiments, the alkyl is substituted with one, two, three, four, five, or six halogen halogens. Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro or iodo. In some embodiments, halogen is fluoro or chloro. In some embodiments, halogen is fluoro. In some embodiments, halogen is chloro. In some embodiments, halogen is bromo. In some embodiments, halogen is iodo.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In some embodiments, a heteroalkyl is a C₁-C₆ heteroalkyl comprising one to four heteroatoms selected from the group consisting of oxygen, nitrogen, and sulfur wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen. In some embodiments, the heteroalkyl is optionally substituted with —COOH, —COOMe, —CONH₂, —CONHMe, or —CONMe₂.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a 3- to 24-membered partially or fully saturated, not fully aromatic ring radical comprising 2 to 23 carbon atoms and from one to 8 heteroatoms selected from the group consisting of nitrogen, oxygen, phosphorous, and sulfur. In some embodiments, the heterocycloalkyl comprises 1 to 3 heteroatoms selected from the group consisting of nitrogen, oxygen, and sulfur. In some embodiments, the heterocycloalkyl comprises 1 or 2 heteroatoms selected from the group consisting of nitrogen and oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to six carbon atoms (C₂-C₆ heterocycloalkyl or C₂-C₆ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen. In some embodiments, the heterocycloalkyl is optionally substituted with —COOH, —COOMe, —CONH₂, —CONHMe, or —CONMe₂.

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring comprising at least one heteroatom. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 5- to 6-membered heteroaryl comprising 1 to 3 heteroatoms selected from the group consisting of nitrogen, oxygen, and sulfur. In some embodiments, the heteroaryl is a 5- to 6-membered heteroaryl comprising 1 to 3 heteroatoms selected from the group consisting of nitrogen and oxygen. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen. In some embodiments, the heteroaryl is optionally substituted with —COOH, —COOMe, —CONH₂, —CONHMe, or —CONMe₂.

The term “one or more” when referring to an optional substituent means that the subject group is optionally substituted with one, two, three, four, or more substituents. In some embodiments, the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The terms “treat,” “treated,” “treatment,” or “treating” as used herein refers to therapeutic treatment, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms, diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “treat,” “treated,” “treatment,” or “treating” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the disclosed methods can provide any amount of any level of treatment of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “ecDNA signature” as used herein, generally refers to one or more characteristics common to tumors or tumor cells that are ecDNA+. In some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof. In some cases, ecDNA signature includes a detection or identification of ecDNA using an imaging technology. In some cases, ecDNA signature does not include any imaging or direct detection of ecDNA.

### Compounds

Described herein are WEE1 inhibitor that are useful for the treatment of cancer. In some embodiments, the cancer is a brain tumor or is a cancer that has metastasized in the brain.

Disclosed herein is a compound of Formula (I), or a pharmaceutically acceptable salt, solvate, or stereoisomer thereof:

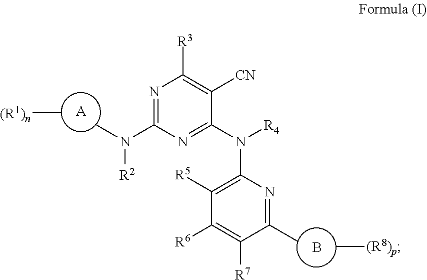

wherein:
Ring A is a monocyclic or a bicyclic ring;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1a}
or two R¹ on the same atom form an oxo;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1b};
or two R^{1a} on the same atom form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R^{1b} on the same atom form an oxo,
n is 0, 1, 2, 3, 4, 5, or 6;
R² is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R³ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁷ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring B is a cycloalkyl, heterocycloalkyl, aryl, or heteroaryl;
each R⁸ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R⁸ on the same carbon form an oxo;
p is 0, 1, 2, 3, 4, 5, or 6;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom form an oxo.

In some embodiments of a compound of Formula (I), Ring B is a cycloalkyl or heterocycloalkyl. In some embodiments of a compound of Formula (I), Ring B is a heterocycloalkyl. In some embodiments of a compound of Formula (I), Ring B is a 5- or 6-membered heterocycloalkyl. In some embodiments of a compound of Formula (I), Ring B is a 5- or 6-membered N-linked heterocycloalkyl. In some embodiments of a compound of Formula (I), Ring B is a 5-membered heterocycloalkyl. In some embodiments of a compound of Formula (I), Ring B is a 5-membered N-linked heterocycloalkyl. In some embodiments of a compound of Formula (I), Ring B is a 6-membered heterocycloalkyl. In some embodiments of a compound of Formula (I), Ring B is a 6-membered N-linked heterocycloalkyl.

In some embodiments of a compound of Formula (I), each R⁸ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R; or two R⁸ on the same carbon form an oxo. In some embodiments of a compound of Formula (I), each R⁸ is independently deuterium, halogen, —CN, —OH, —OR^{a}, —NR^{c}R^{d}, C₁-C₆alkyl, or C₁-C₆haloalkyl; or two R⁸ on the same carbon form an oxo. In some embodiments of a compound of Formula (I), each R⁸ is independently deuterium, halogen, C₁-C₆alkyl, or C₁-C₆haloalkyl; or two R⁸ on the same carbon form an oxo. In some embodiments of a compound of Formula (I), each R⁸ is independently deuterium, halogen, C₁-C₆alkyl, or C₁-C₆haloalkyl; or two R⁸ on the same carbon form an oxo. In some embodiments of a compound of Formula (I), each R⁸ is independently C₁-C₆alkyl or C₁-C₆haloalkyl; or two R⁸ on the same carbon form an oxo. In some embodiments of a compound of Formula (I), two R⁸ on the same carbon form an oxo.

In some embodiments of a compound of Formula (I), p is 0, 1, or 2. In some embodiments of a compound of Formula (I), p is 0, 1, 2, or 3. In some embodiments of a compound of Formula (I), p is 0, 1, 2, or 3. In some embodiments of a compound of Formula (I), p is 2 or 3. In some embodiments of a compound of Formula (I), p is 0. In some embodiments of a compound of Formula (I), p is 1. In some embodiments of a compound of Formula (I), p is 2. In some embodiments of a compound of Formula (I), p is 3. In some embodiments of a compound of Formula (I), p is 4.

In some embodiments of a compound of Formula (I), the compound is of Formula (Ia):

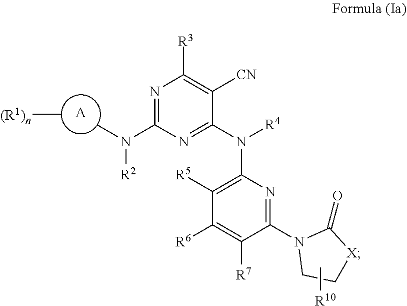

wherein:
X is —O—, —S—, or —C(R⁹)₂—;
each R⁹ is independently hydrogen deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R¹⁰ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₁-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R.

In some embodiments of a compound of Formula (Ia), X is —O—. In some embodiments of a compound of Formula (Ia), X is —S—. In some embodiments of a compound of Formula (Ia), X is —C(R⁹)₂—.

In some embodiments of a compound of Formula (Ia), each R⁹ is independently hydrogen, deuterium, halogen, C₁-C₆alkyl, or C₁-C₆haloalkyl. In some embodiments of a compound of Formula (Ia), each R⁹ is independently hydrogen deuterium, halogen, —CN, —OH, —OR^{a}, —NR^{c}R^{d}, C₁-C₆alkyl, or C₁-C₆haloalkyl. In some embodiments of a compound of Formula (Ia), each R⁹ is independently hydrogen, halogen, C₁-C₆alkyl, or C₁-C₆haloalkyl. In some embodiments of a compound of Formula (Ia), each R⁹ is independently hydrogen, halogen, or C₁-C₆alkyl. In some embodiments of a compound of Formula (Ia), each R⁹ is hydrogen.

In some embodiments of a compound of Formula (Ia), R¹⁰ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl; wherein the alkyl, cycloalkyl, and heterocycloalkyl is optionally substituted with one or more R. In some embodiments of a compound of Formula (Ia), R¹⁰ is hydrogen, deuterium, halogen, —CN, —OH, —OR^{a}, —NR^{c}R^{d}, C₁-C₆alkyl, or C₁-C₆haloalkyl. In some embodiments of a compound of Formula (Ia), R¹⁰ is hydrogen, halogen, C₁-C₆alkyl, or C₁-C₆haloalkyl. In some embodiments of a compound of Formula (Ia), R¹⁰ is hydrogen, halogen, or C₁-C₆alkyl. In some embodiments of a compound of Formula (Ia), R¹⁰ is hydrogen or C₁-C₆alkyl. In some embodiments of a compound of Formula (Ia), R¹⁰ is hydrogen.

In some embodiments of a compound of Formula (I) or (Ia), R² is hydrogen, C₁-C₆alkyl, C₁-C₆haloalkyl, cycloalkyl, or heterocycloalkyl. In some embodiments of a compound of Formula (I) or (Ia), R² is hydrogen, C₁-C₆alkyl, or C₁-C₆haloalkyl. In some embodiments of a compound of Formula (I) or (Ia), R² is hydrogen or C₁-C₆alkyl. In some embodiments of a compound of Formula (I) or (Ia), R² is hydrogen.

In some embodiments of a compound of Formula (I) or (Ia), R³ is hydrogen, deuterium, halogen, —CN, —OH, —OR^{a}, —NR^{c}R^{d}, C₁-C₆alkyl, C₁-C₆haloalkyl, cycloalkyl, or heterocycloalkyl. In some embodiments of a compound of Formula (I) or (Ia), R³ is hydrogen, deuterium, halogen, C₁-C₆alkyl, C₁-C₆haloalkyl, cycloalkyl, or heterocycloalkyl. In some embodiments of a compound of Formula (I) or (Ia), R³ is hydrogen, deuterium, halogen, C₁-C₆alkyl, or C₁-C₆haloalkyl. In some embodiments of a compound of Formula (I) or (Ia), R³ is hydrogen, halogen, or C₁-C₆alkyl. In some embodiments of a compound of Formula (I) or (Ia), R³ is hydrogen or C₁-C₆alkyl. In some embodiments of a compound of Formula (I) or (Ia), R³ is hydrogen or halogen. In some embodiments of a compound of Formula (I) or (Ia), R³ is hydrogen.

In some embodiments of a compound of Formula (I) or (Ia), R⁴ is hydrogen, C₁-C₆alkyl, C₁-C₆haloalkyl, cycloalkyl, or heterocycloalkyl. In some embodiments of a compound of Formula (I) or (Ia), R⁴ is hydrogen, C₁-C₆alkyl, or C₁-C₆haloalkyl. In some embodiments of a compound of Formula (I) or (Ia). R⁴ is hydrogen or C₁-C₆alkyl. In some embodiments of a compound of Formula (I) or (Ia). R⁴ is hydrogen.

In some embodiments of a compound of Formula (I) or (Ia), R⁵ is hydrogen, deuterium, halogen, —CN, —OH, —OR^{a}, —NR^{c}R^{d}, C₁-C₆alkyl, C₁-C₆haloalkyl, cycloalkyl, or heterocycloalkyl. In some embodiments of a compound of Formula (I) or (Ia), R⁵ is hydrogen, deuterium, halogen, C₁-C₆alkyl, C₁-C₆haloalkyl, cycloalkyl, or heterocycloalkyl. In some embodiments of a compound of Formula (I) or (Ia). R is hydrogen, deuterium, halogen, C₁-C₆alkyl, or C₁-C₆haloalkyl. In some embodiments of a compound of Formula (I) or (Ia), R⁵ is hydrogen, halogen, or C₁-C₆alkyl. In some embodiments of a compound of Formula (I) or (Ia), R⁵ is hydrogen or C₁-C₆alkyl. In some embodiments of a compound of Formula (I) or (Ia), R⁵ is hydrogen or halogen. In some embodiments of a compound of Formula (I) or (Ia), R⁵ is hydrogen.

In some embodiments of a compound of Formula (I) or (Ia), R⁶ is hydrogen, deuterium, halogen, —CN, —OH, —OR^{a}, —NR^{c}R^{d}, C₁-C₆alkyl, C₁-C₆haloalkyl, cycloalkyl, or heterocycloalkyl. In some embodiments of a compound of Formula (I) or (Ia), R⁶ is hydrogen, deuterium, halogen, C₁-C₆alkyl, C₁-C₆haloalkyl, cycloalkyl, or heterocycloalkyl. In some embodiments of a compound of Formula (I) or (Ia), R⁶ is hydrogen, deuterium, halogen, C₁-C₆alkyl, or C₁-C₆haloalkyl. In some embodiments of a compound of Formula (I) or (Ia), R⁶ is hydrogen, halogen, or C₁-C₆alkyl. In some embodiments of a compound of Formula (I) or (Ia), R⁶ is hydrogen or C₁-C₆alkyl. In some embodiments of a compound of Formula (I) or (Ia), R⁶ is hydrogen or halogen. In some embodiments of a compound of Formula (I) or (Ia), R⁶ is hydrogen.

In some embodiments of a compound of Formula (I) or (Ia), R⁷ is hydrogen, deuterium, halogen, —CN, —OH, —OR^{a}, —NR^{c}R^{d}, C₁-C₆alkyl, C₁-C₆haloalkyl, cycloalkyl, or heterocycloalkyl. In some embodiments of a compound of Formula (I) or (Ia), R⁷ is hydrogen, deuterium, halogen, C₁-C₆alkyl, C₁-C₆haloalkyl, cycloalkyl, or heterocycloalkyl. In some embodiments of a compound of Formula (I) or (Ia), R⁷ is hydrogen, deuterium, halogen, C₁-C₆alkyl, or C₁-C₆haloalkyl. In some embodiments of a compound of Formula (I) or (Ia), R⁷ is hydrogen, halogen, or C₁-C₆alkyl. In some embodiments of a compound of Formula (I) or (Ia), R⁷ is hydrogen or C₁-C₆alkyl. In some embodiments of a compound of Formula (I) or (Ia), R⁷ is hydrogen or halogen. In some embodiments of a compound of Formula (I) or (Ia), R⁷ is hydrogen.

In some embodiments of a compound of Formula (I) or (Ia), Ring A is a monocyclic or a bicyclic cycloalkyl, a monocyclic or a bicyclic heterocycloalkyl, a monocyclic or a bicyclic aryl, or a monocyclic or a bicyclic heteroaryl. In some embodiments of a compound of Formula (I) or (Ia), Ring A is a monocyclic or a bicyclic aryl or a monocyclic or a bicyclic heteroaryl. In some embodiments of a compound of Formula (I) or (Ia), Ring A is a monocyclic aryl or a monocyclic heteroaryl. In some embodiments of a compound of Formula (I) or (Ia), Ring A is a phenyl. In some embodiments of a compound of Formula (I) or (Ia), Ring A is a bicyclic heteroaryl. In some embodiments of a compound of Formula (I) or (Ia), Ring A is 1,2,3,4-tetrahydroisoquinolinyl.

In some embodiments of a compound of Formula (I) or (Ia), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{1a}. In some embodiments of a compound of Formula (I) or (Ia), each R¹ is independently deuterium, halogen. C₁-C₆alkyl, C₁-C₆haloalkyl, C₁-C₆heteroalkyl, cycloalkyl, or heterocycloalkyl; wherein the alkyl, cycloalkyl, and heterocycloalkyl is optionally substituted with one or more R^{1a}. In some embodiments of a compound of Formula (I) or (Ia), each R¹ is independently halogen, C₁-C₆alkyl, C₁-C₆heteroalkyl, cycloalkyl, or heterocycloalkyl; wherein the alkyl, cycloalkyl, and heterocycloalkyl is optionally substituted with one or more R^{1a}. In some embodiments of a compound of Formula (I) or (Ia), each R¹ is independently halogen, C₁-C₆alkyl, C₁-C₆heteroalkyl, or heterocycloalkyl; wherein the alkyl, and heterocycloalkyl is optionally substituted with one or more R^{1a}. In some embodiments of a compound of Formula (I) or (Ia), each R¹ is independently heterocycloalkyl optionally substituted with one or more R^{1a}. In some embodiments of a compound of Formula (I) or (Ia), each R¹ is independently heterocycloalkyl optionally substituted with one or more R^{1a}; wherein the heterocycloalkyl is monocyclic or bicyclic. In some embodiments of a compound of Formula (I) or (Ia), each R¹ is independently heterocycloalkyl optionally substituted with one or more R^{1a}; wherein the heterocycloalkyl is a bridged heterocycloalkyl, fused heterocycloalkyl, or spirocyclic heterocycloalkyl. In some embodiments of a compound of Formula (I) or (Ia), each R¹ is independently heterocycloalkyl optionally substituted with one or more R^{1a}; wherein the heterocycloalkyl is 2,5-diazabicyclo[2.2.1]heptanyl, 3,8-diazabicyclo[3.2.1]octanyl, 3,8-diazabicyclo[3.2.1]octanyl, or 2,5-diazabicyclo[2.2.2]octanyl. In some embodiments of a compound of Formula (I) or (Ia), each R¹ is independently heterocycloalkyl optionally substituted with one or more R^{1a}; wherein the heterocycloalkyl is octahydropyrrolo[3,4-c]pyrrolyl. In some embodiments of a compound of Formula (I) or (Ia), each R¹ is independently heterocycloalkyl optionally substituted with one or more R^{1a}; wherein the heterocycloalkyl is 4,7-diazaspiro[2.5]octanyl or 3,9-diazaspiro[5.5]undecanyl. In some embodiments of a compound of Formula (I) or (Ia), each R¹ is independently halogen or C₁-C₆alkyl.

In some embodiments of a compound of Formula (I) or (Ia), each R^{1a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, is heterocycloalkyl is optionally substituted with one or more R^{1b}; or two R^{1a} on the same atom form an oxo. In some embodiments of a compound of Formula (I) or (Ia), each R^{1a} is independently deuterium, halogen, C₁-C₆alkyl, C₁-C₆haloalkyl, or C₁-C₆heteroalkyl; wherein the alkyl is optionally substituted with one or more R^{1b}; or two R^{1a} on the same atom form an oxo.

In some embodiments of a compound of Formula (I) or (Ia), each R^{1a} is independently deuterium, halogen, C₁-C₆alkyl, C₁-C₆haloalkyl, or C₁-C₆heteroalkyl; wherein the alkyl is optionally substituted with one or more R^{1b}; or two R^{1a} on the same atom form an oxo. In some embodiments of a compound of Formula (I) or (Ia), each R^{1a} is independently C₁-C₆alkyl, C₁-C₆haloalkyl, or C₁-C₆heteroalkyl; or two R^{1a} on the same atom form an oxo. In some embodiments of a compound of Formula (I) or (Ia), each R^{1a} is independently C₁-C₆alkyl, C₁-C₆haloalkyl, or C₁-C₆heteroalkyl. In some embodiments of a compound of Formula (I) or (Ia), each R^{1a} is independently C₁-C₆alkyl.

In some embodiments of a compound of Formula (I) or (Ia), each R^{1b} is independently deuterium, halogen, —CN, —OH, —OR^{a}, —NR^{c}R^{d}, C₁-C₆alkyl, or C₁-C₆haloalkyl. In some embodiments of a compound of Formula (I) or (Ia), each R^{1b} is independently deuterium, halogen, —OH, C₁-C₆alkyl, or C₁-C₆haloalkyl. In some embodiments of a compound of Formula (I) or (Ia), each R^{1b} is independently C₁-C₆alkyl.

In some embodiments of a compound of Formula (I) or (Ia), n is 0, 1, 2, 3, or 4. In some embodiments of a compound of Formula (I) or (Ia), n is 1, 2, or 3. In some embodiments of a compound of Formula (I) or (Ia), n is 1 or 2. In some embodiments of a compound of Formula (I) or (Ia), n is 0, 1, or 2. In some embodiments of a compound of Formula (I) or (Ia), n is 0 or 1. In some embodiments of a compound of Formula (I) or (Ia), n is 0. In some embodiments of a compound of Formula (I) or (Ia), n is 1. In some embodiments of a compound of Formula (I) or (Ia), n is 2. In some embodiments of a compound of Formula (I) or (Ia), n is 3.

In some embodiments of a compound of Formula (I) or (Ia),

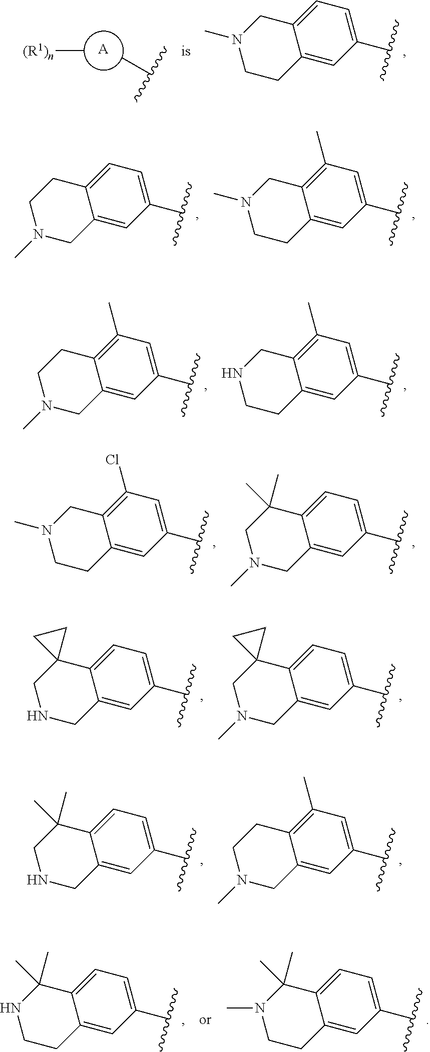

In some embodiments of a compound of Formula (I) or (Ia),

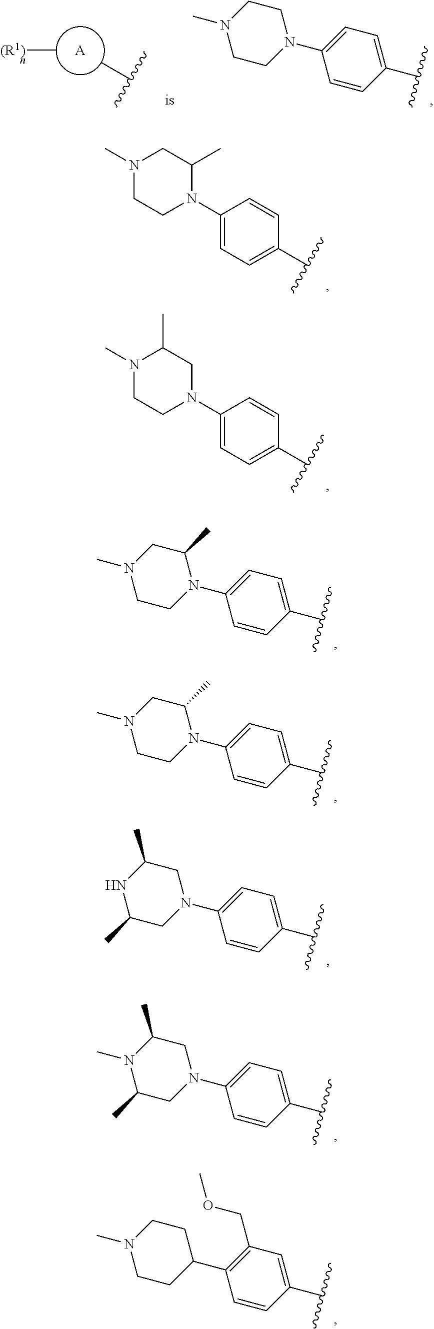

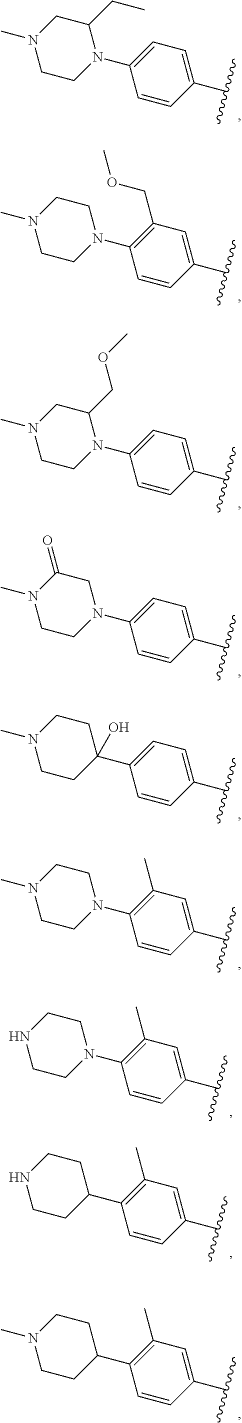

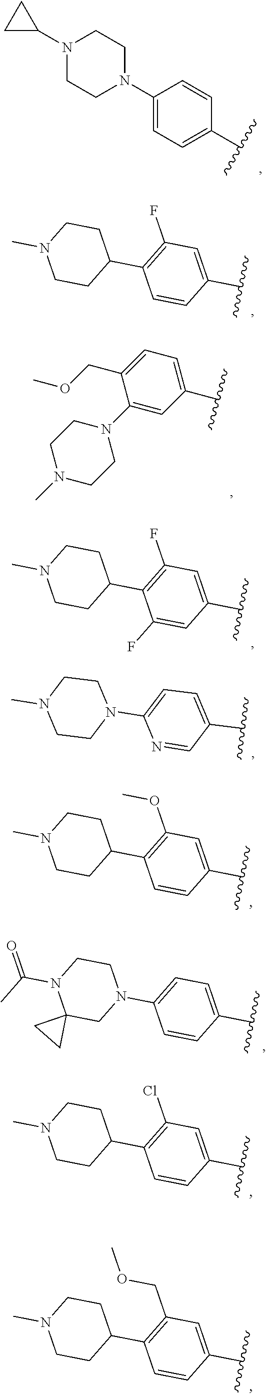

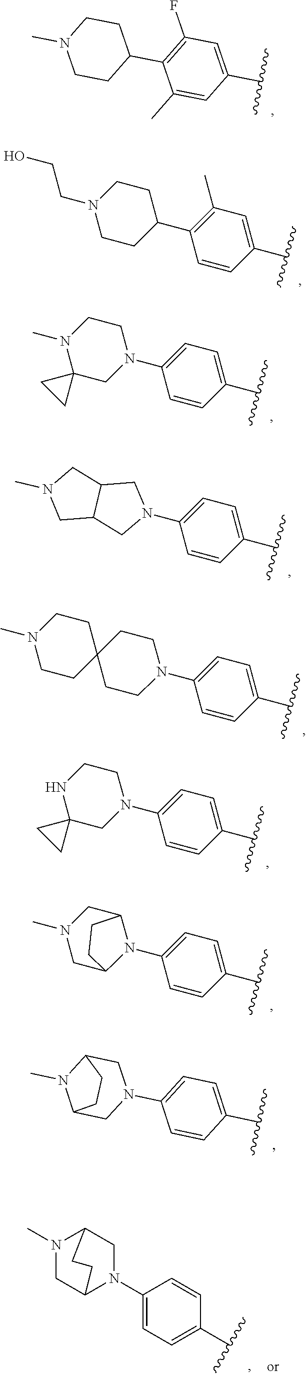

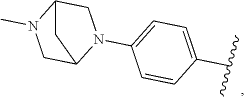 [SMILES: *c1ccc(N2CC3CC2CN3C)cc1]

Also disclosed herein is a compound of Formula (II), or a pharmaceutically acceptable salt, solvate, or stereoisomer thereof:

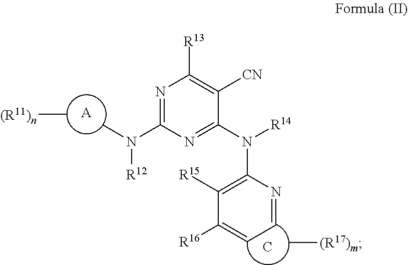

wherein:
Ring A is cycloalkyl, heterocycloalkyl, aryl, or heteroaryl;
each R¹¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{11a};
or two R¹¹ on the same atom form an oxo;
each R^{11a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{11b};
or two R^{11a} on the same atom form an oxo;
each R^{11b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R^{11b} on the same atom form an oxo;
n is 0, 1, 2, 3, 4, 5, or 6;
R¹² is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R¹³ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R¹⁴ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R¹⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R¹⁶ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring C is cycloalkyl or heterocycloalkyl;
each R¹⁷ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}—, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R¹⁷ on the same carbon form an oxo;
m is 0, 1, 2, 3, 4, 5, or 6;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom form an oxo;
provided that the compound is not

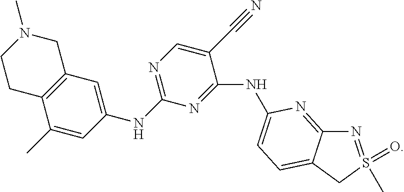 [SMILES: Cc1cc(Nc2ncc(C#N)c(Nc3ccc4c(n3)N=S(C)(=O)C4)n2)cc2c1CCN(C)C2]

In some embodiments of a compound of Formula (II). Ring C is cycloalkyl. In some embodiments of a compound of Formula (II), Ring C is 5- or 6-membered cycloalkyl. In some embodiments of a compound of Formula (II), Ring C is 5-membered cycloalkyl. In some embodiments of a compound of Formula (II), Ring C is heterocycloalkyl. In some embodiments of a compound of Formula (II), Ring C is heterocycloalkyl comprising 1 or 2 heteroatoms selected from the group consisting of N and O.

In some embodiments of a compound of Formula (II), each R¹⁷ is independently deuterium, halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl. In some embodiments of a compound of Formula (II), each R¹⁷ is independently halogen, —CN, —OH, —OR^{a}, C₁-C₆alkyl, or C₁-C₆haloalkyl. In some embodiments of a compound of Formula (II), each R¹⁷ is independently —OH, —OR^{a}, or C₁-C₆alkyl.

In some embodiments of a compound of Formula (II), m is 0, 1, 2, or 3. In some embodiments of a compound of Formula (II), m is 0, 1, or 2. In some embodiments of a compound of Formula (II), m is 1, 2, or 3. In some embodiments of a compound of Formula (II), m is 2 or 3. In some embodiments of a compound of Formula (II), m is 0. In some embodiments of a compound of Formula (II), m is 1. In some embodiments of a compound of Formula (II), m is 2. In some embodiments of a compound of Formula (II), m is 3. In some embodiments of a compound of Formula (II), m is 4.

In some embodiments of a compound of Formula (II), the compound is of Formula (IIa):

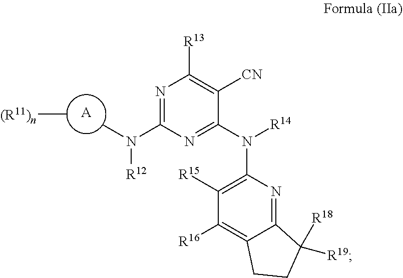

wherein:
R¹⁸ and R¹⁹ are independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R.

In some embodiments of a compound of Formula (IIa), R¹⁸ and R¹⁹ are independently hydrogen, deuterium, halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl. In some embodiments of a compound of Formula (IIa). R¹⁸ and R¹⁹ are independently hydrogen, halogen, —CN, —OH, —OR^{a}, C₁-C₆alkyl, or C₁-C₆haloalkyl. In some embodiments of a compound of Formula (IIa), R¹⁸ and R¹⁹ are independently hydrogen, —OH, —OR^{a}, or C₁-C₆alkyl. In some embodiments of a compound of Formula (IIa), R¹⁸ and R¹⁹ are independently —OH or C₁-C₆alkyl.

In some embodiments of a compound of Formula (II) or (IIa), R¹² is hydrogen, C₁-C₆alkyl, C₁-C₆haloalkyl, cycloalkyl, or heterocycloalkyl. In some embodiments of a compound of Formula (II) or (IIa), R¹² is hydrogen, C₁-C₆alkyl, or C₁-C₆haloalkyl. In some embodiments of a compound of Formula (II) or (IIa), R¹² is hydrogen or C₁-C₆alkyl. In some embodiments of a compound of Formula (II) or (IIa), R¹² is hydrogen.

In some embodiments of a compound of Formula (II) or (IIa), R¹³ is hydrogen, deuterium, halogen, —CN, —OH, —OR^{a}, —NR^{c}R^{d}, C₁-C₆alkyl, C₁-C₆haloalkyl, cycloalkyl, or heterocycloalkyl. In some embodiments of a compound of Formula (II) or (IIa), R¹³ is hydrogen, deuterium, halogen, C₁-C₆alkyl, C₁-C₆haloalkyl, cycloalkyl, or heterocycloalkyl. In some embodiments of a compound of Formula (II) or (IIa), R¹³ is hydrogen, deuterium, halogen, C₁-C₆alkyl, or C₁-C₆haloalkyl. In some embodiments of a compound of Formula (II) or (IIa), R¹³ is hydrogen, halogen, or C₁-C₆alkyl. In some embodiments of a compound of Formula (II) or (IIa), R¹³ is hydrogen or C₁-C₆alkyl. In some embodiments of a compound of Formula (II) or (IIa), R¹³ is hydrogen or halogen. In some embodiments of a compound of Formula (II) or (IIa), R¹³ is hydrogen.

In some embodiments of a compound of Formula (II) or (IIa), R¹⁴ is hydrogen, C₁-C₆alkyl, C₁-C₆haloalkyl, cycloalkyl, or heterocycloalkyl. In some embodiments of a compound of Formula (II) or (IIa), R¹⁴ is hydrogen, C₁-C₆alkyl, or C₁-C₆haloalkyl. In some embodiments of a compound of Formula (II) or (IIa). R¹⁴ is hydrogen or C₁-C₆alkyl. In some embodiments of a compound of Formula (II) or (IIa). R¹⁴ is hydrogen.

In some embodiments of a compound of Formula (II) or (IIa), R¹⁵ is hydrogen, deuterium, halogen, —CN, —OH, —OR^{a}, —NR^{c}R^{d}, C₁-C₆alkyl, C₁-C₆haloalkyl, cycloalkyl, or heterocycloalkyl. In some embodiments of a compound of Formula (II) or (IIa), R¹⁵ is hydrogen, deuterium, halogen, C₁-C₆alkyl, C₁-C₆haloalkyl, cycloalkyl, or heterocycloalkyl. In some embodiments of a compound of Formula (II) or (IIa), R¹⁵ is hydrogen, deuterium, halogen, C₁-C₆alkyl, or C₁-C₆haloalkyl. In some embodiments of a compound of Formula (II) or (IIa). R¹⁵ is hydrogen, halogen, or C₁-C₆alkyl. In some embodiments of a compound of Formula (II) or (IIa), R¹⁵ is hydrogen or C₁-C₆alkyl. In some embodiments of a compound of Formula (II) or (IIa), R¹⁵ is hydrogen or halogen. In some embodiments of a compound of Formula (II) or (IIa), R¹⁵ is hydrogen.

In some embodiments of a compound of Formula (II) or (IIa), R¹⁶ is hydrogen, deuterium, halogen, —CN, —OH, —OR^{a}, —NR^{c}R^{d}, C₁-C₆alkyl, C₁-C₆haloalkyl, cycloalkyl, or heterocycloalkyl. In some embodiments of a compound of Formula (II) or (IIa), R¹⁶ is hydrogen, deuterium, halogen, C₁-C₆alkyl, C₁-C₆haloalkyl, cycloalkyl, or heterocycloalkyl. In some embodiments of a compound of Formula (II) or (IIa), R¹⁶ is hydrogen, deuterium, halogen, C₁-C₆alkyl, or C₁-C₆haloalkyl. In some embodiments of a compound of Formula (II) or (IIa), R¹⁶ is hydrogen, halogen, or C₁-C₆alkyl. In some embodiments of a compound of Formula (II) or (IIa), R¹⁶ is hydrogen or C₁-C₆alkyl. In some embodiments of a compound of Formula (II) or (IIa), R¹⁶ is hydrogen or halogen. In some embodiments of a compound of Formula (II) or (II), R¹⁶ is hydrogen.

In some embodiments of a compound of Formula (II) or (IIa), Ring A is a monocyclic or a bicyclic cycloalkyl, a monocyclic or a bicyclic heterocycloalkyl, a monocyclic or a bicyclic aryl, or a monocyclic or a bicyclic heteroaryl. In some embodiments of a compound of Formula (II) or (IIa), Ring A is a monocyclic or a bicyclic aryl or a monocyclic or a bicyclic heteroaryl. In some embodiments of a compound of Formula (II) or (IIa), Ring A is a monocyclic aryl or a monocyclic heteroaryl.

In some embodiments of a compound of Formula (II) or (IIa), Ring A is aryl or heteroaryl. In some embodiments of a compound of Formula (II) or (IIa), Ring A is aryl. In some embodiments of a compound of Formula (II) or (IIa), Ring A is heteroaryl. In some embodiments of a compound of Formula (II) or (IIa), Ring A is a monocyclic aryl or a monocyclic heteroaryl. In some embodiments of a compound of Formula (II) or (IIa), Ring A is a phenyl. In some embodiments of a compound of Formula (II) or (IIa), Ring A is a bicyclic heteroaryl. In some embodiments of a compound of Formula (II) or (IIa), Ring A is 1,2,3,4-tetrahydroisoquinolinyl.

In some embodiments of a compound of Formula (II) or (IIa), each R¹¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{11a}. In some embodiments of a compound of Formula (II) or (IIa), each R¹¹ is independently deuterium, halogen, —OH, —OR^{a}, —NR^{c}R^{d}, C₁-C₆alkyl, C₁-C₆haloalkyl, C₁-C₆heteroalkyl, cycloalkyl, heterocycloalkyl; wherein the alkyl, cycloalkyl, and heterocycloalkyl is optionally substituted with one or more R^{11a}. In some embodiments of a compound of Formula (II) or (IIa), each R¹¹ is independently deuterium, halogen, C₁-C₆alkyl, C₁-C₆haloalkyl, C₁-C₆heteroalkyl, cycloalkyl, or heterocycloalkyl; wherein the alkyl, cycloalkyl, and heterocycloalkyl is optionally substituted with one or more R^{11a}. In some embodiments of a compound of Formula (II) or (IIa), each R¹¹ is independently halogen, C₁-C₆alkyl, C₁-C₆heteroalkyl, cycloalkyl, or heterocycloalkyl; wherein the alkyl, cycloalkyl, and heterocycloalkyl is optionally substituted with one or more R^{11a}. In some embodiments of a compound of Formula (II) or (IIa), each R¹¹ is independently halogen, —OR^{a}, C₁-C₆alkyl, C₁-C₆heteroalkyl, or heterocycloalkyl; wherein the alkyl, and heterocycloalkyl is optionally substituted with one or more R^{11a}. In some embodiments of a compound of Formula (II) or (IIa), each R¹¹ is independently halogen, C₁-C₆alkyl, C₁-C₆heteroalkyl, or heterocycloalkyl; wherein the alkyl, and heterocycloalkyl is optionally substituted with one or more R^{11a}. In some embodiments of a compound of Formula (II) or (IIa), each R¹¹ is independently heterocycloalkyl optionally substituted with one or more R^{11a}. In some embodiments of a compound of Formula (II) or (IIa), each R¹¹ is independently heterocycloalkyl optionally substituted with one or more R^{11a}; wherein the heterocycloalkyl is monocyclic or bicyclic. In some embodiments of a compound of Formula (II) or (IIa), each R¹¹ is independently heterocycloalkyl optionally substituted with one or more R^{11a}; wherein the heterocycloalkyl is a bridged heterocycloalkyl, fused heterocycloalkyl, or spirocyclic heterocycloalkyl. In some embodiments of a compound of Formula (II) or (IIa), each R¹¹ is independently heterocycloalkyl optionally substituted with one or more R^{11a}; wherein the heterocycloalkyl is 2,5-diazabicyclo[2.2.1]heptanyl, 3,8-diazabicyclo[3.2.1]octanyl, 3,8-diazabicyclo[3.2.1]octanyl, or 2,5-diazabicyclo[2.2.2]octanyl. In some embodiments of a compound of Formula (II) or (IIa), each R¹¹ is independently heterocycloalkyl optionally substituted with one or more R^{11a}; wherein the heterocycloalkyl is octahydropyrrolo[3,4-c]pyrrolyl. In some embodiments of a compound of Formula (II) or (IIa), each R¹¹ is independently heterocycloalkyl optionally substituted with one or more R^{1a}; wherein the heterocycloalkyl is 4,7-diazaspiro[2.5]octanyl or 3,9-diazaspiro[5.5]undecanyl. In some embodiments of a compound of Formula (II) or (IIa), each R¹¹ is independently halogen or C₁-C₆alkyl.

In some embodiments of a compound of Formula (II) or (IIa), each R^{11a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, is heterocycloalkyl is optionally substituted with one or more R^{11b}; or two R^{1a} on the same atom form an oxo. In some embodiments of a compound of Formula (II) or (IIa), each R^{11a} is independently deuterium, halogen, C₁-C₆alkyl, C₁-C₆haloalkyl, or C₁-C₆heteroalkyl; wherein the alkyl is optionally substituted with one or more R^{11b}; or two R^{11a} on the same atom form an oxo. In some embodiments of a compound of Formula (II) or (IIa), each R^{11a} is independently deuterium, halogen, C₁-C₆alkyl, C₁-C₆haloalkyl, or C₁-C₆heteroalkyl; wherein the alkyl is optionally substituted with one or more R^{11b}; or two R^{11a} on the same atom form an oxo. In some embodiments of a compound of Formula (II) or (IIa), each R^{11a} is independently C₁-C₆alkyl, C₁-C₆haloalkyl, or C₁-C₆heteroalkyl; or two R^{11a} on the same atom form an oxo. In some embodiments of a compound of Formula (II) or (IIa), each R^{11a} is independently C₁-C₆alkyl, C₁-C₆haloalkyl, or C₁-C₆heteroalkyl. In some embodiments of a compound of Formula (II) or (IIa), each R^{11a} is independently C₁-C₆alkyl.

In some embodiments of a compound of Formula (II) or (IIa), each R^{11b} is independently deuterium, halogen, —CN, —OH, —OR^{a}, —NR^{c}R^{d}, C₁-C₆alkyl, or C₁-C₆haloalkyl. In some embodiments of a compound of Formula (II) or (IIa), each R^{11b} is independently deuterium, halogen, —OH, C₁-C₆alkyl, or C₁-C₆haloalkyl. In some embodiments of a compound of Formula (II) or (IIa), each R^{11b} is independently C₁-C₆alkyl.

In some embodiments of a compound of Formula (II) or (IIa), n is 0, 1, 2, 3, or 4. In some embodiments of a compound of Formula (II) or (IIa), n is 1, 2, or 3. In some embodiments of a compound of Formula (II) or (IIa), n is 2 or 3. In some embodiments of a compound of Formula (II) or (IIa), n is 1 or 2. In some embodiments of a compound of Formula (II) or (IIa), n is 0. In some embodiments of a compound of Formula (II) or (IIa), n is 1. In some embodiments of a compound of Formula (II) or (IIa), n is 2. In some embodiments of a compound of Formula (II) or (IIa), n is 3. In some embodiments of a compound of Formula (II) or (IIa), n is 4.

In some embodiments of a compound of Formula (II) or (IIa),

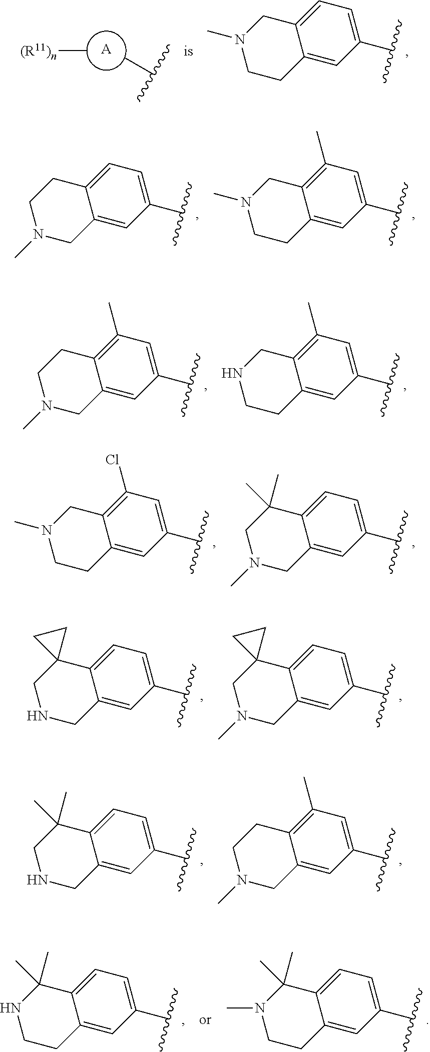

In some embodiments of a compound of Formula (II) or (IIa)

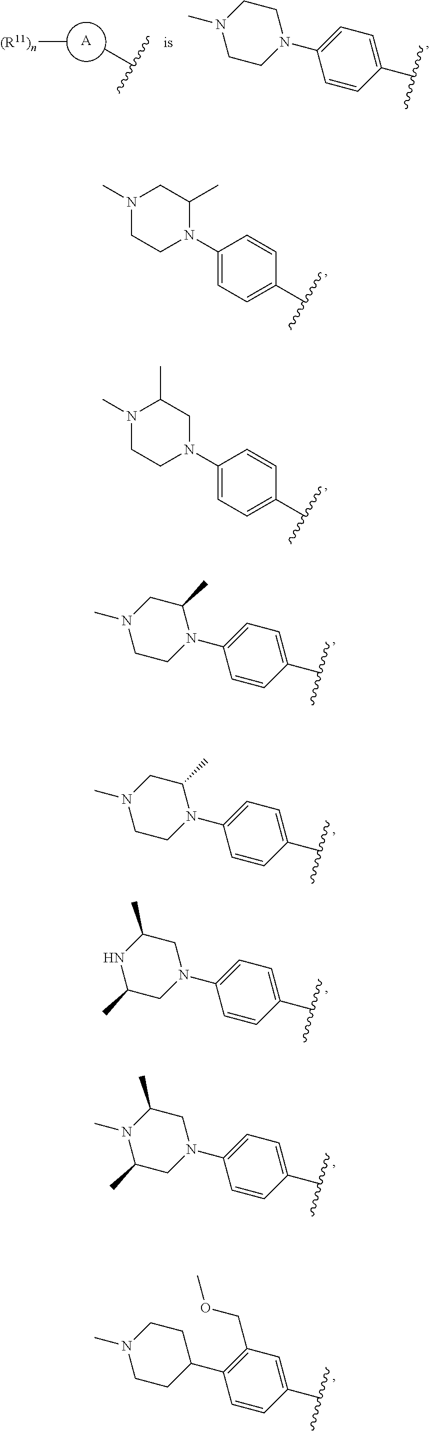

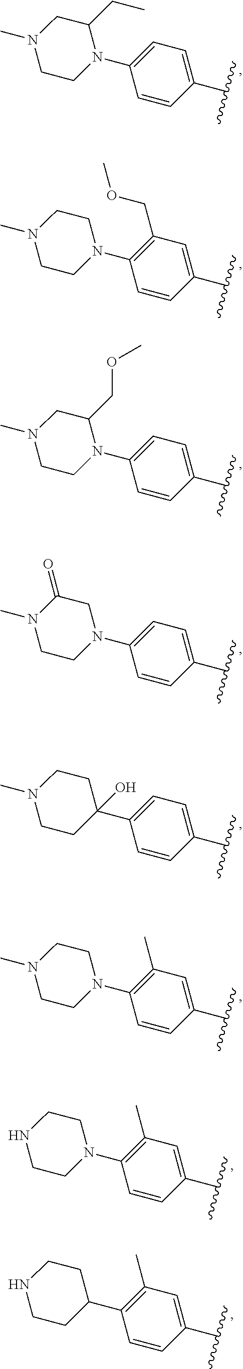

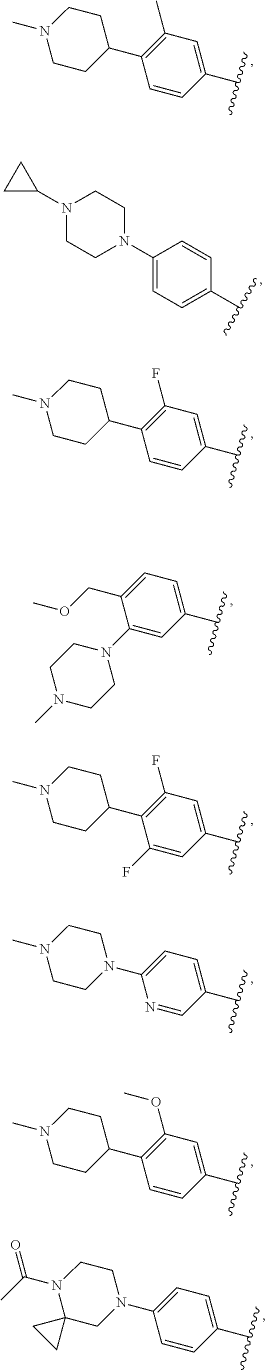

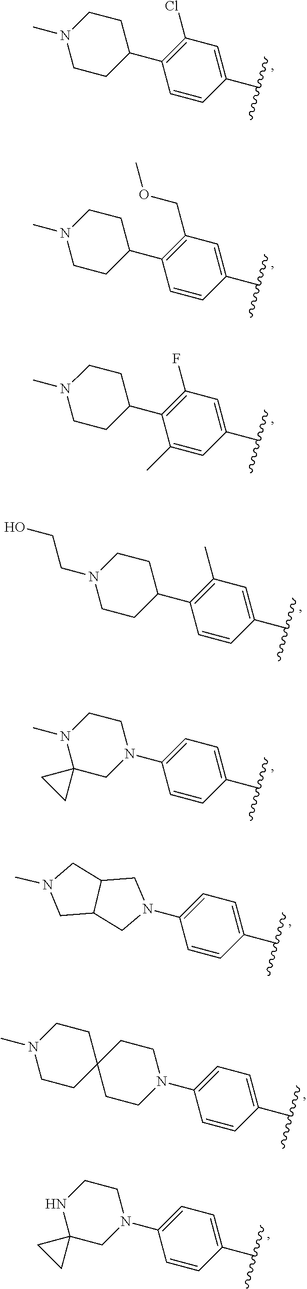

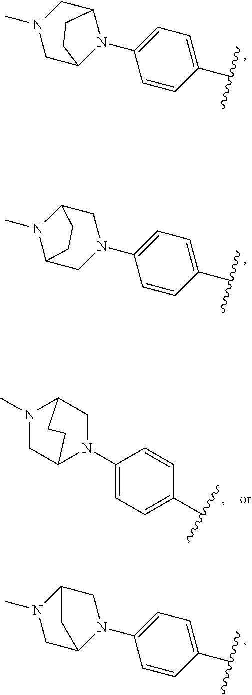 [SMILES: *c1ccc(N2C3CCC2CN(C)C3)cc1 ; *c1ccc(N2CC3CCC(C2)N3C)cc1 ; *c1ccc(N2CC3CCC2CN3C)cc1 ; *c1ccc(N2CC3CC2CN3C)cc1]

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl; wherein each alkyl, cycloalkyl, and heterocycloalkyl is independently optionally substituted with one or more R. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl; or two R on the same atom form an oxo. In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl; or two R on the same atom form an oxo. In some embodiments of a compound disclosed herein, each R is independently halogen, —CN, —OH, —NH₂, C₁-C₃alkyl, C₁-C₃haloalkyl, C₃-C₆cycloalkyl, or heterocycloalkyl; or two R on the same atom form an oxo. In some embodiments of a compound disclosed herein, each R is independently halogen, —CN, —OH, —NH₂, C₁-C₃alkyl, or C₁-C₃haloalkyl; or two R on the same atom form an oxo. In some embodiments of a compound disclosed herein, each R is independently halogen, C₁-C₃alkyl, or C₁-C₃haloalkyl; or two R on the same atom form an oxo. In some embodiments of a compound disclosed herein, each R is independently halogen or C₁-C₃alkyl; or two R on the same atom form an oxo.

In some embodiments of a compound disclosed herein, the compound is selected from a compound of Table 1:

TABLE 1

| Ex. | Structure | Name |
|---|---|---|
| 1 | 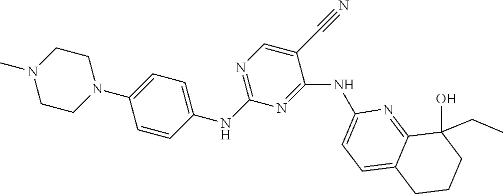 [SMILES: CCC1(O)CCCc2ccc(Nc3nc(Nc4ccc(N5CCN(C)CC5)cc4)ncc3C#N)nc21] | 4-[(8-ethyl-8-hydroxy-6,7-dihydro-5H- quinolin-2-yl)amino]-2-[4-(4- methylpiperazin-1-yl)anilino]pyrimidine- 5-carbonitrile |
| 2 | 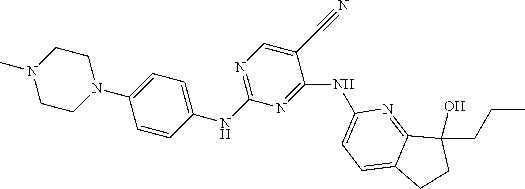 [SMILES: CCCC1(O)CCc2ccc(Nc3nc(Nc4ccc(N5CCN(C)CC5)cc4)ncc3C#N)nc21] | 4-[(7-hydroxy-7-propyl-5,6- dihydrocyclopenta[b]pyridin-2-yl)amino]- 2-[4-(4-methylpiperazin-1- yl)anilino]pyrimidine-5-carbonitrile |
| 3 | 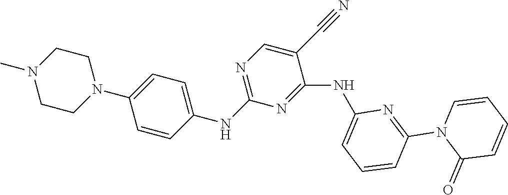 [SMILES: CN1CCN(c2ccc(Nc3ncc(C#N)c(Nc4cccc(-n5ccccc5=O)n4)n3)cc2)CC1] | 2-[4-(4-methylpiperazin-1-yl)anilino]-4- [[6-(2-oxo-1-pyridyl)-2- pyridyl]amino]pyrimidine-5-carbonitrile |
| 4 | 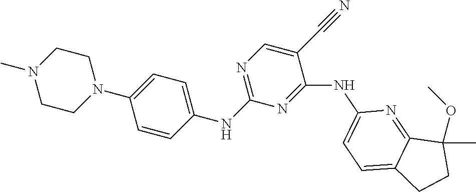 | 4-[(7-methoxy-7-methyl-5,6- dihydrocyclopenta[b]pyridin-2-yl)amino]- 2-[4-(4-methylpiperazin-1- yl)anilino]pyrimidine-5-carbonitrile |
| 5 | 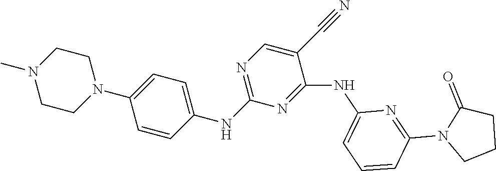 [SMILES: CN1CCN(c2ccc(Nc3ncc(C#N)c(Nc4cccc(N5CCCC5=O)n4)n3)cc2)CC1] | 2-[4-(4-methylpiperazin-1-yl)anilino]-4- [[6-(2-oxopyrrolidin-1-yl)-2- pyridyl]amino]pyrimidine-5-carbonitrile |
| 6 | 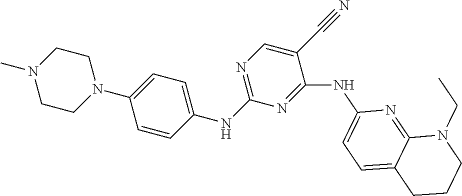 [SMILES: CCN1CCCc2ccc(Nc3nc(Nc4ccc(N5CCN(C)CC5)cc4)ncc3C#N)nc21] | 4-[(8-ethyl-6,7-dihydro-5H-1,8- naphthyridin-2-yl)amino]-2-[4-(4- methylpiperazin-1-yl)anilino]pyrimidine- 5-carbonitrile |
| 7 | 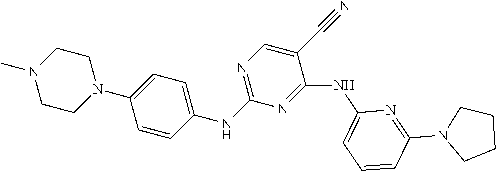 [SMILES: CN1CCN(c2ccc(Nc3ncc(C#N)c(Nc4cccc(N5CCCC5)n4)n3)cc2)CC1] | 2-[4-(4-methylpiperazin-1-yl)anilino]-4- [(6-pyrrolidin-1-yl-2- pyridyl)amino]pyrimidine-5-carbonitrile |
| 8 | 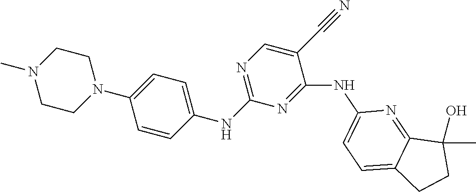 | 4-[(7-hydroxy-7-methyl-5,6- dihydrocyclopenta[b]pyridin-2-yl)amino]- 2-[4-(4-methylpiperazin-1- yl)anilino]pyrimidine-5-carbonitrile |
| 9 | 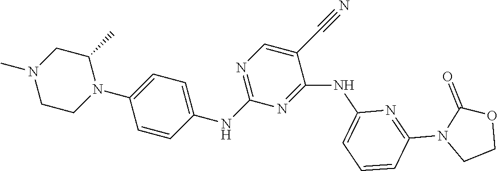 [SMILES: C[C@H]1CN(C)CCN1c1ccc(Nc2ncc(C#N)c(Nc3cccc(N4CCOC4=O)n3)n2)cc1] | 4-[[6-(2-oxooxazolidin-3-yl)-2- pyridyl]amino]-2-[4-[(2S)-2,4- dimethylpiperazin-1- yl]anilino]pyrimidine-5-carbonitrile |
| 10 | 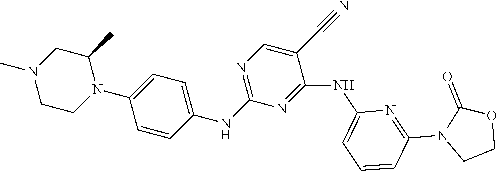 [SMILES: C[C@@H]1CN(C)CCN1c1ccc(Nc2ncc(C#N)c(Nc3cccc(N4CCOC4=O)n3)n2)cc1] | 4-[[6-(2-oxooxazolidin-3-yl)-2- pyridyl]amino]-2-[4-[rac-(2R)-2,4- dimethylpiperazin-1- yl]anilino]pyrimidine-5-carbonitrile |
| 11 | 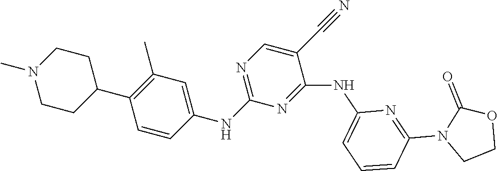 [SMILES: Cc1cc(Nc2ncc(C#N)c(Nc3cccc(N4CCOC4=O)n3)n2)ccc1C1CCN(C)CC1] | 2-[3-methyl-4-(1-methyl-4- piperidyl)anilino]-4-[[6-(2-oxooxazolidin- 3-yl)-2-pyridyl]amino]pyrimidine-5- carbonitrile |
| 12 | 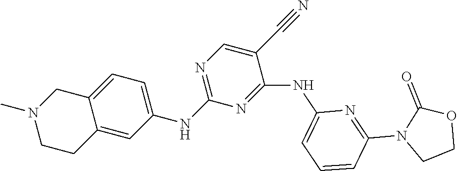 [SMILES: CN1CCc2cc(Nc3ncc(C#N)c(Nc4cccc(N5CCOC5=O)n4)n3)ccc2C1] | 2-[(2-methyl-3,4-dihydro-1H-isoquinolin- 6-yl)amino]-4-[[6-(2-oxooxazolidin-3-yl)- 2-pyridyl]amino]pyrimidine-5-carbonitrile |
| 13 | 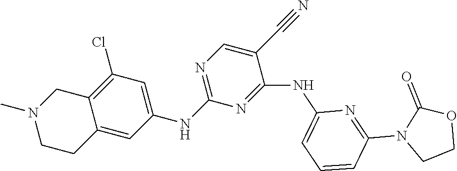 [SMILES: CN1CCc2cc(Nc3ncc(C#N)c(Nc4cccc(N5CCOC5=O)n4)n3)cc(Cl)c2C1] | 2-[(8-chloro-2-methyl-3,4-dihydro-1H- isoquinolin-6-yl)amino]-4-[[6-(2- oxooxazolidin-3-yl)-2- pyridyl]amino]pyrimidine-5-carbonitrile |
| 14 | 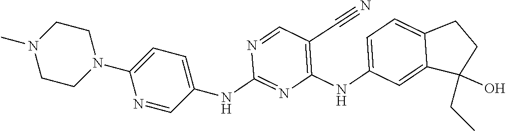 [SMILES: CCC1(O)CCc2ccc(Nc3nc(Nc4ccc(N5CCN(C)CC5)nc4)ncc3C#N)cc21] | 4-[(7-ethyl-7-hydroxy-5,6- dihydrocyclopenta[b]pyridin-2-yl)amino]- 2-[[6-(4-methylpiperazin-1-yl)-3- pyridyl]amino]pyrimidine-5-carbonitrile |
| 15 | 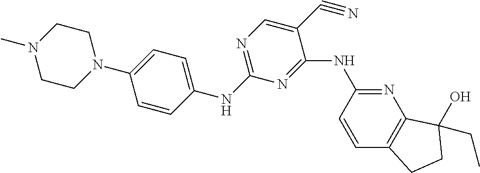 [SMILES: CCC1(O)CCc2ccc(Nc3nc(Nc4ccc(N5CCN(C)CC5)cc4)ncc3C#N)nc21] | 4-[(7-ethyl-7-hydroxy-5,6- dihydrocyclopenta[b]pyridin-2-yl)amino]- 2-[4-(4-methylpiperazin-1- yl)anilino]pyrimidine-5-carbonitrile |
| 16 | 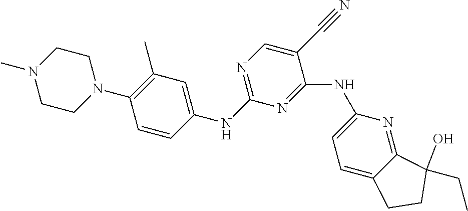 [SMILES: CCC1(O)CCc2ccc(Nc3nc(Nc4ccc(N5CCN(C)CC5)c(C)c4)ncc3C#N)nc21] | 4-[(7-ethyl-7-hydroxy-5,6- dihydrocyclopenta[b]pyridin-2-yl)amino]- 2-[3-methyl-4-(4-methylpiperazin-1- yl)anilino]pyrimidine-5-carbonitrile |
| 17 | 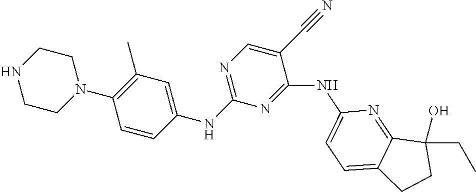 [SMILES: CCC1(O)CCc2ccc(Nc3nc(Nc4ccc(N5CCNCC5)c(C)c4)ncc3C#N)nc21] | 4-[(7-ethyl-7-hydroxy-5,6- dihydrocyclopenta[b]pyridin-2-yl)amino]- 2-(3-methyl-4-piperazin-1-yl- anilino)pyrimidine-5-carbonitrile |
| 18 | 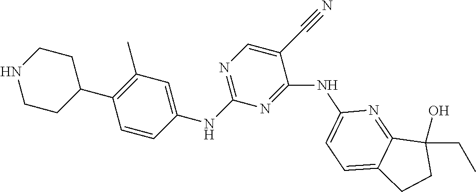 [SMILES: CCC1(O)CCc2ccc(Nc3nc(Nc4ccc(C5CCNCC5)c(C)c4)ncc3C#N)nc21] | 4-[(7-ethyl-7-hydroxy-5,6- dihydrocyclopenta[b]pyridin-2-yl)amino]- 2-[3-methyl-4-(4- piperidyl)anilino]pyrimidine-5-carbonitrile |
| 19 | 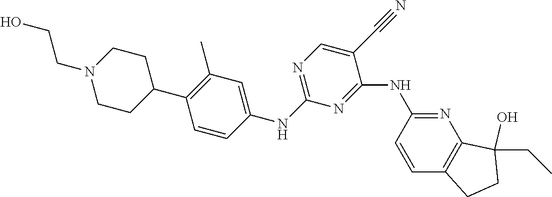 [SMILES: CCC1(O)CCc2ccc(Nc3nc(Nc4ccc(C5CCN(CCO)CC5)c(C)c4)ncc3C#N)nc21] | 4-[(7-ethyl-7-hydroxy-5,6- dihydrocyclopenta[b]pyridin-2-yl)amino]- 2-[4-[1-(2-hydroxyethyl)-4-piperidyl]-3- methyl-anilino]pyrimidine-5-carbonitrile |
| 20 | 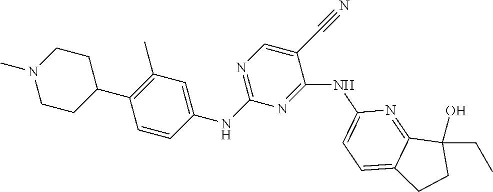 [SMILES: CCC1(O)CCc2ccc(Nc3nc(Nc4ccc(C5CCN(C)CC5)c(C)c4)ncc3C#N)nc21] | 4-[(7-ethyl-7-hydroxy-5,6- dihydrocyclopenta[b]pyridin-2-yl)amino]- 2-[3-methyl-4-(1-methyl-4- piperidyl)anilino]pyrimidine-5-carbonitrile |
| 21 | 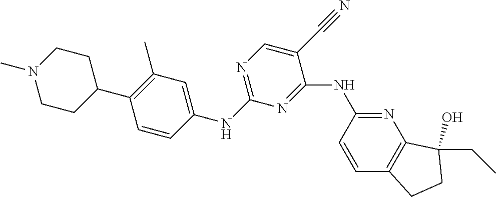 [SMILES: CC[C@@]1(O)CCc2ccc(Nc3nc(Nc4ccc(C5CCN(C)CC5)c(C)c4)ncc3C#N)nc21] | 2-[3-methyl-4-(1-methyl-4- piperidyl)anilino]-4-[[(7R)-7-ethyl-7- hydroxy-5,6-dihydrocyclopenta[b]pyridin- 2-yl]amino]pyrimidine-5-carbonitrile* |
| 22 | 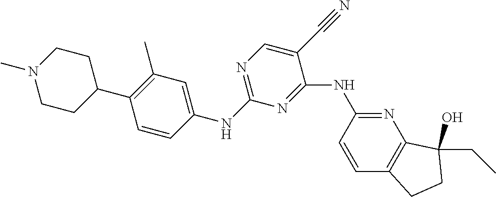 [SMILES: CC[C@]1(O)CCc2ccc(Nc3nc(Nc4ccc(C5CCN(C)CC5)c(C)c4)ncc3C#N)nc21] | 2-[3-methyl-4-(1-methyl-4- piperidyl)anilino]-4-[[(7S)-7-ethyl-7- hydroxy-5,6-dihydrocyclopenta[b]pyridin- 2-yl]amino]pyrimidine-5-carbonitrile* |
| 23 | 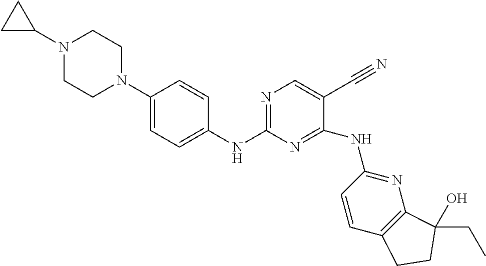 [SMILES: CCC1(O)CCc2ccc(Nc3nc(Nc4ccc(N5CCN(C6CC6)CC5)cc4)ncc3C#N)nc21] | 2-[4-(4-cyclopropylpiperazin-1- yl)anilino]-4-[(7-ethyl-7-hydroxy-5,6- dihydrocyclopenta[b]pyridin-2- yl)amino]pyrimidine-5-carbonitrile |
| 24 | 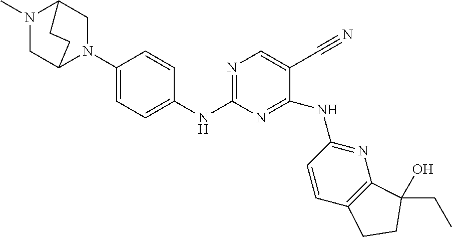 [SMILES: CCC1(O)CCc2ccc(Nc3nc(Nc4ccc(N5CC6CCC5CN6C)cc4)ncc3C#N)nc21] | 4-[(7-ethyl-7-hydroxy-5,6- dihydrocyclopenta[b]pyridin-2-yl)amino]- 2-[4-(5-methyl-2,5- diazabicyclo[2.2.2]octan-2- yl)anilino]pyrimidine-5-carbonitrile |
| 25 | 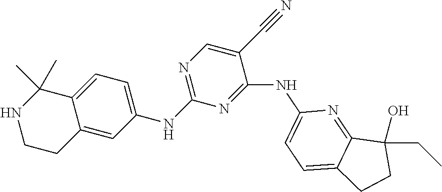 [SMILES: CCC1(O)CCc2ccc(Nc3nc(Nc4ccc5c(c4)CCNC5(C)C)ncc3C#N)nc21] | 2-[(1,1-dimethyl-3,4-dihydro-2H- isoquinolin-6-yl)amino]-4-[(7-ethyl-7- hydroxy-5,6-dihydrocyclopenta[b]pyridin- 2-yl)amino]pyrimidine-5-carbonitrile |
| 26 | 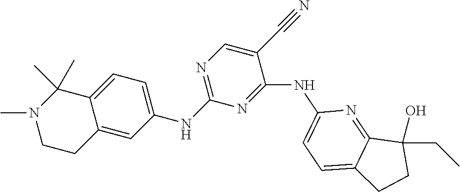 [SMILES: CCC1(O)CCc2ccc(Nc3nc(Nc4ccc5c(c4)CCN(C)C5(C)C)ncc3C#N)nc21] | 4-[(7-ethyl-7-hydroxy-5,6- dihydrocyclopenta[b]pyridin-2-yl)amino]- 2-[(1,1,2-trimethyl-3,4- dihydroisoquinolin-6- yl)amino]pyrimidine-5-carbonitrile |
| 27 | 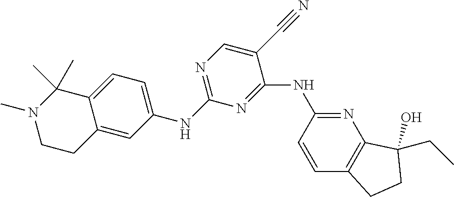 [SMILES: CC[C@@]1(O)CCc2ccc(Nc3nc(Nc4ccc5c(c4)CCN(C)C5(C)C)ncc3C#N)nc21] | 4-[[rac-(7R)-7-ethyl-7-hydroxy-5,6- dihydrocyclopenta[b]pyridin-2-yl]amino]- 2-[(1,1,2-trimethyl-3,4- dihydroisoquinolin-6- yl)amino]pyrimidine-5-carbonitrile* |
| 28 | 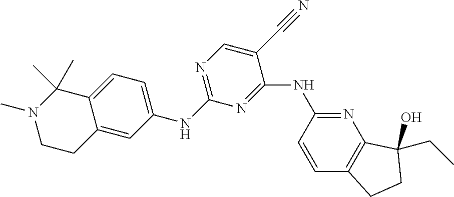 [SMILES: CC[C@]1(O)CCc2ccc(Nc3nc(Nc4ccc5c(c4)CCN(C)C5(C)C)ncc3C#N)nc21] | 4-[[rac-(7S)-7-ethyl-7-hydroxy-5,6- dihydrocyclopenta[b]pyridin-2-yl]amino]- 2-[(1,1,2-trimethyl-3,4- dihydroisoquinolin-6- yl]amino]pyrimidine-5-carbonitrile* |
| 29 | 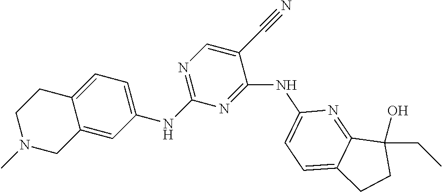 [SMILES: CCC1(O)CCc2ccc(Nc3nc(Nc4ccc5c(c4)CN(C)CC5)ncc3C#N)nc21] | 4-[(7-ethyl-7-hydroxy-5,6- dihydrocyclopenta[b]pyridin-2-yl)amino]- 2-[(2-methyl-3,4-dihydro-1H-isoquinolin- 6-yl)amino]pyrimidine-5-carbonitrile |
| 30 | 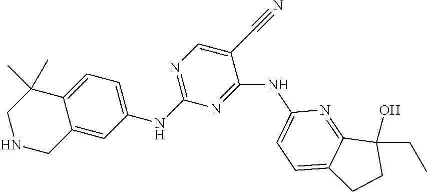 [SMILES: CCC1(O)CCc2ccc(Nc3nc(Nc4ccc5c(c4)CNCC5(C)C)ncc3C#N)nc21] | 2-[(4,4-dimethyl-2,3-dihydro-1H- isoquinolin-7-yl)amino]-4-[(7-ethyl-7- hydroxy-5,6-dihydrocyclopenta[b]pyridin- 2-yl)amino]pyrimidine-5-carbonitrile |
| 31 | 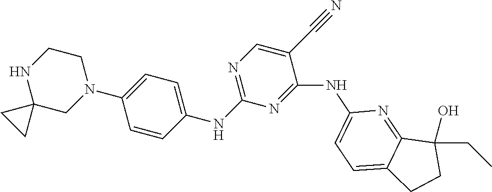 [SMILES: CCC1(O)CCc2ccc(Nc3nc(Nc4ccc(N5CCNC6(CC6)C5)cc4)ncc3C#N)nc21] | 2-[4-(4,7-diazaspiro[2.5]octan-7- yl)anilino]-4-[(7-ethyl-7-hydroxy-5,6- dihydrocyclopenta[b]pyridin-2- yl)amino]pyrimidine-5-carbonitrile |
| 32 | 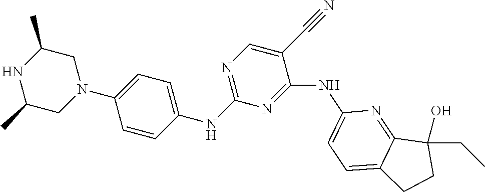 [SMILES: CCC1(O)CCc2ccc(Nc3nc(Nc4ccc(N5C[C@@H](C)N[C@@H](C)C5)cc4)ncc3C#N)nc21] | 4-[(7-ethyl-7-hydroxy-5,6- dihydrocyclopenta[b]pyridin-2-yl)amino]- 2-[4-[rac-(3R,5S)-3,5-dimethylpiperazin- 1-yl]anilino]pyrimidine-5-carbonitrile |
| 33 | 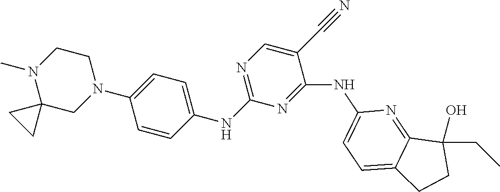 [SMILES: CCC1(O)CCc2ccc(Nc3nc(Nc4ccc(N5CCN(C)C6(CC6)C5)cc4)ncc3C#N)nc21] | 4-[(7-ethyl-7-hydroxy-5,6- dihydrocyclopenta[b]pyridin-2-yl)amino]- 2-[4-(4-methyl-4,7-diazaspiro[2.5]octan-7- yl)anilino]pyrimidine-5-carbonitrile |
| 34 | 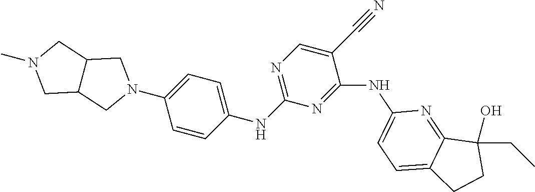 [SMILES: CCC1(O)CCc2ccc(Nc3nc(Nc4ccc(N5CC6CN(C)CC6C5)cc4)ncc3C#N)nc21] | 4-[(7-ethyl-7-hydroxy-5,6- dihydrocyclopenta[b]pyridin-2-yl)amino]- 2-[4-(2-methyl-1,3,3a,4,6,6a- hexahydropyrrolo[3,4-c]pyrrol-5- yl)anilino]pyrimidine-5-carbonitrile |
| 35 | 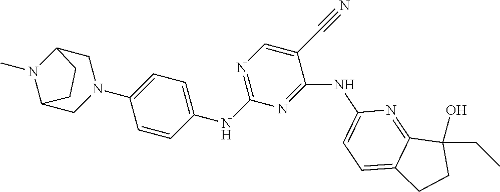 [SMILES: CCC1(O)CCc2ccc(Nc3nc(Nc4ccc(N5CC6CCC(C5)N6C)cc4)ncc3C#N)nc21] | 4-[(7-ethyl-7-hydroxy-5,6- dihydrocyclopenta[b]pyridin-2- yl)amino]-2-[4-(8-methyl-3,8- diazabicyclo[3.2.1]octan-3- yl)anilino]pyrimidine-5-carbonitrile |
| 36 | 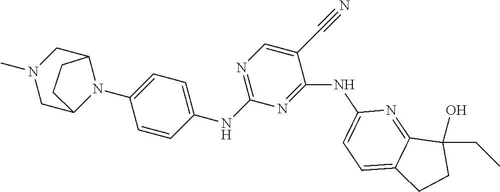 [SMILES: CCC1(O)CCc2ccc(Nc3nc(Nc4ccc(N5C6CCC5CN(C)C6)cc4)ncc3C#N)nc21] | 4-[(7-ethyl-7-hydroxy-5,6- dihydrocyclopenta[b]pyridin-2-yl)amino]- 2-[4-(3-methyl-3,8- diazabicyclo[3.2.1]octan-8- yl)anilino]pyrimidine-5-carbonitrile |
| 37 | 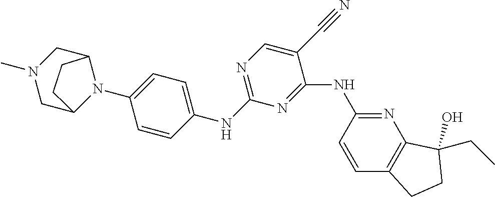 [SMILES: CC[C@@]1(O)CCc2ccc(Nc3nc(Nc4ccc(N5C6CCC5CN(C)C6)cc4)ncc3C#N)nc21] | 2-[4-(3-methyl-3,8- diazabicyclo[3.2.1]octan-8-yl)anilino]- 4-[[rac-(7R)-7-ethyl-7-hydroxy-5,6- dihydrocyclopenta[b]pyridin-2- yl]amino]pyrimidine-5-carbonitrile* |
| 38 | 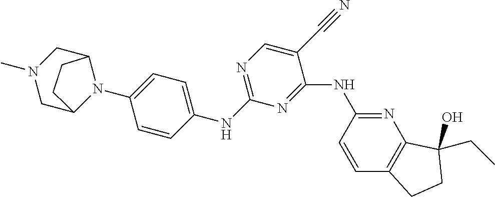 [SMILES: CC[C@]1(O)CCc2ccc(Nc3nc(Nc4ccc(N5C6CCC5CN(C)C6)cc4)ncc3C#N)nc21] | 2-[4-(3-methyl-3,8- diazabicyclo[3.2.1]octan-8-yl)anilino]- 4-[[rac-(7S)-7-ethyl-7-hydroxy-5,6- dihydrocyclopenta[b]pyridin-2- yl]amino]pyrimidine-5-carbonitrile* |
| 39 | 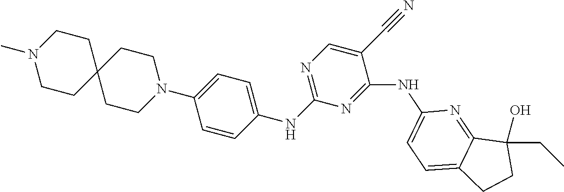 [SMILES: CCC1(O)CCc2ccc(Nc3nc(Nc4ccc(N5CCC6(CCN(C)CC6)CC5)cc4)ncc3C#N)nc21] | 4-[(7-ethyl-7-hydroxy-5,6- dihydrocyclopenta[b]pyridin-2-yl)amino]- 2-[4-(9-methyl-3,9- diazaspiro[5.5]undecan-3- yl)anilino]pyrimidine-5-carbonitrile |
| 40 | 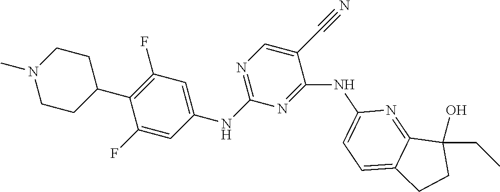 [SMILES: CCC1(O)CCc2ccc(Nc3nc(Nc4cc(F)c(C5CCN(C)CC5)c(F)c4)ncc3C#N)nc21] | 2-[3,5-difluoro-4-(1-methyl-4- piperidyl)anilino]-4-[(7-ethyl-7-hydroxy- 5,6-dihydrocyclopenta[b]pyridin-2- yl)amino]pyrimidine-5-carbonitrile |
| 41 | 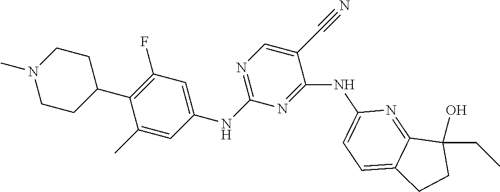 [SMILES: CCC1(O)CCc2ccc(Nc3nc(Nc4cc(C)c(C5CCN(C)CC5)c(F)c4)ncc3C#N)nc21] | 4-[(7-ethyl-7-hydroxy-5,6- dihydrocyclopenta[b]pyridin-2-yl)amino]- 2-[3-fluoro-5-methyl-4-(1-methyl-4- piperidyl)anilino]pyrimidine-5-carbonitrile |
| 42 | 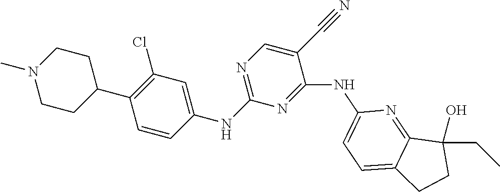 [SMILES: CCC1(O)CCc2ccc(Nc3nc(Nc4ccc(C5CCN(C)CC5)c(Cl)c4)ncc3C#N)nc21] | 2-[3-chloro-4-(1-methyl-4- piperidyl)anilino]-4-[(7-ethyl-7-hydroxy- 5,6-dihydrocyclopenta[b]pyridin-2- yl)amino]pyrimidine-5-carbonitrile |
| 43 | 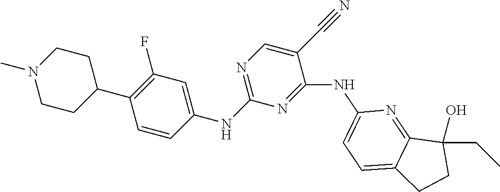 [SMILES: CCC1(O)CCc2ccc(Nc3nc(Nc4ccc(C5CCN(C)CC5)c(F)c4)ncc3C#N)nc21] | 4-[(7-ethyl-7-hydroxy-5,6- dihydrocyclopenta[b]pyridin-2-yl)amino]- 2-[3-fluoro-4-(1-methyl-4- piperidyl)anilino]pyrimidine-5-carbonitrile |
| 44 | 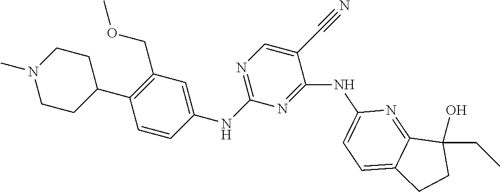 [SMILES: CCC1(O)CCc2ccc(Nc3nc(Nc4ccc(C5CCN(C)CC5)c(COC)c4)ncc3C#N)nc21] | 4-[(7-ethyl-7-hydroxy-5,6- dihydrocyclopenta[b]pyridin-2-yl)amino]- 2-[3-(methoxymethyl)-4-(1-methyl-4- piperidyl)anilino]pyrimidine-5-carbonitrile |
| 45 | 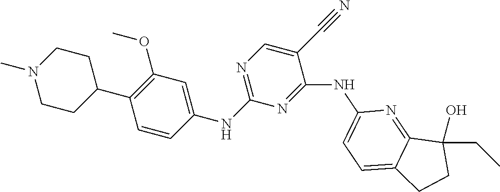 [SMILES: CCC1(O)CCc2ccc(Nc3nc(Nc4ccc(C5CCN(C)CC5)c(OC)c4)ncc3C#N)nc21] | 4-[(7-ethyl-7-hydroxy-5,6- dihydrocyclopenta[b]pyridin-2-yl)amino]- 2-[3-methoxy-4-(1-methyl-4- piperidyl)anilino]pyrimidine-5-carbonitrile |
| 46 | 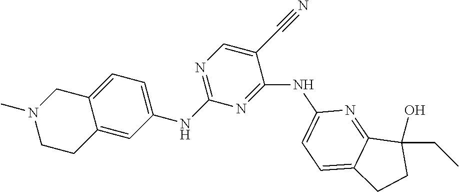 [SMILES: CCC1(O)CCc2ccc(Nc3nc(Nc4ccc5c(c4)CCN(C)C5)ncc3C#N)nc21] | 4-[(7-ethyl-7-hydroxy-5,6- dihydrocyclopenta[b]pyridin-2-yl)amino]- 2-[(2-methyl-3,4-dihydro-1H-isoquinolin- 6-yl)amino]pyrimidine-5-carbonitrile |
| 47 | 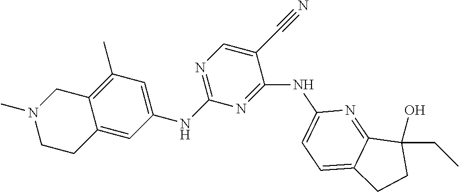 [SMILES: CCC1(O)CCc2ccc(Nc3nc(Nc4cc(C)c5c(c4)CCN(C)C5)ncc3C#N)nc21] | 2-[(2,8-dimethyl-3,4-dihydro-1H- isoquinolin-6-yl)amino]-4-[(7-ethyl-7- hydroxy-5,6-dihydrocyclopenta[b]pyridin- 2-yl)amino]pyrimidine-5-carbonitrile |
| 48 | 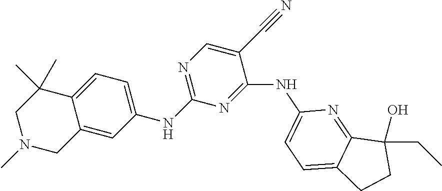 [SMILES: CCC1(O)CCc2ccc(Nc3nc(Nc4ccc5c(c4)CN(C)CC5(C)C)ncc3C#N)nc21] | 4-[(7-ethyl-7-hydroxy-5,6- dihydrocyclopenta[b]pyridin-2-yl)amino]- 2-[(2,4,4-trimethyl-1,3- dihydroisoquinolin-7- yl)amino]pyrimidine-5-carbonitrile |
| 49 | 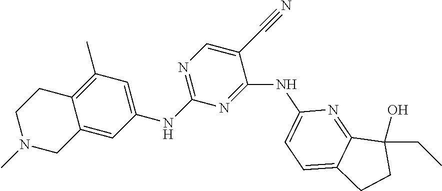 [SMILES: CCC1(O)CCc2ccc(Nc3nc(Nc4cc(C)c5c(c4)CN(C)CC5)ncc3C#N)nc21] | 2-[(2,5-dimethyl-3,4-dihydro-1H- isoquinolin-7-yl)amino]-4-[(7-ethyl-7- hydroxy-5,6-dihydrocyclopenta[b]pyridin- 2-yl)amino]pyrimidine-5-carbonitrile |
| 50 | 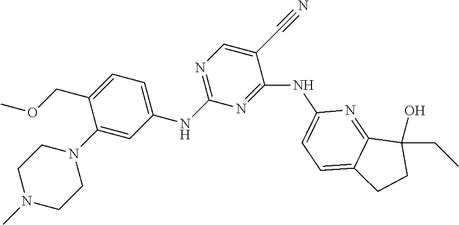 [SMILES: CCC1(O)CCc2ccc(Nc3nc(Nc4ccc(COC)c(N5CCN(C)CC5)c4)ncc3C#N)nc21] | 4-[(7-ethyl-7-hydroxy-5,6- dihydrocyclopenta[b]pyridin-2-yl)amino]- 2-[4-(methoxymethyl)-3-(4- methylpiperazin-1-yl)anilino]pyrimidine- 5-carbonitrile |
| 51 | 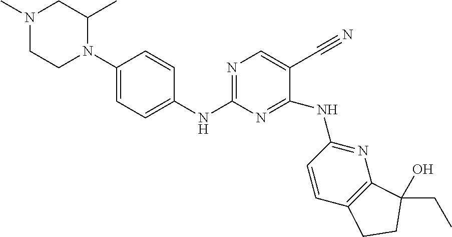 [SMILES: CCC1(O)CCc2ccc(Nc3nc(Nc4ccc(N5CCN(C)CC5C)cc4)ncc3C#N)nc21] | 2-[4-(2,4-dimethylpiperazin-1-yl)anilino]- 4-[(7-ethyl-7-hydroxy-5,6- dihydrocyclopenta[b]pyridin-2- yl)amino]pyrimidine-5-carbonitrile |
| 52 | 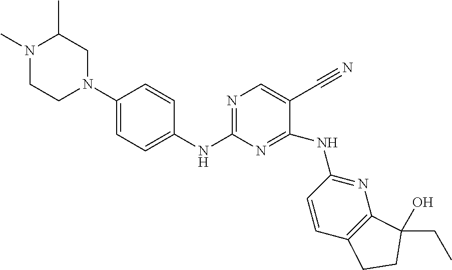 [SMILES: CCC1(O)CCc2ccc(Nc3nc(Nc4ccc(N5CCN(C)C(C)C5)cc4)ncc3C#N)nc21] | 2-[4-(3,4-dimethylpiperazin-1-yl)anilino]- 4-[(7-ethyl-7-hydroxy-5,6- dihydrocyclopenta[b]pyridin-2- yl)amino]pyrimidine-5-carbonitrile |
| 53 | 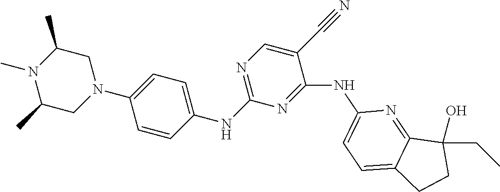 [SMILES: CCC1(O)CCc2ccc(Nc3nc(Nc4ccc(N5C[C@@H](C)N(C)[C@@H](C)C5)cc4)ncc3C#N)nc21] | 4-[(7-ethyl-7-hydroxy-5,6- dihydrocyclopenta[b]pyridin-2- yl)amino]-2-[4-[rac-(3R,5S)-3,4,5- trimethylpiperazin-1- yl]anilino]pyrimidine-5-carbonitrile |
| 54 | 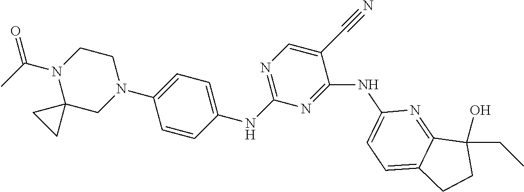 [SMILES: CCC1(O)CCc2ccc(Nc3nc(Nc4ccc(N5CCN(C(C)=O)C6(CC6)C5)cc4)ncc3C#N)nc21] | 2-[4-(4-acetyl-4,7-diazaspiro[2.5]octan-7- yl)anilino]-4-[(7-ethyl-7-hydroxy-5,6- dihydrocyclopenta[b]pyridin-2- yl)amino]pyrimidine-5-carbonitrile |
| 55 | 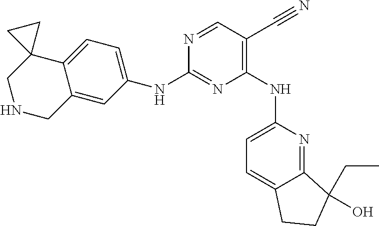 [SMILES: CCC1(O)CCc2ccc(Nc3nc(Nc4ccc5c(c4)CNCC54CC4)ncc3C#N)nc21] | 4-[(7-ethyl-7-hydroxy-5,6- dihydrocyclopenta[b]pyridin-2-yl)amino]- 2-(spiro[2,3-dihydro-1H-isoquinoline-4,1′- cyclopropane]-7-ylamino)pyrimidine-5- carbonitrile |
| 56 | 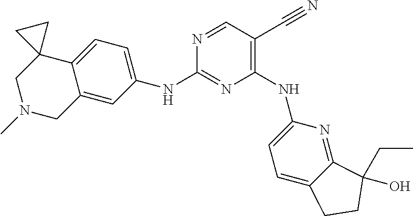 [SMILES: CCC1(O)CCc2ccc(Nc3nc(Nc4ccc5c(c4)CN(C)CC54CC4)ncc3C#N)nc21] | 4-[(7-ethyl-7-hydroxy-5,6- dihydrocyclopenta[b]pyridin-2-yl)amino]- 2-[(2-methylspiro[1,3- dihydroisoquinoline-4,1′-cyclopropane]-7- yl)amino]pyrimidine-5-carbonitrile |
| 57 | 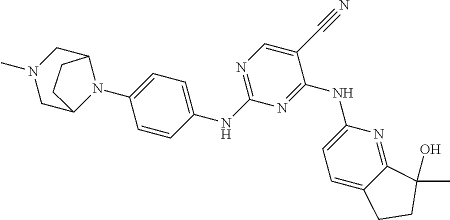 | 4-[(7-hydroxy-7-methyl-5,6- dihydrocyclopenta[b]pyridin-2-yl)amino]- 2-[4-(3-methyl-3,8- diazabicyclo[3.2.1]octan-8- yl)anilino]pyrimidine-5-carbonitrile |
| 58 | 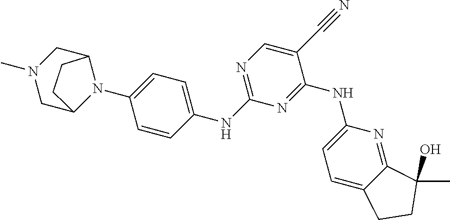 | 4-(((S)-7-hydroxy-7-methyl-6,7-dihydro- 5H-cyclopenta[b]pyridin-2-yl)amino)-2- ((4-(3-methyl-3,8- diazabicyclo[3.2.1]octan-8- yl)phenyl)amino)pyrimidine-5- carbonitrile* |
| 59 | 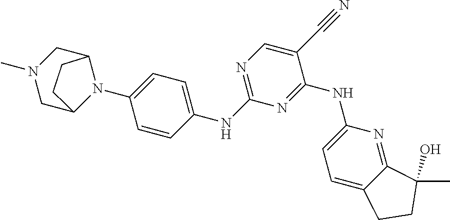 | 4-(((R)-7-hydroxy-7-methyl-6,7-dihydro- 5H-cyclopenta[b]pyridin-2-yl)amino)-2- ((4-(3-methyl-3,8- diazabicyclo[3.2.1]octan-8- yl)phenyl)amino)pyrimidine-5- carbonitrile* |
| 60 | 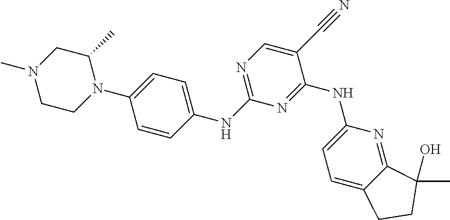 | 4-[(7-hydroxy-7-methyl-5,6- dihydrocyclopenta[b]pyridin-2-yl)amino]- 2-[4-[rac-(2S)-2,4-dimethylpiperazin-1- yl]anilino]pyrimidine-5-carbonitrile |
| 61 | 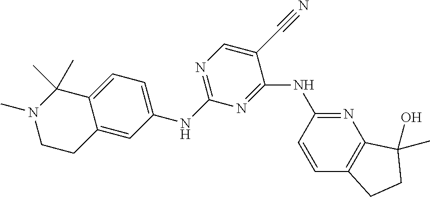 | 4-[(7-hydroxy-7-methyl-5,6- dihydrocyclopenta[b]pyridin-2- yl)amino]-2-[(1,1,2-trimethyl-3,4- dihydroisoquinolin-6- yl)amino]pyrimidine-5-carbonitrile |
| 62 | 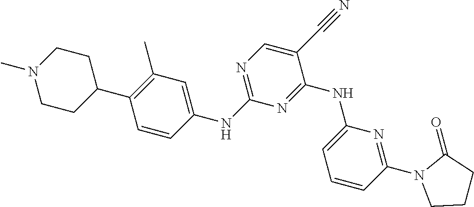 [SMILES: Cc1cc(Nc2ncc(C#N)c(Nc3cccc(N4CCCC4=O)n3)n2)ccc1C1CCN(C)CC1] | 2-[3-methyl-4-(1-methyl-4- piperidyl)anilino]-4-[[6-(2-oxopyrrolidin- 1-yl)-2-pyridyl]amino]pyrimidine-5- carbonitrile |
| 63 | 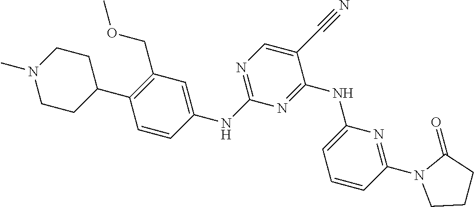 [SMILES: COCc1cc(Nc2ncc(C#N)c(Nc3cccc(N4CCCC4=O)n3)n2)ccc1C1CCN(C)CC1] | 2-[3-(methoxymethyl)-4-(1-methyl-4- piperidyl)anilino]-4-[[6-(2-oxopyrrolidin- 1-yl)-2-pyridyl]amino]pyrimidine-5- carbonitrile |
| 64 | 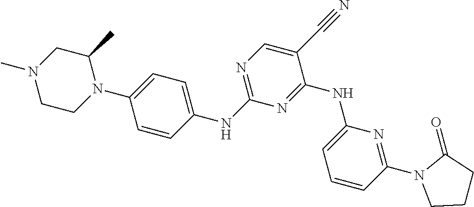 [SMILES: C[C@@H]1CN(C)CCN1c1ccc(Nc2ncc(C#N)c(Nc3cccc(N4CCCC4=O)n3)n2)cc1] | 4-[[6-(2-oxopyrrolidin-1-yl)-2- pyridyl]amino]-2-[4-[(2R)-2,4- dimethylpiperazin-1- yl]anilino]pyrimidine-5-carbonitrile |
| 65 | 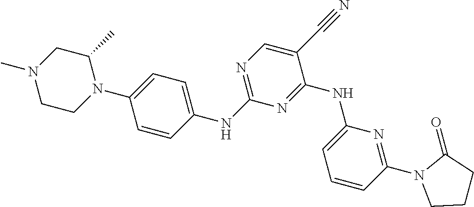 [SMILES: C[C@H]1CN(C)CCN1c1ccc(Nc2ncc(C#N)c(Nc3cccc(N4CCCC4=O)n3)n2)cc1] | 4-[[6-(2-oxopyrrolidin-1-yl)-2- pyridyl]amino]-2-[4-[(2S)-2,4- dimethylpiperazin-1- yl]anilino]pyrimidine-5-carbonitrile |
| 66 | 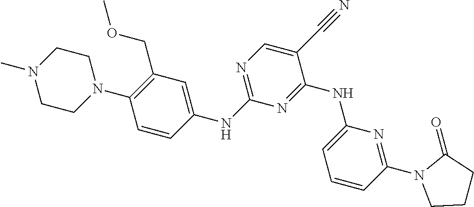 [SMILES: COCc1cc(Nc2ncc(C#N)c(Nc3cccc(N4CCCC4=O)n3)n2)ccc1N1CCN(C)CC1] | 2-[3-(methoxymethyl)-4-(4- methylpiperazin-1-yl)anilino]-4- [[6-(2-oxopyrrolidin-1-yl)-2- pyridyl]amino]pyrimidine-5-carbonitrile |
| 67 | 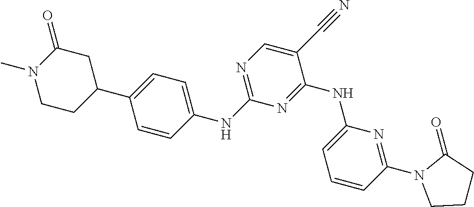 [SMILES: CN1CCC(c2ccc(Nc3ncc(C#N)c(Nc4cccc(N5CCCC5=O)n4)n3)cc2)CC1=O] | 2-[4-(1-methyl-2-oxo-4-piperidyl)anilino]- 4-[[6-(2-oxopyrrolidin-1-yl)-2- pyridyl]amino]pyrimidine-5-carbonitrile |
| 68 | 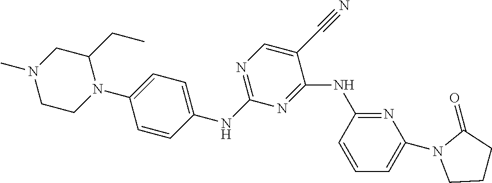 [SMILES: CCC1CN(C)CCN1c1ccc(Nc2ncc(C#N)c(Nc3cccc(N4CCCC4=O)n3)n2)cc1] | 2-[4-(2-ethyl-4-methyl-piperazin-1- yl)anilino]-4-[[6-(2-oxopyrrolidin-1- yl)-2-pyridyl]amino]pyrimidine-5- carbonitrile |
| 69 | 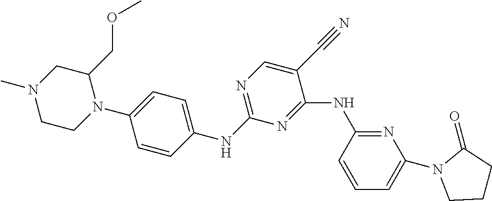 [SMILES: COCC1CN(C)CCN1c1ccc(Nc2ncc(C#N)c(Nc3cccc(N4CCCC4=O)n3)n2)cc1] | 2-[4-[2-(methoxymethyl)-4-methyl- piperazin-1-yl]anilino]-4-[[6-(2- oxopyrrolidin-1-yl)-2- pyridyl]amino]pyrimidine-5-carbonitrile |
| 70 | 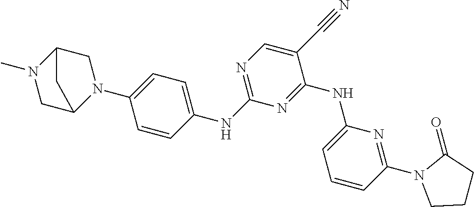 [SMILES: CN1CC2CC1CN2c1ccc(Nc2ncc(C#N)c(Nc3cccc(N4CCCC4=O)n3)n2)cc1] | 2-[4-(5-methyl-2,5- diazabicyclo[2.2.1]heptan-2-yl)anilino]- 4-[[6-(2-oxopyrrolidin-1-yl)-2- pyridyl]amino]pyrimidine-5-carbonitrile |
| 71 | 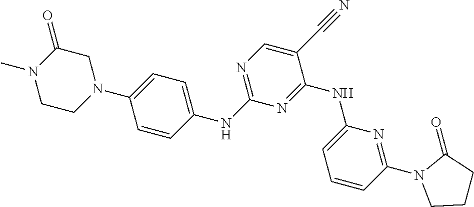 [SMILES: CN1CCN(c2ccc(Nc3ncc(C#N)c(Nc4cccc(N5CCCC5=O)n4)n3)cc2)CC1=O] | 2-[4-(4-methyl-3-oxo-piperazin-1- yl)anilino]-4-[[6-(2-oxopyrrolidin-1- yl)-2-pyridyl]amino]pyrimidine-5- carbonitrile |
| 72 | 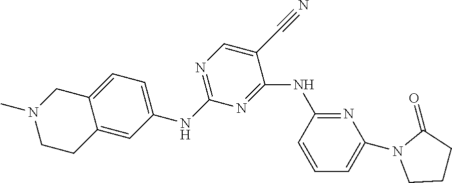 [SMILES: CN1CCc2cc(Nc3ncc(C#N)c(Nc4cccc(N5CCCC5=O)n4)n3)ccc2C1] | 2-[(2-methyl-3,4-dihydro-1H-isoquinolin- 6-yl)amino]-4-[[6-(2-oxopyrrolidin-1-yl)- 2-pyridyl]amino]pyrimidine-5-carbonitrile |
| 73 | 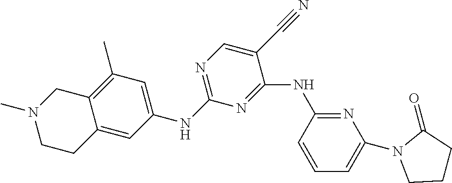 [SMILES: Cc1cc(Nc2ncc(C#N)c(Nc3cccc(N4CCCC4=O)n3)n2)cc2c1CN(C)CC2] | 2-[(2,8-dimethyl-3,4-dihydro-1H- isoquinolin-6-yl)amino]-4-[[6-(2- oxopyrrolidin-1-yl)-2- pyridyl]amino]pyrimidine-5-carbonitrile |
| 74 | 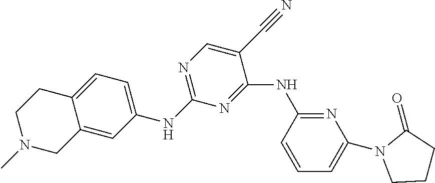 [SMILES: CN1CCc2ccc(Nc3ncc(C#N)c(Nc4cccc(N5CCCC5=O)n4)n3)cc2C1] | 2-[(2-methyl-3,4-dihydro-1H-isoquinolin- 7-yl)amino]-4-[[6-(2-oxopyrrolidin-1-yl)- 2-pyridyl]amino]pyrimidine-5-carbonitrile |
| 75 | 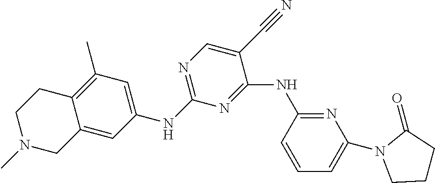 [SMILES: Cc1cc(Nc2ncc(C#N)c(Nc3cccc(N4CCCC4=O)n3)n2)cc2c1CCN(C)C2] | 2-[(2,5-dimethyl-3,4-dihydro-1H- isoquinolin-7-yl)amino]-4-[[6-(2- oxopyrrolidin-1-yl)-2- pyridyl]amino]pyrimidine-5-carbonitrile |
| 76 | 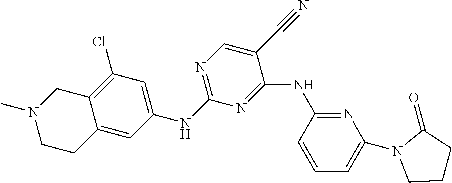 [SMILES: CN1CCc2cc(Nc3ncc(C#N)c(Nc4cccc(N5CCCC5=O)n4)n3)cc(Cl)c2C1] | 2-[(8-chloro-2-methyl-3,4-dihydro-1H- isoquinolin-6-yl)amino]-4-[[6-(2- oxopyrrolidin-1-yl)-2- pyridyl]amino]pyrimidine-5-carbonitrile |
| 77 | 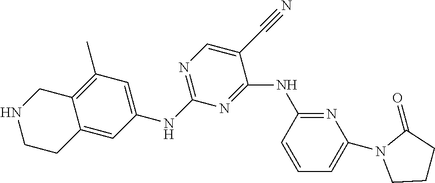 [SMILES: Cc1cc(Nc2ncc(C#N)c(Nc3cccc(N4CCCC4=O)n3)n2)cc2c1CNCC2] | 2-[(8-methyl-1,2,3,4- tetrahydroisoquinolin-6-yl)amino]-4-[[6- (2-oxopyrrolidin-1-yl)-2- pyridyl]amino]pyrimidine-5-carbonitrile |
| 78 | 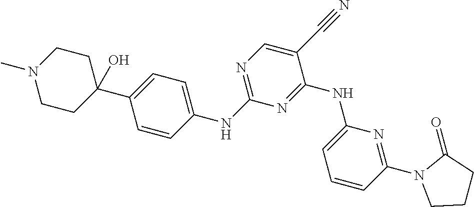 [SMILES: CN1CCC(O)(c2ccc(Nc3ncc(C#N)c(Nc4cccc(N5CCCC5=O)n4)n3)cc2)CC1] | 2-[4-(4-hydroxy-1-methyl-4- piperidyl)anilino]-4-[[6-(2-oxopyrrolidin- 1-yl)-2-pyridyl]amino]pyrimidine-5- carbonitrile |

*chirality arbitrarily assigned

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, tautomer, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof that contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, tautomer, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein that comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like. Representative salts include the alkali or alkaline earth salts, like lithium, sodium, potassium, calcium, and magnesium, and aluminum salts and the like of the tetrazole.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Preparation of the Compounds

The compounds used in the reactions described herein are made according to organic synthesis techniques known to those skilled in this art, starting from commercially available chemicals and/or from compounds described in the chemical literature.

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions. Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry. An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt, solvate, tautomer, or stereoisomer thereof, and a pharmaceutically acceptable excipient.

In certain embodiments, the compound provided herein is substantially pure, in that it contains less than about 5%, or less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as increased overall response rate, increased duration of response, more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal, epidural, or intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached.

### Methods of Treatment

Disclosed herein are methods for treating cancer in a subject in need thereof, including administering to the subject a therapeutically effective amount of a compound disclosed herein, or a pharmaceutically acceptable salt, solvate, tautomer, or stereoisomer thereof. Disclosed herein are methods for treating a WEE1-related cancer in a subject in need thereof, including administering to the subject a therapeutically effective amount of a compound disclosed herein, or a pharmaceutically acceptable salt, solvate, tautomer, or stereoisomer thereof.

In some embodiments, the cancer includes malignant tumors whose size can be decreased, whose growth or spread can be halted, or whose symptom is in remission or alleviated and/or completely cured by deleting or suppressing and/or inhibiting functions of WEE1. Malignant tumors of interest are, but not limited to, head and neck cancer, gastrointestinal cancer (esophageal cancer, gastric cancer, duodenal cancer, liver cancer, biliary tract cancer (gallbladder, bile duct cancer, etc.), pancreatic cancer, colorectal cancer (colon cancer, rectal cancer, etc.), etc.), lung cancer (non-small cell lung cancer, small cell lung cancer, squamous cell lung carcinoma, mesothelioma, etc.), breast cancer, genital cancer (ovarian cancer, uterine cancer, cervical cancer, endometrial cancer, etc.), urinary cancer (kidney cancer, bladder cancer, prostate cancer, testicular tumor, etc.), hematopoietic tumors (leukemia, malignant lymphoma, multiple myeloma, etc.), bone and soft tissue tumors (e.g., soft tissue sarcomas and osteosarcomas), skin cancer, brain tumor (e.g., glioblastoma) and the like. In some embodiments, the cancer is a brain tumor (e.g., glioblastoma). In some embodiments, the cancer has metastasized in the brain.

In some embodiments, the term cancer is used in accordance with its plain ordinary meaning in light of the present disclosure and refers to all types of cancer, neoplasm or malignant tumors found in mammals, including leukemias, lymphomas, melanomas, neuroendocrine tumors, carcinomas, and sarcomas. Exemplary cancers that may be treated with a compound disclosed herein, or a pharmaceutically acceptable salt, solvate, tautomer, or stereoisomer thereof, pharmaceutical compositions include acute myeloid leukemia, adrenal cortical cancer, adrenal gland cancer, bladder cancer, bone cancer, brain cancer, breast cancer (e.g., ductal carcinoma, lobular carcinoma, primary, metastatic), breast cancer, cancer of the endocrine system, cancer of the hepatic stellate cells, cancer of the pancreatic stellate cells, cervical cancer, colon cancer, colorectal cancer, ductal carcinoma, endometrial cancer, esophageal cancer, gastric cancer, genitourinary tract cancer, glioblastoma, glioma, head and neck cancer, hepatocellular carcinoma, Hodgkin's Disease, kidney cancer, leukemia (e.g., lymphoblastic leukemia, chronic lymphocytic leukemia, hairy cell leukemia), liver cancer (e.g., hepatocellular carcinoma), lobular carcinoma, lung cancer (e.g., non-small cell lung carcinoma, squamous cell lung carcinoma, adenocarcinoma, large cell lung carcinoma, small cell lung carcinoma, carcinoid, sarcoma), lymph node cancer, lymphoma (e.g., Mantel cell lymphoma, follicular lymphoma, diffuse large B-cell lymphoma, marginal zona lymphoma, Burkitt's lymphoma, Non-Hodgkin's Lymphoma) malignant carcinoid, malignant hypercalcemia, malignant pancreatic insulinoma, medullary thyroid cancer, Medulloblastoma, melanoma, mesothelioma, multiple myeloma muscle cancer, neoplasms of the endocrine or exocrine pancreas, neuroblastoma, ovarian cancer, Paget's Disease of the Nipple, pancreatic cancer, papillary thyroid cancer, Phyllodes Tumors, premalignant skin lesions, primary thrombocytosis, prostate cancer (e.g. castration-resistant prostate cancer) rhabdomyosarcoma, salivary gland cancer, sarcoma, soft tissue sarcoma, squamous cell carcinoma (e.g., head, neck, or esophagus), stomach cancer, testicular cancer, thyroid cancer, urinary bladder cancer, or uterine cancer. In embodiments, the cancer is selected from bladder cancer, breast cancer, colon cancer, esophageal cancer, esophageal cancer, glioblastoma, liver cancer, lung cancer, ovarian cancer, pancreatic cancer, prostate cancer, salivary gland cancer, soft tissue sarcoma, squamous cell lung carcinoma, stomach cancer, and uterine cancer.

ecDNA mediates an important and clinically distinct mechanism of resistance to targeted therapies. There are immediate therapeutic opportunities for utility of the one or more WEE1 inhibitor described herein as a single agent or in combination with other therapies. In some embodiments, the one or more WEE1 inhibitor described herein may be used to treat an ecDNA+ cancer, ecDNA+ tumor or ecDNA+ tumor cells. One or more WEE1 inhibitor described herein may be used to treat tumors, such as with one or more amplified oncogenes (e.g. FGFR, EGFR, MET, KRAS, MDM2 amplifications), in some cases, the one or more amplified oncogenes comprise non-mutant forms of the oncogene and in some cases, the amplified oncogenes comprises mutant forms of the oncogenes. One or more WEE1 inhibitor described herein may be used to treat tumors for which there are no approved targeted therapies or for which highly efficacious therapies are lacking. One or more WEE1 inhibitor described herein may be used to treat tumors that have developed resistance to another therapy such as a resistance to a targeted agent. In some cases, a tumor (or tumor cells) treated with one or more targeted agents develops resistance to a targeted agent, such as a targeted agent directed to an oncogene or a targeted agent that directly inhibits activating mutant forms of certain oncoproteins (e.g. KRAS, BRAF, EGFR) or as a consequence of focal amplification such as ecDNA-based amplification of the target gene itself, and the one or more WEE1 inhibitor described herein may be used to treat such tumors or tumor cells.

Provided herein are methods wherein inhibition of WEE1 by the one or more WEE1 inhibitors described herein exhibits synthetic lethality with a cancer-targeted agent. In some embodiments, synthetic lethality arises with one or more WEE1 inhibitors described herein in combination with a cancer targeted agent. In some cases, a tumor background is identified as hyper-sensitive to a WEE1 inhibitor and allows a sufficient therapeutic index to enable tolerated doses that are efficacious. In some embodiments, synthetic lethality arises with one or more WEE1 inhibitors described herein in combination with a cancer targeted agent where the tumor or tumor cells are ecDNA+. In some cases, WEE1 inhibition results in reduced ecDNA copy number. In some cases, WEE1 inhibition results in enhanced cytotoxicity in ecDNA+ cells. In some cases, enhanced cytotoxicity results from the combination of WEE1 inhibition and inhibition of a cancer-target, such as an oncogene.

In an aspect of methods herein, a tumor or tumor cells to be treated are ecDNA+. In some cases, such tumor or tumor cells are determined to have an ecDNA signature. In some cases, a tumor or tumor cells are determined to have an ecDNA signature when the tumor or tumor cells have one or more characteristics associated with ecDNA+ tumors or tumor cells. For example, in some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof.

### Combination Therapy

In certain instances, the compound described herein, or a pharmaceutically acceptable salt, solvate, tautomer, or stereoisomer thereof, is administered in combination with a second therapeutic agent or a cancer-targeted agent.

In an aspect of methods herein, the method further comprises administering a cancer-targeted therapeutic agent, directed to an activity of a protein product of a target gene. In some cases, the treatment with the cancer-targeted therapeutic agent and the WEE1 inhibitor disclosed herein reduces amplification or expression of the target gene in the tumor or tumor cells. In some cases, the cancer-targeted therapeutic agent is administered prior to the WEE1 inhibitor. In some cases, the cancer-targeted therapeutic agent is administered concurrently with the WEE1 inhibitor.

In an aspect of methods herein, the tumor or tumor cells have an ecDNA signature. In some cases, the tumor or tumor cells develop the ecDNA signature after administration of the cancer-targeted therapeutic agent. In some cases, the tumor or tumor cells develop the ecDNA signature prior to treatment. In some cases, the method prevents an increase of ecDNA in the tumor or tumor cells.

Disclosed herein is a method of treating a tumor or tumor cells, comprising administering a compound disclosed herein, or a pharmaceutically acceptable salt, solvate, tautomer, or stereoisomer thereof, and a cancer-targeted therapeutic agent to a subject identified as having the tumor or tumor cells, wherein the tumor or tumor cells comprise a gene amplification, whereby growth or size of the tumor or growth or number of tumor cells is reduced.

Also disclosed herein is a method of delaying resistance to a cancer-targeted therapeutic agent, the method comprising: administering a compound disclosed herein, or a pharmaceutically acceptable salt, solvate, tautomer, or stereoisomer thereof, to a subject in an amount sufficient to induce replication stress in tumor or tumor cells and administering a cancer-targeted therapeutic agent concurrently or subsequent to the administration of the compound disclosed herein, or a pharmaceutically acceptable salt, solvate, tautomer, or stereoisomer thereof.

Also disclosed herein is a method of treating a subject having a tumor or tumor cells that are non-responsive to a first cancer-targeted therapeutic agent comprising administering a compound disclosed herein, or a pharmaceutically acceptable salt, solvate, tautomer, or stereoisomer thereof, and a second cancer-targeted therapeutic agent to the subject.

Also disclosed herein is a method of provoking a response in a subject having a tumor or tumor cells that are non-responsive or have decreased responsiveness to a first cancer-targeted therapeutic agent comprising administering a compound disclosed herein, or a pharmaceutically acceptable salt, solvate, tautomer, or stereoisomer thereof, and the first cancer-targeted therapeutic agent to the subject.

In some embodiments of a method disclosed herein, the cancer-targeted therapeutic agent is administered concurrent or subsequent to the administration of the compound disclosed herein, or a pharmaceutically acceptable salt, solvate, tautomer, or stereoisomer thereof.

In some embodiments of a method disclosed herein, the first cancer-targeted therapeutic agent is administered concurrent or subsequent to the administration of the compound disclosed herein, or a pharmaceutically acceptable salt, solvate, tautomer, or stereoisomer thereof.

In some embodiments of a method disclosed herein, the second cancer-targeted therapeutic agent is administered concurrent or subsequent to the administration of the compound disclosed herein, or a pharmaceutically acceptable salt, solvate, tautomer, or stereoisomer thereof.

In some embodiments of a method disclosed herein, the cancer-targeted therapeutic agent is administered prior to the administration of the compound disclosed herein, or a pharmaceutically acceptable salt, solvate, tautomer, or stereoisomer thereof.

In some embodiments of a method disclosed herein, the first cancer-targeted therapeutic agent is administered prior to the administration of the compound disclosed herein, or a pharmaceutically acceptable salt, solvate, tautomer, or stereoisomer thereof.

In some embodiments of a method disclosed herein, the second cancer-targeted therapeutic agent is administered prior to the administration of the compound disclosed herein, or a pharmaceutically acceptable salt, solvate, tautomer, or stereoisomer thereof.

In some embodiments of a method disclosed herein, the compound disclosed herein, or a pharmaceutically acceptable salt, solvate, tautomer, or stereoisomer thereof, is administered in an amount sufficient to induce replication stress in the tumor or tumor cells.

In some embodiments of a method disclosed herein, the subject is identified as having the tumor or tumor cells comprising a gene amplification.

In some embodiments of a method disclosed herein, the gene amplification is a focal gene amplification.

In some embodiments of a method disclosed herein, the gene amplification is an ecDNA-derived amplification.

In some embodiments of a method disclosed herein, the gene amplification is comprised on ecDNA or an HSR (homogeneously staining region).

In some embodiments of a method disclosed herein, the cancer-targeted therapeutic agent is directed against a protein encoded by a gene contained within the gene amplification.

In some embodiments of a method disclosed herein, the first cancer-targeted therapeutic agent is directed against a protein encoded by a gene contained within the gene amplification.

In some embodiments of a method disclosed herein, the second cancer-targeted therapeutic agent is directed against a protein encoded by a gene contained within the gene amplification.

In some embodiments of a method disclosed herein, the tumor or tumor cells comprise an ecDNA signature.

In some embodiments of a method disclosed herein, cells comprised within the tumor or the tumor cells are ecDNA competent. For example, an ecDNA competent tumor cell may have detectable levels of ecDNA amplification or an ecDNA competent tumor cell has the capacity to generate ecDNA amplification in response to selective pressure such as targeted therapy (e.g., a cancer-targeted therapeutic agent described herein).

In some embodiments of a method disclosed herein, treatment with both the compound disclosed herein, or a pharmaceutically acceptable salt, solvate, tautomer, or stereoisomer thereof, and the cancer-targeted therapeutic agent has a greater effect or a longer duration of effect on reduction of one or more of tumor growth, tumor size, number of tumor cells or tumor metastasis as compared to the treatment with either the compound disclosed herein or the cancer-targeted therapeutic agent when administered alone.

In some embodiments of a method disclosed herein, treatment with both the compound disclosed herein, or a pharmaceutically acceptable salt, solvate, tautomer, or stereoisomer thereof, and the first cancer-targeted therapeutic agent has a greater effect or a longer duration of effect on reduction of one or more of tumor growth, tumor size, number of tumor cells or tumor metastasis as compared to the treatment with either the compound disclosed herein or the first cancer-targeted therapeutic agent when administered alone.

In some embodiments of a method disclosed herein, treatment with both the compound disclosed herein, or a pharmaceutically acceptable salt, solvate, tautomer, or stereoisomer thereof, and the second cancer-targeted therapeutic agent has a greater effect or a longer duration of effect on reduction of one or more of tumor growth, tumor size, number of tumor cells or tumor metastasis as compared to the treatment with either the compound disclosed herein or the second cancer-targeted therapeutic agent when administered alone.

In some embodiments of a method disclosed herein, the greater effect is a synergistic effect.

In some embodiments of a method disclosed herein, the compound disclosed herein, or a pharmaceutically acceptable salt, solvate, tautomer, or stereoisomer thereof, and/or the cancer-targeted therapeutic agent is administered orally.

In some embodiments of a method disclosed herein, the compound disclosed herein, or a pharmaceutically acceptable salt, solvate, tautomer, or stereoisomer thereof, and/or the first cancer-targeted therapeutic agent is administered orally.

In some embodiments of a method disclosed herein, the compound disclosed herein, or a pharmaceutically acceptable salt, solvate, tautomer, or stereoisomer thereof, and/or the second cancer-targeted therapeutic agent is administered orally.

In some embodiments of a method disclosed herein, the compound disclosed herein, or a pharmaceutically acceptable salt, solvate, tautomer, or stereoisomer thereof, and/or the cancer-targeted therapeutic agent is administered parentally.

In some embodiments of a method disclosed herein, the compound disclosed herein, or a pharmaceutically acceptable salt, solvate, tautomer, or stereoisomer thereof, and/or the first cancer-targeted therapeutic agent is administered parentally.

In some embodiments of a method disclosed herein, the compound disclosed herein, or a pharmaceutically acceptable salt, solvate, tautomer, or stereoisomer thereof, and/or the second cancer-targeted therapeutic agent is administered parentally.

In some embodiments of a method disclosed herein, treatment comprises administering multiple doses of the compound disclosed herein, or a pharmaceutically acceptable salt, solvate, tautomer, or stereoisomer thereof, and/or the cancer-targeted therapeutic agent over a treatment period.

In some embodiments of a method disclosed herein, treatment comprises administering multiple doses of the compound disclosed herein, or a pharmaceutically acceptable salt, solvate, tautomer, or stereoisomer thereof, and/or the first cancer-targeted therapeutic agent over a treatment period.

In some embodiments of a method disclosed herein, treatment comprises administering multiple doses of the compound disclosed herein, or a pharmaceutically acceptable salt, solvate, tautomer, or stereoisomer thereof, and/or the second cancer-targeted therapeutic agent over a treatment period.

In some embodiments of a method disclosed herein, the gene amplification comprises an amplification of a gene selected from the group consisting of ABCB1, AKT, ALK, AR, BCL-2, BCR-ABL, BRAF, CDK4, CDK6, c-MET, EGFR, ER, ERBB3, ERRB2, AK, FGFR1, FGFR2, FGFR3, FGFR4, FLT3, GR, HER2, HRAS, IGF1R, KIT, KRAS, MCL-1, MDM2, MDM4, MTOR, MYC, MYCL, MYCN, NRAS, NRG1, NTRK1, NTRK2, NTRK3, PDGFR, PIK3Cδ, PIK3CA/B, RET, and ROS1.

In some embodiments of a method disclosed herein, the first cancer-targeted therapeutic agent is different than the second cancer-targeted therapeutic agent.

In some embodiments of a method disclosed herein, the first cancer-targeted therapeutic agent is the same as the second cancer-targeted therapeutic agent.

In some embodiments of a method disclosed herein, the cancer-targeted therapeutic agent, the first cancer-targeted therapeutic agent, or the second cancer-targeted therapeutic agent is selected from the group consisting of abemaciclib, ado-trastuzumab emtansine, afatinib, alectinib, ALRN-6924, AMG232, AMG-510, apatinib, ARS-3248, AXL1717, AZD-3759, bevacizumab, BI 1701963, bortezomib, brigatinib, cabozantinib, capmatinib, ceritinib, cetuximab, CGM097, crizotinib, dabrafenib, dacomitinib, dasatinib, DS-3032b, encorafenib, entrectinib, ERAS-801, erdafitinib, erlotinib, everolimus, fam-trastuzumab deruxtecan, figitumumab, futibatinib, gefitinib, gossypol, HDM201, idasanutlin, imatinib, infigratinib, iniparib, lapatinib, larotrectinib, LEE011, lenvatinib, LGX818, lorlatinib, MEK162, MK-8242 SCH 900242, MRTX849, navitoclax, necitumumab, nilotinib, obatoclax, olaparib, OSI-906, osimertinib, palbociclib, panitumumab, PD-0332991, perisofine, pertuzumab, PF-06873600, PF-07220060, PL225B, repotrectinib, ribociclib. RLY-4008, RO5045337, salinomycin, salirasib, SAR405838 MI-77301, sorafenib, sotorasib, sunitinib, tamoxifen, temsirolimus, tipifarnib, tivanitab, tofacitinib, trametinib, trastuzumab, tucatinib, UPR1376, VAL-083, vemurafenib, vemurafenib, vintafolide, and zoptarelin doxorubicin.

In some embodiments of a method disclosed herein, the cancer-targeted therapeutic agent, the first cancer-targeted therapeutic agent, or the second cancer-targeted therapeutic agent is an inhibitor of EFGR, FGFR, CDK4/CDK6 or KRAS. In some cases, the EGFR inhibitor is erlotinib, gefitinib, or an analog thereof, or an antibody such as cetuximab, necitumumab, nimotuzumab, and panitumumab. In some cases, the FGFR inhibitor is erdafitinib, futibatinib, infigratinib, pemigatinib, RLY-4008, or an analog thereof. In some cases, the KRAS inhibitor is adagrasib, BI 1701963, sotorasib, or an analog thereof. In some cases, the CDK4/CDK6 inhibitor is abemaciclib, palbociclib, ribociclib, PF-06873600, PF-07220060, or an analog thereof.

In some embodiments of a method disclosed herein, the cancer-targeted therapeutic agent, the first cancer-targeted therapeutic agent, or the second cancer-targeted therapeutic agent is an inhibitor of BRAF. In some embodiments, the BRAF inhibitors include ASN-003, AZ-304, AZ-628, DP-2874, EBI-907, EBI-945, GDC-0879, LYN 204, NMS-P285, NMS-P730, PF-04880594, TL-241, UAI-201, and UB-941. In some embodiments, the BRAF inhibitors include ABM-1310, agerafenib (RXDX-105), ARQ-736, BAL-3833, belvarafenib, BGB-3245, BI-882370, DAY101, lifirafenib, LUT-014, PF-07284890, PLX-8394, RX-208, VS-6766, and XL-281. In some embodiments, the BRAF inhibitors include dabrafenib, encorafenib, and vemurafenib.

In some embodiments of a method disclosed herein, the cancer-targeted therapeutic agent, the first cancer-targeted therapeutic agent, or the second cancer-targeted therapeutic agent is an inhibitor of MDM2 or MDM4. In some embodiments, MDM2 inhibitors include AD-021.32, CYC700, DS-5272, MI-1061, MI-219, MI-43, MD-224, MK-8242, NU-8231, OM-301, PXN-527, Rigel-3, RO-2468, RO-5353, RO-5963, and SIL-43. In some embodiments, MDM2 inhibitors include ALRN-6924, APG-115, ASTX-295, ATSP-7041, BI-907828, CGM-097, idasanutlin, KRT-232 (AMG-232), MI-77301 (SAR405838, SAR299155), NVP-CGM097, RAIN-32 (milademetan), RG7112 (RO5045337), RG7388 (RG7775), serdemetan (JNJ-26854165), siremadlin, and UBX-0101. In some embodiments, the MDM4 inhibitors include 17AAG, 489-PXN, CTX1, FL-118, Inulanolide A, K-178, and SAH-p53-8. In some embodiments, the MDM4 inhibitors include APG-115, ALRN-6924, ATSP-7041, and BI-907828.

In some embodiments of a method disclosed herein, the cancer-targeted therapeutic agent, the first cancer-targeted therapeutic agent, or the second cancer-targeted therapeutic agent is an inhibitor of MET. In some embodiments, the MET small molecule inhibitors such as ABP-1130, BPI-1831, BPI-2021, BYON-3521, CG-203306, CX-1003, Debio-1144, EMD-94283, EMT-100, EMT-101, HE-003, LMV-12, LS-177, NX-125, OMO-2, PF-4254644, PRX-MET, PTX-2173, QBH-196, RP-1400, SAB-Y14, SAR-125844, SGX-126, SYD-3521, WXSH-0011, X-379, and XL-265, and anti-MET antibodies such as ABX-900, GB-263, FS-101, LY-3164530, LY-3343544, PMC-002, and SAIT-301. In some embodiments, the MET small molecule inhibitors such as ABN-401, ABT-700, AMG-208, AMG-337, ARGX-111, BAY-85-3474, BMS-817378, bozitinib, BPI-9016M, glumetinib, golvatinib tartrate, GST-HG161, HQP-8361, I-020, JNJ-38877605, kanitinib, merestinib, MK-2461, MK-8033, OMO-1, pamufetinib, S-49076, savolitinib, SPH-3348, tivantinib, SAR-125844, SCR-1515, and TPX-0022, and anti-MET antibodies such as APL-101, CKD-702, EMB-01, EMI-137, ficlatuzumab, HLX-55, HS-10241, MCLA-129, MT-8633, NOV-1105, RC-108, REGN-5093, SHR-A1403, Sym-015, telisotuzumab vedotin. In some embodiments, the MET small molecule inhibitors such as amivantamab, capmatinib, crizotinib, and tepotinib.

In some embodiments of a method disclosed herein, the cancer-targeted therapeutic agent, the first cancer-targeted therapeutic agent, or the second cancer-targeted therapeutic agent is an inhibitor of CDK4/6. In some embodiments, the CDK4/6 inhibitors include AG-122275, AM-5992, AU2-94, IIIM-985, IIIM-290, GW-491619, HEC-80797, MM-D37K, MS-140, NP-102, QHRD-110, R-547, RGB-286199, RGT-419B, riviciclib, RO-0505124, THR-53, THR-79, TQB-3303, TY-302, VS2-370, XH-30002, and WXWH-0240. In some embodiments, the CDK4/6 inhibitors include auceliciclib, AT-7519, BEBT-209, BPI-1178, BPI-16350, CS-3002, fascaplysin, FCN-437, FN-1501, GLR-2007, HS-10342, lerociclib, milciclib maleate, NUV-422, ON-123300, PF-06842874, PF-06873600, PF-07220060, SHR-6390, TQB-3616, TY-302, voruciclib, and XZP-3287. In some embodiments, the CDK4/6 inhibitors include abemaciclib, palbociclib, ribociclib, and trilaciclib.

In some embodiments of a method disclosed herein, the cancer-targeted therapeutic agent, the first cancer-targeted therapeutic agent, or the second cancer-targeted therapeutic agent is an inhibitor of EGFR. In some embodiments, the EGFR inhibitors include small molecule inhibitors such as APL-1898, BDTX-1535, BLU-701. BPI-361175, CH-7233163, DS-2087, E-10C, FWD-1509, IN-A008, JS-111, JS-113, LL-191, LYN 205, neptinib, NT-004, ORIC-114, PRB-001, SIM-200, TGRX-360, WJ-13404, yinlitinib maleate, and ZSP-0391, and anti-EGFR antibodies such as 705, 707, ABX-900, CMAB-017, GB-263, KN-023, SSGJ-612, and SHR-A1307. In some embodiments, the EGFR inhibitors include small molecule inhibitors such as abivertinib, alflutinib mesylate, agerafenib (RXDX-105), ASK-120067, BBT-176, BDTX-189, BEBT-109, befortinib mesylate, beitatini, BPI-7711, BPI-D0316, BLU-945, CK-101, dositinib, DFP-17729, DZD-9008, epertinib, epitinib (HMPL-813), ES-072, FCN-411, FHND-9041, furmonertinib, GMA-204, Hemay-022, JRF-103, KP-673, larotinib, lazertinib, maihuatinib, marizomib, mobocertinib, naputinib tosilate, nazartinib, NRC-2694-A, OBX1-012, olafertinib, olmutinib, oritinib, pirotinib, poziotinib, SPH-1188, tarloxotinib, theliatinib (HMPL-309), TAS-6417, TPC-064, TQB-3804, TY-9591, WSD-0922, XZP-5809, YK-029A, YZJ-0318, and zorifertinib, and anti-EGFR antibodies such as 602, C-005, CDP1, depatuxizumab, E01001, GC-1118A, GR-1401, HLX-07, HS-627, I-010, imgatuzumab, JMT-101, JZB-28, KN-026, MP-0274, QL-1203, SCT-200, serclutamab, SYN-004, and TAD-011. In some embodiments, the EGFR inhibitors include small molecule inhibitors such as afatinib, amivantamab, aumolertinib (almonertinib), dacomitinib, erlotinib, gefitinib, icotinib, lapatinib, osimertinib, and pyrotinib, and anti-EGFR antibodies such as cetuximab, necitumumab, nimotuzumab, and panitumumab.

In some embodiments of a method disclosed herein, the cancer-targeted therapeutic agent, the first cancer-targeted therapeutic agent, or the second cancer-targeted therapeutic agent is an inhibitor of FGFR.

In some embodiments, FGFR inhibitors include small molecule inhibitors such as ABSK-012, ABSK-061, AST-56100, BIO-1262, BGS-2219, EVT-601, FPI-1966, JAB-6000, KIN-3248, SAR-439115, SC-0011, and WXSH-0011, and anti-FGFR antibodies such as M-6123, OM-RCA-001. In some embodiments, FGFR inhibitors include small molecule inhibitors such as 3D-185, ABSK-011, ABSK-091, aldafermin, alofanib, AZD-4547, BFKB-8488A, BPI-17509, BPI-43487, CPL-304-110, derazantinib, E-7090, EVER-4010001. FGF-401, fisogatinib, futibatinib, gunagratinib, H3B-6527, HH-185, HMPL-453, HS-236, ICP-105, ICP-192, infigratinib, MAX-40279, RLY-4008, rogaratinib, SAR-442501, SY-4798, TT-00434, and zoligratinib (FF-284), and anti-FGFR antibodies such as bemarituzumab. In some embodiments, FGFR inhibitors include small molecule inhibitors such as erdafitinib and pemigatinib.

In some embodiments of a method disclosed herein, the cancer-targeted therapeutic agent, the first cancer-targeted therapeutic agent, or the second cancer-targeted therapeutic agent is an inhibitor of KRAS. In some embodiments, the KRAS inhibitor include small molecule inhibitors such as ABREV01, ARS-1620, APG-1842, ATG-012, BBP-454, BEPT-607, BI-2852, BI-1823911, BPI-421286, BTX-2541, COTI-219, IMM-1811900, JAB-21000, JAB-22000, JAB-23000, JAB-BX300, JP-002, KR-12, LYN 202, MRTX-1133, RAS-F, RMC-6236, RMC-6291, SDGR 5, STX-301, and YL-15293, and anti-KRAS antibodies such as SBT-100, SBT-102, and SBT-300. In some embodiments, the KRAS include small molecule inhibitors such as adagrasib, ARS-3248, D-1553, GDC-6036, JDQ-443, LY3537982, sotorasib (AMG 510), and BI 1701963.

In some embodiments of a method disclosed herein, the cancer-targeted therapeutic agent, the first cancer-targeted therapeutic agent, or the second cancer-targeted therapeutic agent is an inhibitor of c-MET, HER2, androgen receptor (AR), KIT, PDGFRA PI3K, AKT, BCL2, or MCL1. In some embodiments, the c-MET inhibitor includes crizotinib, tivantinib, cabozantinib, foretinib), or a monoclonal antibody against c-MET such as onartuzumab.

In some embodiments of a method disclosed herein, the cancer-targeted therapeutic agent, the first cancer-targeted therapeutic agent, or the second cancer-targeted therapeutic agent targets a protein encoded by one or more genes provided in Table 3.

TABLE 3
Example Genes

| Gene | Description |
|---|---|
| ABCB(1-11) | ABC binding cassette subfamily A |
| ABCC(1-13) | ABC binding cassette subfamily C |
| ABCG(1-5) | ABC binding cassette subfamily G |
| ABL1 | encodes ABL kinase |
| AKT(1-3) | family encoding AKT serine/threonine kinases |
| ALK | anaplastic lymphoma kinase |
| NRC3C4 (AR) | androgen receptor |
| BCL2 | encodes BCL-2 apoptosis regulator |
| BCR | encodes breakpoint cluster region protein |
| BRAF | encodes B-raf serine/threonine kinase |
| CCND1 | Cyclin D1 |
| CCNE1 | Cyclin E1 |
| CDK4 | cell division protein kinase 4 |
| CDK6 | cell division protein kinase 6 |
| EGFR | Epidermal growth factor receptor (also known as |
| | ERBBI and HER1) |
| ERBB(1-4) | HER1-4 family of receptor proteins, including EGFR |
| ESR(1-2) | estrogen receptor (alpha and beta) |
| FAK | focal adhesion kinase |
| FGFR(1-4) | FGFR1-4 family of receptor proteins |
| FLT3 | FMS like tyrosine kinase 3, aka CD135 |
| NR3C1 (GR) | glucocorticoid receptor |
| HRAS | encodes RAS GTPase/signaling protein HRAS |
| IGF1R | insulin like growth factor receptor (IGF-1R) |
| KIT | encodes c-Kit, aka CD117 |
| KRAS | encodes RAS GTPase/signaling protein KRAS |
| MCL1 | encodes MCL-1, myeloid leukemia cell |
| | differentiation protein |
| MDM2 | mouse double minute 2 |
| MDM4 | mouse double minute 4 |
| MET | encodes c-Met protein (aka HGFR) |
| MTOR | encodes mechanistic target of rapamycin (mTOR) |
| MYC | encodes c-Myc |
| MYCL | encodes l-Myc |
| MYCN | encodes n-Myc |
| NRAS | encodes RAS GTPase/signaling protein NRAS |
| NRG1 | encodes neuregulin 1 |
| NTRK(1-3) | neurotrophic tyrosine receptor kinase |
| PDGFR | encodes platelet derived growth factor receptor |
| NR3C3 (PGR) | progesterone receptor |
| PIK3CA/B/D/G | encodes phosphatidylinositol 3-kinase subunits |
| | alpha/beta/delta/gamma |
| PIK3Cδ | encodes phosphatidylinositol 3-kinase delta |
| RET | encodes RET proto-oncogene |
| ROS1 | encodes ROS proto-oncogene 1 |
| S100A8(MRP8) | encodes S100 calcium binding protein A8 |

In some embodiments of a method disclosed herein, prior to treatment with the compound disclosed herein, or a pharmaceutically acceptable salt, solvate, tautomer, or stereoisomer thereof, the tumor or tumor cells comprise a gene amplification and wherein the cancer-targeted therapeutic agent has an activity directed against a protein encoded by a gene contained within the gene amplification.

In some embodiments of a method disclosed herein, prior to treatment with the compound disclosed herein, or a pharmaceutically acceptable salt, solvate, tautomer, or stereoisomer thereof, the tumor or tumor cells comprise a gene amplification and wherein the first cancer-targeted therapeutic agent has an activity directed against a protein encoded by a gene contained within the gene amplification.

In some embodiments of a method disclosed herein, prior to treatment with the compound disclosed herein, or a pharmaceutically acceptable salt, solvate, tautomer, or stereoisomer thereof, the tumor or tumor cells comprise a gene amplification and wherein the second cancer-targeted therapeutic agent has an activity directed against a protein encoded by a gene contained within the gene amplification.

In some embodiments of a method disclosed herein, the subject received one or more administrations of the first cancer-targeted therapeutic agent prior to the administration of the compound disclosed herein, or a pharmaceutically acceptable salt, solvate, tautomer, or stereoisomer thereof, and wherein the tumor or tumor cells developed resistance to the first cancer-targeted therapeutic agent.

In some embodiments of a method disclosed herein, the compound disclosed herein, or a pharmaceutically acceptable salt, solvate, tautomer, or stereoisomer thereof, is administered concurrently or prior to the administration of the cancer-targeted therapeutic agent.

In some embodiments of a method disclosed herein, the compound disclosed herein, or a pharmaceutically acceptable salt, solvate, tautomer, or stereoisomer thereof, is administered concurrently or prior to the administration of the first cancer-targeted therapeutic agent.

In some embodiments of a method disclosed herein, the compound disclosed herein, or a pharmaceutically acceptable salt, solvate, tautomer, or stereoisomer thereof, is administered concurrently or prior to the administration of the second cancer-targeted therapeutic agent.

In some embodiments of a method disclosed herein, the subject received one or more administrations of the first cancer-targeted therapeutic agent prior to the administration of the compound disclosed herein, or a pharmaceutically acceptable salt, solvate, tautomer, or stereoisomer thereof, and wherein the tumor or tumor cells developed decreased responsiveness or resistance to the first cancer-targeted therapeutic agent.

In some embodiments of a method disclosed herein, treatment with both the compound disclosed herein, or a pharmaceutically acceptable salt, solvate, tautomer, or stereoisomer thereof, and the cancer-targeted therapeutic agent has a greater effect or a longer duration of effect on reduction of one or more of tumor growth, tumor size, number of tumor cells or tumor metastasis as compared to the treatment with either the compound disclosed herein or the cancer-targeted therapeutic agent when administered alone.

In some embodiments of a method disclosed herein, treatment with both the compound disclosed herein, or a pharmaceutically acceptable salt, solvate, tautomer, or stereoisomer thereof, and the first cancer-targeted therapeutic agent has a greater effect or a longer duration of effect on reduction of one or more of tumor growth, tumor size, number of tumor cells or tumor metastasis as compared to the treatment with either the compound disclosed herein or the first cancer-targeted therapeutic agent when administered alone.

In some embodiments of a method disclosed herein, treatment with both the compound disclosed herein, or a pharmaceutically acceptable salt, solvate, tautomer, or stereoisomer thereof, and the second cancer-targeted therapeutic agent has a greater effect or a longer duration of effect on reduction of one or more of tumor growth, tumor size, number of tumor cells or tumor metastasis as compared to the treatment with either the compound disclosed herein or the second cancer-targeted therapeutic agent when administered alone.

In certain embodiments, different therapeutically effective dosages of the compounds disclosed herein will be utilized in formulating a pharmaceutical composition and/or in treatment regimens when the compounds disclosed herein are administered in combination with a second therapeutic agent. Therapeutically effective dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, tautomer, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, tautomer, or stereoisomer thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g., the disease, disorder, or condition from which the subject suffers; the age, weight, sex, diet, and medical condition of the subject). Thus, in some instances, the dosage regimen employed varies and, in some embodiments, deviates from the dosage regimens set forth herein. For combination therapies described herein, dosages of the co-administered compounds vary depending on the type of co-drug employed, on the specific drug employed, on the disease or condition being treated, and so forth.

The compounds described herein, or a pharmaceutically acceptable salt, solvate, tautomer, or stereoisomer thereof, as well as combination therapies, are administered before, during, or after the occurrence of a disease or condition, and the timing of administering the composition containing a compound varies.

In some embodiments, the compound described herein, or a pharmaceutically acceptable salt, solvate, tautomer, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

In some embodiments, the subject identified as having the tumor or tumor cells has cancer. In some embodiments, the cancer includes malignant tumors whose size can be decreased, whose growth or spread can be slowed or halted, or whose symptom is in remission or alleviated, reduced, and/or completely cured by deleting or suppressing and/or inhibiting functions of WEE1. Malignant tumors of interest are, but not limited to, head and neck cancer, gastrointestinal cancer (esophageal cancer, gastric cancer, duodenal cancer, liver cancer, biliary tract cancer (gallbladder, bile duct cancer, etc.), pancreatic cancer, colorectal cancer (colon cancer, rectal cancer, etc.), etc.), lung cancer (non-small cell lung cancer, small cell lung cancer, squamous cell lung carcinoma, mesothelioma, etc.), breast cancer, genital cancer (ovarian cancer, uterine cancer, cervical cancer, endometrial cancer, etc.), urinary cancer (kidney cancer, bladder cancer, prostate cancer, testicular tumor, etc.), hematopoietic tumors (leukemia, malignant lymphoma, multiple myeloma, etc.), bone and soft tissue tumors (e.g., soft tissue sarcomas and osteosarcomas), skin cancer, brain tumor (e.g., glioblastoma) and the like.

In some embodiments, the term cancer is used in accordance with its plain ordinary meaning in light of the present disclosure and refers to all types of cancer, neoplasm or malignant tumors found in mammals, including leukemias, lymphomas, melanomas, neuroendocrine tumors, carcinomas, and sarcomas. Exemplary cancers that may be treated with a compound disclosed herein, or a pharmaceutically acceptable salt, solvate, tautomer, or stereoisomer thereof, pharmaceutical compositions include acute myeloid leukemia, adrenal cortical cancer, adrenal gland cancer, bladder cancer, bone cancer, brain cancer, breast cancer (e.g., ductal carcinoma, lobular carcinoma, primary, metastatic), breast cancer, cancer of the endocrine system, cancer of the hepatic stellate cells, cancer of the pancreatic stellate cells, cervical cancer, colon cancer, colorectal cancer, ductal carcinoma, endometrial cancer, esophageal cancer, gastric cancer, genitourinary tract cancer, glioblastoma, glioma, head and neck cancer, hepatocellular carcinoma, Hodgkin's Disease, kidney cancer, leukemia (e.g., lymphoblastic leukemia, chronic lymphocytic leukemia, hairy cell leukemia), liver cancer (e.g., hepatocellular carcinoma), lobular carcinoma, lung cancer (e.g., non-small cell lung carcinoma, squamous cell lung carcinoma, adenocarcinoma, large cell lung carcinoma, small cell lung carcinoma, carcinoid, sarcoma), lymph node cancer, lymphoma (e.g., Mantel cell lymphoma, follicular lymphoma, diffuse large B-cell lymphoma, marginal zona lymphoma, Burkitt's lymphoma, Non-Hodgkin's Lymphoma) malignant carcinoid, malignant hypercalcemia, malignant pancreatic insulinoma, medullary thyroid cancer, Medulloblastoma, melanoma, mesothelioma, multiple myeloma muscle cancer, neoplasms of the endocrine or exocrine pancreas, neuroblastoma, ovarian cancer, Paget's Disease of the Nipple, pancreatic cancer, papillary thyroid cancer, Phyllodes Tumors, premalignant skin lesions, primary thrombocytosis, prostate cancer (e.g. castration-resistant prostate cancer) rhabdomyosarcoma, salivary gland cancer, sarcoma, soft tissue sarcoma, squamous cell carcinoma (e.g., head, neck, or esophagus), stomach cancer, testicular cancer, thyroid cancer, urinary bladder cancer, or uterine cancer. In embodiments, the cancer is selected from bladder cancer, breast cancer, colon cancer, esophageal cancer, esophageal cancer, glioblastoma, liver cancer, lung cancer, ovarian cancer, pancreatic cancer, prostate cancer, salivary gland cancer, soft tissue sarcoma, squamous cell lung carcinoma, stomach cancer, and uterine cancer.

Gene amplification plays a role in the response of a tumor or tumor cells to cancer-directed therapies and in the development of resistance to targeted therapies. In some instances, gene amplification includes amplification of one or more genes, such as oncogenes, in a focal amplification, where the one or more oncogenes are in higher copy number, whereas surrounding genetic material (e.g., from the chromosomal location of such amplified gene) is not amplified or not at the same level of amplification. Focal amplifications may be located on ecDNA or ecDNA-derived, (i.e., derived from ecDNA, such as ecDNA that has reintegrated into a chromosomal location). ecDNA mediates an important and clinically distinct mechanism of resistance to targeted therapies. Tumor cells with ecDNA and/or ecDNA-derived amplifications may become non-responsive, less responsive, or resistant to a targeted therapy. There are immediate therapeutic opportunities for utility of the compound disclosed herein, or a pharmaceutically acceptable salt, solvate, tautomer, or stereoisomer thereof in combination with other therapies. In some embodiments, the compound disclosed herein, or a pharmaceutically acceptable salt, solvate, tautomer, or stereoisomer thereof may be used in combination with a therapeutic agent to treat an ecDNA+ cancer, ecDNA+ tumor or ecDNA+ tumor cells (i.e., tumor cells containing gene amplifications on ecDNA or derived from ecDNA). The combination of the compound disclosed herein, or a pharmaceutically acceptable salt, solvate, tautomer, or stereoisomer thereof and a therapeutic agent may be used to treat tumors, such as with one or more amplified oncogenes (e.g., BRAF, CDK4, CDK6, EGFR, FGFR, HER2, KRAS, MET, MDM2 amplifications); in some cases, the one or more amplified oncogenes comprise non-mutant forms of the oncogene and in some cases, the amplified oncogenes comprises mutant forms of the oncogenes. In some cases, the one or more amplified oncogenes are extrachromosomal (i.e., on ecDNA) and/or are ecDNA-derived and located on a chromosome. In some embodiments herein, the therapeutic agent used in combination with the compound disclosed herein, or a pharmaceutically acceptable salt, solvate, tautomer, or stereoisomer thereof is an inhibitor of a protein encoded by a gene that is amplified on ecDNA or the ecDNA-derived amplification.

The combinations described herein may be used to treat tumors that have developed resistance to another therapy such as a resistance to a targeted agent. In some cases, a tumor (or tumor cells) treated with a first targeted agent develops resistance to the first targeted agent or becomes less responsive or non-responsive to the first targeted agent. In some cases, the first therapeutic agent is an inhibitor of a protein encoded by a gene that is amplified on ecDNA or the ecDNA-derived amplification and the compound disclosed herein, or a pharmaceutically acceptable salt, solvate, tautomer, or stereoisomer thereof and the first targeted agent may be used to treat such tumors or tumor cells. In some cases, the first therapeutic agent is an inhibitor of a protein encoded by a gene that is amplified on ecDNA or the ecDNA-derived amplification and the compound disclosed herein, or a pharmaceutically acceptable salt, solvate, tautomer, or stereoisomer thereof and a second targeted agent may be used to treat such tumors or tumor cells, where the second targeted agent is an inhibitor of a different protein from the protein target of the first targeted agent.

Provided herein are methods wherein inhibition of WEE1 by the compound disclosed herein, or a pharmaceutically acceptable salt, solvate, tautomer, or stereoisomer thereof exhibits synthetic lethality with a cancer-targeted agent. In some embodiments, synthetic lethality arises with the compound disclosed herein, or a pharmaceutically acceptable salt, solvate, tautomer, or stereoisomer thereof in combination with a cancer targeted agent. In some cases, a tumor background is identified as hyper-sensitive to a WEE1 inhibitor and allows a sufficient therapeutic index to enable tolerated doses that are efficacious. In some embodiments, synthetic lethality arises with the compound disclosed herein, or a pharmaceutically acceptable salt, solvate, tautomer, or stereoisomer thereof in combination with a cancer-targeted agent where the tumor or tumor cells are ecDNA+ (i.e., contain ecDNA or ecDNA-derived gene amplification). In some cases, WEE1 inhibition results in reduced ecDNA copy number. In some cases, WEE1 inhibition results in enhanced cytotoxicity in ecDNA+ cells. In some cases, enhanced cytotoxicity results from the combination of WEE1 inhibition and inhibition of a cancer-target, such as an oncogene.

In some embodiments of a method disclosed herein, prior to treatment the tumor or tumor cells comprise ecDNA and wherein the treatment results in a reduction in the amount of ecDNA in the tumor or tumor cells.

In some embodiments of a method disclosed herein, prior to treatment the tumor or tumor cells comprise ecDNA and wherein level or amount of ecDNA subsequent to the treatment is not increased as compared to prior to treatment.

In some embodiments of a method disclosed herein, the method further comprises assessing a sample from a subject for the presence or level of one or more of a gene amplification, a focal gene amplification, ecDNA. HSR or an ecDNA signature. In some cases, such assessment is performed prior to treatment, during a course of treatment or subsequent to treatment.

In some embodiments of a method disclosed herein, the method further comprises obtaining information of the presence or level of one or more of a gene amplification, a focal gene amplification, ecDNA, HSR or an ecDNA signature in the tumor or tumor cells from the subject prior to, during or subsequent to the administration of the compound disclosed herein, or a pharmaceutically acceptable salt, solvate, tautomer, or stereoisomer thereof. In some cases, such information is obtained prior to treatment with the compound disclosed herein, or a pharmaceutically acceptable salt, solvate, tautomer, or stereoisomer thereof and a targeted cancer therapeutic agent. In some cases, such information is obtained during the course of treatment with the compound disclosed herein, or a pharmaceutically acceptable salt, solvate, tautomer, or stereoisomer thereof inhibitor and a targeted cancer therapeutic agent. In some cases, such information is obtained after treatment with a first targeted cancer therapeutic agent but prior to treatment with the compound disclosed herein, or a pharmaceutically acceptable salt, solvate, tautomer, or stereoisomer thereof, and if the information indicates the presence of ecDNA, or an ecDNA-derived gene amplification, the subject is treated with a combination of the compound disclosed herein, or a pharmaceutically acceptable salt, solvate, tautomer, or stereoisomer thereof and the first targeted cancer therapeutic agent or with a combination of the compound disclosed herein, or a pharmaceutically acceptable salt, solvate, tautomer, or stereoisomer thereof and a second targeted cancer therapeutic agent.

In an aspect of methods herein, a tumor or tumor cells are determined to have an ecDNA signature. In some cases, a tumor or tumor cells are determined to have an ecDNA signature when the tumor or tumor cells have one or more characteristics associated with ecDNA+ tumors or tumor cells. For example, in some cases, the ecDNA signature is selected from the group consisting of a gene amplification; a focal gene amplification; characterization of a structural variation; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof.

In an aspect of methods herein, the tumor or tumor cells have an ecDNA signature. In some cases, the tumor or tumor cells develop the ecDNA signature after administration of a first cancer-targeted therapeutic agent. In some cases, the tumor or tumor cells develop the ecDNA signature prior to treatment.

In some embodiments of a method disclosed herein, the information of the presence or level of one or more of a gene amplification, a focal gene amplification, ecDNA, HSR or an ecDNA signature in the tumor or tumor cells is obtained from blood, tissue or one or more cells.

In some embodiments of a method disclosed herein, the information of the presence or level of one or more of a gene amplification, a focal gene amplification, ecDNA, HSR or an ecDNA signature in the tumor or tumor cells is obtained by liquid biopsy or tissue biopsy.

### EXAMPLES

All final compounds were purified by either high-performance liquid chromatography (HPLC) or supercritical fluid chromatography (SFC) and were characterized by proton (¹H) NMR. All chemicals were purchased from commercial suppliers and used as received unless otherwise indicated. Proton nuclear magnetic resonance (¹H NMR) spectra were recorded on Bruker AVANCE 300 or 400 MHz spectrometers. Chemical shifts are expressed in S ppm and are calibrated to the residual solvent peak. Coupling constants (J), when given, are reported in hertz. Multiplicities are reported using the following abbreviations: s=singlet, d=doublet, dd=doublet of doublets, t=triplet, q=quartet, m=multiplet (range of multiplet is given), br=broad signal, and dt=doublet of triplets.

### LC/MS Conditions:

Method 1: Agilent 6125; Column: Waters Cortecs C18+, 2.7 um 30 mm; Mobile phase: ACN (0.05% FA)-Water (0.05% FA); Gradient: 5% ACN to 95% ACN in 1.0 min, hold 1.0 min, total 2.5 min; Flow 1.8 mL/min; Column Temp: 45° C.

Method 2: Waters Cortecs C18+ column, 2.7 μm, 2.1 mm×30 mm; column temperature 45° C.; mobile phase, acetonitrile (+0.05% formic acid):water (+0.05% formic acid); gradient, 5% acetonitrile to 95% acetonitrile in 1.0 min, hold 1.0 min, total 2.5 min; flow rate 1.8 mL/min; UV detection (λ=214, 254 nm).

### Preparative HPLC Methods:

Method A: Daisogel-C18-10-100, 30×250 mm, 5 um, mobile phase: ACN-H₂O (0.1% FA), gradient: 5˜95)

Method B: RediSep C18 column, 100 Å, 5 μm, length 150 mm, ID 10 mm; Mobile phase: 0-100% acetonitrile in H₂O with 0.1% formic acid

Method C (PHL): column: C18 silica gel; mobile phase: McCN in Water (0.1% FA), 0% to 100% gradient in 20 min; detector, UV 254 nm.

Method D (PHL): column: C18 silica gel; mobile phase, MeCN in Water (10 mmol/L NH₄HCO₃), 0% to 100% gradient in 10 min; detector, UV 254 nm.

### Chiral HPLC Methods:

Method A: Column: XBridge Prep OBD C18 Column, 30*150 mm, 5 μm; mobile phase: 10 mmol NH₄HCO₃+0.05% NH₃H₂O and ACN (28% ACN up to 58% in 8 min)

### Preparation of Intermediate

### Preparation of 2-amino-8-ethyl-5,6,7,8-tetrahydroquinolin-8-ol

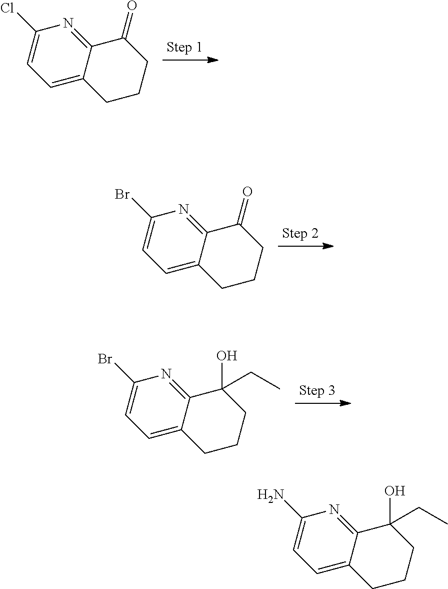

### Step 1: Preparation of 2-bromo-6,7-dihydroquinolin-8(5H)-one

A mixture of 2-chloro-6,7-dihydroquinolin-8(5H)-one (400 mg, 2.21 mmol) and bromotrimethylsilane (676 mg, 4.42 mmol) in MeCN (5 mL) was stirred at 80° C. for 5 hours. The solvent was removed in vacuo to give the crude, which was purified by silica gel flash column chromatography (PE/EA: 0-50%) to afford 2-bromo-6,7-dihydroquinolin-8(5H)-one (310 mg, 62.3% yield) as a yellow solid. MS (ESI): mass calcd. for C₉H₈BrNO 224.98 and 226.98; [M+H]⁺ found 226.0 and 227.9. LCMS (method 1): Rt=1.061 min.

### Step 2: Preparation of 2-bromo-8-ethyl-5,6,7,8-tetrahydroquinolin-8-ol

To a mixture of 2-bromo-6,7-dihydroquinolin-8(5H)-one (280 mg, 1.10 mmol) in THF (5 mL) at 0° C. was added ethylmagnesium bromide (0.4 mL, 1.20 mmol, 3 mol/L). The mixture was stirred at 0° C. for 1 hour, then quenched with saturated ammonium chloride solution (3 mL) and extracted with EA (3×30 mL). The combined organic phases were concentrated in vacuo to give the crude product, which was purified by silica gel flash column chromatography (PE/EA: 0-50%) to afford 2-bromo-8-ethyl-5,6,7,8-tetrahydroquinolin-8-ol (150 mg, 47.3% yield) as a yellow solid. MS (ESI): mass calcd. for C₁₁H₁₄BrNO 255.03 and 257.02; [M+H]⁺ found 256.0 and 258.0. LCMS (method 1): Rt=1.285 min.

### Step 3: Preparation of 2-amino-8-ethyl-5,6,7,8-tetrahydroquinolin-8-ol

A mixture of 2-bromo-8-ethyl-5,6,7,8-tetrahydroquinolin-8-ol (130 mg, 0.51 mmol), Cu₂O (7 mg, 0.05 mmol), K₂CO₃ (105 mg, 0.76 mmol), N1,N1,N2,N2-tetramethylethane-1,2-diamine (4 mg, 0.05 mmol) in mixture solvent (NH₄OH:MEG=1:1, 3.0 mL) was stirred at 60° C. overnight. The reaction mixture was cooled to room temperature and filtered. After the solid was washed with DCM (20 mL), the organic was collected and the solvent was removed in vacu. The resulting crude product was purified by silica gel flash column chromatography (PE/EA (from 0% to 50%)) to afford 2-amino-8-ethyl-5,6,7,8-tetrahydroquinolin-8-ol (50 mg, 51.1% yield) as yellow solid. MS (ESI): mass calcd. for C₁₁H₁₆N₂O 192.13; [M+H]⁺ found 193.2. LCMS (method 1): Rt=0.867 min.

### Preparation of 2-amino-7-propyl-6,7-dihydro-5H-cyclopenta[b]pyridin-7-ol

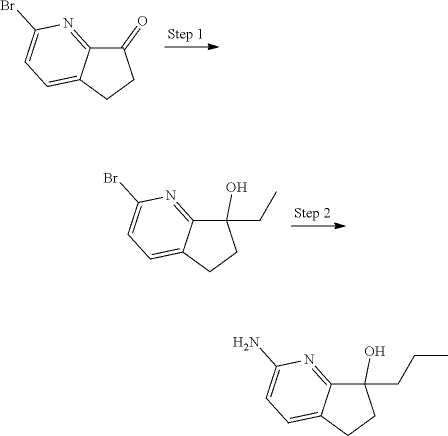

### Step 1: Preparation of 2-bromo-7-propyl-6,7-dihydro-5H-cyclopenta[b]pyridin-7-ol

To a solution of 2-bromo-5,6-dihydro-7H-cyclopenta[b]pyridin-7-one (400.0 mg, 1.89 mmol) in anhydrous THF (20 mL) at 0° C. was added propylmagnesium bromide (2.0 mL, 1 mol/L, 2.00 mmol). The resulting mixture was stirred at this temperature for 1 h under nitrogen atmosphere, and then quenched with water (20 mL). The mixture was extracted with EA (20 mL×3), and the combined organic layers were washed with brine (30 mL), dried over anhydrous sodium sulfate, filtered, and concentrated to obtain the crude product, which was purified by silica gel column chromatography (EA in PE, 0% to 35%) to afford 2-bromo-7-propyl-6,7-dihydro-5H-cyclopenta[b]pyridin-7-ol (200 mg, 41.4% yield) as a colorless oil. MS (ESI): mass calcd. for C₁₁H₁₄BrNO 255.0 and 257.0; [M+H]⁺ found 256.0 and 258.1. LCMS (method 1): Rt=1.238 min.

### Step 2: Preparation of 2-amino-7-propyl-6,7-dihydro-5H-cyclopenta[b]pyridin-7-ol

A mixture of 2-bromo-7-propyl-6,7-dihydro-5H-cyclopenta[b]pyridin-7-ol (200.0 mg, 0.78 mmol), K₂CO₃ (323.0 mg, 2.34 mmol), Cu₂O (56.0 mg, 0.39 mmol) and DMEDA (35.0 mg, 0.39 mmol) in ethylene glycol (1.5 mL) and 28% NH₄OH (1.5 mL) was stirred at 70° C. in a sealed tube under N₂ overnight. The mixture was filtered and the filtrate was concentrated to obtain the crude product, which was purified by prep-HPLC under method A conditions to afford 2-amino-7-propyl-6,7-dihydro-5H-cyclopenta[b]pyridin-7-ol (100.0 mg, 66.6% yield) as a yellow oil. MS (ESI): mass calcd. for C₁₁H₁₆N₂O 192.1: [M+H]⁺ found 193.2. LCMS (method 1): Rt=0.896 min.

### Preparation of 2-amino-7-methyl-6,7-dihydro-5H-cyclopenta[b]pyridin-7-ol

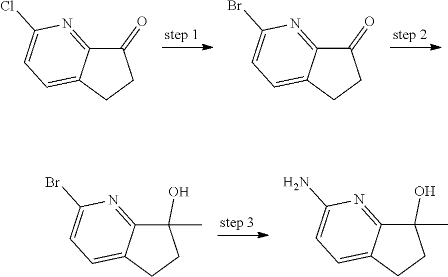

### Step 1: 2-bromo-5,6-dihydro-7H-cyclopenta[b]pyridin-7-one

A solution of 2-chloro-5,6-dihydro-7H-cyclopenta[b]pyridin-7-one (1 g, 0.006 mol), TMS-Br (2.76 g, 0.018 mol) in ACN was stirred at 80° C. under nitrogen for 2 h. The resulting reaction solution was concentrated in vacuo to give the crude product, which was purified by silica gel flash column chromatography (EA in PE: 0% to 50%) to afford 2-bromo-5,6-dihydro-7H-cyclopenta[b]pyridin-7-one (1.05 g, 83.3% yield). MS (ESI): mass calcd. for C₈H₆BrNO 210.96 and 212.96; [M+H]⁺ found 211.9 and 213.9. LCMS (method 1): Rt=1.011 min.

### Step 2: 2-bromo-7-methyl-6,7-dihydro-5H-cyclopenta[b]pyridin-7-ol

To a solution of 2-bromo-5H,6H-cyclopenta[b]pyridin-7-one (500 mg, 2.358 mmol) in THF (5 mL) was added MgMeBr (281.2 mg, 2.358 mmol, 4M) at 0° C. under nitrogen. The reaction mixture was stirred at 0° C. for 2 h. LCMS show the reaction was finished. The reaction mixture was quenched by NH₄Cl (aq. 0.2 mL) and concentrated to give a crude product, which was purified by silica gel flash column chromatography (EA in PE: 0% to 50%) to afford 2-bromo-7-methyl-6,7-dihydro-5H-cyclopenta[b]pyridin-7-ol (230 mg, 42.8% yield) as white solid. MS (ESI): mass calcd. for C₉H₁₀BrNO 226.99 and 228.99; [M+H]⁺ found 228.0 and 230.0. LCMS (method 1): Rt=1.096 min.

### Step 3: Preparation of 2-amino-7-methyl-6,7-dihydro-5H-cyclopenta[b]pyridin-7-ol

A mixture of 2-bromo-7-methyl-6,7-dihydro-5H-cyclopenta[b]pyridin-7-ol (100 mg, 0.441 mmol), K₂CO₃ (179.7 mg, 1.322 mmol), Cu₂O (18.89 mg, 0.132 mmol), DMEDA (11.62 mg, 0.132 mmol) in ethylene glycol (3 mL) and NH₃·H₂O (3 mL) was stirred in a seal tube at 70° C. for 6 h. LCMS shows the reaction was finished. The reaction mixture was filtered and the filtrate was concentrated in vacuo to give a crude product, which was purified by silica gel flash column chromatography (MeOH in DCM: 0% to 10%) to afford 2-amino-7-methyl-6,7-dihydro-5H-cyclopenta[b]pyridin-7-ol (40 mg, 56.3% yield). MS (ESI): mass calcd. for C₉H₁₂N₂O 164.09; [M+H]⁺ found 165.1. LCMS (method 1): Rt=0.245 min.

### Preparation of 2-amino-7-ethyl-6,7-dihydro-5H-cyclopenta[b]pyridin-7-ol

2-amino-7-ethyl-6,7-dihydro-5H-cyclopenta[b]pyridin-7-ol was prepared under the same reaction conditions as the preparation of 2-amino-7-methyl-6,7-dihydro-5H-cyclopenta[b]pyridin-7-ol

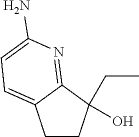

### Preparation of 7-methoxy-7-methyl-6,7-dihydro-5H-cyclopenta[b]pyridin-2-amine

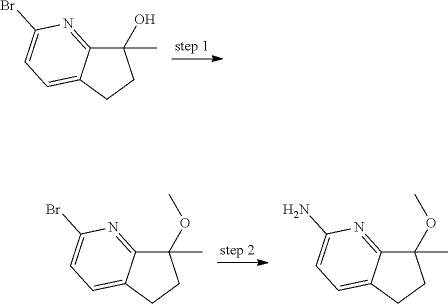

### Step 1: Preparation of 2-bromo-7-methoxy-7-methyl-6,7-dihydro-5H-cyclopenta[b]pyridine

To a mixture of 2-bromo-7-methyl-5H,6H-cyclopenta[b]pyridin-7-ol (500 mg, 2.192 mmol) in THF (5 mL) at 0° C. was added NaH (105.2 mg, 2.630 mmol, 60%). The resulting mixture was stirred at 0° C. for 30 min. A solution of MeI (373.50 mg, 2.630 mmol) in THF (2 mL) was added. Then the reaction mixture was warmed to 25° C. and stirred for 2 h. Then it was quenched by MeOH (2 mL) and concentrated in vacuo to give the crude product, which was purified by silica gel flash column chromatography (EA in PE: 0% to 50%) to afford 2-bromo-7-methoxy-7-methyl-6,7-dihydro-5H-cyclopenta[b]pyridine (400 mg, 75.4% yield). MS (ESI): mass calcd. for C₁₀H₁₂BrNO 241.01 and 243.01; [M+H]⁺ found 242.0 and 244.0. LCMS (method 1): Rt=1.248 min.

### Step 2: Preparation of 7-methoxy-7-methyl-6,7-dihydro-5H-cyclopenta[b]pyridin-2-amine

7-methoxy-7-methyl-6,7-dihydro-5H-cyclopenta[b]pyridin-2-amine was prepared and purified under conditions similar to 2-amino-7-propyl-6,7-dihydro-5H-cyclopenta[b]pyridin-7-ol by using 2-bromo-7-methoxy-7-methyl-6,7-dihydro-5H-cyclopenta[b]pyridine (200 mg, 0.826 mmol) to afford the title compound (100 mg, 67.9% yield). MS (ESI): mass calcd. for C₁₀H₁₄N₂O 178.11; [M+H]⁺ found 179.2. LCMS (method 1): Rt=0.197 min.

### Preparation of 6′-amino-2H-[1,2′-bipyridin]-2-one

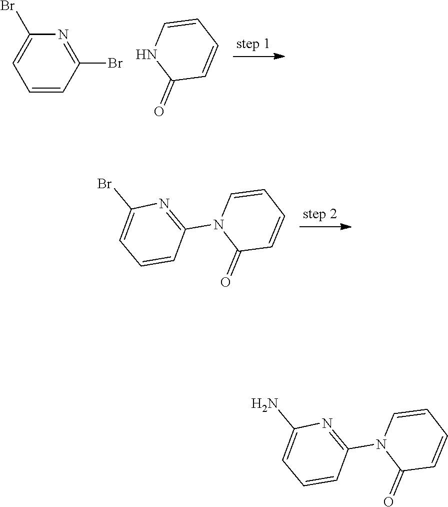

### Step 1: Preparation of 6′-bromo-2H-[1,2′-bipyridin]-2-one

A mixture of 2,6-dibromopyridine (1 g, 4.2 mol), pyridin-2(1H)-one (0.40 g, 4.2 mol) and K₂CO₃ (1.74 g, 12.6 mol) in NMP (10 mL) was stirred at 150° C. under nitrogen for 5 h. The reaction mixture was quenched with water (30 mL) and extracted with EA (30 mL×3). The combined organic phases were removed in vacuo to give the crude product, which was purified by silica gel flash column chromatography (EA in PE: 0% to 50%) to afford 6′-bromo-2H-[1,2′-bipyridin]-2-one (300 mg, 27.57% yield). MS (ESI): mass calcd. for C₁₀H₇BrN₂O 249.97 and 251.97; [M+H]⁺ found 250.9 and 252.9. LCMS (method 1): Rt=1.082 min.

### Step 2: Preparation of 6′-amino-2H-[1,2′-bipyridin]-2-one

A mixture of 1-(6-bromopyridin-2-yl)pyridin-2-one (100 mg, 0.398 mmol) and K₂CO₃ (165.15 mg, 1.195 mmol), Cu₂O (11.40 mg, 0.08 mmol), DMEDA (14.04 mg, 0.159 mmol) in Ethylene glycol (3 mL) and NH₄OH (3 mL, 28% in H₂O) was stirred at 70° C. under nitrogen for 12 h in a seal tube. The reaction mixture was filtered and the filtrate was concentrated in vacuo to give the crude product, which was purified by silica gel flash column chromatography (MeOH in DCM: 0% to 10%) to afford 6′-amino-2H-[1,2′-bipyridin]-2-one (50 mg, 67.1% yield). MS (ESI): mass calcd. for C₁₀H₉N₃O 187.07; [M+H]⁺ found 188.1. LCMS (method 1): Rt=0.795 min.

### Preparation of 1-(6-aminopyridin-2-yl)pyrrolidin-2-one

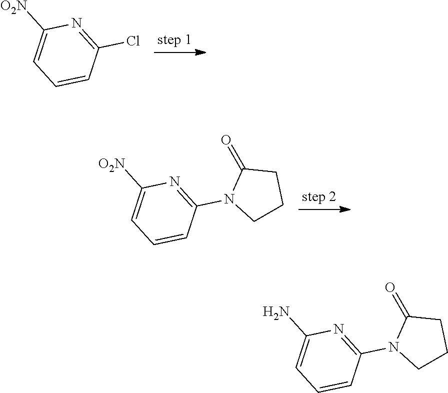 [SMILES: O=[N+]([O-])c1cccc(Cl)n1 ; O=C1CCCN1c1cccc([N+](=O)[O-])n1 ; Nc1cccc(N2CCCC2=O)n1]

### Step 1: Preparation of 1-(6-nitropyridin-2-yl)pyrrolidin-2-one

A mixture of 2-chloro-6-nitropyridine (1.49 g, 9.4 mmol), pyrrolidin-2-one (8 g, 94 mmol) and Pd₂(dba)₃ (0.86 g, 0.94 mmol), Xantphos (1.09 g, 1.8 mmol) in dioxane (30 mL) was stirred at 90° C. under nitrogen for 5 h. The resulting reaction mixture was filtered and the filtrated was concentrated in vacuo to give the crude product, which was purified by silica gel flash column chromatography (EA in PE: 0% to 50%) to afford 1-(6-nitropyridin-2-yl)pyrrolidin-2-one (0.25 g, 12.8% yield). MS (ESI): mass calcd. for C₉H₉N₃O₃ 207.06; [M+H]⁺ found 208.0. LCMS (method 1): Rt=1.136 min.

### Step 2: Preparation of 1-(6-aminopyridin-2-yl)pyrrolidin-2-one

A mixture of 1-(6-nitropyridin-2-yl)pyrrolidin-2-one (250 mg, 1.207 mmol) and Pd/C (64.20 mg, 0.603 mmol) in MeOH (6 mL) was stirred at room temperature under H₂ (1 atm) for 2 hours. The reaction mixture was filtered and the filtrate was concentrated in vacuo to give 1-(6-aminopyridin-2-yl)pyrrolidin-2-one (205 mg, 96.2% yield), which was used in the next step without any further purification. MS (ESI): mass calcd. for C₉H₁₁N₃O 177.09; [M+H]⁺ found 178.1. LCMS (method 1): Rt=0.342 min.

### Preparation of 8-ethyl-5,6,7,8-tetrahydro-1,8-naphthyridin-2-amine

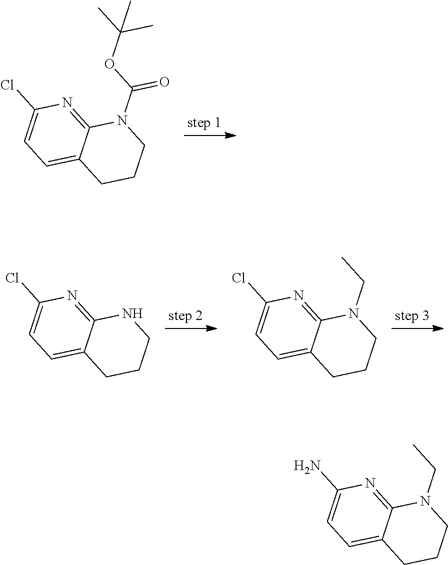 [SMILES: CC(C)(C)OC(=O)N1CCCc2ccc(Cl)nc21 ; Clc1ccc2c(n1)NCCC2 ; CCN1CCCc2ccc(Cl)nc21 ; CCN1CCCc2ccc(N)nc21]

### Step 1: Preparation of 7-chloro-1,2,3,4-tetrahydro-1,8-naphthyridine

Tert-butyl 7-chloro-3,4-dihydro-1,8-naphthyridine-1(2H)-carboxylate (1.1 g, 4.10 mmol) was dissolved in a solution of HCl in dioxane (4 mL, 4 N) and the resulting mixture was stirred at room temperature for 1 h. The solvent was removed in vacuo to give 7-chloro-1,2,3,4-tetrahydro-1,8-naphthyridine (660 mg, 95.7% yield) as a yellow solid, which was used in the next step without any further purification. MS (ESI): mass calcd. for C₈H₉ClN₂ 168.05; [M+H]⁺ found 169.1. LCMS (method 1): Rt=1.150 min.

### Step 2: Preparation of 7-chloro-1-ethyl-1,2,3,4-tetrahydro-1,8-naphthyridine

To a solution of 7-chloro-1,2,3,4-tetrahydro-1,8-naphthyridine (660 mg, 3.92 mmol) in DMF (6 mL) at 0° C. was added NaH (314 mg, 7.84 mmol, 60%), then the reaction mixture was stirred at 0° C. for 30 min. Iodoethane (611 mg, 3.92 mmol) was added and the mixture was stirred at 0° C. for another 1.5 h, then it was quenched with ice water (10 mL) and extracted with EA (3×20 mL). The organic phases were collected and concentrated in vacuo to give the crude product, which was purified by silica gel flash column chromatography (EA in PE: 0% to 25%) to afford 7-chloro-1-ethyl-1,2,3,4-tetrahydro-1,8-naphthyridine (400 mg, 51.9% yield) as a yellow solid. MS (ESI): mass calcd. for C₁₀H₁₃ClN₂ 196.08; [M+H]⁺ found 197.0. LCMS (method 1): Rt=1.444 min.

### Step 3: Preparation of 8-ethyl-5,6,7,8-tetrahydro-1,8-naphthyridin-2-amine

To a mixture of 7-chloro-1-ethyl-1,2,3,4-tetrahydro-1,8-naphthyridine (340 mg, 1.73 mmol), Pd₂(dba)₃ (320 mg, 0.35 mmol) and Johnphos (104 mg, 0.35 mmol) in THF (5 mL) stirred under nitrogen at room temperature was added LiHMDS (0.9 mL, 2.70 mmol, 3 mol/L) dropwise. The reaction mixture was stirred at 75° C. overnight. Then it was acidified with 1 N HCl to pH=5 and extracted with EA (20 mL×3). The aqueous solution was basified with 2N Na₂CO₃ to pH=8 The resulting mixture was extracted with EA (20 mL×3). The combined organic layers were washed with water (30 mL), brine solution (30 mL), dried over anhydrous Na₂SO₄ and concentrated under reduced pressure to give 8-ethyl-5,6,7,8-tetrahydro-1,8-naphthyridin-2-amine (250 mg, 81.4% yield) as a yellow oil, which was used in next step without any further purification. MS (ESI): mass calcd. for C₁₀H₁₅N₃ 177.13; [M+H]⁺ found 178.1. LCMS (method 1): Rt=0.904 min.

### Preparation of 4-(9-methyl-3,9-diazaspiro[5.5]undecan-3-yl)aniline

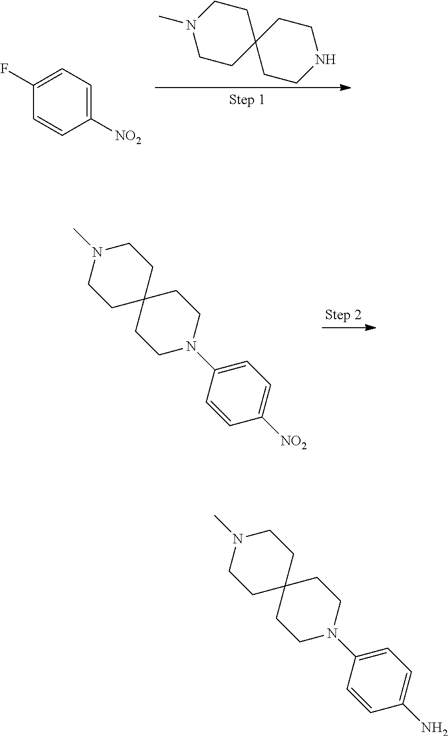 [SMILES: O=[N+]([O-])c1ccc(F)cc1 ; CN1CCC2(CCNCC2)CC1 ; CN1CCC2(CC1)CCN(c1ccc([N+](=O)[O-])cc1)CC2 ; CN1CCC2(CC1)CCN(c1ccc(N)cc1)CC2]

### Step 1: Preparation of 3-methyl-9-(4-nitrophenyl)-3,9-diazaspiro[5.5]undecane

To a stirred mixture of 4-fluoronitrobenzene (503.08 mg, 3.565 mmol, 1.2 eq.) and K₂CO₃ (1231.89 mg, 8.913 mmol, 3 eq.) in DMSO was added 3-methyl-3,9-diazaspiro[5.5]undecane (500 mg, 2.971 mmol, 1.00 eq.) dropwise at 0° C. The resulting mixture was stirred at 80° C. under nitrogen atmosphere for 3 h. Then it was quenched with water (30 ml) and extracted with EtOAc (2×30 mL). The combined organic phases were washed with brine (1×10 mL), and dried over anhydrous Na₂SO₄. After filtration, the filtrate was concentrated under reduced pressure. The residue was purified by silica gel column chromatography (EA in PE: 30%) to afford 3-methyl-9-(4-nitrophenyl)-3,9-diazaspiro [5.5] undecane (600 mg, 69.78%). MS(ESI): mass calcd. for C₁₆H₂₃N₃O₂ 289.17; [M+H]⁺ found: 290.0.

### Step 2: Preparation of 4-(9-methyl-3,9-diazaspiro[5.5]undecan-3-yl)aniline

To a stirred solution of 3-methyl-9-(4-nitrophenyl)-3,9-diazaspiro[5.5]undecane (180 mg, 0.622 mmol, 1 eq.) and NH₄Cl (133.09 mg, 2.488 mmol, 4 eq.) in ethanol were added Fe (138.95 mg, 2.488 mmol, 4 eq.) and H₂O (36.00 mL) in portions at rt. The resulting mixture was stirred at 85° C. under nitrogen atmosphere for 3 h. The mixture was filtered, and the filter cake was washed with ethanol (2×10 mL) and concentrated under reduced pressure. The crude product was purified by reverse phase flash column chromatography afford 4-(9-methyl-3,9-diazaspiro[5.5]undecan-3-ylaniline (150 mg, 92.97%). MS(ESI): mass calcd. for C₁₆H₂₅N₃ 259.20; [M+H]⁺ found, 260.20.

Intermediates below were prepared under the same conditions as 4-(9-methyl-3,9-diazaspiro[5.5]undecan-3-yl)aniline:
4-(5-methylhexahydropyrrolo[3,4-c]pyrrol-2(1H)-yl)aniline

### Preparation of (R)-4-(2,4-dimethylpiperazin-1-yl)aniline

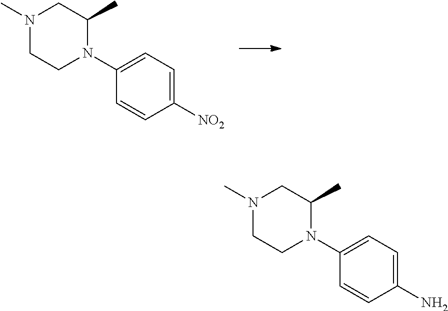 [SMILES: C[C@@H]1CN(C)CCN1c1ccc([N+](=O)[O-])cc1 ; C[C@@H]1CN(C)CCN1c1ccc(N)cc1]

(R)-2,4-dimethyl-1-(4-nitrophenyl)piperazine was prepared starting with (3R)-1,3-dimethylpiperazine and 1-fluoro-4-nitrobenzene under the same conditions as the preparation of 3-methyl-9-(4-nitrophenyl)-3,9-diazaspiro[5.5]undecane.

Into a 50 mL round-bottom flask were added (R)-2,4-dimethyl-1-(4-nitrophenyl)piperazine (180 mg, 0.89 mmol, 1.0 eq.), MeOH (5 mL) and Pd/C (180 mg, 1.691 mmol, 1.90 eq.) at room temperature. The resulting mixture was stirred for 1 h under hydrogen atmosphere. Then it was filtered, and the filter cake was washed with MeOH (3×10 mL). The filtrate was concentrated under reduced pressure to give 4-[(2R)-2,4-dimethylpiperazin-1-yl]aniline (150 mg, 82.13%) as a brown solid, which was directly used in the next step without further purification. MS (ESI): [M+H]⁺ found, 206.10.

Intermediates below were prepared under the same conditions as (R)-4-(2,4-dimethylpiperazin-1-yl)aniline:
(S)-4-(2,4-dimethylpiperazin-1-yl)aniline
(R/S)-4-(2,4-dimethylpiperazin-1-yl)aniline
(R/S)-4-(3,4-dimethylpiperazin-1-yl)aniline
Cis-(3,4,5-trimethylpiperazin-1-yl)aniline
Racemic 4-(2-ethyl-4-methylpiperazin-1-yl)aniline
4-(5-methyl-2,5-diazabicyclo[2.2.2]octan-2-yl)aniline
tert-butyl 7-(4-aminophenyl)-4,7-diazaspiro[2.5]octane-4-carboxylate
4-(4-methyl-4,7-diazaspiro[2.5]octan-7-yl)aniline
4-(8-methyl-3,8-diazabicyclo[3.2.1]octan-3-yl)aniline
4-(3-methyl-3,8-diazabicyclo[3.2.1]octan-8-yl)aniline
4-(4-isopropylpiperazin-1-yl)aniline
4-(5-methyl-2,5-diazabicyclo[2.2.1]heptan-2-yl)aniline
4-(4-aminophenyl)-1-methylpiperazin-2-one

### Preparation of 1-(7-(4-aminophenyl)-4,7-diazaspiro[2.5]octan-4-yl)ethan-1-one

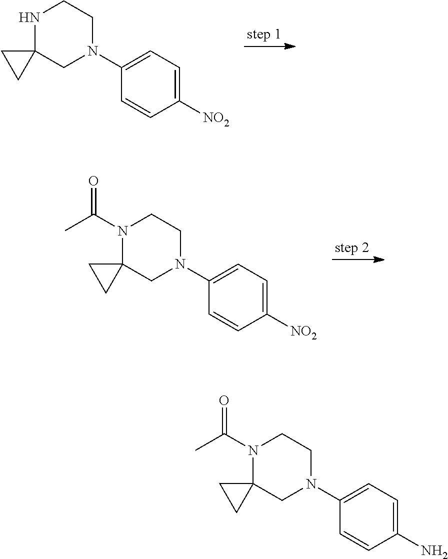 [SMILES: O=[N+]([O-])c1ccc(N2CCNC3(CC3)C2)cc1 ; CC(=O)N1CCN(c2ccc([N+](=O)[O-])cc2)CC12CC2 ; CC(=O)N1CCN(c2ccc(N)cc2)CC12CC2]

### Step 1: Preparation of 1-(7-(4-nitrophenyl)-4,7-diazaspiro[2.5]octan-4-yl)ethan-1-one

To a stirred solution of 7-(4-nitrophenyl)-4,7-diazaspiro[2.5]octane (700 mg, 3.001 mmol, 1 eq.) and TEA (1668.46 μL, 12.00 mmol, 4 eq.) in DCM (7 mL) was added acetyl chloride (259.12 mg, 3.30 mmol, 1.1 eq.) dropwise at 0° C. under nitrogen atmosphere. The resulting mixture was stirred at rt under nitrogen atmosphere for 1 h. The resulting mixture was quenched with water (10 ml) and extracted with DCM (2×10 mL). The combined organic layers were washed with brine (1×10 mL) and dried over anhydrous Na₂SO₄. After filtration, the filtrate was concentrated under reduced pressure. The residue was purified by silica gel column chromatography (EA in PE: 33%) to afford 1-[7-(4-nitrophenyl)-4,7-diazaspiro[2.5]octan-4-yl]ethanone (520 mg, 62.94%). MS(ESI): mass calcd. for C₁₄H₁₇N₃O₃ 275.12; [M+H]⁺ found, 276.0.

### Step 2: Preparation of 1-(7-(4-aminophenyl)-4,7-diazaspiro[2.5]octan-4-yl)ethan-1-one

To a solution of 1-[7-(4-nitrophenyl)-4,7-diazaspiro[2.5]octan-4-yl]ethanone (500 mg, 1.82 mmol, 1 eq.) in EA (5 mL) was added Pd/C (10%, 0.1 g) under nitrogen atmosphere in a 25 mL round-bottom flask. The mixture was hydrogenated at room temperature under hydrogen atmosphere using a hydrogen balloon for 1 h, then was filtered through a Celite pad and concentrated under reduced pressure. The crude product was used in the next step directly without further purification. MS(ESI): mass calcd. for C₁₄H₁₉N₃O 245.15; [M+H]⁺ found, 246.0.

### Preparation of 3,5-difluoro-4-(1-methylpiperidin-4-yl)aniline

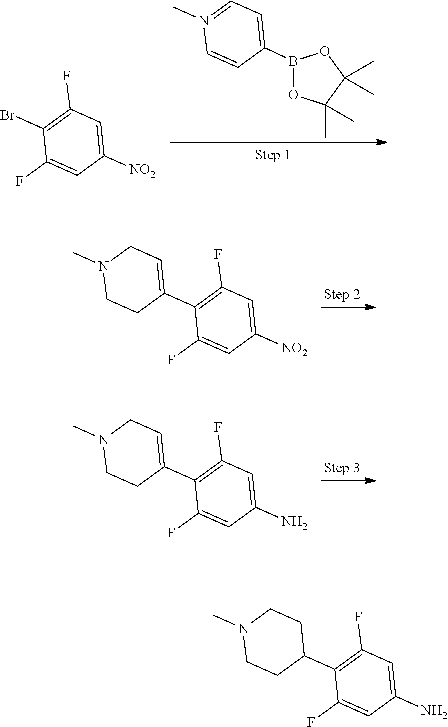

### Step 1: Preparation of 4-(2,6-difluoro-4-nitrophenyl)-1-methyl-1,2,3,6-tetrahydropyridine

Into a 20 mL sealed tube were added 2-bromo-1,3-difluoro-5-nitrobenzene (1 g, 4.202 mmol, 1.0 eq.), dioxane (18 mL), H₂O (2 mL), 1-methyl-4-(4,4,5,5-tetramethyl-1,3,2-dioxaborolan-2-yl)-3,6-dihydro-2H-pyridine (1.59 g, 7.143 mmol, 1.7 eq.), K₂CO₃ (1.39 g, 10.085 mmol, 2.4 eq.) and Pd(dppf)Cl₂ CH₂Cl₂ (171.15 mg, 0.210 mmol, 0.05 eq.) at room temperature. The resulting mixture was stirred at 110° C. under nitrogen atmosphere for 2 h. The mixture was filtered, and the filter cake was washed with ethyl acetate (3×10 mL). The filtrate was extracted with EtOAc (3×10 mL). The combined organic phases were washed with brine (3×10 mL) and dried over anhydrous Na₂SO₄. After filtration, the filtrate was concentrated under reduced pressure. The residue was purified by reversed-phase flash chromatography under Method C conditions to give 4-(2,6-difluoro-4-nitrophenyl)-1-methyl-3,6-dihydro-2H-pyridine (1.017 g, 95.20%) as a black oil. MS (ESI): [M+H]⁺ found, 255.05.

### Step 2: Preparation of 3,5-difluoro-4-(1-methyl-1,2,3,6-tetrahydropyridin-4-yl)aniline

Into a 50 mL round-bottom flask were added 4-(2,6-difluoro-4-nitrophenyl)-1-methyl-3,6-dihydro-2H-pyridine (1 g, 3.933 mmol, 1.0 eq.), MeOH (5 L) and Pd/C (1 g, 9.397 mmol, 2.39 eq.) at room temperature. The resulting mixture was stirred at room temperature under hydrogen atmosphere for 2 h. The mixture was filtered and the filter cake was washed with MeOH (3×10 mL). The filtrate was concentrated under reduced pressure to give crude 3,5-difluoro-4-(1-methylpiperidin-4-yl) aniline (500 mg, 56.69%), which was directly used in the next step without further purification. MS (ESI): [M+H]⁺ found: 225.05.

### Step 3: Preparation of 3,5-difluoro-4-(1-methylpiperidin-4-yl)aniline

Into a 50 mL round-bottom flask were added 4-(2,6-difluoro-4-nitrophenyl)-1-methyl-3,6-dihydro-2H-pyridine (500 mg, 3.933 mmol, 1.0 eq.), MeOH (5 mL) and Pd/C (500 mg, 9.397 mmol, 2.39 eq.) at room temperature. The resulting mixture was stirred at 50° C. under hydrogen atmosphere for 2 h. The mixture was filtered and the filter cake was washed with MeOH (3×10 mL). The filtrate was concentrated under reduced pressure. The residue was purified by reversed-phase flash chromatography under Method D conditions to give 3,5-difluoro-4-(1-methylpiperidin-4-yl) aniline (100 mg, 19.82%), which was directly used in the next step without further purification. MS (ESI): [M+H]⁺ found: 227.10.

The intermediates below were prepared by using the same reaction sequence as 3,5-difluoro-4-(1-methylpiperidin-4-yl)aniline
3-fluoro-5-methyl-4-(1-methylpiperidin-4-yl)aniline
3-fluoro-4-(1-methylpiperidin-4-yl)aniline
3-chloro-4-(1-methylpiperidin-4-yl)aniline via step 1 and 2 by using Rh/C/H2 in step 2
3-methyl-4-(1-methylpiperidin-4-yl)aniline
3-(methoxymethyl)-4-(1-methylpiperidin-4-yl)aniline via step 1 and 2 starting with 1-bromo-2-(methoxymethyl)-4-nitrobenzene
3-methoxy-4-(1-methylpiperidin-4-yl)aniline via step 1 and 2 starting with 1-bromo-2-methoxy-4-nitrobenzene

### Preparation of 8-chloro-2-methyl-1,2,3,4-tetrahydroisoquinolin-6-amine

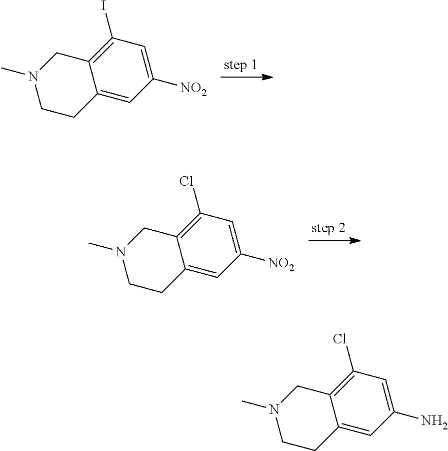

### Step 1: Preparation of 8-chloro-2-methyl-6-nitro-1,2,3,4-tetrahydroisoquinoline

A mixture of 8-iodo-2-methyl-6-nitro-3,4-dihydro-1H-isoquinoline (1 g, 3.14 mmol, 1 eq.) and CuCl (933.63 mg, 9.432 mmol, 3 eq.) in DMF (15 mL) was stirred at 12 5° C. under nitrogen atmosphere for 3 h. The resulting mixture was extracted with EtOAc (3×10 mL). The combined organic layers were washed with brine (1×10 mL) and dried over anhydrous Na₂SO₄. After filtration, the filtrate was concentrated under reduced pressure. The residue was purified by reversed phase flash chromatography (MeCN in Water (10 mmol/L NH₄HCO₃), 10% to 50% gradient in 10 min) to give 8-chloro-2-methyl-6-nitro-3,4-dihydro-1H-isoquinoline (262 mg, 36.77%) as a yellow solid. MS (ESI), [M+H]⁺, 227.

### Step 2: Preparation of 8-chloro-2-methyl-1,2,3,4-tetrahydroisoquinolin-6-amine

To a stirred mixture of 8-chloro-2-methyl-6-nitro-3,4-dihydro-1H-isoquinoline (262 mg, 1.16 mmol, 1 eq.) and NH₄Cl (370.98 mg, 6.94 mmol, 6 eq.) in EtOH (10 mL) and H₂O (2 mL) was added Fe (256.24 mg, 4.59 mmol, 4 eq.) in portions at room temperature. The resulting mixture was stirred at rt for 1 h. Then it was filtered, and the filter cake was washed with MeOH (3×10 mL). The filtrate was concentrated under reduced pressure. The residue was purified by reversed phase flash chromatography (MeCN in Water (10 mmol/L NH₄HCO₃), 10% to 50% gradient in 10 min) to give 8-chloro-2-methyl-3,4-dihydro-1H-isoquinolin-6-amine (100 mg, 43.99%) as a yellow solid. MS (ESI), [M+H]⁺ 197.

### Preparation of 3-(methoxymethyl)-4-(4-methylpiperazin-1-yl)aniline

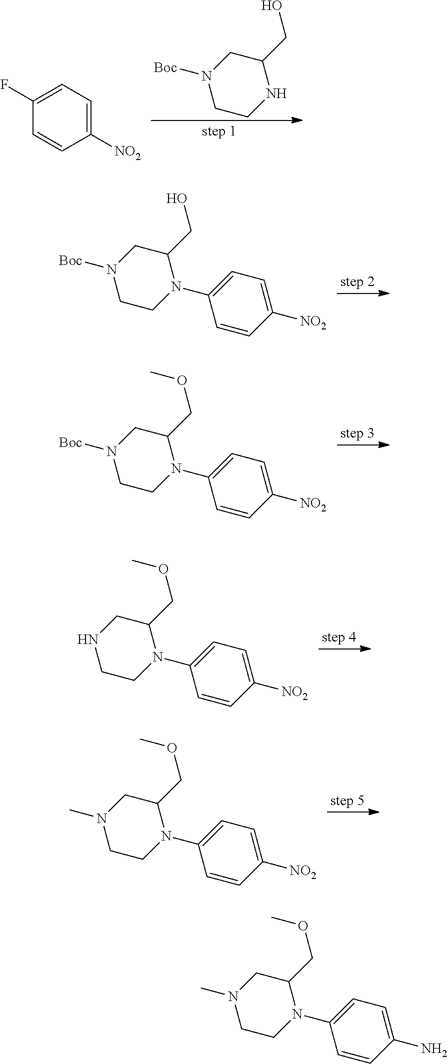

### Step 1: Preparation of tert-butyl 3-(hydroxymethyl)-4-(4-nitrophenyl)piperazine-1-carboxylate

To a stirred solution of 4-fluoronitrobenzene (1 g, 7.087 mmol, 1.00 eq.) and tert-butyl 3-(hydroxymethyl)piperazine-1-carboxylate (1.53 g, 7.087 mmol, 1 eq.) in DMSO (20 mL) was added Cs₂CO₃ (6.93 g, 21.261 mmol, 3 eq.) in portions at rt. The resulting mixture was stirred at 100° C. overnight, then it was quenched with water (10 mL) and extracted with EtOAc (3×10 mL). The combined organic layers were washed with brine (2×10 mL) and dried over anhydrous Na₂SO₄. After filtration, the filtrate was concentrated under reduced pressure. The residue was purified by silica gel column chromatography (EA in PE: 20%) to afford tert-butyl 3-(hydroxymethyl)-4-(4-nitrophenyl)piperazine-1-carboxylate (1.1 g, 46.01%) as a yellow oil. MS(ESI): mass calcd. for C₁₆H₂₃N₃O₅ 337.16; [M+H]⁺ found 338.0.

### Step 2: Preparation of tert-butyl 3-(methoxymethyl)-4-(4-nitrophenyl)piperazine-1-carboxylate

To a stirred solution of tert-butyl 3-(hydroxymethyl)-4-(4-nitrophenyl)piperazine-1-carboxylate (1 g, 2.964 mmol, 1 eq.) in THF (10 mL) was added NaH (142.26 mg, 5.928 mmol, 2 eq.) in portions at 0° C. under nitrogen atmosphere. The mixture was stirred at 0° C. under nitrogen atmosphere for 30 min. Then methyl iodide (2.52 g, 17.784 mmol, 6 eq.) was added dropwise at 0° C. The resulting mixture was stirred at rt overnight, then quenched with water (10 mL) and extracted with EtOAc (3×5 mL). The combined organic layers were washed with brine (1×5 mL) and dried over anhydrous Na₂SO₄. After filtration, the filtrate was concentrated under reduced pressure. The residue was purified by silica gel column chromatography (EA in PE: 33%) to afford tert-butyl 3-(methoxymethyl)-4-(4-nitrophenyl)piperazine-1-carboxylate (830 mg, 79.69%) as a yellow oil. MS(ESI): mass calcd. for C₁₇H₂₅N₃O₅ 351.18; [M+H]⁺ found, 352.0.

### Step 3: Preparation of 2-(methoxymethyl)-1-(4-nitrophenyl)piperazine

A solution of tert-butyl 3-(methoxymethyl)-4-(4-nitrophenyl)piperazine-1-carboxylate (837 mg, 2.382 mmol, 1 eq.) in mixed DCM (9 mL) and TFA (3 mL) was stirred at rt for 1.0 h. The resulting solution was concentrated under reduced pressure. The residue was purified by reversed phase flash chromatography (MeCN in Water (10 mmol/L NH₄HCO₃), 10% to 50% gradient in 10 min) to give 2-(methoxymethyl)-1-(4-nitrophenyl)piperazine (550 mg, 91.89%) as a yellow oil. MS(ESI): mass calcd. for C₁₂H₁₇N₃O₃ 251.13; [M+H]⁺ found, 252.0.

### Step 4: Preparation of 2-(methoxymethyl)-4-methyl-1-(4-nitrophenyl)piperazine

To a stirred solution of 2-(methoxymethyl)-1-(4-nitrophenyl)piperazine (650 mg, 2.587 mmol, 1 eq.) and paraformaldehyde (1.40 g, 15.522 mmol, 6 eq.) in DCE (15 mL) was added NaBH(OAc)₃ (1.10 g, 5.174 mmol, 2 eq.) in portions at rt under nitrogen atmosphere. The resulting mixture was stirred at 60° C. under nitrogen atmosphere for 2 h. The precipitated solids were filtered off and washed with ethyl acetate (3×10 mL). The resulting mixture was quenched with water (10 ml) and extracted with EtOAc (3×10 mL). The combined organic layers were washed with brine (1×10 mL) and dried over anhydrous Na₂SO₄. After filtration, the filtrate was concentrated under reduced pressure. The residue was purified by reversed phase flash chromatography (MeCN in Water (10 mmol/L NH₄HCO₃), 10% to 50% gradient in 10 min) to give 2-(methoxymethyl)-4-methyl-1-(4-nitrophenyl)piperazine (450 mg, 65.57%) as a yellow solid. MS(ESI): mass calcd. for C₁₃H₁₉N₃O₃ 265.14; [M+H]⁺ found, 266.0.

### Step 5: Preparation of 4-(2-(methoxymethyl)-4-methylpiperazin-1-yl)aniline

To a solution of 2-(methoxymethyl)-4-methyl-1-(4-nitrophenyl)piperazine (50 mg, 0.188 mmol, 1 eq.) in EA (4 mL) was added Pd/C (50 mg) at it. The resulting mixture was stirred at rt under hydrogen atmosphere for 1 h. Then it was filtered, the filter cake was washed with ethyl acetate (3×5 mL) and concentrated under reduced pressure. The crude product was used in the next step directly without further purification. MS(ESI): mass calcd. for C₁₃H₂₁N₃O 235.17; [M+H]⁺ found, 236.0.

### Preparation of 4-(4-aminophenyl)-1-methylpiperidin-4-ol

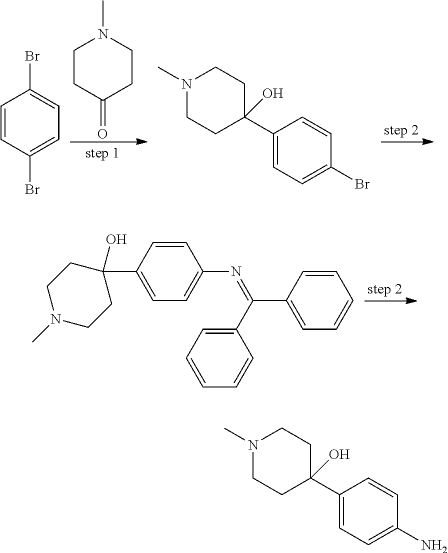

### Step 1: Preparation of 4-(4-bromophenyl)-1-methylpiperidin-4-ol

To a stirred solution of dibromobenzene (5 g, 21.195 mmol, 1 eq.) in THF (15 mL) at −78° C. was added n-BuLi (8.48 mL, 21.195 mmol, 1 eq.) dropwise under nitrogen atmosphere. The resulting mixture was stirred at −78° C. for 30 min. Then 1-methylpiperidin-4-one (2.40 g, 21.195 mmol, 1 eq.) was added dropwise over 5 min at −78° C. The resulting mixture was stirred at rt for 1 h. Then it was quenched with MeOH (5 ml) and concentrated under reduced pressure. The residue was purified by reverse phase flash chromatography (MeCN in Water (10 mmol/L NH₄HCO₃), 10% to 50% gradient in 10 min) to afford 4-(4-bromophenyl)-1-methylpiperidin-4-ol (2 g, 34.93%) as a yellow oil. MS(ESI): mass calcd. for C₂H₁₆BrNO 269.04; [M+H]⁺ found, 270.1.

### Step 2: Preparation of 4-(4-((diphenylmethylene)amino)phenyl)-1-methylpiperidin-4-ol

A mixture of 4-(4-bromophenyl)-1-methylpiperidin-4-ol (1 g, 3.701 mmol, 1 eq.), diphenylmethanimine (931.71 μL, 5.551 mmol, 1.5 eq.), Cs₂CO₃ (2.41 g, 7.402 mmol, 2 eq.), Xantphos (214.17 mg, 0.370 mmol, 0.1 eq.) and Pd₂(dba)₃ (338.95 mg, 0.370 mmol, 0.1 eq.) in Dioxane (10 mL) was stirred at 100° C. under nitrogen atmosphere for 4 h. The resulting mixture was filtered, and the filter cake was washed with dichloromethane (20 ml) and methanol (20 ml). The filtrate was concentrated under reduced pressure. The residue was purified by silica gel column chromatography ((0.2M NH₃ in MeOH))/CH₂Cl₂=1:10) to afford 4-(4-((diphenylmethylene)amino)phenyl)-1-methylpiperidin-4-ol (400 mg, 29.17%) as a yellow solid. MS(ESI): mass calcd. for C₂₅H₂₆N₂O 370.20; [M+H]⁺ found, 371.3.

### Step 3: Preparation of 4-(4-aminophenyl)-1-methylpiperidin-4-ol

To a stirred solution of 4-(4-((diphenylmethylene)amino)phenyl)-1-methylpiperidin-4-ol (360 mg, 0.972 mmol, 1 eq.) in MeOH (4 mL) were added NaOAc (79.71 mg, 0.972 mmol, 1 eq.) and H₂NOH—HCl (67.53 mg, 0.972 mmol, 1 eq.) in portions at it. The resulting mixture was stirred at rt for 1 h. Then it was concentrated under reduced pressure. The residue was purified by prep-TLC (((0.2M NH₃ in MeOH))/CH₂Cl₂=1:20) to afford 4-(4-aminophenyl)-1-methylpiperidin-4-ol (170 mg, 84.81%) as a yellow solid. MS(ESI): mass calcd. for C₁₂H₁₈N₂O 206.14; [M+H]⁺ found, 207.0.

### Preparation of 3-(methoxymethyl)-4-(1-methylpiperidin-4-yl)aniline

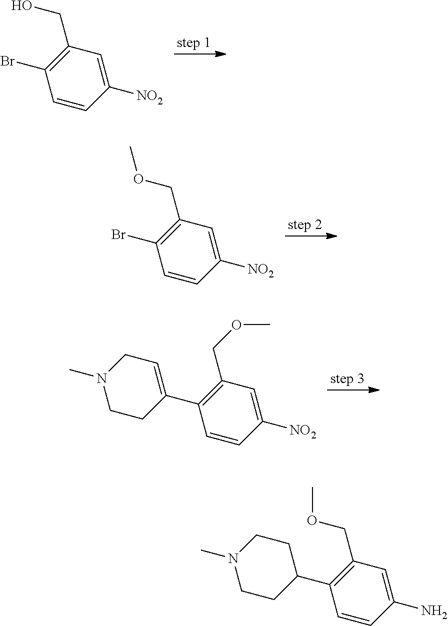 [SMILES: O=[N+]([O-])c1ccc(Br)c(CO)c1 ; COCc1cc([N+](=O)[O-])ccc1Br ; COCc1cc([N+](=O)[O-])ccc1C1=CCN(C)CC1 ; COCc1cc(N)ccc1C1CCN(C)CC1]

### Step 1: Preparation of 1-bromo-2-(methoxymethyl)-4-nitrobenzene

To a solution of (2-bromo-5-nitrophenyl)methanol (700 mg, 3.02 mmol, 1 eq.) in THF (10 mL) at 0° C. was added NaH (241.32 mg, 6.03 mmol, 2.0 eq., 60%) dropwise. The resulting mixture was stirred for an additional 30 min at 0° C. To the above mixture was added methyl iodide (2.57 g, 18.10 mmol, 6.0 eq.) dropwise at 0° C. The resulting mixture was stirred overnight at rt, then was quenched with water (25 ml) at 0° C. The resulting mixture was extracted with EtOAc (3×25 mL). The combined organic layers were washed with brine (1×35 mL) and dried over anhydrous Na₂SO₄. After filtration, the filtrate was concentrated under reduced pressure. The resulting mixture was concentrated under vacuum. The residue was purified by silica gel column chromatography (EA in PE: 10%) to afford 1-bromo-2-(methoxymethyl)-4-nitrobenzene (686 mg, 92.41%).

### Step 2: Preparation of 4-(2-(methoxymethyl)-4-nitrophenyl)-1-methyl-1,2,3,6-tetrahydropyridine

Into a 100 mL vial were added 1-bromo-2-(methoxymethyl)-4-nitrobenzene (686 mg, 2.79 mmol, 1 eq.), K₂CO₃ (924.74 mg, 6.691 mmol, 2.4 eq.), Pd(dppf)Cl₂CH₂Cl₂ (113.56 mg, 0.14 mmol, 0.05 eq.) and dioxane (10 mL) and H₂O (1 mL) at rt. The resulting mixture was stirred at 110° C. under nitrogen atmosphere for 2 h. The residue was purified by reversed-phase flash chromatography under basic conditions to give 4-[2-(methoxymethyl)-4-nitrophenyl]-1-methyl-3,6-dihydro-2H-pyridine (630 mg, 86.15%) as a brown solid. MS (ESI): [M+H]⁺ found, 263.

### Step 3: Preparation of 3-(methoxymethyl)-4-(1-methylpiperidin-4-yl)aniline

Into a 50 mL round-bottom flask were added 4-[2-(methoxymethyl)-4-nitrophenyl]-1-methyl-3,6-dihydro-2H-pyridine (638 mg, 2.432 mmol, 1 eq.), EtOAc (5 mL) and Pd/C (636.75 mg, 5.983 mmol, 2.46 eq.) at room temperature. The resulting mixture was stirred at 50° C. under hydrogen atmosphere overnight. The resulting mixture was filtered, and the filter cake was washed with MeOH. The filtrate was concentrated under reduced pressure. The crude product was purified by Chiral Prep-HPLC under Method b conditions to give 3-(methoxymethyl)-4-(1-methylpiperidin-4-yl)aniline (300 mg, 51.95%) as a yellow oil. MS (ESI): [M+H]⁺ found, 235.

### Preparation of 4-(4-aminophenyl)-1-methylpiperidin-2-one

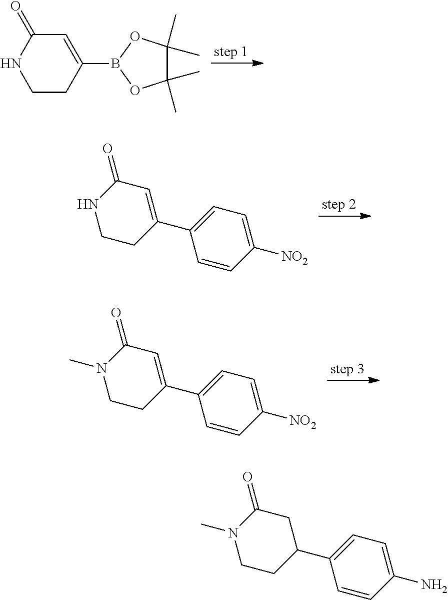

### Step 1: Preparation of 4-(4-nitrophenyl)-5,6-dihydropyridin-2(1H)-one

To a stirred solution of 4-(4,4,5,5-tetramethyl-1,3,2-dioxaborolan-2-yl)-5,6-dihydro-1H-pyridin-2-one (700 mg, 3.14 mmol, 1 eq.) and 4-bromo-1-nitrobenzene (0.63 g, 3.14 mmol, 1 eq.) in dioxane (20 mL) and H₂O (4 mL) were added Na₂CO₃ (1.00 g, 9.414 mmol, 3 eq.) and Pd(PPh₃)₄ (10.36 mg, 0.009 mmol, 0.1 eq.) at room temperature. The resulting mixture was stirred at 120° C. for 3 h and then quenched with water (20 ml) at room temperature. The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed phase flash chromatography with (MeCN in Water (0.1% FA), 10% to 50% gradient in 10 min) to give 4-(4-nitrophenyl)-5,6-dihydro-1H-pyridin-2-one (110 mg, 16.06%) as a yellow solid. MS (ESI): [M+H]⁺ found, 219.

### Step 2: Preparation of 1-methyl-4-(4-nitrophenyl)-5,6-dihydropyridin-2(1H)-one

To a stirred solution of 4-(4-nitrophenyl)-5,6-dihydro-1H-pyridin-2-one (110 mg, 0.50 mmol, 1 eq.) and methyl iodide (37.66 μL, 0.61 mmol, 1.20 eq.) in THF was added NaH (18.15 mg, 0.756 mmol, 1.5 eq.) in portions at room temperature. The resulting mixture was stirred at rt for 3 h and then quenched with water (10 ml). The resulting mixture was extracted with EtOAc (3×30 mL). The combined organic layers were washed with brine (1×30 mL) and dried over anhydrous Na₂SO₄. After filtration, the filtrate was concentrated under reduced pressure. The residue was purified by reversed phase flash chromatography with (McCN in Water (10 mmol/L NH₄HCO₃), 10% to 50% gradient in 10 min) to give 1-methyl-4-(4-nitrophenyl)-5,6-dihydropyridin-2-one (50 mg, 42.71%). MS (ESI): [M+H]⁺ found, 233.

### Step 3: Preparation of 4-(4-aminophenyl)-1-methylpiperidin-2-one

To a stirred solution of 1-methyl-4-(4-nitrophenyl)-5,6-dihydropyridin-2-one (45 mg, 0.194 mmol, 1 eq.) in MeOH (1 mL) was added Pd/C (45 mg, 0.423 mmol, 2.18 eq.) under N₂ atmosphere at rt. The resulting mixture was stirred at 50° C. under hydrogen atmosphere for 2 h. The resulting mixture was filtered, and the filter cake was washed with MeOH (3×10 mL). The combined filtrate was concentrated under reduced pressure. The residue was purified by reversed phase flash chromatography (MeCN in Water (10 mmol/L NH₄HCO₃), 0% to 100% gradient in 10 min) to give 4-(4-aminophenyl)-1-methylpiperidin-2-one (16 mg, 40.42%). MS (ESI): [M+H]⁺ found, 205.

### Preparation of 2,8-dimethyl-1,2,3,4-tetrahydroisoquinolin-6-amine

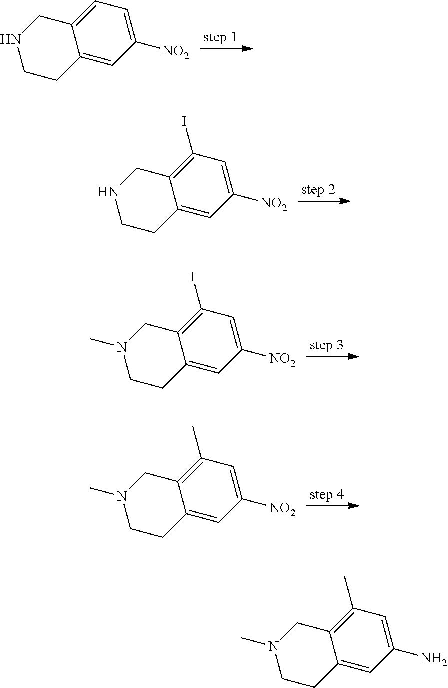

### Step 1: Preparation of 8-iodo-6-nitro-1,2,3,4-tetrahydroisoquinoline

To a stirred solution of 6-nitro-1,2,3,4-tetrahydroisoquinoline (2 g, 11.224 mmol, 1 eq.) in trifluoromethanesulfonic acid (25 mL) was added NIS (3.03 g, 13.469 mmol, 1.2 eq.) in portions at 0° C. The resulting mixture was stirred at rt overnight, and then was quenched by ice and stirred for 10 minutes. To this mixture was added sodium hydroxide (6 M) until a yellow precipitate was formed. The precipitated solids were collected by filtration and washed with water (2×5 mL). The crude product was purified by Prep-HPLC with the following conditions under Method C conditions to afford 8-iodo-6-nitro-1,2,3,4-tetrahydroisoquinoline (1 g, 29.30%) as a light brown solid. MS(ESI): mass calcd. for C₉H₉IN₂O₂ 303.97; [M+H]⁺ found 305.

### Step 2: Preparation of 8-iodo-2-methyl-6-nitro-1,2,3,4-tetrahydroisoquinoline

To a stirred mixture of 8-iodo-6-nitro-1,2,3,4-tetrahydroisoquinoline (1 g, 3.289 mmol, 1 eq.) and NaBH(OAc)₃ (1.39 g, 6.578 mmol, 2 eq.) in DCE (10 mL) was added (HCHO)n (869.21 mg, 19.734 mmol, 6.00 eq.) in portions at 0° C. under nitrogen atmosphere. The reaction was quenched with Water (10 ml) at 0° C. and extracted with CH₂Cl₂ (2×20 mL). The combined organic layers were washed with brine (1×10 mL) and dried over anhydrous Na₂SO₄. After filtration, the filtrate was concentrated under reduced pressure. The residue was purified by Prep-TLC (EA in PE: 50%) to afford 8-iodo-2-methyl-6-nitro-3,4-dihydro-1H-isoquinoline (250 mg, 23.90%) as a light brown solid. MS(ESI): mass calcd. for C₁₀H₁₁IN₂O₂ 317.99; [M+H]⁺ found 319.0.

### Step 3: Preparation of 2,8-dimethyl-6-nitro-1,2,3,4-tetrahydroisoquinoline

To a stirred solution of 8-iodo-2-methyl-6-nitro-3,4-dihydro-1H-isoquinoline (200 mg, 0.629 mmol, 1 eq.) and K₃PO₄ (320.29 mg, 1.510 mmol, 2.4 eq.) in dioxane (I mL) and H₂O (9 mL) was added Pd(dppf)Cl₂·CH₂Cl₂ (25.61 mg, 0.031 mmol, 0.05 eq.) at room temperature under nitrogen atmosphere. The resulting mixture was stirred at 110° C. under nitrogen atmosphere for 2 h. The resulting mixture was concentrated under reduced pressure. The residue was purified by Prep-TLC (MeOH in CH₂Cl₂: 5%) to afford 2,8-dimethyl-6-nitro-3,4-dihydro-1H-isoquinoline (100 mg, 77.12%). MS(ESI): mass calcd. for C₁₁H₁₄N₂O₂ 206.11; [M+H]⁺ found 207.0.

### Step 4: Preparation of 2,8-dimethyl-1,2,3,4-tetrahydroisoquinolin-6-amine

Into a 25 mL round-bottom flask were added 2,8-dimethyl-6-nitro-3,4-dihydro-1H-isoquinoline (100 mg, 0.485 mmol, 1 eq.), Fe (108.31 mg, 1.940 mmol, 4 eq.), NH₄Cl (103.74 mg, 1.940 mmol, 4 eq.) and EtOH (2 mL) at room temperature. The resulting mixture was stirred at 60° C. for 3 h, filtered through a Celite pad and concentrated under reduced pressure to afford 2,8-dimethyl-3,4-dihydro-1H-isoquinolin-6-amine (65 mg, 76.06%) as a light brown solid. MS(ESI): mass calcd. for C₁₁H₁₆N₂ 176.13; [M+H]⁺ found 177.

### Preparation of 2,5-dimethyl-1,2,3,4-tetrahydroisoquinolin-7-amine

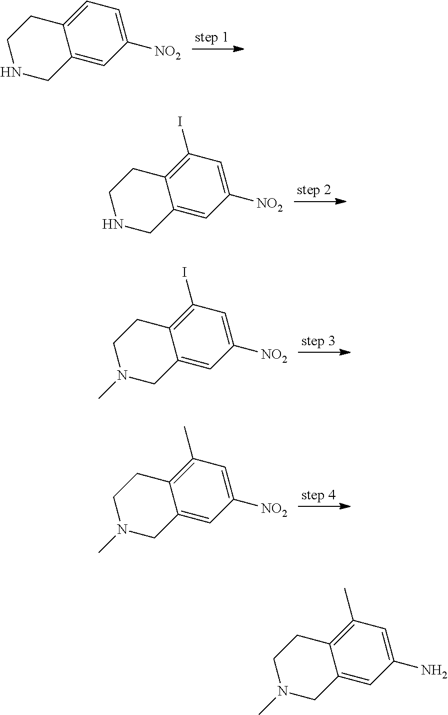

2,5-dimethyl-1,2,3,4-tetrahydroisoquinolin-7-amine was prepared by using the same reaction sequence as the preparation of 2,8-dimethyl-1,2,3,4-tetrahydroisoquinolin-6-amine.

### Preparation of tert-butyl 6-amino-8-methyl-3,4-dihydroisoquinoline-2(1H)-carboxylate

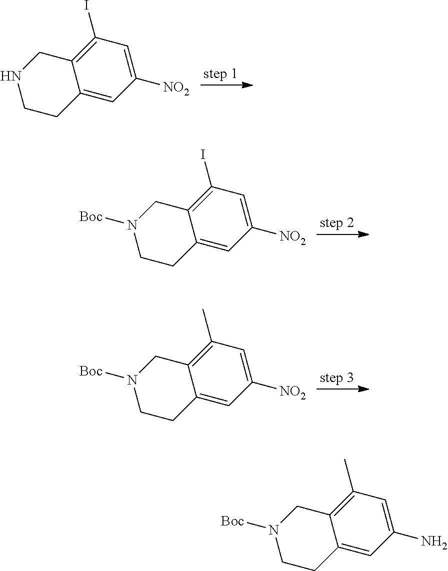 [SMILES: O=[N+]([O-])c1cc(I)c2c(c1)CCNC2 ; CC(C)(C)OC(=O)N1CCc2cc([N+](=O)[O-])cc(I)c2C1 ; Cc1cc([N+](=O)[O-])cc2c1CN(C(=O)OC(C)(C)C)CC2 ; Cc1cc(N)cc2c1CN(C(=O)OC(C)(C)C)CC2]

### Step 1: Preparation of tert-butyl 8-iodo-6-nitro-3,4-dihydroisoquinoline-2(1H)-carboxylate

To a stirred mixture of 8-iodo-6-nitro-1,2,3,4-tetrahydroisoquinoline (520 mg, 1.710 mmol, 1 eq.) and di-tert-butyl dicarbonate (559.82 mg, 2.565 mmol, 1.5 eq.) in DCM (5 mL) was added Et₃N (519.13 mg, 5.130 mmol, 3 eq.) dropwise at rt. The resulting mixture was stirred at rt for 1 h, and then concentrated under reduced pressure. The residue was purified by silica gel column chromatography (EA in PE: 20%) to afford tert-butyl 8-iodo-6-nitro-3,4-dihydro-1H-isoquinoline-2-carboxylate (600 mg, 86.81%). MS(ESI): mass calcd. for C₁₄H₁₇IN₂O₄ 404.02, [M+H]⁺ found, 403.05.

### Step 2: Preparation of tert-butyl 8-methyl-6-nitro-3,4-dihydroisoquinoline-2(1H)-carboxylate

To a stirred mixture of tert-butyl 8-iodo-6-nitro-3,4-dihydro-1H-isoquinoline-2-carboxylate (555 mg, 1.373 mmol, 1 eq.) and methylboronic acid (205.48 mg, 3.433 mmol, 2.5 eq.) in dioxane (7 mL) and H₂O (0.7 mL) were added Cs₂CO₃ (24.18 mg, 0.075 mmol, 3 eq.) and Pd(dppf)Cl₂·CH₂Cl₂ (111.85 mg, 0.137 mmol, 0.1 eq.) in portions at it under nitrogen atmosphere. The resulting mixture was stirred at 110° C. under nitrogen atmosphere for 2 h. The reaction was quenched with Water (10 ml) at 0° C. The resulting mixture was extracted with EtOAc (3×10 mL). The combined organic layers were washed with brine (1×7 mL) and dried over anhydrous Na₂SO₄. After filtration, the filtrate was concentrated under reduced pressure. The residue was purified by silica gel column chromatography (EA in PE: 30%) to afford tert-butyl 8-methyl-6-nitro-3,4-dihydro-1H-isoquinoline-2-carboxylate (340 mg, 84.70%) as a yellow solid.

### Step 3: Preparation of tert-butyl 6-amino-8-methyl-3,4-dihydroisoquinoline-2(1H)-carboxylate

To a stirred mixture of tert-butyl 8-methyl-6-nitro-3,4-dihydro-1H-isoquinoline-2-carboxylate (340 mg, 1.163 mmol, 1 eq.) in EA (6 mL) was added Pd/C (340.37 mg, 3.198 mmol, 2.75 eq.) in portions at room temperature. The resulting mixture was stirred under hydrogen atmosphere overnight. Then it was filtered, and the filter cake was washed with ethyl acetate (3×5 mL). The filtrate was concentrated under reduced pressure to give the crude product (300 mg, 98.32%) as a yellow solid, which was used in the next step directly without further purification MS(ESI): mass calcd. for C₁₅H₂₂N₂O₂ 262.17; [M+H]⁺ found, 263.20.

### Preparation of 4-chloro-2-((4-(4-methylpiperazin-1-yl)phenyl)amino)pyrimidine-5-carbonitrile

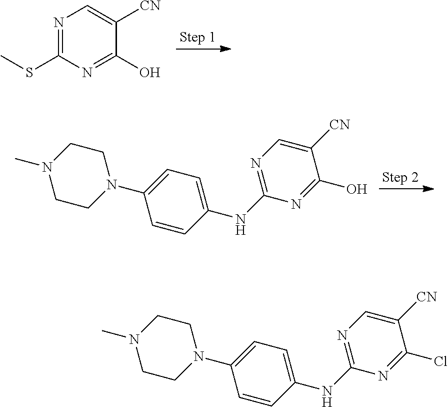 [SMILES: CSc1ncc(C#N)c(O)n1 ; CN1CCN(c2ccc(Nc3ncc(C#N)c(O)n3)cc2)CC1 ; CN1CCN(c2ccc(Nc3ncc(C#N)c(Cl)n3)cc2)CC1]

### Step 1: Preparation of 4-hydroxy-2-((4-(4-methylpiperazin-1-yl)phenyl)amino)pyrimidine-5-carbonitrile

To a solution 4-hydroxy-2-(methylthio)pyrimidine-5-carbonitrile (1.0 g, 5.981 mmol), 4-(4-methylpiperazin-1-yl)aniline (1.14 g, 5.981 mmol) and AcOH (0.718 g, 119.620 mmol) in t-BuOH (15 mL) was stirred in microwave at 130° C. for 3 h. The solvent was removed in vacuo to give 4-hydroxy-2-((4-(4-methylpiperazin-1-yl)phenyl)amino)pyrimidine-5-carbonitrile (1.9 g, 102.7% yield) as a orange crude solid which was used directly in the next step without any further purification. MS (ESI): mass calcd. for C₁₆H₁₈N₆O 310.4; m/z found 311.0 [M+H]⁺. LCMS (method 1): Rt=0.442 min.

### Step 2: preparation of 4-chloro-2-((4-(4-methylpiperazin-1-yl)phenyl)amino)pyrimidine-5-carbonitrile

4-hydroxy-2-((4-(4-methylpiperazin-1-yl)phenyl)amino)pyrimidine-5-carbonitrile (1.9 g, 6.129 mmol) was dissolved in POCl₃ (20 mL) and stirred at 80° C. for 2 h. The mixture was concentrated under reduced pressure at 40° C. The residue was quenched by saturated NaHCO₃ solution to pH=8, and extracted with DCM (50 mL×3). The organic phases were combined, dried over Na₂SO₄, and concentrated under reduced pressure at 30° C. The residue was purified by silica gel flash column chromatography (MeOH in DCM: 0% to 15%) to give 4-chloro-2-((4-(4-methylpiperazin-1-yl)phenyl)amino)pyrimidine-5-carbonitrile (1.1 g, 55.2% yield) as a yellow solid. MS (ESI): mass calcd. for C₁₆H₁₇ClN₆ 328.1; m/z found 329.1 [M+H]⁺. LCMS (method 1): Rt=0.958 min.

### Preparation of 2-chloro-4-((7-ethyl-7-hydroxy-6,7-dihydro-5H-cyclopenta[b]pyridin-2-yl)amino)pyrimidine-5-carbonitrile

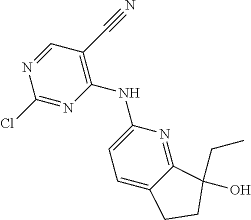 [SMILES: CCC1(O)CCc2ccc(Nc3nc(Cl)ncc3C#N)nc21]

A solution of 2-amino-7-ethyl-5H,6H-cyclopenta[b]pyridin-7-ol (1.5 g, 8.42 mmol, 1 eq.), 2,4-dichloropyrimidine-5-carbonitrile (1.46 g, 8.42 mmol, 1 eq.) and DIEA (2.18 g, 16.832 mmol, 2 eq.) in IPA (10 mL) was stirred at 100° C. for 1 h. The resulting mixture was concentrated under reduced pressure. The residue was purified by reverse phase flash chromatography (MeCN in Water (0.1% FA), 10% to 50% gradient in 10 min) to give 2-chloro-4-({7-ethyl-7-hydroxy-5H,6H-cyclopenta[b]pyridin-2-yl}amino)pyrimidine-5-carbonitrile (1 g, 37.63%) as a yellow solid. MS (ESI): mass calcd. for C₁₅H₁₄ClN₅O 315.09; [M+H]⁺ found 316.

### Preparation of 2-chloro-4-((7-hydroxy-7-methyl-6,7-dihydro-5H-cyclopenta[b]pyridin-2-yl)amino)pyrimidine-5-carbonitrile

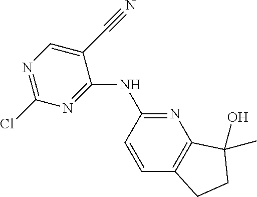 [SMILES: CC1(O)CCc2ccc(Nc3nc(Cl)ncc3C#N)nc21]

2-chloro-4-((7-hydroxy-7-methyl-6,7-dihydro-5H-cyclopenta[b]pyridin-2-yl)amino)pyrimidine-5-carbonitrile was prepared under the same conditions as preparation of 2-chloro-4-((7-ethyl-7-hydroxy-6,7-dihydro-5H-cyclopenta[b]pyridin-2-yl)amino)pyrimidine-5-carbonitrile

### Preparation of 2-chloro-4-((6-(2-oxooxazolidin-3-yl)pyridin-2-yl)amino)pyrimidine-5-carbonitrile

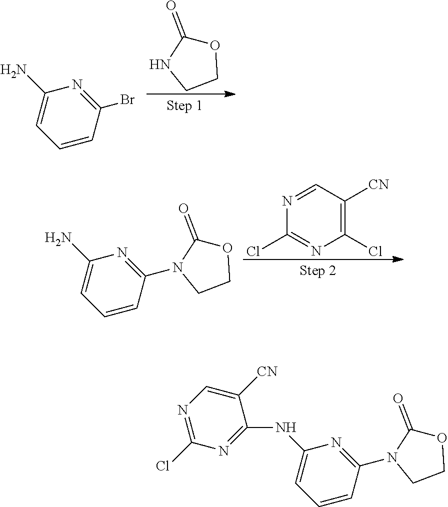

### Step 1: Preparation of 3-(6-aminopyridin-2-yl)oxazolidin-2-one

A mixture of 6-bromopyridin-2-amine (3 g, 0.0173 mol), 1,3-oxazolidin-2-one (1.51 g, 0.0173 mol), K₂CO₃ (7.17 g, 0.0519 mol), DMEDA (0.76 g, 0.00865 mol), CuI (1.65 g, 0.00865 mol) in dioxane (30 mL) was stirred at 100° C. under nitrogen for 2 h. The resulting mixture was concentrated in vacuo to give the crude product, which was purified by silica gel flash column chromatography (MeOH in DCM: 0% to 10%) to afford 3-(6-aminopyridin-2-yl)oxazolidin-2-one (2.2 g, 71.2% yield). MS (ESI): mass calcd. for C₈H₉N₃O₂ 179.07; m/z found 180.1, [M+H]⁺. LCMS (method 1): Rt=0.366 min.

### Step 2: Preparation of 2-chloro-4-((6-(2-oxooxazolidin-3-yl)pyridin-2-yl)amino)pyrimidine-5-carbonitrile

A mixture of 3-(6-aminopyridin-2-yl)oxazolidin-2-one (2.2 g, 12.3 mmol), 2,4-dichloropyrimidine-5-carbonitrile (2.14 g, 12.3 mmol) and DIEA (3.18 g, 24.6 mmol) in THF (22 mL) was stirred at 40° C. for 2 h. LCMS shows that the reaction was finished. The resulting mixture was concentrated in vacuo to give the crude product, which was purified by silica gel flash column chromatography (EA in PE: 0% to 50%) to afford 2-chloro-4-((6-(2-oxooxazolidin-3-yl)pyridin-2-yl)amino)pyrimidine-5-carbonitrile (2.1 g, 48.8% yield). MS (ESI): mass calcd. for C₁₃H₉ClN₆O₂ 316.05; m/z found 317.1 [M+H]⁺. LCMS (method 1): Rt=1.192 min.

### Preparation of 2-chloro-4-((6-(2-oxopyrrolidin-1-yl)pyridin-2-yl)amino)pyrimidine-5-carbonitrile

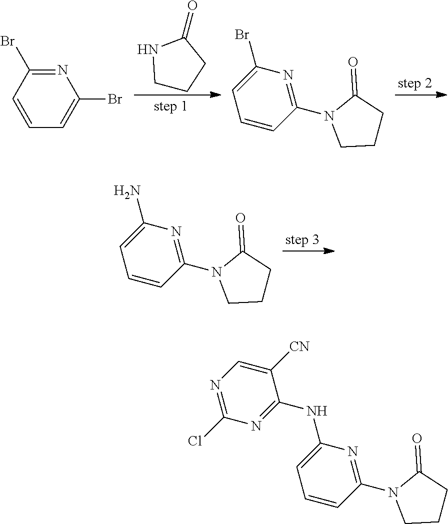

### Step 1: Preparation of N-(6-bromopyridin-2-yl)pyrrolidin-2-one

To a stirred mixture of, 2,6-dibromopyridine (20 g, 84.426 mmol, 1 eq.) and pyrrolidone (35.92 g, 42.21 mmol, 0.5 eq.) in dioxane (30 mL) were added Cs₂CO₃ (55.02 g, 168.85 mmol, 2 eq.), Xantphos (48.85 g, 8.44 mmol, 0.1 eq.) and Pd(OAc)₂ (947.72 mg, 4.22 mmol, 0.05 eq.) in portions at room temperature under nitrogen atmosphere. The resulting mixture was stirred at 80° C. under nitrogen atmosphere overnight. Then it was quenched with water (20 ml) at room temperature and extracted with EtOAc (3×20 mL). The combined organic phases were washed with brine (1×50 mL) and dried over anhydrous Na₂SO₄. After filtration, the filtrate was concentrated under reduced pressure. The residue was purified by reversed phase flash column chromatography (column, C18 silica gel; mobile phase, MeCN in Water (10 mmol/L NH₄HCO₃), 10% to 50% gradient in 10 min; detector. UV 254 nm) to give 1-(6-bromopyridin-2-yl)pyrrolidin-2-one (7.4 g, 36.36%) as a yellow solid. MS (ESI): [M+H]⁺ found, 24.

### Step 2: Preparation of 1-(6-aminopyridin-2-yl)pyrrolidin-2-one

To a stirred mixture of 1-(6-bromopyridin-2-yl)pyrrolidin-2-one (8.8 g, 36.50 mmol, 1 eq.) and methyl[2-(methylamino)ethyl]amine (257.42 mg, 2.92 mmol, 0.08 eq.) in ethane-1,2-diol (40 mL) were added K₂CO₃ (7.57 g, 54.751 mmol, 1.5 eq.) and Cu₂O (417.84 mg, 2.920 mmol, 0.08 eq.) and NH₃·H₂O (40 mL) in portions at room temperature. The resulting mixture was stirred at 60° C. overnight. Then it was quenched with H₂O (10 ml) and extracted with CH₂Cl₂ (3×10 mL). The combined organic layers were washed with brine (1×10 mL) and dried over anhydrous MgSO₄. After filtration, the filtrate was concentrated under reduced pressure. The residue was purified by reversed phase flash chromatography to give 1-(6-aminopyridin-2-yl)pyrrolidin-2-one (2.8 g, 43.29%). MS (ESI): [M+H]⁺ found, 178.

### Step 3: Preparation of 2-chloro-4-((6-(2-oxopyrrolidin-1-yl)pyridin-2-yl)amino)pyrimidine-5-carbonitrile

To a stirred mixture of 1-(6-aminopyridin-2-yl)pyrrolidin-2-one (2 g, 11.29 mmol, 1.0 eq.) and 2,4-dichloropyrimidine-5-carbonitrile (1.96 g, 11.29 mmol, 1.0 eq.) in propan-2-ol (20 mL) was added DIEA (2.92 g, 22.57 mmol, 2.0 eq.) dropwise at room temperature. The resulting mixture was stirred at 100° C. overnight, then it was concentrated under reduced pressure. The residue was purified by reversed phase flash chromatography under basic conditions to give 2-chloro-4-([6-(2-oxopyrrolidin-1-yl)pyridin-2-yl]aminopyrimidine-5-carbonitrile (1.8 g, 50.67%) as a yellow solid. MS (ESI): [M+H]⁺ found, 315.

### Example 1: Preparation of 4-((8-ethyl-8-hydroxy-5,6,7,8-tetrahydroquinolin-2-yl)amino)-2-((4-(4-methylpiperazin-1-yl)phenyl)amino)pyrimidine-5-carbonitrile

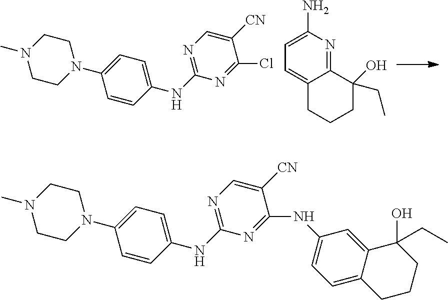

A mixture of 4-chloro-2-((4-(4-methylpiperazin-1-yl)phenyl)amino)pyrimidine-5-carbonitrile (60 mg, 0.18 mmol), 2-amino-8-ethyl-5,6,7,8-tetrahydroquinolin-8-ol (42 mg, 0.22 mmol), RuPhos (16 mg, 0.04 mmol), Pd₂(dba)₃ (32 mg, 0.04 mmol), K₂CO₃ (50 mg, 0.36 mmol) in toluene (5 mL) was stirred under reflux for 2 h under N₂. After the solvent was removed in vacuo, the resulting crude product was purified by silica gel flash column chromatography (PE/EA: 0-50%) to afford 4-((8-ethyl-8-hydroxy-5,6,7,8-tetrahydroquinolin-2-yl)amino)-2-((4-(4-methylpiperazin-1-yl)phenyl)amino)pyrimidine-5-carbonitrile (12.7 mg, 8.4% yield). MS (ESI): mass calcd. for C₂₇H₃₂N₈O 484.27; [M+H]⁺ found 485.3. LCMS (method 1): Rt=1.034 min. ¹H NMR (400 MHz, DMSO-d₆) d ppm 9.88 (bs, 1H), 8.91 (bs, 1H), 8.53 (s, 1H), 7.65 (s, 1H), 7.45-7.41 (m, 3H), 6.88 (d, J=8.4 Hz, 2H), 4.57 (s, 1H), 3.12-3.05 (m, 4H), 2.76-2.59 (m, 2H), 2.47-2.40 (m, 4H), 2.22 (s, 31H), 1.90-1.80 (m, 4H), 1.81-1.63 (m, 2H), 0.79 (t, J=7.2 Hz, 3H).

### Example 2: Preparation of 4-((7-hydroxy-7-propyl-6,7-dihydro-5H-cyclopenta[b]pyridin-2-yl)amino)-2-((4-(4-methylpiperazin-1-yl)phenyl)amino)pyrimidine-5-carbonitrile

4-((7-hydroxy-7-propyl-6,7-dihydro-5H-cyclopenta[b]pyridin-2-yl)amino)-2-((4-(4-methylpiperazin-1-yl)phenyl)amino)pyrimidine-5-carbonitrile was prepared and purified under conditions similar to example 1 by using 2-amino-7-propyl-6,7-dihydro-5H-cyclopenta[b]pyridin-7-ol (70.0 mg, 0.36 mmol) and 4-chloro-2-((4-(4-methylpiperazin-1-yl)phenyl)amino)pyrimidine-5-carbonitrile (144.0 mg, 0.44 mmol) to afford the title compound (70.0 mg, 39.7% yield) as a yellow solid. MS (ESI): mass calcd. for C₂₇H₃₂N₈O 484.3; [M+H]⁺ found 485.3. LCMS (method 1): Rt=1.028 min. ¹H NMR (400 MHz, DMSO-d₆) d ppm 9.86 (bs, 1H), 9.09 (bs, 1H), 8.52 (s, 1H), 7.72-7.33 (m, 4H), 6.86-6.83 (m, 2H), 4.92 (s, 1H), 3.13-3.09 (m, 4H), 2.91-2.85 (m, 1H), 2.71-2.64 (m, 1H), 2.54-2.52 (m, 4H), 2.28 (s, 3H), 2.18-2.13 (m, 1H), 2.04-1.97 (m, 1H), 1.86-1.79 (m, 1H), 1.64-1.57 (m, 1H), 1.49-1.41 (m, 1H), 1.24-1.14 (m, 1H), 0.85 (t, J=6.8 Hz, 3H).

### Example 3: Preparation of 2-((4-(4-methylpiperazin-1-yl)phenyl)amino)-4-((2-oxo-2H-[1,2′-bipyridin]-6′-yl)amino)pyrimidine-5-carbonitrile

2-((4-(4-methylpiperazin-1-yl)phenyl)amino)-4-((2-oxo-2H-[1,2′-bipyridin]-6′-yl)amino)pyrimidine-5-carbonitrile was prepared and purified under conditions similar to example 1 by using 6′-amino-2H-[1,2′-bipyridin]-2-one (50 mg, 0.267 mmol) and 4-chloro-2-((4-(4-methylpiperazin-1-yl)phenyl)amino)pyrimidine-5-carbonitrile (87.83 mg, 0.267 mmol) to afford the title compound (31.2 mg, 24.40% yield). MS (ESI): mass calcd. for C₂₆H₂₅N₉O 479.22; [M+H]⁺ found 480.0. LCMS (method 1): Rt=0.975 min. ¹H NMR (400 MHz, DMSO-d₆) δ ppm 9.94 (s, 1H), 8.57 (s, 1H), 8.39-8.18 (m, 1H), 8.12-7.85 (m, 2H), 7.58-7.35 (m, 4H), 6.94-6.78 (m, 2H), 6.52 (d, J=8.0 Hz, 1H), 6.42-6.25 (m, 1H), 3.10-3.02 (m, 4H), 2.49-2.44 (m, 4H), 2.23 (s, 3H).

### Example 4: Preparation of 4-((7-methoxy-7-methyl-6,7-dihydro-5H-cyclopenta[b]pyridin-2-yl)amino)-2-((4-(4-methylpiperazin-1-yl)phenyl)amino)pyrimidine-5-carbonitrile

4-((7-methoxy-7-methyl-6,7-dihydro-5H-cyclopenta[b]pyridin-2-yl)amino)-2-((4-(4-methylpiperazin-1-yl)phenyl)amino)pyrimidine-5-carbonitrile was prepared and purified under conditions similar to example 1 by using 7-methoxy-7-methyl-6,7-dihydro-5H-cyclopenta[b]pyridin-2-amine (80 mg, 0.449 mmol) and 4-chloro-2-((4-(4-methylpiperazin-1-yl)phenyl)amino)pyrimidine-5-carbonitrile (147.60 mg, 0.449 mmol) to afford the title compound (40.1 mg, 18.95% yield). MS (ESI): mass calcd. for C₂₆H₃₀N₈O 470.25; [M+H]⁺ found 471.2. LCMS (method 1): Rt=1.020 min. ¹H NMR (400 MHz, DMSO-d₆) d ppm 9.83 (bs, 1H), 9.35 (bs, 1H), 8.49 (s, 1H), 7.73-7.65 (m, 2H), 7.52-7.25 (m, 2H), 6.80-6.75 (m, 2H), 3.25-3.18 (m, 4H), 2.99 (s, 3H), 2.95-2.78 (m, 4H), 2.75-2.61 (m, 1H), 2.56-2.51 (m, 4H), 2.28-2.26 (m, 1H), 1.98-1.93 (m, 1H), 1.42 (s, 3H).

### Example 5: Preparation of 2-((4-(4-methylpiperazin-1-yl)phenyl)amino)-4-((6-(2-oxopyrrolidin-1-yl)pyridin-2-yl)amino)pyrimidine-5-carbonitrile

2-((4-(4-methylpiperazin-1-yl)phenyl)amino)-4-((6-(2-oxopyrrolidin-1-yl)pyridin-2-yl)amino)pyrimidine-5-carbonitrile was prepared and purified under conditions similar to example 1 by using 1-(6-aminopyridin-2-yl)pyrrolidin-2-one (100 mg, 0.564 mmol) and 4-chloro-2-((4-(4-methylpiperazin-1-yl)phenyl)amino)pyrimidine-5-carbonitrile (111.32 mg, 0.339 mmol) to afford the title compound (43.4 mg, 16.4% yield) as white solid. MS (ESI): mass calcd. for C₂₅H₂₇N₉O 469.23; [M+H]⁺ found 470.0. LCMS (method 1): Rt=1.015 min. ¹H NMR (400 MHz, DMSO-d₆) δ ppm 9.90 (s, 1H), 9.49 (s, 1H), 8.54 (s, 1H), 8.04 (d, J=7.2 Hz, 1H), 7.81-7.75 (m, 1H), 7.61-7.25 (m, 3H), 6.91-6.68 (m, 2H), 4.05-3.81 (m, 2H), 3.08-3.02 (m, 4H), 2.61-2.52 (m, 2H), 2.49-2.41 (m, 4H), 2.23 (s, 3H), 2.11-1.89 (m, 2H).

### Example 6: Preparation of 4-((8-ethyl-5,6,7,8-tetrahydro-1,8-naphthyridin-2-yl)amino)-2-((4-(4-methylpiperazin-1-yl)phenyl)amino)pyrimidine-5-carbonitrile

4-((8-ethyl-5,6,7,8-tetrahydro-1,8-naphthyridin-2-yl)amino)-2-((4-(4-methylpiperazin-1-yl)phenyl)amino)pyrimidine-5-carbonitrile was prepared and purified under conditions similar to example 1 by using 4-chloro-2-((4-(4-methylpiperazin-1-yl)phenyl)amino)pyrimidine-5-carbonitrile (100 mg, 0.30 mmol) and 8-ethyl-5,6,7,8-tetrahydro-1,8-naphthyridin-2-amine (65 mg, 0.37 mmol) to afford the title compound (20.5 mg, 14.3% yield). MS (ESI): mass calcd. for C₂₆H₃₁N₉ 469.27; [M+H]⁺ found 470.3. LCMS (method 1): Rt=1.058 min. ¹H NMR (400 MHz, DMSO-d₆) d ppm 9.80 (s, 11H), 8.69 (s, 1H), 8.47 (s, 1H), 8.23 (s, 1H), 7.46 (d, J=8.0 Hz, 2H), 7.16-7.08 (m, 1H), 6.91-6.78 (m, 2H), 3.58-3.51 (m, 3H), 3.36-3.31 (m, 2H), 3.11-3.04 (m, 4H), 2.67 (t, J=6.0 Hz, 2H), 2.48-2.41 (m, 4H), 2.22 (s, 3H), 1.89-1.81 (m, 2H), 1.05 (t, J=6.4 Hz, 3H).

### Example 7: Preparation of 2-((4-(4-methylpiperazin-1-yl)phenyl)amino)-4-((6-(pyrrolidin-1-yl)pyridin-2-yl)amino)pyrimidine-5-carbonitrile

2-((4-(4-methylpiperazin-1-yl)phenyl)amino)-4-((6-(pyrrolidin-1-yl)pyridin-2-yl)amino)pyrimidine-5-carbonitrile was prepared and purified under conditions similar to example 1 by using 4-chloro-2-((4-(4-methylpiperazin-1-yl)phenyl)amino)pyrimidine-5-carbonitrile (100 mg, 0.30 mmol) and 6-(pyrrolidin-1-yl)pyridin-2-amine (60 mg, 0.37 mmol) to afford the title compound (15 mg, 10.8% yield). MS (ESI): mass calcd. for C₂₅H₂₉N₉ 455.25; [M+H]⁺ found 456.2. LCMS (method 1): Rt=0.999 min. ¹H NMR (400 MHz, DMSO-d₆) d ppm 9.85 (s, 1H), 8.60 (s, 11H), 8.50 (s, 11H), 7.45-7.42 (m, 3H), 6.86-6.81 (m, 3H), 6.17 (d, J=8.0 Hz, 1H), 3.38-3.34 (m, 4H), 3.12-3.08 (m, 4H), 2.60-2.55 (m, 4H), 2.30 (s, 3H), 1.92-1.88 (m, 4H).

### Example 8: Preparation of 4-((7-hydroxy-7-methyl-6,7-dihydro-5H-cyclopenta[b]pyridin-2-yl)amino)-2-((4-(4-methylpiperazin-1-yl)phenyl)amino)pyrimidine-5-carbonitrile

4-((7-hydroxy-7-methyl-6,7-dihydro-5H-cyclopenta[b]pyridin-2-yl)amino)-2-((4-(4-methylpiperazin-1-yl)phenyl)amino)pyrimidine-5-carbonitrile was prepared and purified under conditions similar to example 1 by using 4-chloro-2-((4-(4-methylpiperazin-1-yl)phenyl)amino)pyrimidine-5-carbonitrile (80.10 mg, 0.244 mmol) and 2-amino-7-methyl-5H,6H-cyclopenta[b]pyridin-7-ol (40 mg, 0.244 mmol) to afford the title compound (5.5 mg, 4.9% yield). MS (ESI): mass calcd. for C₂₅H₂₈N₈O 456.24; [M+H]⁺ found 457.2. LCMS (method 1): Rt=0.984 min. ¹H NMR (400 MHz, DMSO-d₆) δ ppm 9.86 (s, 1H), 9.15 (s, 1H), 8.52 (s, 1H), 8.21 (s, 1H), 7.68-7.61 (m, 1H), 7.51-7.31 (m, 2H), 6.91-6.75 (m, 2H), 5.08 (s, 1H), 3.31-3.08 (m, 4H), 2.85-2.81 (m, 1H), 2.77-2.65 (m, 1H), 2.51-2.46 (m, 4H), 2.15 (s, 3H), 2.14-2.07 (m, 2H), 1.42 (s, 3H).

### Example 9: Preparation of (S)-2-((4-(2,4-dimethylpiperazin-1-yl)phenyl)amino)-4-((6-(2-oxooxazolidin-3-yl)pyridin-2-yl)amino)pyrimidine-5-carbonitrile

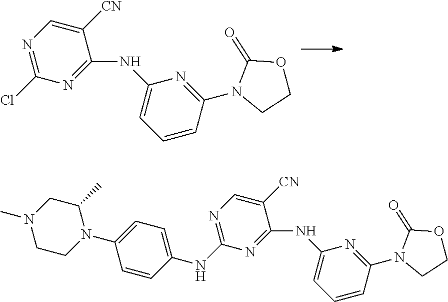 [SMILES: N#Cc1cnc(Cl)nc1Nc1cccc(N2CCOC2=O)n1 ; C[C@H]1CN(C)CCN1c1ccc(Nc2ncc(C#N)c(Nc3cccc(N4CCOC4=O)n3)n2)cc1]

A mixture of 2-chloro-4-((6-(2-oxooxazolidin-3-yl)pyridin-2-yl)amino)pyrimidine-5-carbonitrile (40 mg, 0.13 mmol), (S)-4-(2,4-dimethylpiperazin-1-yl)aniline (39 mg, 0.19 mmol) and AcOH (152 mg, 2.53 mmol) in t-BuOH (2 mL) was stirred at 130° C. under microwave for 2 h. Then the mixture was filtered and the filtrate was concentrated in vacuo to give the crude product, which was purified by prep-HPLC under method A conditions to afford (S)-2-((4-(2,4-dimethylpiperazin-1-yl)phenyl)amino)-4-((6-(2-oxooxazolidin-3-yl)pyridin-2-yl)amino)pyrimidine-5-carbonitrile (7 mg, 11.4% yield) as a yellow solid. MS (ESI): mass calcd. for C₂₅H₂₇N₉O₂ 485.23; [M+H]⁺ found 486.2. LCMS (method 1): Rt=1.039 min. ¹H NMR (400 MHz, CD₃OD) d 8.41 (s, 2H), 7.82 (d, J=8.8 Hz, 1H), 7.68 (s, 1H), 7.55-7.45 (m, 2H), 7.02 (d, J=8.8 Hz, 2H), 4.44 (t, J=8.0 Hz, 2H), 4.25-4.15 (m, 2H), 3.71-3.61 (m, 1H), 3.22-3.16 (m, 2H), 3.12-2.98 (m, 3H), 2.87-2.79 (m, 1H), 2.66 (s, 3H), 1.00 (d, J=6.8 Hz, 3H).

### Example 10: Preparation of (R)-2-((4-(2,4-dimethylpiperazin-1-yl)phenyl)amino)-4-((6-(2-oxooxazolidin-3-yl)pyridin-2-yl)amino)pyrimidine-5-carbonitrile

(R)-2-((4-(2,4-dimethylpiperazin-1-yl)phenyl)amino)-4-((6-(2-oxooxazolidin-3-yl)pyridin-2-yl)amino)pyrimidine-5-carbonitrile was prepared and purified under conditions similar to example 9 by using 2-chloro-4-((6-(2-oxooxazolidin-3-yl)pyridin-2-yl)amino)pyrimidine-5-carbonitrile (40 mg, 0.1 mmol) and (R)-4-(2,4-dimethylpiperazin-1-yl)aniline (33.9 mg, 0.1 mmol) to afford the title compound (14 mg, 22.79% yield) as a yellow solid. MS (ESI): mass calcd. for C₂₅H₂₇N₉O₂ 485.2; [M+H]⁺ found 486.2. LCMS (method 1): Rt=1.040 min. ¹H NMR (400 MHz, DMSO-d₆) δ ppm 9.63 (s, 1H), 8.51 (s, 1H), 7.79-7.76 (m, 2H), 7.66-7.56 (m, 1H), 7.44 (d, J=8.8 Hz, 2H), 6.83 (d, J=9.2 Hz, 2H), 4.44-4.40 (m, 2H), 4.17-4.13 (m, 2H), 3.82-3.78 (m, 1H), 3.17-3.13 (m, 2H), 3.01-2.98 (m, 1H), 2.72-2.65 (m, 1H), 2.54 (s, 3H), 2.37-2.33 (m, 1H), 2.22 (s, 3H), 2.17-2.14 (m 1H), 1.00 (d, J=6.4 Hz, 3H).

### Example 11: Preparation of 2-((3-methyl-4-(1-methylpiperidin-4-yl)phenyl)amino)-4-((6-(2-oxooxazolidin-3-yl)pyridin-2-yl)amino)pyrimidine-5-carbonitrile

2-((3-methyl-4-(1-methylpiperidin-4-yl)phenyl)amino)-4-((6-(2-oxooxazolidin-3-yl)pyridin-2-yl)amino)pyrimidine-5-carbonitrile was prepared and purified under conditions similar to example 9 by using 2-chloro-4-((6-(2-oxooxazolidin-3-yl)pyridin-2-yl)amino)pyrimidine-5-carbonitrile (30 mg, 0.095 mmol) and 3-methyl-4-(1-methylpiperidin-4-yl)aniline (19.35 mg, 0.095 mmol) to afford the title compound (10.4 mg, 23.2% yield). MS (ESI): mass calcd. for C₂₆H₂₈N₈O₂ 484.23; [M+H]⁺ found 185.2. LCMS (method 1): Rt=1.037 min. ¹H NMR (400 MHz, DMSO-d₆) δ ppm 9.95 (s, 1H), 9.53 (s, 1H), 8.55 (s, 1H), 8.20 (s, 2H), 7.81-7.75 (m, 2H), 7.40-7.33 (m, 2H), 7.06-7.01 (m, 1H), 4.37-4.30 (m, 2H), 4.10-4.05 (m, 2H), 3.05-2.90 (m, 2H), 2.67-2.62 (m, 1H), 2.34-2.28 (m, 3H), 2.28-2.24 (m, 1H), 2.19-2.14 (m, 3H), 1.71-1.56 (m, 4H).

### Example 12: Preparation of 2-((2-methyl-1,2,3,4-tetrahydroisoquinolin-6-yl)amino)-4-((6-(2-oxooxazolidin-3-yl)pyridin-2-yl)amino)pyrimidine-5-carbonitrile

A mixture of 2-chloro-4-((6-(2-oxooxazolidin-3-yl)pyridin-2-yl)amino)pyrimidine-5-carbonitrile (40 mg, 0.13 mmol), 2-methyl-1,2,3,4-tetrahydroisoquinolin-6-amine (25 mg, 0.15 mmol) and p-Toluenesulfonic acid (45 mg, 0.26 mmol) in isopropyl alcohol (5 mL) was heated to 100° C. and stirred under N₂ overnight. The mixture was treated with saturated sodium carbonate solution (2 mL) and stirred at room temperature for 1 hour, then formic acid (0.5 mL) was added. The resulting mixture was purified by prep-HPLC under method A conditions to afford 2-((2-methyl-1,2,3,4-tetrahydroisoquinolin-6-yl)amino)-4-((6-(2-oxooxazolidin-3-yl)pyridin-2-yl)amino)pyrimidine-5-carbonitrile (19 mg, 33.98% yield) as a yellow solid. MS (ESI): mass calcd. for C₂₃H₂₂N₈O₂ 442.19; [M+H]⁺ found 443.1. LCMS (method 1): Rt=0.985 min. ¹H NMR (400 MHz, CD₃OD) d ppm 8.45 (s, 1H), 8.36 (s, 11H), 7.84 (d, J=8.8 Hz, 1H), 7.78-7.75 (m, 1H), 7.68-7.65 (m, 1H), 7.58 (s, 1H), 7.48-7.40 (m, 1H), 7.22-7.18 (m, 1H), 7.09 (d, J=8.4 Hz, 1H), 4.48-4.44 (m, 2H), 4.24-4.19 (m, 4H), 3.41-3.35 (m, 2H), 3.10-3.02 (m, 2H), 2.91 (s, 3H).

### Example 13: Preparation of 2-((8-chloro-2-methyl-1,2,3,4-tetrahydroisoquinolin-6-yl)amino)-4-((6-(2-oxooxazolidin-3-yl)pyridin-2-yl)amino)pyrimidine-5-carbonitrile

2-((8-chloro-2-methyl-1,2,3,4-tetrahydroisoquinolin-6-yl)amino)-4-((6-(2-oxooxazolidin-3-yl)pyridin-2-yl)amino)pyrimidine-5-carbonitrile was prepared and purified under conditions similar to example 12 by using 2-chloro-4-((6-(2-oxooxazolidin-3-yl)pyridin-2-yl)amino)pyrimidine-5-carbonitrile ((38.0 mg, 0.120 mmol) and 8-chloro-2-methyl-1,2,3,4-tetrahydroisoquinolin-6-amine (23.5 mg, 0.120 mmol) to afford the title compound (6.5 mg, 11.35% yield) as a yellow solid. MS (ESI): mass calcd. for C₂₃H₂₁ClN₈O₂ 476.1. [M+H]⁺ found 477.1. LCMS (method 1): Rt=1.021 min. ¹H NMR (400 MHz, DMSO-d₆) d ppm 10.13 (s, 11H), 8.63 (s, 1H), 7.88-7.82 (m, 2H), 7.64 (s, 1H), 7.47-7.33 (m, 2H), 4.40-4.36 (m, 2H), 4.11-4.10 (m, 2H), 3.43-3.35 (m, 2H), 2.76-2.67 (m, 2H), 2.56-2.53 (m, 2H), 2.37 (s, 3H).

### Example 14: Preparation of 4-((7-ethyl-7-hydroxy-6,7-dihydro-5H-cyclopenta[b]pyridin-2-yl)amino)-2-((6-(4-methylpiperazin-1-yl)pyridin-3-yl)amino)pyrimidine-5-carbonitrile

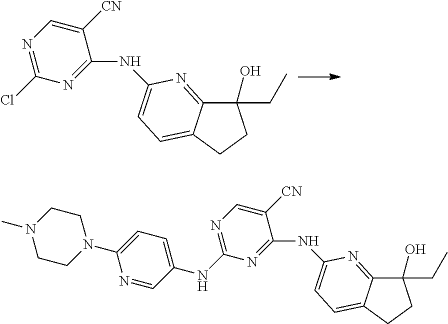

2-chloro-4-[(7-ethyl-7-hydroxy-5,6-dihydrocyclopenta[b]pyridin-2-yl)amino]pyrimidine-5-carbonitrile (1.00 eq, 20 mg, 0.0633 mmol) and 6-(4-methylpiperazin-1-yl)pyridin-3-amine (2.00 eq, 24 mg, 0.127 mmol) were suspended in a solution of Trifluoroacetic acid, 99% (1.00 eq, 0.0049 mL, 0.0633 mmol) in 1-butanol (2 ml). The reaction was stirred at rt overnight. The reaction mixture was quenched with saturated NaHCO₃ solution and extracted with EtOAc. The combined organic phases were purified on pre-HPLC under method B conditions to give 4-[(7-ethyl-7-hydroxy-5,6-dihydrocyclopenta[b]pyridin-2-yl)amino]-2-[[6-(4-methylpiperazin-1-yl)-3-pyridyl]amino]pyrimidine-5-carbonitrile (2.01 mg, 6.7%). ¹H NMR (400 MHz, DMSO) δ 0.85 (3H, t, J=6.5 Hz), 1.67 (1H, m), 1.86 (11H, m), 2.00 (1H, m), 2.15 (11H, m), 2.22 (3H, s), 2.40 (4H, t, J=5 Hz), 2.67 (1H, m), 2.90 (1H, m), 3.43 (4H, t, J=5 Hz), 4.98 (1H, s), 6.80 (1H, d, J=2.5 Hz), 7.53 (1H, m), 7.67 (1H, s), 7.77 (1H, s), 8.29 (11H, s), 8.30 (1H, d, J=2.5 Hz), 8.52 (1H, s), 9.25 (1H, s), 9.87 (1H, s). ESI-LCMS calcd. for C₂₅H₂₉N₉O⁺ [M+H]⁺ 471.25, found 472.12.

### Example 15: Preparation of 4-((7-ethyl-7-hydroxy-6,7-dihydro-5H-cyclopenta[b]pyridin-2-yl)amino)-2-((4-(4-methylpiperazin-1-yl)phenyl)amino)pyrimidine-5-carbonitrile

4-((7-ethyl-7-hydroxy-6,7-dihydro-5H-cyclopenta[b]pyridin-2-yl)amino)-2-((4-(4-methylpiperazin-1-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (100 mg, 0.317 mmol, 1 eq.) and 4-(4-methylpiperazin-1-yl)aniline (90.87 mg, 0.476 mmol, 1.5 eq.) to afford the title compound (20.5 mg, 13.40%) as a yellow solid. MS (ESI) [M+H]⁺ found, 471. ¹H NMR (300 MHz, Methanol-d4): δ 8.51-7.85 (m, 2H), 7.70-7.31 (m, 3H), 7.04-6.93 (m, 2H), 3.28 (s, 4H), 2.94 (s, 5H), 2.86-2.70 (m, 11H), 2.59 (s, 3H), 2.33 (ddd, J=13.4, 8.4, 5.0 Hz, 1H), 2.11 (ddd, J=14.1, 8.9, 6.1 Hz, 1H), 1.95 (dt, J=15.0, 7.5 Hz, 1H), 1.82 (dd, J=13.9, 7.2 Hz, 1H), 0.92 (t, J=7.5 Hz, 3H).

### Example 16: Preparation of 4-((7-ethyl-7-hydroxy-6,7-dihydro-5H-cyclopenta[b]pyridin-2-yl)amino)-2-((3-methyl-4-(4-methylpiperazin-1-yl)phenyl)amino)pyrimidine-5-carbonitrile

4-((7-ethyl-7-hydroxy-6,7-dihydro-5H-cyclopenta[b]pyridin-2-yl)amino)-2-((3-methyl-4-(4-methylpiperazin-1-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (100 mg, 0.317 mmol, 1 eq.) and 3-methyl-4-(4-methylpiperazin-1-yl)aniline (97.53 mg, 0.476 mmol, 1.5 eq.) to afford the title compound (15.7 mg, 10.07%). MS (ESI) [M+H]⁺ found, 485. ¹H NMR (300 MHz, Methanol-d4) δ 8.42 (t, J=1.6 Hz, 1H), 8.09 (s, 1H), 7.57 (s, 1H), 7.43 (s, 1H), 7.34 (s, 1H), 7.05 (d, J=8.8 Hz, 1H), 3.05 (s, 9H), 2.78 (dt, J=15.7, 7.3 Hz, 1H), 2.67 (s, 3H), 2.41-2.23 (m, 4H), 2.11 (dt, J=14.0, 7.4 Hz, 1M), 1.95 (p, J=7.1 Hz, 1H), 1.82 (dd, J=14.2, 7.2 Hz, 11H), 0.92 (t, J=7.5 Hz, 3H).

### Example 17: Preparation of 4-((7-ethyl-7-hydroxy-6,7-dihydro-5H-cyclopenta[b]pyridin-2-yl)amino)-2-((3-methyl-4-(piperazin-1-yl)phenyl)amino)pyrimidine-5-carbonitrile carbonitrile

### Step 1: Preparation of tert-butyl 4-(4-((5-cyano-4-((7-ethyl-7-hydroxy-6,7-dihydro-5H-cyclopenta[b]pyridin-2-yl)amino)pyrimidin-2-yl)amino)-2-methylphenyl)piperazine-1-carboxylate

4-((7-ethyl-7-hydroxy-6,7-dihydro-5H-cyclopenta[b]pyridin-2-yl)amino)-2-((3-methyl-4-(4-methylpiperazin-1-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (100 mg, 0.317 mmol, 1 eq.) and tert-butyl 4-(4-amino-2-methylphenyl)piperazine-1-carboxylate (101.51 mg, 0.349 mmol, 1.1 eq.) to afford the title compound (100 mg, 55.33%) as a yellow solid. MS (ESI): mass calcd. for C₃₁H₃₈N₈O₃ 570.31; [M+H]⁺ found, 571.

### Step 2: Preparation of 4-((7-ethyl-7-hydroxy-6,7-dihydro-5H-cyclopenta[b]pyridin-2-yl)amino)-2-((3-methyl-4-(piperazin-1-yl)phenyl)amino)pyrimidine-5-carbonitrile

To a solution of tert-butyl 4-(4-([5-cyano-4-((7-ethyl-7-hydroxy-5H,6H-cyclopenta[b]pyridin-2-ylamino)pyrimidin-2-yl]amino-2-methylphenyl)piperazine-1-carboxylate (50 mg, 0.088 mmol, 1 eq.) and 2,6-lutidine (121 μL, 1.039 mmol, 11.86 eq.) in DCM (2 mL) at 0° C. was added TMSOTf (190 μL, 1.050 mmol, 11.98 eq.) dropwise. The resulting mixture was stirred at room temperature overnight, quenched with water (20 ml) at 0° C., then extracted with CH₂Cl₂ (3×15 mL). The combined organic layers were washed with brine (1×25 mL) and dried over anhydrous Na₂SO₄. After filtration, the filtrate was concentrated under reduced pressure. The crude product was purified by Chiral-Prep-HPLC under Method a conditions to give 4-((7-ethyl-7-hydroxy-5H,6H-cyclopenta[b]pyridin-2-ylamino)-2-([3-methyl-4-(piperazin-1-yl)phenyl]aminopyrimidine-5-carbonitrile (6.1 mg, 14.35%). MS (ESI) [M+H]⁺ found, 471. ¹H NMR (300 MHz, Methanol-d4) δ 8.43 (s, 11H), 8.12 (s, 1H), 7.60 (s, 1H), 7.48 (d, J=2.5 Hz, 11H), 7.38 (s, 11H), 7.07 (d, J=8.6 Hz, 1H), 3.39 (s, 4H), 3.13 (t, J=5.0 Hz, 4H), 3.05-2.89 (m, 1H), 2.87-2.70 (m, 1H), 2.41-2.25 (m, 4H), 2.12 (ddd, J=14.1, 8.8, 6.0 Hz, 1H), 1.97 (dq, J=14.9, 7.4 Hz, 1H), 1.83 (dq, J=14.4, 7.4 Hz, 1H), 0.92 (t, J=7.5 Hz, 3H).

### Example 18: Preparation of 4-((7-ethyl-7-hydroxy-6,7-dihydro-5H-cyclopenta[b]pyridin-2-yl)amino)-2-((3-methyl-4-(piperidin-4-yl)phenyl)amino)pyrimidine-5-carbonitrile

4-((7-ethyl-7-hydroxy-6,7-dihydro-5H-cyclopenta[b]pyridin-2-yl)amino)-2-((3-methyl-4-(piperidin-4-yl)phenyl)amino)pyrimidine-5-carbonitrile was prepared and purified under conditions similar to example 17 by using 2-Chloro-4-[(7-ethyl-7-hydroxy-5,6-dihydrocyclopenta[b]pyridin-2-yl)amino]pyrimidine-5-carbonitrile (83.72 mg, 0.265 mmol, 1.1 eq.) and tert-butyl 4-(4-amino-2-methylphenyl)piperidine-1-carboxylate (70 mg, 0.241 mmol, 1 eq.) to afford the title compound (2.5 mg, 3.78%). MS (ESI) [M+H]⁺ found, 470. ¹H NMR (300 MHz, Methanol-d4): δ 8.55 (s, 1H), 8.43 (s, 1H), 8.12 (s, 1H), 7.59 (s, 11H), 7.39 (d, J=14.6 Hz, 2H), 7.16 (d, J=8.4 Hz, 1H), 3.42 (d, J=12.5 Hz, 21-1), 3.18-2.99 (m, 3H), 3.00-2.89 (m, 1H), 2.79 (dt, J=15.7, 7.3 Hz, 1H), 2.43-2.25 (m, 4H), 2.12 (ddd, J=13.4, 8.9, 6.0 Hz, 1H), 2.04-1.73 (m, 6H), 0.92 (t, J=7.4 Hz, 3H).

### Example 19: Preparation of 4-((7-ethyl-7-hydroxy-6,7-dihydro-5H-cyclopenta[b]pyridin-2-yl)amino)-2-((4-(1-(2-hydroxyethyl)piperidin-4-yl)-3-methylphenyl)amino)pyrimidine-5-carbonitrile

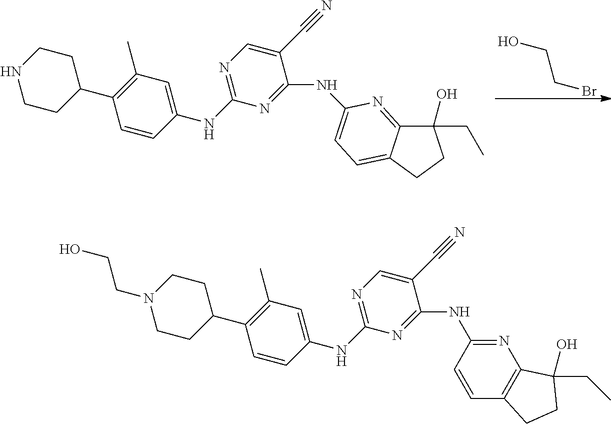

To a stirred mixture of 4-((7-ethyl-7-hydroxy-5H,6H-cyclopenta[b]pyridin-2-ylamino)-2-([3-methyl-4-(piperidin-4-yl)phenyl]aminopyrimidine-5-carbonitrile (35 mg, 0.075 mmol, 1 eq.) and 2-bromoethanol (23.28 mg, 0.188 mmol, 2.5 eq.) in ACN (3 mL) were added K₂CO₃ (30.90 mg, 0.225 mmol, 3 eq.) and KI (1.24 mg, 0.007 mmol, 0.1 eq.) in portions at room temperature. The resulting mixture was stirred at 80° C. overnight, then it was filtered, and the filter cake was washed with acetonitrile (3×5 mL). The filtrate was concentrated under reduced pressure. The crude product was purified by chiral Prep-HPLC to give 4-((7-ethyl-7-hydroxy-5H,6H-cyclopenta[b]pyridin-2-ylamino)-2-((4-[1-(2-hydroxyethyl)piperidin-4-yl]-3-methylphenylamino)pyrimidine-5-carbonitrile (11.6 mg, 29.39%). MS (ESI): [M+H]⁺ found, 514.15. ¹H NMR (300 MHz, Methanol-d₄) δ 8.44 (s, 1H), 8.11 (s, 1H), 7.59 (s, 1H), 7.38 (s, 2H), 7.20 (d, J=8.2 Hz, 1H), 3.77 (t, J=6.1 Hz, 2H), 3.18 (d, J=11.7 Hz, 2H), 3.08-2.92 (m, 1H), 2.81 (dt, J=15.9, 6.9 Hz, 2H), 2.65 (t, J=6.1 Hz, 2H), 2.44-2.23 (m, 6H), 2.14 (ddd, J=14.1, 8.8, 6.1 Hz, 1H), 2.06-1.74 (m, 6H), 0.94 (t, J=7.4 Hz, 3H).

### Example 20: Preparation of 4-((7-ethyl-7-hydroxy-6,7-dihydro-5H-cyclopenta[b]pyridin-2-yl)amino)-2-((3-methyl-4-(1-methylpiperidin-4-yl)phenyl)amino)pyrimidine-5-carbonitrile

4-((7-ethyl-7-hydroxy-6,7-dihydro-5H-cyclopenta[b]pyridin-2-yl)amino)-2-((3-methyl-4-(1-methylpiperidin-4-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (92 mg, 0.294 mmol, 1 eq.) and 3-methyl-4-(1-methylpiperidin-4-yl)aniline (60 mg, 0.294 mmol, 1 eq.) to afford the title compound (6.5 mg, 4.51%). MS (ESI) [M+H]⁺ found, 484. ¹H NMR (300 MHz, Methanol-d4): δ 8.58-7.94 (m, 2H), 7.58 (s, 1H), 7.38 (d, J=16.1 Hz, 2H), 7.16 (d, J=8.3 Hz, 1H), 3.39 (d, J=12.0 Hz, 2H), 2.96 (d, J=8.9 Hz, 2H), 2.80 (d, J=11.4 Hz, 3H), 2.71 (s, 3H), 2.32 (s, 4H), 2.13 (q, J=7.3 Hz, 1H), 2.03-1.71 (m, 6H), 0.92 (t, J=7.1 Hz, 3H).

### Examples 21 and 22: Preparation of (R)-4-((7-ethyl-7-hydroxy-6,7-dihydro-5H-cyclopenta[b]pyridin-2-yl)amino)-2-((3-methyl-4-(1-methylpiperidin-4-yl)phenyl)amino)pyrimidine-5-carbonitrile and (R)-4-((7-ethyl-7-hydroxy-6,7-dihydro-5H-cyclopenta[b]pyridin-2-yl)amino)-2-((3-methyl-4-(1-methylpiperidin-4-yl)phenyl)amino)pyrimidine-5-carbonitrile

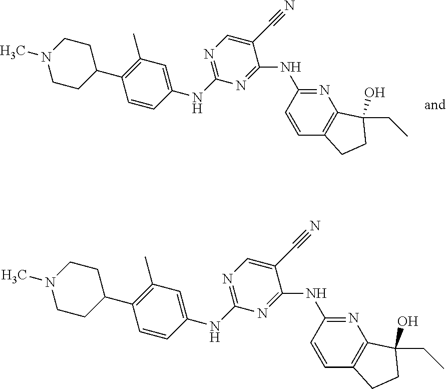

Racemic 4-((7-ethyl-7-hydroxy-6,7-dihydro-5H-cyclopenta[b]pyridin-2-yl)amino)-2-((3-methyl-4-(1-methylpiperidin-4-yl)phenyl)amino)pyrimidine-5-carbonitrile (1.8 g) was separated by chiral-prep-HPLC with the following conditions: Column, CHIRAL ART Cellulose-SB, 2*25 cm, 5 um; mobile phase, Hex (0.5% 2M NH3-MeOH)- and IPA- (hold 30% IPA—in 21 min); Detector, UV 254 nm to give one enantiomer: 4-([(7R)-7-ethyl-7-hydroxy-5H,6H-cyclopenta[b]pyridin-2-yl]amino-2-([3-methyl-4-(1-methylpiperidin-4-yl)phenyl]aminopyrimidine-5-carbonitrile (chirality is arbitrarily assigned) (684.4 mg, 67.34%). MS (ESI) [M+H]⁺ found, 484. ¹H NMR (300 MHz, Methanol-d4) δ 8.40 (s, 1H), 8.10 (s, 1H), 7.56 (s, 1H), 7.34 (d, J=12.7 Hz, 2H), 7.16 (d, J=8.3 Hz, 1H), 3.13 (d, J=11.7 Hz, 2H), 3.05-2.85 (m, 1H), 2.79 (td, J=15.7, 14.6, 6.2 Hz, 2H), 2.67-2.22 (m, 9H), 2.19-1.72 (m, 7H), 0.92 (t, J=7.4 Hz, 3H).

And another enantiomer: 4-([(7S)-7-ethyl-7-hydroxy-5H,6H-cyclopenta[b]pyridin-2-yl]amino-2-([3-methyl-4-(1-methylpiperidin-4-yl)phenyl]aminopyrimidine-5-carbonitrile (chirality is arbitrarily assigned) (642.5 mg, 127.47%). MS (ESI) [M+H]⁺ found, 484. ¹H NMR (300 MHz, Methanol-d4): δ 8.40 (s, 1H), 8.10 (s, 1H), 7.56 (s, 1H), 7.34 (d, J=12.7 Hz, 2H), 7.16 (d, J=8.3 Hz, 1H), 3.13 (d, J=11.7 Hz, 2H), 3.05-2.71 (m, 3H), 2.67-2.22 (m, 9H), 2.19-1.72 (m, 7H), 0.92 (t, J=7.4 Hz, 3H).

### Example 23: Preparation of 2-((4-(4-cyclopropylpiperazin-1-yl)phenyl)amino)-4-((7-ethyl-7-hydroxy-6,7-dihydro-5H-cyclopenta[b]pyridin-2-yl)amino)pyrimidine-5-carbonitrile

2-((4-(4-cyclopropylpiperazin-1-yl)phenyl)amino)-4-((7-ethyl-7-hydroxy-6,7-dihydro-5H-cyclopenta[b]pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (60 mg, 0.190 mmol, 1.00 eq.) and 4-(4-cyclopropylpiperazin-1-yl)aniline (61.94 mg, 0.285 mmol, 1.5 eq.) to afford the title compound (12.7 mg, 12.11%). MS (ESI) [M+H]⁺ found, 495. ¹H NMR (300 MHz, DMSO-d6): δ 9.82 (d, J=13.8 Hz, 1H), 8.94 (d, J=73.0 Hz, 1H), 8.52 (s, 1H), 7.64 (s, 2H), 7.43 (s, 2H), 6.88 (d, J=8.6 Hz, 2H), 4.92 (s, 1H), 3.06 (s, 4H), 2.90 (ddd, J=14.8, 8.6, 5.5 Hz, 2H), 2.81-2.58 (m, 4H), 2.16 (ddd, J=13.8, 8.5, 5.5 Hz, 1H), 1.99 (ddd, J=13.6, 8.6, 5.6 Hz, 1H), 1.86 (dq, J=14.8, 7.3 Hz, 1H), 1.67 (dq, J=14.3, 7.2 Hz, 2H), 0.85 (t, J=7.4 Hz, 3H), 0.43 (d, J=30.3 Hz, 4H).

### Example 24: Preparation of 4-((7-ethyl-7-hydroxy-6,7-dihydro-5H-cyclopenta[b]pyridin-2-yl)amino)-2-((4-(5-methyl-2,5-diazabicyclo[2.2.2]octan-2-yl)phenyl)amino)pyrimidine-5-carbonitrile

4-((7-ethyl-7-hydroxy-6,7-dihydro-5H-cyclopenta[b]pyridin-2-yl)amino)-2-((4-(5-methyl-2,5-diazabicyclo[2.2.2]octan-2-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (70 mg, 0.222 mmol, 1.00 eq.) and 4-(5-methyl-2,5-diazabicyclo[2.2.2]octan-2-ylaniline (52.99 mg, 0.244 mmol, 1.1 eq.) to afford the title compound (16.2 mg, 14.45%). MS (ESI) [M+H]⁺ found, 471. ¹H NMR (300 MHz, Methanol-d4): δ 8.38 (s, 1H), 7.80 (d, J=144.0 Hz, 2H), 7.42 (d, J=8.3 Hz, 2H), 6.82-6.73 (m, 2H), 4.20 (s, 1H), 3.94 (d, J=11.5 Hz, 1H), 3.70 (s, 1H), 3.65-3.40 (m, 3H), 2.98 (s, 4H), 2.78 (dt, J=15.8, 6.9 Hz, 1H), 2.33 (ddd, J=13.6, 8.5, 5.0 Hz, 2H), 2.18-1.69 (m, 6H), 0.92 (t, J=7.5 Hz, 3H).

### Example 25: Preparation of 2-((1,1-dimethyl-1,2,3,4-tetrahydroisoquinolin-6-yl)amino)-4-((7-ethyl-7-hydroxy-6,7-dihydro-5H-cyclopenta[b]pyridin-2-yl)amino)pyrimidine-5-carbonitrile

2-((1,1-dimethyl-1,2,3,4-tetrahydroisoquinolin-6-yl)amino)-4-((7-ethyl-7-hydroxy-6,7-dihydro-5H-cyclopenta[b]pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (134.36 mg, 0.426 mmol, 1.5 eq.) and 1,1-dimethyl-3,4-dihydro-2H-isoquinolin-6-amine (50 mg, 0.284 mmol, 1 eq.) to afford the title compound (11.1 mg, 6.86%). MS(ESI): mass calcd. for C₂₆H₂₉N₇O 455.24; [M+1]⁺ found, 456.10. ¹H NMR (400 MHz, DMSO-d₆) δ 10.07 (s, 1H), 9.43 (s, 1H), 9.12 (s, 2H), 8.59 (s, 1H), 7.69 (d, J=7.8 Hz, 2H), 7.45 (d, J=23.5 Hz, 2H), 7.32 (d, J=8.4 Hz, 1H), 4.96 (s, 1H), 3.43 (t, J=6.0 Hz, 2H), 3.01-2.78 (m, 3H), 2.73 (ddd, J=16.0, 8.4, 5.4 Hz, 1H), 2.18 (ddd, J=13.8, 8.5, 5.5 Hz, 1H), 2.00 (ddd, J=13.6, 8.7, 5.4 Hz, 1H), 1.87 (dq, J=14.7, 7.3 Hz, 1H), 1.75-1.64 (m, 1H), 1.62 (s, 6H), 0.85 (t, J=7.4 Hz, 3H).

### Example 26: Preparation of 4-((7-ethyl-7-hydroxy-6,7-dihydro-5H-cyclopenta[b]pyridin-2-yl)amino)-2-((1,1,2-trimethyl-1,2,3,4-tetrahydroisoquinolin-6-yl)amino)pyrimidine-5-carbonitrile

4-((7-ethyl-7-hydroxy-6,7-dihydro-5H-cyclopenta[b]pyridin-2-yl)amino)-2-((1,1,2-trimethyl-1,2,3,4-tetrahydroisoquinolin-6-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (82.71 mg, 0.263 mmol, 1 eq.) and 1,1,2-trimethyl-3,4-dihydroisoquinolin-6-amine (50 mg, 0.263 mmol, 1 eq.) to afford the title compound (6.9 mg, 5.08%). MS(ESI): mass calcd. for C₂₇H₃₁N₇O 469.26; [M+1]⁺ found, 470.10. ¹H NMR (300 MHz, Methanol-d₄) δ 8.50 (d, J=12.1 Hz, 2H), 8.13 (s, 1H), 7.68 (d, J=8.3 Hz, 1H), 7.58-7.46 (m, 2H), 7.37 (d, J=8.7 Hz, 1H), 3.50 (s, 2H), 3.07 (t, J=7.0 Hz, 2H), 3.04-2.94 (m, 1H), 2.89 (d, J=2.7 Hz, 3H), 2.87-2.72 (m, 1H), 2.37 (ddd, J=13.5, 8.5, 5.0 Hz, 1H), 2.15 (ddd, J=13.4, 8.8, 5.9 Hz, 1H), 2.06-1.93 (m, 1H), 1.85 (dd, J=13.9, 7.3 Hz, 1H), 1.72 (d, J=1.6 Hz, 6H), 0.95 (t, J=7.5 Hz, 3H).

### Examples 27 and 28: Preparation of (R)-4-((7-ethyl-7-hydroxy-6,7-dihydro-5H-cyclopenta[b]pyridin-2-yl)amino)-2-((1,1,2-trimethyl-1,2,3,4-tetrahydroisoquinolin-6-yl)amino)pyrimidine-5-carbonitrile and (S)-4-((7-ethyl-7-hydroxy-6,7-dihydro-5H-cyclopenta[b]pyridin-2-yl)amino)-2-((1,1,2-trimethyl-1,2,3,4-tetrahydroisoquinolin-6-yl)amino)pyrimidine-5-carbonitrile

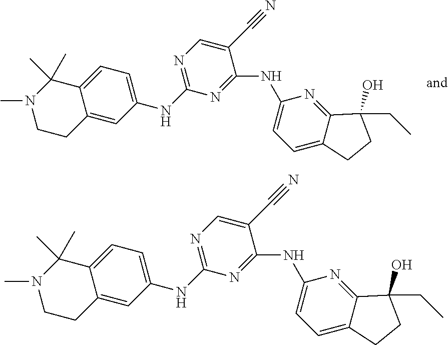

Racemic 4-((7-ethyl-7-hydroxy-6,7-dihydro-5H-cyclopenta[b]pyridin-2-yl)amino)-2-((1,1,2-trimethyl-1,2,3,4-tetrahydroisoquinolin-6-yl)amino)pyrimidine-5-carbonitrile was purified by chiral prep-HPLC with the following conditions (column: CHIRALPAK AD-H, 2*25 cm, 5 μm; Mobile Phase A: Hex (0.5% 2M NH₃-MeOH)-HPLC, Mobile Phase B: IPA-HPLC; Flow rate: 20 ML/MIN mL/min; Gradient: isocratic) to give one enantiomer with retention time=6.89 mins: 4-([(7R)-7-ethyl-7-hydroxy-5H,6H-cyclopenta[b]pyridin-2-yl]amino-2-[(1,1,2-trimethyl-3,4-dihydroisoquinolin-6-yl)amino]pyrimidine-5-carbonitrile (20.5 mg, 20.50%). MS (ESI) [M+1]⁺ found, 470. ¹H NMR (400 MHz, Methanol-d4): δ 8.46 (s, 1H), 8.09 (s, 1H), 7.65 (d, J=8.5 Hz, 1H), 7.45-7.22 (m, 3H), 3.17-2.94 (m, 3H), 2.90-2.76 (m, 3H), 2.53 (s, 3H), 2.37 (ddd, J=13.5, 8.5, 4.9 Hz, 1H), 2.20-2.08 (m, 1H), 1.92 (ddd, J=56.3, 13.8, 7.1 Hz, 2H), 1.50 (d, J=2.1 Hz, 6H), 0.95 (t, J=7.5 Hz, 3H).

Another enantiomer with retention time=10.2 mins: 4-([(7S)-7-ethyl-7-hydroxy-5H,6H-cyclopenta[b]pyridin-2-yl]amino-2-[(1,1,2-trimethyl-3,4-dihydroisoquinolin-6-yl)amino]pyrimidine-5-carbonitrile (20 mg, 20.00%). MS (ESI) [M+1]⁺ found, 470. ¹H NMR (400 MHz, Methanol-d4): δ 8.46 (s, 2H), 7.65 (d, J=8.3 Hz, 1H), 7.47-7.24 (m, 3H), 3.06-2.73 (m, 6H), 2.53 (s, 3H), 2.37 (ddd, J=13.4, 8.6, 4.9 Hz, 1H), 2.14 (ddd, J=14.0, 8.8, 6.0 Hz, 11H), 1.99 (dq, J=14.7, 7.3 Hz, 1H), 1.85 (dq, J=14.7, 7.4 Hz, 1H), 1.50 (s, 61H), 0.95 (t, J=7.5 Hz, 31H).

### Example 29: Preparation of 4-((7-ethyl-7-hydroxy-6,7-dihydro-5H-cyclopenta[b]pyridin-2-yl)amino)-2-((2-methyl-1,2,3,4-tetrahydroisoquinolin-7-yl)amino)pyrimidine-5-carbonitrile

4-((7-ethyl-7-hydroxy-6,7-dihydro-5H-cyclopenta[b]pyridin-2-yl)amino)-2-((2-methyl-1,2,3,4-tetrahydroisoquinolin-7-yl)amino)pyrimidine-5-carbonitrile was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (97.01 mg, 0.308 mmol, 1 eq.) and 2-methyl-3,4-dihydro-1H-isoquinolin-7-amine (50 mg, 0.308 mmol, 1 eq.) to afford the title compound (8.7 mg, 6.16%) as a yellow solid. MS(ESI): mass calcd. for C₂₅H₂₇N₇O 441.23; [M+1]⁺ found, 442.15. ¹H NMR (300 MHz, DMSO-d6) δ 9.92 (s, 1H), 9.31 (s, 1H), 8.54 (s, 1H), 7.63 (d, J=7.3 Hz, 2H), 7.29 (d, J=28.0 Hz, 2H), 6.98 (d, J=8.2 Hz, 1H), 4.93 (s, 1H), 3.28 (s, 2H), 2.91 (m, 1H), 2.79-2.63 (m, 3H), 2.55 (d, J=5.7 Hz, 2H), 2.31 (s, 3H), 2.17 (ddd, J=13.6, 8.5, 5.5 Hz, 11H), 1.99 (m, 11H), 1.84 (dq, J=14.7, 7.6 Hz, 1H), 1.67 (dq, J=14.4, 7.3 Hz, 1H), 0.83 (t, J=7.4 Hz, 3H).

### Example 30: Preparation of 2-((4,4-dimethyl-1,2,3,4-tetrahydroisoquinolin-7-yl)amino)-4-((7-ethyl-7-hydroxy-6,7-dihydro-5H-cyclopenta[b]pyridin-2-yl)amino)pyrimidine-5-carbonitrile

### Step 1: Preparation of tert-butyl 7-((5-cyano-4-((7-ethyl-7-hydroxy-6,7-dihydro-5H-cyclopenta[b]pyridin-2-yl)amino)pyrimidin-2-yl)amino)-4,4-dimethyl-3,4-dihydroisoquinoline-2(1H)-carboxylate

Tert-butyl 7-((5-cyano-4-((7-ethyl-7-hydroxy-6,7-dihydro-5H-cyclopenta[b]pyridin-2-yl)amino)pyrimidin-2-yl)amino)-4,4-dimethyl-3,4-dihydroisoquinoline-2(1H)-carboxylate was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (60 mg, 0.190 mmol, 1.00 eq.) and tert-butyl 7-amino-4,4-dimethyl-1,3-dihydroisoquinoline-2-carboxylate (52.52 mg, 0.190 mmol, 1 eq.) to afford the tide compound (60 mg, 56.82%). MS (ESI): mass calcd. for C₃₁H₃₇N₇O₃ 555.30; [M+1] found 556.

### Step 2: Preparation of 2-((4,4-dimethyl-1,2,3,4-tetrahydroisoquinolin-7-yl)amino)-4-((7-ethyl-7-hydroxy-6,7-dihydro-5H-cyclopenta[b]pyridin-2-yl)amino)pyrimidine-5-carbonitrile

To a solution of tert-butyl 7-([5-cyano-4-((7-ethyl-7-hydroxy-5H,6H-cyclopenta[b]pyridin-2-ylamino)pyrimidin-2-yl]amino-4,4-dimethyl-1,3-dihydroisoquinoline-2-carboxylate (45 mg, 0.081 mmol, 1 eq.) in DCM (3 mL) at 0° C. was added TFA (0.3 mL) dropwise. The resulting mixture was stirred at 0° C. for 40 min. Then it was concentrated under reduced pressure. The residue was purified by reversed phase flash chromatography (MeCN in Water (0.1% FA), 5% to 100% gradient in 40 min) to give 2-[(4,4-dimethyl-2,3-dihydro-1H-isoquinolin-7-yl)amino]-4-((7-ethyl-7-hydroxy-5H,6H-cyclopenta[b]pyridin-2-ylamino)pyrimidine-5-carbonitrile, formic acid (18.0 mg, 44.36%). MS (ESI) [M+1]⁺ found, 456. ¹H NMR (300 MHz, Methanol-d4): δ 8.53 (s, 1H), 8.45 (s, 1H), 8.08 (s, 1H), 7.65 (d, J=8.3 Hz, 1H), 7.39 (td, J=14.8, 13.8, 8.6 Hz, 3H), 4.12 (s, 2H), 3.14 (s, 2H), 3.07-2.91 (m, 1H), 2.81 (ddd, J=15.7, 8.5, 6.0 Hz, 1H), 2.35 (ddd, J=13.5, 8.5, 5.0 Hz, 1H), 2.12 (ddd, J=14.0, 8.8, 5.9 Hz, 1H), 2.03-1.90 (m, 1H), 1.88-1.73 (m, 11H), 1.39 (s, 6H), 1.30 (d, J=4.4 Hz, 1H), 0.93 (t, J=7.5 Hz, 3H).

### Example 31: Preparation of 2-((4-(4,7-diazaspiro[2.5]octan-7-yl)phenyl)amino)-4-((7-ethyl-7-hydroxy-6,7-dihydro-5H-cyclopenta[b]pyridin-2-yl)amino)pyrimidine-5-carbonitrile

2-((4-(4,7-diazaspiro[2.5]octan-7-yl)phenyl)amino)-4-((7-ethyl-7-hydroxy-6,7-dihydro-5H-cyclopenta[b]pyridin-2-yl)amino)pyrimidine-5-carbonitrile was prepared and purified under conditions similar to example 30 by using 2-Chloro-4-[(7-ethyl-7-hydroxy-5,6-dihydrocyclopenta[b]pyridin-2-yl)amino]pyrimidine-5-carbonitrile carbonitrile (70 mg, 0.222 mmol, 1.00 eq.) and tert-butyl 7-(4-aminophenyl)-4,7-diazaspiro[2.5]octane-4-carboxylate (100.89 mg, 0.333 mmol, 1.5 eq.) to afford the title compound (26.9 mg, 28.67%). MS (ESI) [M+1]⁺ found, 481. ¹H NMR (400 MHz, DMSO-d6): δ 9.84 (s, 1H), 8.92 (d, J=143.0 Hz, 1H), 8.52 (s, 1H), 8.30 (s, 1H), 7.62 (s, 2H), 7.36 (s, 2H), 6.82 (s, 2H), 4.94 (s, 1H), 3.02 (dd, J=6.3, 3.7 Hz, 2H), 2.89 (d, J=7.5 Hz, 5H), 2.77-2.66 (m, 1H), 2.16 (ddd, J=13.8, 8.5, 5.5 Hz, 1H), 1.98 (ddd, J=13.6, 8.7, 5.5 Hz, 1H), 1.85 (dt, J=14.5, 7.3 Hz, 1H), 1.67 (dd, J=13.7, 7.2 Hz, 1H), 0.85 (t, J=7.4 Hz, 3H), 0.50 (q, J=2.1 Hz, 4H).

### Example 32: Preparation of 2-((4-(cis-3,5-dimethylpiperazin-1-yl)phenyl)amino)-4-((7-ethyl-7-hydroxy-6,7-dihydro-5H-cyclopenta[b]pyridin-2-yl)amino)pyrimidine-5-carbonitrile

2-((4-(cis-3,5-dimethylpiperazin-1-yl)phenyl)amino)-4-((7-ethyl-7-hydroxy-6,7-dihydro-5H-cyclopenta[b]pyridin-2-yl)amino)pyrimidine-5-carbonitrile was prepared and purified under conditions similar to example 30 by using 2-Chloro-4-[(7-ethyl-7-hydroxy-5,6-dihydrocyclopenta[b]pyridin-2-yl)amino]pyrimidine-5-carbonitrile (93.05 mg, 0.294 mmol, 1.5 eq.) and tert-butyl cis-4-(4-aminophenyl)-2,6-dimethylpiperazine-1-carboxylate (10 mg, 0.033 mmol, 1 eq.) to afford the title compound (17.3 mg, 34.58). MS (ESI) [M+1]⁺ found, 485. ¹H NMR (300 MHz, Methanol-d4): δ 8.39 (s, 1H), 8.03 (s, 1H), 7.56 (s, 1H), 7.43 (d, J=8.2 Hz, 2H), 7.03-6.92 (m, 2H), 3.67-3.56 (m, 2H), 3.16 (ddd, J=10.0, 6.5, 3.0 Hz, 2H), 3.05-2.89 (m, 1H), 2.86-2.70 (m, 1H), 2.45-2.24 (m, 3H), 2.11 (ddd, J=13.4, 8.8, 6.0 Hz, 1H), 1.89 (ddt, J=36.3, 13.8, 7.2 Hz, 2H), 1.23 (d, J=6.5 Hz, 6H), 0.92 (t, J=7.4 Hz, 3H).

### Example 33: Preparation of 4-((7-ethyl-7-hydroxy-6,7-dihydro-5H-cyclopenta[b]pyridin-2-yl)amino)-2-((4-(4-methyl-4,7-diazaspiro[2.5]octan-7-yl)phenyl)amino)pyrimidine-5-carbonitrile

4-((7-ethyl-7-hydroxy-6,7-dihydro-5H-cyclopenta[b]pyridin-2-yl)amino)-2-((4-(4-methyl-4,7-diazaspiro[2.5]octan-7-yl)phenyl)amino)pyrimidine-5-carbonitrile was prepared and purified under conditions similar to example 30 by using 2-Chloro-4-[(7-ethyl-7-hydroxy-5,6-dihydrocyclopenta[b]pyridin-2-yl)amino]pyrimidine-5-carbonitrile carbonitrile (130.77 mg, 0.414 mmol, 1.5 eq.) and 4-(4-methyl-4,7-diazaspiro[2.5]octan-7-ylaniline (60 mg, 0.276 mmol, 1 eq.) to afford the title compound (40.4 mg, 29.11%). MS (ESI): mass calcd. for C₂₈H₃₂N₈O 496.62; [M+1]⁺ found, 495.10. ¹H NMR (300 MHz, DMSO-d6) δ 9.82 (s, 1H), 9.07 (s, 1H), 8.52 (s, 1H), 7.63 (s, 2H), 7.39 (s, 2H), 6.85 (d, J=8.5 Hz, 2H), 4.92 (s, 1H), 3.15-3.06 (m, 2H), 2.98-2.82 (m, 5H), 2.78-2.62 (m, 1H), 2.28 (s, 3H), 2.16 (ddd, J=13.7, 8.4, 5.5 Hz, 1H), 2.06 (s, 1H), 1.77 (s, 1H), 1.67 (dq, J=14.2, 7.3 Hz, 1H), 0.85 (t, J=7.4 Hz, 3H), 0.66 (s, 2H), 0.50 (d, J=4.9 Hz, 2H).

### Example 34: Preparation of 4-((7-ethyl-7-hydroxy-6,7-dihydro-5H-cyclopenta[b]pyridin-2-yl)amino)-2-((4-(5-methylhexahydropyrrolo[3,4-c]pyrrol-2(1H)-yl)phenyl)amino)pyrimidine-5-carbonitrile

4-((7-ethyl-7-hydroxy-6,7-dihydro-5H-cyclopenta[b]pyridin-2-yl)amino)-2-((4-(5-methylhexahydropyrrolo[3,4-c]pyrrol-2(1H)-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (130.77 mg, 0.414 mmol, 1.5 eq.) and 4-(5-methyl-hexahydropyrrolo[3,4-c]pyrrol-2-ylaniline (60 mg, 0.276 mmol, 1 eq.) to afford the title compound (2.2 mg, 1.56%). MS(ESI): mass calcd. for C₂₈H₃₂N₈O 496.62; [M+1]⁺ found, 497.25. ¹H NMR (400 MHz, DMSO-d₆) δ 10.07 (s, 1H), 9.43 (s, 1H), 9.12 (s, 1H), 8.59 (s, 2H), 7.69 (d, J=7.8 Hz, 2H), 7.45 (d, J=23.5 Hz, 2H), 4.96 (s, 1H), 3.43 (t, J=6.0 Hz, 2H), 3.01-2.78 (m, 3H), 2.73 (ddd, J=16.0, 8.4, 5.4 Hz, 1H), 2.67 (m, 2H), 2.61 (m, 2H), 2.31 (m, 2H), 2.01 (m, 4H), 2.00 (ddd, J=13.6, 8.7, 5.4 Hz, 1H), 1.87 (dq, J=14.7, 7.3 Hz, 1H), 1.75-1.64 (m, 1H), 0.85 (t, J=7.4 Hz, 3H).

### Example 35: Preparation of 4-((7-ethyl-7-hydroxy-6,7-dihydro-5H-cyclopenta[b]pyridin-2-yl)amino)-2-((4-(8-methyl-3,8-diazabicyclo[3.2.1]octan-3-yl)phenyl)amino)pyrimidine-5-carbonitrile

4-((7-ethyl-7-hydroxy-6,7-dihydro-5H-cyclopenta[b]pyridin-2-yl)amino)-2-((4-(8-methyl-3,8-diazabicyclo[3.2.1]octan-3-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (130.77 mg, 0.414 mmol, 1.5 eq.) and 4-(8-methyl-3,8-diazabicyclo[3.2.1]octan-3-ylaniline (60 mg, 0.276 mmol, 1 eq.) to afford the title compound (43.1 mg, 31.4%). MS(ESI): mass calcd. for C₂₈H₃₂N₈O 496.62; [M+1]⁺ found, 495.05. ¹H NMR (300 MHz, DMSO-d6) δ 9.83 (s, 1H), 9.07 (s, 1H), 8.52 (s, 1H), 7.62 (s, 2H), 7.40 (s, 2H), 6.80 (d, J=8.5 Hz, 2H), 4.92 (s, 1H), 3.62 (s, 2H), 3.49 (d, J=11.5 Hz, 2H), 2.96-2.82 (m, 3H), 2.79-2.63 (m, 1H), 2.16 (m, 3H), 2.09-1.96 (m, 4H), 2.00-1.77 (m, 3H), 1.68 (dt, J=13.7, 7.2 Hz, 1H), 0.85 (t, J=7.3 Hz, 3H).

### Example 36: Preparation of 4-((7-ethyl-7-hydroxy-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile

4-((7-ethyl-7-hydroxy-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (59.99 mg, 0.190 mmol, 1.00 eq.) and 4-(3-methyl-3,8-diazabicyclo[3.2.1]octan-8-ylaniline (41.29 mg, 0.190 mmol, 1.00 eq.) to afford the title compound (36.4 mg, 38.19%). MS(ESI): mass calcd. for C₂₈H₃₂N₈O 496.62; [M+1]⁺ found, 497.20. ¹H NMR (300 MHz, DMSO-d6) δ 9.75 (s, 1H), 9.03 (s, 1H), 8.50 (s, 1H), 7.69-7.60 (m, 2H), 7.31 (s, 2H), 6.75 (d, J=8.5 Hz, 2H), 4.92 (s, 1H), 4.19 (s, 2H), 2.89 (ddd, J=14.8, 8.6, 5.5 Hz, 1H), 2.75-2.61 (m, 1H), 2.43 (m, 2H), 2.28 (d, J=10.4 Hz, 2H), 2.16 (ddd, J=13.7, 8.4, 5.5 Hz, 1H), 2.06 (s, 3H), 2.00 (dd, J=8.4, 5.4 Hz, 11H), 1.99-1.79 (m, 5H), 1.67 (dd, J=13.8, 7.2 Hz, 1H), 0.85 (t, J=7.4 Hz, 3H).

### Examples 37 and 38: Preparation of 4-(((R)-7-ethyl-7-hydroxy-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile and 4-(((S)-7-ethyl-7-hydroxy-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile

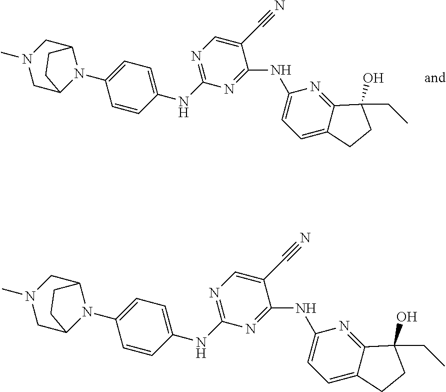

Racemic 4-((7-ethyl-7-hydroxy-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile was purified by chiral prep-HPLC with the following conditions (column: CHIRALPAK IH 3*25 cm, 5 um; Mobile Phase A: CO2, Mobile Phase B: MeOH (1%-2M-NH3-MeOH); Flow rate: 85 mL/min; Gradient: isocratic 35% B) to give one enantiomer with retention time=6.8 mins: 4-(((R)-7-ethyl-7-hydroxy-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile (988.1 mg, 21.42%) as a yellow solid. MS(ESI): mass calcd. for C₂₈H₃₂N₈O 496.27; [M+1]⁺ found, 497.25. ¹H NMR (400 MHz, DMSO-d6) δ 9.76 (s, 1H), 8.87 (d, J=133.9 Hz, 1H), 8.50 (s, 1H), 7.89 (d, J=228.8 Hz, 2H), 7.31 (s, 2H), 6.75 (d, J=8.3 Hz, 2H), 4.92 (s, 1H), 4.19 (s, 2H), 2.89 (dt, J=15.1, 7.5 Hz, 1H), 2.78-2.63 (m, 1H), 2.45 (dd, J=10.9, 2.4 Hz, 2H), 2.28 (d, J=10.6 Hz, 2H), 2.22-2.10 (m, 1H), 2.06 (s, 3H), 1.98 (ddd, J=13.5, 8.6, 5.4 Hz, 11H), 1.86 (dd, J=17.5, 6.6 Hz, 5H), 1.74-1.57 (m, 1H), 0.85 (t, J=7.4 Hz, 3H).

Another enantiomer with retention time=10.2 mins: 4-(((S)-7-ethyl-7-hydroxy-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile (997.3 mg, 31.58%) as a yellow solid. MS(ESI): mass calcd. for C₂₈H₃₂N₈O 496.27; [M+1]⁺ found, 495.35. ¹H NMR (400 MHz, DMSO-d6) δ 9.78 (s, 1H), 8.88 (d, J=139.6 Hz, 1H), 8.51 (s, 1H), 7.65 (d, J=35.3 Hz, 2H), 7.32 (s, 2H), 6.77 (s, 2H), 4.93 (s, 1H), 4.21 (s, 2H), 2.89 (ddd, J=15.1, 8.7, 5.5 Hz, 1H), 2.77-2.62 (m, 1H), 2.45 (s, 2H), 2.30 (s, 2H), 2.21-1.76 (m, 10H), 1.75-1.61 (m, 1H), 0.85 (t, J=7.4 Hz, 3H).

### Example 39: Preparation of 4-((7-ethyl-7-hydroxy-6,7-dihydro-5H-cyclopenta[b]pyridin-2-yl)amino)-2-((4-(9-methyl-3,9-diazaspiro[5.5]undecan-3-yl)phenyl)amino)pyrimidine-5-carbonitrile

4-((7-ethyl-7-hydroxy-6,7-dihydro-5H-cyclopenta[b]pyridin-2-yl)amino)-2-((4-(9-methyl-3,9-diazaspiro[5.5]undecan-3-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (50 mg, 0.158 mmol, 1.00 eq.) and 4-(9-methyl-3,9-diazaspiro [5.5] undecan-3-ylaniline (49.29 mg, 0.190 mmol, 1.2 eq.) to afford the title compound (20.8 mg, 24.16%). MS(ESI): mass calcd. for C₃₁H₃₈N₈O 538.31; [M+1]⁺ found, 539.30. ¹H NMR (300 MHz, DMSO-d₆) δ 9.82 (s, 1H), 9.07 (s, 1H), 8.50 (s, 1H), 7.50 (d, J=72.6 Hz, 4H), 6.85 (d, J=8.4 Hz, 2H), 4.92 (s, 1H), 3.07 (t, J=5.6 Hz, 4H), 2.88 (ddd, J=14.9, 8.4, 5.4 Hz, 1H), 2.77-2.61 (m, 1H), 2.31 (t, J=5.4 Hz, 4H), 2.17 (s, 4H), 1.97 (ddd, J=13.7, 8.6, 5.5 Hz, 1H), 1.85 (dd, J=13.8, 7.3 Hz, 1H), 1.65 (dd, J=13.7, 7.1 Hz, 1H), 1.49 (dt, J=17.7, 5.7 Hz, 8H), 0.83 (t, J=7.4 Hz, 3H).

### Example 40: Preparation of 2-((3,5-difluoro-4-(1-methylpiperidin-4-yl)phenyl)amino)-4-((7-ethyl-7-hydroxy-6,7-dihydro-5H-cyclopenta[b]pyridin-2-yl)amino)pyrimidine-5-carbonitrile

4-((7-ethyl-7-hydroxy-6,7-dihydro-5H-cyclopenta[b]pyridin-2-yl)amino)-2-((4-(9-methyl-3,9-diazaspiro[5.5]undecan-3-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (50 mg, 0.158 mmol, 1.00 eq.) and 3,5-difluoro-4-(1-methylpiperidin-4-yl)aniline (43.00 mg, 0.190 mmol, 1.2 eq.) to afford the title compound (28.9 mg, 32.99%). MS (ESI): [M+1]⁺ found, 504.10. ¹H NMR (300 MHz, DMSO-d₆) δ 10.22 (s, 1H), 9.37 (s, 1H), 8.60 (d, J=2.6 Hz, 1H), 7.63 (t, J=17.5 Hz, 2H), 7.29 (d, J=11.8 Hz, 2H), 4.91 (s, 1H), 2.99-2.80 (m, 3H), 2.71 (ddd, J=16.4, 8.5, 5.6 Hz, 2H), 2.28-2.08 (m, 4H), 2.06-1.84 (m, 5H), 1.81 (d, J=7.4 Hz, 1H), 1.72-1.48 (m, 3H), 0.82 (t, J=7.4 Hz, 3H).

### Example 41: Preparation of 4-((7-ethyl-7-hydroxy-6,7-dihydro-5H-cyclopenta[b]pyridin-2-yl)amino)-2-((3-fluoro-5-methyl-4-(1-methylpiperidin-4-yl)phenyl)amino)pyrimidine-5-carbonitrile

4-((7-ethyl-7-hydroxy-6,7-dihydro-5H-cyclopenta[b]pyridin-2-yl)amino)-2-((3-fluoro-5-methyl-4-(1-methylpiperidin-4-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (106 mg, 0.336 mmol, 1.00 eq.) and 3-fluoro-5-methyl-4-(1-methylpiperidin-4-yl)aniline (89.55 mg, 0.403 mmol, 1.2 eq.) to afford the title compound (46.1 mg, 27.38%). MS(ESI): mass calcd. for C₂H₃₂FN₇O 501.27; [M+1]⁺ found, 502.20. ¹H NMR (300 MHz, DMSO-d6) δ 10.03 (s, 1H), 9.41 (s, 1H), 8.58 (s, 1H), 7.65 (d, J=7.4 Hz, 2H), 7.31 (s, 1H), 7.16 (s, 1H), 4.93 (s, 1H), 2.99-2.81 (m, 3H), 2.71 (ddd, J=24.4, 11.1, 6.9 Hz, 2H), 2.21 (d, J=13.3 Hz, 7H), 2.07-1.79 (m, 6H), 1.69 (dt, J=13.7, 7.3 Hz, 1H), 1.53 (d, J=11.6 Hz, 2H), 0.84 (t, J=7.4 Hz, 3H).

### Example 42: Preparation of 2-((3-chloro-4-(1-methylpiperidin-4-yl)phenyl)amino)-4-((7-ethyl-7-hydroxy-6,7-dihydro-5H-cyclopenta[b]pyridin-2-yl)amino)pyrimidine-5-carbonitrile

2-((3-chloro-4-(1-methylpiperidin-4-yl)phenyl)amino)-4-((7-ethyl-7-hydroxy-6,7-dihydro-5H-cyclopenta[b]pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (126.46 mg, 0.401 mmol, 1.5 eq.) and 3-chloro-4-(1-methylpiperidin-4-yl)aniline (60 mg, 0.267 mmol, 1 eq.) to afford the title compound (18.7 mg, 13.84%). MS(ESI): mass calcd. for C₂₇H₃₀ClN₇O 504.04; [M+1]⁺ found, 504.20. ¹H NMR (300 MHz, DMSO-d6) δ 10.11 (s, 1H), 9.43 (s, 1H), 8.60 (s, 1H), 7.77-7.46 (m, 3H), 7.25 (d, J=8.5 Hz, 1H), 7.10 (s, 1H), 4.95 (s, 1H), 2.99-2.80 (m, 3H), 2.77-2.70 (m, 2H), 2.20 (s, 3H), 2.25-2.10 (m, 1H), 2.00 (ddd, J=13.6, 8.9, 6.0 Hz, 3H), 1.84 (p, J=7.2 Hz, 1H), 1.73-1.54 (m, 5H), 1.07 (s, 2H), 0.84 (t, J=7.4 Hz, 3H).

### Example 43: Preparation of 4-((7-ethyl-7-hydroxy-6,7-dihydro-5H-cyclopenta[b]pyridin-2-yl)amino)-2-((3-fluoro-4-(1-methylpiperidin-4-yl)phenyl)amino)pyrimidine-5-carbonitrile

4-((7-ethyl-7-hydroxy-6,7-dihydro-5H-cyclopenta[b]pyridin-2-yl)amino)-2-((3-fluoro-4-(1-methylpiperidin-4-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (136.44 mg, 0.432 mmol, 1.5 eq.) and 3-fluoro-4-(1-methylpiperidin-4-yl)aniline (60 mg, 0.288 mmol, 1 eq.) to afford the title compound (40.6 mg, 28.84%). MS(ESI): mass calcd. for C₂₇H₃₀FN₇O 487.25; [M+1]⁺ found, 486.20. ¹H NMR (300 MHz, DMSO-d6) δ 10.11 (s, 1H), 9.47 (s, 1H), 8.59 (s, 1H), 7.67-7.54 (m, 3H), 7.28-7.18 (m, 2H), 4.93 (s, 1H), 3.00-2.84 (m, 3H), 2.70 (m, 2H), 2.25-2.10 (m, 4H), 2.10-1.77 (m, 4H), 1.67 (td, J=9.6, 9.2, 2.8 Hz, 5H), 0.84 (t, J=7.4 Hz, 3H).

### Example 44: Preparation of 4-((7-ethyl-7-hydroxy-6,7-dihydro-5H-cyclopenta[b]pyridin-2-yl)amino)-2-((3-(methoxymethyl)-4-(1-methylpiperidin-4-yl)phenyl)amino)pyrimidine-5-carbonitrile

4-((7-ethyl-7-hydroxy-6,7-dihydro-5H-cyclopenta[b]pyridin-2-yl)amino)-2-((3-(methoxymethyl)-4-(1-methylpiperidin-4-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (60.00 mg, 0.190 mmol, 1.00 eq.) and 3-(methoxymethyl)-4-(1-methylpiperidin-4-yl)aniline (44.53 mg, 0.190 mmol, 1.00 eq.) to afford the title compound (36.6 mg, 37.42%). MS (ESI) [M+1]⁺ found, 514. ¹H NMR (300 MHz, Methanol-d4): δ 8.43 (s, 1H), 8.09 (s, 1H), 7.55 (q, J=14.4, 11.2 Hz, 3H), 7.27 (d, J=8.4 Hz, 1H), 4.45 (s, 2H), 3.36 (s, 3H), 3.14-3.01 (m, 2H), 3.01-2.71 (m, 3H), 2.42 (s, 3H), 2.34-2.21 (m, 3H), 2.15-2.01 (m, 1H), 1.99-1.71 (m, 6H), 0.92 (t, J=7.5 Hz, 3H).

### Example 45: Preparation of 4-((7-ethyl-7-hydroxy-6,7-dihydro-5H-cyclopenta[b]pyridin-2-yl)amino)-2-((3-methoxy-4-(1-methylpiperidin-4-yl)phenyl)amino)pyrimidine-5-carbonitrile

4-((7-ethyl-7-hydroxy-6,7-dihydro-5H-cyclopenta[b]pyridin-2-yl)amino)-2-((3-methoxy-4-(1-methylpiperidin-4-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (128.99 mg, 0.408 mmol, 1.5 eq.) and 3-methoxy-4-(1-methylpiperidin-4-yl)aniline (60 mg, 0.272 mmol, 1 eq.) to afford the title compound (24.8 mg, 17.70%). MS(ESI): mass calcd. for C₂₈H₃₃N₇O₂ 499.27; [M+1]⁺ found, 500.25. ¹H NMR (300 MHz, DMSO-d6) δ 9.93 (s, 1H), 9.21 (s, 1H), 8.57 (s, 1H), 7.60 (s, 2H), 7.19 (s, 2H), 7.04 (d, J=8.3 Hz, 1H), 4.92 (s, 1H), 3.63 (s, 3H), 2.98-2.80 (m, 3H), 2.85-2.63 (m, 2H), 2.24-2.09 (m, 4H), 1.92 (dddd, J=35.9, 21.1, 11.6, 6.4 Hz, 4H), 1.75-1.55 (m, 5H), 0.84 (t, J=7.3 Hz, 3H).

### Example 46: Preparation of 4-((7-ethyl-7-hydroxy-6,7-dihydro-5H-cyclopenta[b]pyridin-2-yl)amino)-2-((2-methyl-1,2,3,4-tetrahydroisoquinolin-6-yl)amino)pyrimidine-5-carbonitrile

4-((7-ethyl-7-hydroxy-6,7-dihydro-5H-cyclopenta[b]pyridin-2-yl)amino)-2-((2-methyl-1,2,3,4-tetrahydroisoquinolin-6-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (50 mg, 0.158 mmol, 1.00 eq.) and 2-methyl-3,4-dihydro-1H-isoquinolin-6-amine (30.83 mg, 0.190 mmol, 1.2 eq.) to afford the title compound (36.8 mg, 52.37) as a brown solid. MS (ESI): [M+1]⁺ found, 439.95. ¹H NMR (300 MHz, DMSO-do) δ 10.00 (s, 1H), 9.30 (s, 1H), 8.56 (s, 1H), 7.64 (d, J=7.5 Hz, 2H), 7.39 (d, J=32.6 Hz, 2H), 7.01 (d, J=8.4 Hz, 1H), 4.95 (s, 1H), 3.88 (s, 2H), 3.01 (d, J=6.2 Hz, 2H), 2.97-2.75 (m, 3H), 2.78-2.62 (m, 1H), 2.62 (s, 3H), 2.16 (ddd, J=13.8, 8.4, 5.5 Hz, 1H), 1.98 (ddd, J=13.6, 8.6, 5.5 Hz, 1H), 1.85 (dq, J=14.7, 7.4 Hz, 1H), 1.66 (dq, J=14.4, 7.4 Hz, 1H), 0.83 (t, J=7.4 Hz, 3H).

### Example 47: Preparation of 2-((2,8-dimethyl-1,2,3,4-tetrahydroisoquinolin-6-yl)amino)-4-((7-ethyl-7-hydroxy-6,7-dihydro-5H-cyclopenta[b]pyridin-2-yl)amino)pyrimidine-5-carbonitrile

2-((2,8-dimethyl-1,2,3,4-tetrahydroisoquinolin-6-yl)amino)-4-((7-ethyl-7-hydroxy-6,7-dihydro-5H-cyclopenta[b]pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (53.57 mg, 0.170 mmol, 1 eq.) and 2-methyl-3,4-dihydro-1H-isoquinolin-7-amine (50 mg, 0.308 mmol, 1 eq.) to afford the title compound (5.1 mg, 6.33%). MS(ESI): mass calcd. for C₂₆H₂₉N₇O 455.24; [M+1]⁺ found, 456.10. ¹H NMR (300 MHz, Methanol-d4) δ 8.47 (s, 1H), 8.20-7.95 (m, 1H), 7.65 (d, J=8.4 Hz, 1H), 7.39 (d, J=15.3 Hz, 2H), 4.60 (s, 1H), 4.29 (s, 2H), 3.48 (s, 2H), 3.29-3.19 (m, 1H), 3.14-3.06 (m, 3H), 3.04-2.91 (m, 1H), 2.89-2.72 (m, 1H), 2.35 (m, 1H), 2.23 (s, 3H), 2.13 (m, 1H), 2.06-1.89 (m, 1H), 1.85 (m, 1H), 0.93 (t, J=7.5 Hz, 3H).

### Example 48: Preparation of 4-((7-ethyl-7-hydroxy-6,7-dihydro-5H-cyclopenta[b]pyridin-2-yl)amino)-2-((2,4,4-trimethyl-1,2,3,4-tetrahydroisoquinolin-7-yl)amino)pyrimidine-5-carbonitrile

4-((7-ethyl-7-hydroxy-6,7-dihydro-5H-cyclopenta[b]pyridin-2-yl)amino)-2-((2,4,4-trimethyl-1,2,3,4-tetrahydroisoquinolin-7-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (60.04 mg, 0.190 mmol, 0.91 eq.) and 2,4,4-trimethyl-1,3-dihydroisoquinolin-7-amine (39.8 mg, 0.209 mmol, 1.1 eq.) to afford the title compound (14.8 mg, 16.51%). MS(ESI): mass calcd. for C₂₇H₃₁N₇O 469.26; [M+1]⁺ found, 468.09. ¹H NMR (300 MHz, Methanol-d4) δ 8.44 (s, 1H), 8.13-8.70 (m, 1H), 7.64 (d, J=8.4 Hz 1H), 7.43-7.36 (m, 3H), 3.91 (2H), 2.98-2.95 (m, 3H), 2.80-2.75 (m, 4H), 2.39-2.30 (m, 1H), 2.17-2.10 (m, 1H), 2.09-1.98 (m, 2H), 1.45 (s, 6H) 0.95 (t, J=2.1 Hz, 3H).

### Example 49: Preparation of 2-((2,5-dimethyl-1,2,3,4-tetrahydroisoquinolin-7-yl)amino)-4-((7-ethyl-7-hydroxy-6,7-dihydro-5H-cyclopenta[b]pyridin-2-yl)amino)pyrimidine-5-carbonitrile

2-((2,5-dimethyl-1,2,3,4-tetrahydroisoquinolin-7-yl)amino)-4-((7-ethyl-7-hydroxy-6,7-dihydro-5H-cyclopenta[b]pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (53.74 mg, 0.170 mmol, 1 eq.) and 2,5-dimethyl-3,4-dihydro-1H-isoquinolin-7-amine (30 mg, 0.170 mmol, 1 eq.) to afford the title compound (11.8 mg, 15.07%). MS (ESI) [M+1]⁺ found, 456. ¹H NMR (300 MHz, Methanol-d4): δ 8.43 (s, 1H), 8.07 (s, 1H), 7.62 (d, J=8.3 Hz, 1H), 7.23 (s, 2H), 3.65 (s, 2H), 3.12-2.90 (m, 3H), 2.88-2.71 (m, 3H), 2.55 (d, J=1.8 Hz, 3H), 2.35 (ddd, J=13.4, 8.5, 5.0 Hz, 1H), 2.21 (s, 3H), 2.12 (ddd, J=13.4, 8.8, 6.0 Hz, 1H), 2.01-1.90 (m, 1H), 1.90-1.73 (m, 1H), 0.92 (t, J=7.4 Hz, 3H).

### Example 50: Preparation of 4-((7-ethyl-7-hydroxy-6,7-dihydro-5H-cyclopenta[b]pyridin-2-yl)amino)-2-((3-(methoxymethyl)-4-(4-methylpiperazin-1-yl)phenyl)amino)pyrimidine-5-carbonitrile

4-((7-ethyl-7-hydroxy-6,7-dihydro-5H-cyclopenta[b]pyridin-2-yl)amino)-2-((3-(methoxymethyl)-4-(4-methylpiperazin-1-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (80 mg, 0.253 mmol, 1.00 eq.) and 4-(methoxymethyl)-3-(4-methylpiperazin-1-yl) aniline (71.55 mg, 0.304 mmol, 1.2 eq.) to afford the title compound (38.5 mg, 26.56%). MS (ESI): [M+1]⁺ found, 515.35. ¹H NMR (300 MHz, Methanol-d₄) δ 8.43 (s, 1H), 8.10 (s, 1H), 7.56 (d, J=28.7 Hz, 3H), 7.13 (d, J=8.7 Hz, 1H), 4.51 (s, 2H), 3.38 (s, 3H), 3.04 (d, J=5.0 Hz, 4H), 3.02-2.91 (m, 1H), 2.82 (d, J=15.3 Hz, 5H), 2.52 (s, 3H), 2.34 (ddd, J=13.4, 8.5, 5.0 Hz, 1H), 2.12 (ddd, J=14.1, 8.9, 6.1 Hz, 1H), 1.94 (td, J=15.1, 14.4, 7.5 Hz, 1H), 1.81 (dt, J=14.7, 7.4 Hz, 1H), 0.93 (t, J=7.5 Hz, 3H).

### Example 51: Preparation of 2-((4-(2,4-dimethylpiperazin-1-yl)phenyl)amino)-4-((7-ethyl-7-hydroxy-6,7-dihydro-5H-cyclopenta[b]pyridin-2-yl)amino)pyrimidine-5-carbonitrile

2-((4-(2,4-dimethylpiperazin-1-yl)phenyl)amino)-4-((7-ethyl-7-hydroxy-6,7-dihydro-5H-cyclopenta[b]pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (69.91 mg, 0.222 mmol, 1.0 eq.) and 4-(2,4-dimethylpiperazin-1-yl)aniline (50 mg, 0.244 mmol, 1.1 eq.) to afford the title compound (12.1 mg, 10.98%). MS(ESI): mass calcd. for C₂₇H₃₂N₈O₂ 484.27; [M+1]⁺ found, 485.33. ¹H NMR (300 MHz, Methanol-d4) δ 8.41 (s, 1H), 8.16-7.87 (m, 1H), 7.09-7.06 (m, 3H), 7.08 (d, J=8.1 Hz, 2H), 4.90-4.60 (m, 11H), 3.47-3.31 (m, 5H), 3.20-3.02 (m, 1H), 3.00-2.93 (m, 4H), 2.83-2.78 (m, 1H), 2.36-2.29 (m, 1H), 2.16-2.11 (m, 1H), 2.00-1.90 (m, 1H), 1.90-1.70 (m, 2H), 1.25-1.00 (m, 3H), 0.90-0.70 (m, 3H).

### Example 52: Preparation of 2-((4-(3,4-dimethylpiperazin-1-yl)phenyl)amino)-4-((7-ethyl-7-hydroxy-6,7-dihydro-5H-cyclopenta[b]pyridin-2-yl)amino)pyrimidine-5-carbonitrile

4-((7-ethyl-7-hydroxy-6,7-dihydro-5H-cyclopenta[b]pyridin-2-yl)amino)-2-((3-(methoxymethyl)-4-(4-methylpiperazin-1-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (70.47 mg, 0.223 mmol, 1.0 eq.) and 4-(3,4-dimethylpiperazin-1-yl)aniline (50.4 mg, 0.245 mmol, 1.1 eq.) to afford the title compound (25.2 mg, 22.74%) as a yellow solid. MS(ESI): mass calcd. for C₂₇H₃₂N₈O 484.27; [M+1]⁺ found, 485.25. ¹H NMR (300 MHz, Methanol-d4) δ 8.39 (s, 2H), 7.80-7.42 (m, 3H), 6.99-6.96 (dd, J=2.1, 2.1 Hz 2H), 3.60-3.57 (m, 2H), 3.16-3.12 (m, 1H), 2.96-2.93 (m, 2H), 2.78-2.53 (m, 7H), 2.33-2.10 (m, 2H), 1.95-1.85 (m, 2H), 1.30-1.20 (d, J=2.1 Hz, 3H), 1.95 (t, J=2.1 Hz, 3H).

### Example 53: Preparation of 4-((7-ethyl-7-hydroxy-6,7-dihydro-5H-cyclopenta[b]pyridin-2-yl)amino)-2-((4-(cis-3,4,5-trimethylpiperazin-1-yl)phenyl)amino)pyrimidine-5-carbonitrile

4-((7-ethyl-7-hydroxy-6,7-dihydro-5H-cyclopenta[b]pyridin-2-yl)amino)-2-((4-(cis-3,4,5-trimethylpiperazin-1-yl)phenyl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (86.31 mg, 0.274 mmol, 1 eq.) and 4-[(3R,5S)-3,4,5-trimethylpiperazin-1-yl]aniline (60 mg, 0.274 mmol, 1 eq.) to afford the title compound (8.1 mg, 10.09%) as a white powder. MS(ESI): mass calcd. for C28H34N8O 498.29 [M+1]⁺ found, 499.15. ¹H NMR (300 MHz, DMSO-d₆) δ 9.10 (s, 1H), 7.39 (s, 2H), 6.88 (s, 2H), 4.93 (s, 1H), 3.52 (s, 2H), 2.87 (d, J=5.5 Hz, 1H), 2.73 (d, J=5.5 Hz, 1H), 2.26-2.23 (m, 2H), 2.17 (s, 1H), 1.98-1.92 (m, 1H), 1.89 (d, J=7.1 Hz, 1H), 1.68 (d, J=6.6 Hz, 1H), 0.83 (s, 3H).

### Example 54: Preparation of 2-((4-(4-acetyl-4,7-diazaspiro[2.5]octan-7-yl)phenyl)amino)-4-((7-ethyl-7-hydroxy-6,7-dihydro-5H-cyclopenta[b]pyridin-2-yl)amino)pyrimidine-5-carbonitrile

2-((4-(4-acetyl-4,7-diazaspiro[2.5]octan-7-yl)phenyl)amino)-4-((7-ethyl-7-hydroxy-6,7-dihydro-5H-cyclopenta[b]pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (64.36 mg, 0.204 mmol, 1 eq.) and 1-[7-(4-aminophenyl)-4,7-diazaspiro[2.5]octan-4-yl]ethanone (50 mg, 0.204 mmol, 1 eq.) to afford the tide compound (20.7 mg, 19.01%). MS(ESI): mass calcd. for C₂₉H₃₂N₈O₂ 524.26; [M+1]⁺ found, 525.25. ¹H NMR (300 MHz, Methanol-d4) δ 8.41 (s, 1H), 8.43-7.53 (m, 1H), 7.79-7.52 (m, 1H), 7.43 (s, 2H), 7.06-6.85 (m, 2H), 3.89 (d, J=24.9 Hz, 2H), 3.24 (d, J=18.5 Hz, 2H), 3.06 (d, J=13.0 Hz, 2H), 2.96 (dd, J=9.0, 5.2 Hz, 1H), 2.89-2.70 (m, 1H), 2.36 (ddd, J=13.4, 8.4, 5.0 Hz, 1H), 2.26 (s, 2H), 2.20-2.07 (m, 2H), 1.98 (dq, J=14.8, 7.3 Hz, 1H), 1.84 (dq, J=14.5, 7.3 Hz, 1H), 1.17 (d, J=12.9 Hz, 3H), 1.06-0.85 (m, 4H).

### Example 55: Preparation of 2-((2′,3′-dihydro-1′H-spiro[cyclopropane-1,4′-isoquinolin]-7′-yl)amino)-4-((7-ethyl-7-hydroxy-6,7-dihydro-5H-cyclopenta[b]pyridin-2-yl)amino)pyrimidine-5-carbonitrile

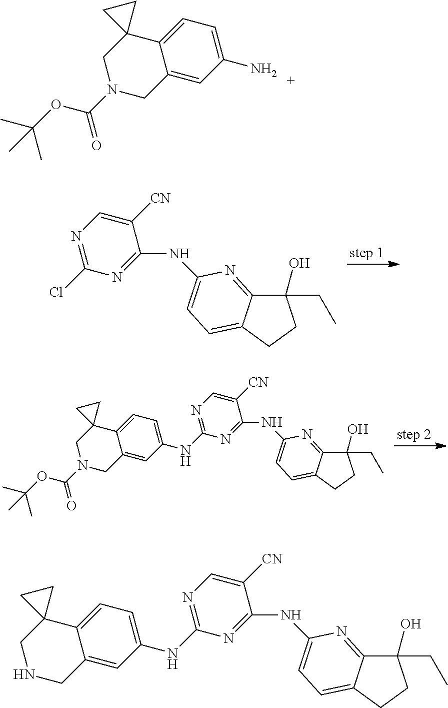

### Step 1: Preparation of tert-butyl 7′-((5-cyano-4-((7-ethyl-7-hydroxy-6,7-dihydro-5H-cyclopenta[b]pyridin-2-yl)amino)pyrimidin-2-yl)amino)-1′H-spiro[cyclopropane-1,4′-isoquinoline]-2′(3′H)-carboxylate

Tert-butyl 7′-((5-cyano-4-((7-ethyl-7-hydroxy-6,7-dihydro-5H-cyclopenta[b]pyridin-2-yl)amino)pyrimidin-2-yl)amino)-1′H-spiro[cyclopropane-1,4′-isoquinoline]-2′(3′H)-carboxylate was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (1.00 eq. 40 mg, 0.127 mmol) and tert-butyl 7-aminospiro[1,3-dihydroisoquinoline-4,1′-cyclopropane]-2-carboxylate (2.00 eq, 70 mg, 0.253 mmol) to afford the title compound (55 mg, 0.0984 mmol, 77.71% yield). ESI-LCMS calcd. for C₃₁H₃₅N₇O₃⁺ 553.28, [M+1]⁺ found, 554.05.

### Step 2: Preparation of 2-((2′,3′-dihydro-1′H-spiro[cyclopropane-1,4′-isoquinolin]-7′-yl)amino)-4-((7-ethyl-7-hydroxy-6,7-dihydro-5H-cyclopenta[b]pyridin-2-yl)amino)pyrimidine-5-carbonitrile

Tert-butyl 7-[[5-cyano-4-[(7-ethyl-7-hydroxy-5,6-dihydrocyclopenta[b]pyridin-2-yl)amino]pyrimidin-2-yl]amino]spiro[1,3-dihydroisoquinoline-4,1′-cyclopropane]-2-carboxylate (1.00 eq, 20 mg, 0.0361 mmol) was suspended in a solution of 2,6-Lutidine (2.00 eq, 0.0084 mL, 0.0722 mmol) in anhydrous THF (0.5 ml) and cooled with ice-bath. A solution of trimethylsilyl methanesulfonate (2.00 eq, 0.0099 mL, 0.0722 mmol) in anhydrous THE (0.5 ml) was added dropwise. The reaction was stirred at rt for 15 min. Another 2 eq of each of lutidine and trimethylsilyl methanesulfonate stock solutions in anhydrous THE (0.5 ml) was added. The reaction mixture was stirred at rt for another 15 min. LCMS indicated a mixture of desired product and side product tert-butyl 7-[[5-cyano-4-[(7-ethyl-7-trimethylsilyloxy-5,6-dihydrocyclopenta[b]pyridin-2-yl)amino]pyrimidin-2-yl]amino]spiro[1,3-dihydroisoquinoline-4,1′-cyclopropane]-2-carboxylate.

The reaction mixture was then stirred with tetrabutylammonium fluoride solution, 1.0 M in THE (48.0 eq, 0.50 mL, 1.73 mmol) at 40° C. for 5 h and cooled to room temperature. The reaction mixture was quenched with water and extracted repeatedly with dichloromethane. The combined organic phases were washed with brine and concentrated. The residue was purified by prep HPLC under method B conditions. The major portion of the resulting intermediate (42 mg, containing TBAF) was used for the next step without further purification. A small portion was repurified to give 3.7 mg of 4-[(7-ethyl-7-hydroxy-5,6-dihydrocyclopenta[b]pyridin-2-yl)amino]-2-(spiro[2,3-dihydro-1H-isoquinoline-4,1′-cyclopropane]-7-ylamino)pyrimidine-5-carbonitrile. ¹H NMR (500 MHz, DMSO) δ 0.86 (3H, t, J=7.0 Hz), 1.68 (1H, s), 1.85 (1H, m), 2.00 (1H, m), 2.16 (1H, m), 2.33 (4H, m), 2.55 (2H, s), 2.68 (1H, m), 2.87 (1H, m), 3.47 (2H, s), 4.97 (1H, s), 6.83 (1H, d, J=2.5 Hz), 7.73-7.59 (2H, m), 7.80 (0H, s), 8.14 (1H, s), 8.32 (1H, d, J=2.5 Hz), 8.51 (1H, s), 9.27 (1H, s), 9.90 (1H, s). ESI-LCMS calcd. for C₂₆H₂₇N₇O⁺ 453.23; [M+1]⁺ found, 454.08.

### Example 56: Preparation of 4-((7-ethyl-7-hydroxy-6,7-dihydro-5H-cyclopenta[b]pyridin-2-yl)amino)-2-((2′-methyl-2′,3′-dihydro-1′H-spiro[cyclopropane-1,4′-isoquinolin]-7′-yl)amino)pyrimidine-5-carbonitrile

4-[(7-ethyl-7-hydroxy-5,6-dihydrocyclopenta[b]pyridin-2-yl)amino]-2-(spiro[2,3-dihydro-1H-isoquinoline-4,1′-cyclopropane]-7-ylamino)pyrimidine-5-carbonitrile (42 mg) and formaldehyde (0.030 mL, 37 wt % in H₂O, 0.396 mmol) were stirred with acetic acid (1 drop) in dichloroethane (2 ml) at room temperature for 5 min. Sodium triacetoxyborohydride (0.041 mL, 0.278 mmol) was added. The reaction mixture was stirred at room temperature for 1 h, then quenched with water and extracted with dichloromethane. The resulting white precipitate was filtered, washed with water and EtOAc separately, and dried. LCMS and ¹H NMR indicate that the dried precipitate is the desired product. The organic phases of the filtrate was separated, dried over MgSO₄ and concentrated. The resulting residue was purified by pre-HPLC under method B conditions. LCMS and ¹H NMR of the resulting white powder indicate that it is the free base of the desired product. The two collections were combined, dissolved in ACN and water, and lyophilized to give the final product 4-[(7-ethyl-7-hydroxy-5,6-dihydrocyclopenta[b]pyridin-2-yl)amino]-2-[(2-methylspiro[1,3-dihydroisoquinoline-4,1′-cyclopropane]-7-yl)amino]pyrimidine-5-carbonitrile (14 mg, 0.0292 mmol, 31.50%% yield) as a white powder. ¹H NMR (500 MHz, DMSO) δ 0.86 (3H, t, J=7.5 Hz), 1.07 (4H, m), 1.69 (1H, dq, J¹=13.5 Hz, J²=7.5 Hz), 1.88 (1H, dq, J¹=13.5 Hz, J²=7.5 Hz), 2.02 (1H, m), 2.18 (1H, m), 2.75 (1H, m), 2.85 (3H, s), 2.93 (1H, m), 3.33 (2H, s), 4.25 (2H, s), 4.95 (1H, s), 6.78 (1H, d, J=8.0 Hz), 7.46 (2H, m), 7.51 (1H, s), 7.71 (11H, d, J=8.0 Hz), 8.58 (1H, s), 10.06 (1H, s), 10.13 (1H, s). ESI-LCMS calcd. for C₂₇H₂₉N₇O⁺467.24; [M+1]⁺ found 470.03.

### Example 57: Preparation of 4-((7-hydroxy-7-methyl-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile

2-((3-methyl-4-(1-methylpiperidin-4-yl)phenyl)amino)-4-((6-(2-oxopyrrolidin-1-yl)pyridin-2-yl)amino)pyrimidine-5-carbonitrile was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (40 mg, 0.133 mmol, 1 eq.) and 4-(3-methyl-3,8-diazabicyclo[3.2.1]octan-8-ylaniline (28.81 mg, 0.133 mmol, 1 eq.) to afford the title compound (11.0 mg, 16.59%). MS(ESI): mass calcd. for C₂₇H₃₀N₈O 482.25; [M+1]⁺ found, 483.35. ¹H NMR (400 MHz, DMSO-d6) δ 9.82 (d, J=44.4 Hz, 1H), 9.11 (s, 1H), 8.50 (s, 1H), 7.64 (d, J=29.8 Hz, 2H), 7.31 (s, 2H), 6.75 (d, J=8.1 Hz, 2H), 5.06 (s, 1H), 4.19 (s, 2H), 2.89 (dt, J=14.8, 7.0 Hz, 1H), 2.72 (dd, J=15.4, 8.0 Hz, 1H), 2.45 (d, J=10.3 Hz, 2H), 2.28 (d, J=10.6 Hz, 2H), 2.08 (d, J=9.5 Hz, 5H), 1.92-1.75 (m, 4H), 1.42 (s, 3H).

### Examples 58 and 59: Preparation of 4-(((S)-7-hydroxy-7-methyl-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile and 4-(((R)-7-hydroxy-7-methyl-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile

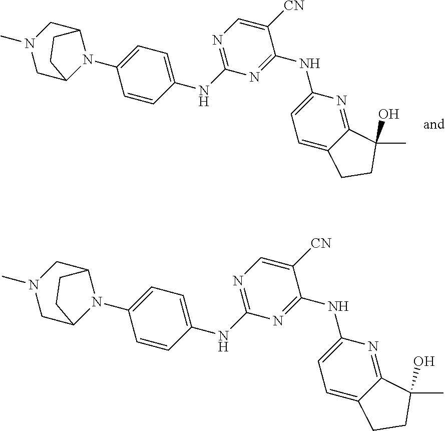

Racemic 2-((3-methyl-4-(1-methylpiperidin-4-yl)phenyl)amino)-4-((6-(2-oxopyrrolidin-1-yl)pyridin-2-yl)amino)pyrimidine-5-carbonitrile was separated by chiral-SFC (eluted with CHIRALPAK IH 3*25 cm, 5 um; Mobile Phase A: CO₂, Mobile Phase B: MeOH (0.1% FA+1%-2M-NH3-MeOH); Flow rate: 85 mL/min; Gradient: isocratic 35% B) to afford one enantiomer with retention time=8.4 min: 4-(((S)-7-hydroxy-7-methyl-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile (arbitrarily assigned) (25.4 mg, 15.88%). MS(ESI): mass calcd. for C₂₇H₃₀N₈O 482.25; [M+1]⁺ found, 481.30. ¹H NMR (400 MHz, DMSO-d6) δ 9.76 (s, 1H), 9.09 (s, 1H), 8.50 (s, 1H), 7.67-7.60 (d, J=29.3 Hz, 2H), 7.40-7.31 (s, 2H), 6.80-6.70 (d, J=8.2 Hz, 2H), 5.05 (s, 1H), 4.19 (s, 2H), 2.90 (dt, J=14.9, 7.0 Hz, 1H), 2.80-2.66 (m, 1H), 2.49-2.39 (m, 2H), 2.28 (d, J=10.6 Hz, 2H), 2.08 (d, J=9.5 Hz, 5H), 1.93-1.77 (m, 4H), 1.42 (s, 3H). And another enantiomer with retention time=9.7 min: 4-(((R)-7-hydroxy-7-methyl-6,7-dihydro-5H-cyclopenta[b]pyridin-2-yl)amino)-2-((4-(3-methyl-3,8-diazabicyclo[3.2.1]octan-8-yl)phenyl)amino)pyrimidine-5-carbonitrile (arbitrarily assigned) (25.5 mg, 15.94%). MS(ESI): mass calcd. for MS(ESI): mass calcd. for C₁₄H₁₂ClN₅O 482.25; [M+1]⁺ found, 481.25. ¹H NMR (400 MHz, DMSO-d6) δ 9.76 (s, 1H), 9.11 (s, 1H), 8.50 (s, 1H), 7.64 (d, J=29.5 Hz, 2H), 7.31 (s, 2H), 6.76 (s, 2H), 5.05 (s, 1H), 4.19 (s, 2H), 2.90 (dt, J=15.0, 7.2 Hz, 1H), 2.72 (d, J=7.4 Hz, 1H), 2.45 (d, J=10.3 Hz, 2H), 2.28 (d, J=10.6 Hz, 2H), 2.08 (d, J=9.4 Hz, 5H), 1.95-1.74 (m, 4H), 1.42 (s, 3H).

### Example 60: Preparation of 2-((4-((S)-2,4-dimethylpiperazin-1-yl)phenyl)amino)-4-((7-hydroxy-7-methyl-6,7-dihydro-5H-cyclopenta[b]pyridin-2-yl)amino)pyrimidine-5-carbonitrile

2-((4-((S)-2,4-dimethylpiperazin-1-yl)phenyl)amino)-4-((7-hydroxy-7-methyl-6,7-dihydro-5H-cyclopenta[b]pyridin-2-yl)amino)pyrimidine-5-carbonitrile was prepared and purified under conditions similar to example 14 by using 2-chloro-4-((7-hydroxy-7-methyl-5H,6H-cyclopenta[b]pyridin-2-ylamino)pyrimidine-5-carbonitrile (50 mg, 0.166 mmol, 1 eq.) and 4-[(2S)-2,4-dimethylpiperazin-1-yl]aniline (40.83 mg, 0.199 mmol, 1.2 eq.) to afford the title compound (24.4 mg, 30.63%). MS(ESI): mass calcd. for C₂₆H₃₀N₈O 470.25; [M+1]⁺ found, 471.35. ¹H NMR (400 MHz, DMSO-d6) δ 9.14 (s, 1H), 8.79 (s, 1H), 8.52 (s, 1H), 7.61 (m, 4H), 7.39 (s, 2H), 5.04 (s, 1H), 3.86 (s, 1H), 3.16 (d, J=11.6 Hz, 1H), 3.00-2.84 (m, 2H), 2.72 (q, J=8.1, 7.4 Hz, 3H), 2.30 (s, 1H), 2.20 (s, 3H), 2.16-2.03 (m, 3H), 1.42 (s, 3H), 0.97 (d, J=6.4 Hz, 3H).

### Example 61: Preparation of 4-((7-hydroxy-7-methyl-6,7-dihydro-5H-cyclopenta[b]pyridin-2-yl)amino)-2-((1,1,2-trimethyl-1,2,3,4-tetrahydroisoquinolin-6-yl)amino)pyrimidine-5-carbonitrile

4-((7-hydroxy-7-methyl-6,7-dihydro-5H-cyclopenta[b]pyridin-2-yl)amino)-2-((1,1,2-trimethyl-1,2,3,4-tetrahydroisoquinolin-6-yl)amino)pyrimidine-5-carbonitrile was prepared and purified under conditions similar to example 14 by using 2-chloro-4-((7-hydroxy-7-methyl-5H,6H-cyclopenta[b]pyridin-2-ylamino)pyrimidine-5-carbonitrile (20 mg, 0.066 mmol, 1.00 eq.) and 1,1,2-trimethyl-3,4-dihydroisoquinolin-6-amine (12.61 mg, 0.066 mmol, 1 eq.) to afford the title compound (9.9 mg, 32.78%). MS (ESI) [M+1]⁺ found, 456. ¹H NMR (400 MHz, Methanol-d4): δ 8.50 (d, J=2.8 Hz, 1H), 8.12 (s, 1H), 7.70 (d, J=8.4 Hz, 1H), 7.62-7.51 (m, 2H), 7.39 (d, J=8.7 Hz, 1H), 3.63 (t, J=6.5 Hz, 2H), 3.13 (s, 2H), 3.08-2.96 (m, 4H), 2.84 (dt, J=15.5, 7.1 Hz, 11H), 2.26 (t, J=6.8 Hz, 2H), 1.80-1.75 (m, 6H), 1.57 (s, 3H).

### Example 62: Preparation of 2-((3-methyl-4-(1-methylpiperidin-4-yl)phenyl)amino)-4-((6-(2-oxopyrrolidin-1-yl)pyridin-2-yl)amino)pyrimidine-5-carbonitrile

2-((3-methyl-4-(1-methylpiperidin-4-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (50 mg, 0.159 mmol, 1 eq.) and 3-methyl-4-(1-methylpiperidin-4-yl)aniline (32.46 mg, 0.159 mmol, 1 eq.) to afford the title compound (39.7 mg, 51.68%) as a light brown solid. MS (ESI): [M+1]⁺ found, 483.10. ¹H NMR (400 MHz, DMSO-d₆) δ 9.97 (s, 1H), 9.55 (s, 1H), 8.59 (s, 1H), 8.06 (d, J=8.2 Hz, 1H), 7.78 (t, J=8.0 Hz, 1H), 7.41 (d, J=2.0 Hz, 3H), 7.05 (s, 1H), 3.95 (t, J=7.2 Hz, 2H), 3.06 (d, J=11.1 Hz, 2H), 2.71-2.63 (m, 1H), 2.56 (t, J=8.0 Hz, 2H), 2.38 (s, 3H), 2.30 (d, J=13.6 Hz, 2H), 2.20 (s, 3H), 2.01-1.92 (m, 2H), 1.72-1.62 (m, 4H).

### Example 63: Preparation of 2-((3-(methoxymethyl)-4-(1-methylpiperidin-4-yl)phenyl)amino)-4-((6-(2-oxopyrrolidin-1-yl)pyridin-2-yl)amino)pyrimidine-5-carbonitrile

2-((3-(methoxymethyl)-4-(1-methylpiperidin-4-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (50 mg, 0.159 mmol, 1 eq.) and 3-(methoxymethyl)-4-(1-methylpiperidin-4-yl)aniline (37.23 mg, 0.159 mmol, 1 eq.) to afford the title compound (25.1 mg, 30.08%) as a yellow solid. MS (ESI): [M+1]⁺ found, 513.10. ¹H NMR (300 MHz, DMSO-d4) δ 10.02 (s, 1H), 9.49 (s, 1H), 8.59 (s, 1H), 8.22-8.02 (m, 1H), 7.88-7.33 (m, 4H), 7.15 (d, J=8.7 Hz, 1H), 4.35 (s, 2H), 3.95 (t, J=6.8 Hz, 2H), 3.26 (s, 3H), 2.97 (d, J=11.1 Hz, 2H), 2.68 (s, 1H), 2.56 (t, J=8.0 Hz, 2H), 2.31 (s, 3H), 2.16 (s, 2H), 1.97 (t, J=7.6 Hz, 2H), 1.74-1.61 (m, 4H).

### Example 64: Preparation of (R)-2-((4-(2,4-dimethylpiperazin-1-yl)phenyl)amino)-4-((6-(2-oxopyrrolidin-1-yl)pyridin-2-yl)amino)pyrimidine-5-carbonitrile

(R)-2-((4-(2,4-dimethylpiperazin-1-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (76.65 mg, 0.244 mmol, 1 eq.) and (R)-4-(2,4-dimethylpiperazin-1-yl)aniline (50 mg, 0.244 mmol, 1 eq.) to afford the title compound (4.7 mg, 3.70%). MS (ESI): [M+1]⁺ found, 484.20. ¹H NMR (300 MHz, DMSO-d₆) δ 9.86 (s, 1H), 9.46 (s, 1H), 8.53 (s, 1H), 8.02 (d, J=8.3 Hz, 1H), 7.75 (s, 1H), 7.43 (s, 3H), 6.78 (s, 2H), 3.88 (d, J=32.2 Hz, 3H), 3.12 (s, 1H), 2.92 (t, J=10.7 Hz, 1H), 2.72 (s, 1H), 2.56 (d, J=8.0 Hz, 3H), 2.21 (s, 4H), 1.97 (s, 3H), 0.95 (d, J=6.4 Hz, 3H).

### Example 65: Preparation of (S)-2-((4-(2,4-dimethylpiperazin-1-yl)phenyl)amino)-4-((6-(2-oxopyrrolidin-1-yl)pyridin-2-yl)amino)pyrimidine-5-carbonitrile

(S)-2-((4-(2,4-dimethylpiperazin-1-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (61.32 mg, 0.195 mmol, 1 eq.) and 4-[(2S)-2,4-dimethylpiperazin-1-yl]aniline (40 mg, 0.195 mmol, 1 eq.) to afford the title compound (25.2 mg, 25.46%) as a yellow solid. MS (ESI): [M+1]⁺ found, 484.35. ¹H NMR (400 MHz, DMSO-d6) δ 9.88 (s, 1H), 9.13 (s, 1H), 8.55 (s, 1H), 8.04 (d, J=8.2 Hz, 1H), 7.77 (s, 1H), 7.43 (s, 3H), 6.78 (s, 2H), 3.95-3.85 (m, 3H), 3.14 (d, J=11.7 Hz, 1H), 2.93 (t, J=10.7 Hz, 1H), 2.72 (d, J=11.1 Hz, 1H), 2.56 (t, J=7.3 Hz, 3H), 2.29 (dd, J=10.9, 3.5 Hz, 1H), 2.20 (s, 3H), 2.11 (td, J=10.6, 3.3 Hz, 1H), 1.98 (s, 2H), 0.96 (d, J=6.4 Hz, 3H).

### Example 66: Preparation of 2-((3-(methoxymethyl)-4-(4-methylpiperazin-1-yl)phenyl)amino)-4-((6-(2-oxopyrrolidin-1-yl)pyridin-2-yl)amino)pyrimidine-5-carbonitrile

2-((3-(methoxymethyl)-4-(4-methylpiperazin-1-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (100 mg, 0.318 mmol, 1 eq.) and 3-(methoxymethyl)-4-(4-methylpiperazin-1-yl)aniline to afford the title compound (16.6 mg, 9.76%) as a yellow green solid. MS(ESI): mass calcd. for C₂₇H₃₁N₉O₂ 513.26; [M+1]⁺ found, 512.20. ¹H NMR (300 MHz, DMSO-d6) δ 10.04 (s, 1H), 9.68 (s, 1H), 9.50 (s, 1H), 8.60 (s, 1H), 8.06 (d, J=8.3 Hz, 1H), 7.78-7.61 (m, 3H), 7.02 (s, 1H), 4.41 (s, 2H), 3.97 (s, 3H), 3.94-3.50 (m, 3H), 3.25 (d, J=31.6 Hz, 4H), 3.15-2.88 (m, 5H), 2.64-2.57 (m, 3H), 1.98 (t, J=7.6 Hz, 2H).

### Example 67: Preparation of 2-((4-(1-methyl-2-oxopiperidin-4-yl)phenyl)amino)-4-((6-(2-oxopyrrolidin-1-yl)pyridin-2-yl)amino)pyrimidine-5-carbonitrile

2-((4-(1-methyl-2-oxopiperidin-4-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (10 mg, 0.033 mmol, 1 eq.) and 4-(3-methyl-3,8-diazabicyclo[3.2.1]octan-8-ylaniline (7.20 mg, 0.033 mmol, 1 eq.) to afford the title compound (3.0 mg, 2.82%) as a off-white solid. MS (ESI): [M+1]⁺ found, 483.35. ¹H NMR (400 MHz, DMSO-d6) δ 10.02 (s, 1H), 9.58 (s, 1H), 8.59 (s, 1H), 8.06 (d, J=8.3 Hz, 1H), 7.80 (t, J=8.0 Hz, 1H), 7.58 (d, J=8.1 Hz, 2H), 7.14 (d, J=7.9 Hz, 2H), 3.96 (t, J=7.1 Hz, 2H), 3.38 (td, J=11.5, 11.1, 5.0 Hz, 3H), 3.17-2.97 (m, 1H), 2.85 (s, 3H), 2.70-2.53 (m, 4H), 2.49-2.24 (m, 4H), 2.06-1.81 (m, 4H).

### Example 68: Preparation of 2-((4-(2-ethyl-4-methylpiperazin-1-yl)phenyl)amino)-4-((6-(2-oxopyrrolidin-1-yl)pyridin-2-yl)amino)pyrimidine-5-carbonitrile

2-((4-(2-ethyl-4-methylpiperazin-1-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (57.40 mg, 0.182 mmol, 2 eq.) and 4-(2-ethyl-4-methylpiperazin-1-yl)aniline (20 mg, 0.091 mmol, 1 eq.) to afford the tide compound (14.2 mg, 29.95%) as a brown solid. MS(ESI): mass calcd. for C₂₇H₃₁N₉O 497.27; [M+1]⁺ found, 496.05. ¹H NMR (300 MHz, DMSO-d6) δ 9.90 (s, 1H), 9.48 (s, 11H), 8.54 (s, 1H), 8.03 (d, J=8.3 Hz, 1H), 7.75 (s, 1H), 7.53 (d, J=43.3 Hz, 3H), 6.83 (s, 2H), 3.94 (s, 2H), 3.26 (s, 1H), 3.06 (s, 2H), 2.82 (s, 2H), 2.55 (s, 5H), 1.98 (d, J=7.5 Hz, 2H), 1.68 (s, 1H), 1.31 (s, 1H), 1.22 (s, 2H), 0.82 (d, J=9.5 Hz, 3H).

### Example 69: Preparation of 2-((4-(2-(methoxymethyl)-4-methylpiperazin-1-yl)phenyl)amino)-4-((6-(2-oxopyrrolidin-1-yl)pyridin-2-yl)amino)pyrimidine-5-carbonitrile

2-((4-(2-(methoxymethyl)-4-methylpiperazin-1-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (20 mg, 0.064 mmol, 1 eq.) and 4-[2-(methoxymethyl)-4-methylpiperazin-1-yl]aniline (14.95 mg, 0.064 mmol, 1 eq.) to afford the title compound (6.0 mg, 18.25%) as a yellow solid. MS (ESI) [M+1]⁺ found, 522.45. ¹H NMR (400 MHz, DMSO-d₆) δ 9.66 (d, J=168.6 Hz, 2H), 8.54 (s, 1H), 8.03 (d, J=8.2 Hz, 1H), 7.76 (s, 1H), 7.42 (s, 3H), 6.79 (s, 2H), 3.89 (d, J=42.0 Hz, 3H), 3.65 (t, J=9.1 Hz, 1H), 3.23 (d, J=12.0 Hz, 1H), 3.17 (s, 3H), 3.09 (d, J=7.7 Hz, 1H), 2.99-2.82 (m, 2H), 2.77 (d, J=11.0 Hz, 1H), 2.56 (t, J=7.9 Hz, 2H), 2.21 (s, 4H), 2.07-1.89 (m, 3H).

### Example 70: Preparation of 2-((4-(5-methyl-2,5-diazabicyclo[2.2.1]heptan-2-yl)phenyl)amino)-4-((6-(2-oxopyrrolidin-1-yl)pyridin-2-yl)amino)pyrimidine-5-carbonitrile

2-((4-(5-methyl-2,5-diazabicyclo[2.2.1]heptan-2-yl)phenyl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (92.89 mg, 0.295 mmol, 1.0 eq.) and 4-(5-methyl-2,5-diazabicyclo[2.2.1]heptan-2-ylaniline (60 mg, 0.295 mmol, 1 eq.) to afford the title compound (12.8 mg, 8.91%). MS(ESI): mass calcd. for C₂H₂₇N₉O 481.23; [M+1]⁺ found, 482.35. ¹H NMR (400 MHz, DMSO-d6) δ 9.79 (s, 1H), 9.38 (s, 1H), 8.52 (s, 1H), 8.03 (d, J=8.2 Hz, 1H), 7.92 (s, 1H), 7.35 (s, 3H), 6.46 (s, 2H), 4.24 (s, 1H), 3.94 (s, 2H), 3.43 (s, 1H), 3.30 (s, 1H), 3.10 (d, J=9.2 Hz, 1H), 2.77 (d, J=9.5 Hz, 1H), 2.27 (s, 3H), 1.99 (s, 3H), 1.87 (d, J=9.3 Hz, 2H), 1.77 (d, J=9.3 Hz, 1H), 1.74 (s, 1H).

### Example 71: Preparation of 2-((4-(4-methyl-3-oxopiperazin-1-yl)phenyl)amino)-4-((6-(2-oxopyrrolidin-1-yl)pyridin-2-yl)amino)pyrimidine-5-carbonitrile

2-((4-(4-methyl-3-oxopiperazin-1-yl)phenyl)amino)-4-((6-(2-oxopyrrolidin-1-yl)pyridin-2-yl)amino)pyrimidine-5-carbonitrile was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (115.00 mg, 0.366 mmol, 1.5 eq.) and 4-(4-aminophenyl)-1-methylpiperazin-2-one (50 mg, 0.244 mmol, 1 eq.) to afford the title compound (6.8 mg, 5.63%) as a yellow solid. LC/MS (ESI): [M+1]⁺ found, 484.10. ¹H NMR (400 MHz, DMSO-d6) δ 9.92 (s, 1H), 9.48 (s, 1H), 8.55 (s, 1H), 8.05-8.03 (d, J=8.2 Hz, 1H), 7.47 (t, J=7.8 Hz, 1H), 7.42 (d, J=44.4 Hz, 3H), 6.84 (s, 2H), 3.94 (s, 2H), 3.70 (s, 2H), 3.41 (s, 4H), 2.89 (s, 3H), 2.57 (d, J=8.0 Hz, 2H), 1.98 (s, 2H).

### Example 72: Preparation of 2-((2-methyl-1,2,3,4-tetrahydroisoquinolin-6-yl)amino)-4-((6-(2-oxopyrrolidin-1-yl)pyridin-2-yl)amino)pyrimidine-5-carbonitrile

2-((2-methyl-1,2,3,4-tetrahydroisoquinolin-6-yl)amino)-4-((6-(2-oxopyrrolidin-1-yl)pyridin-2-yl)amino)pyrimidine-5-carbonitrile was prepared and purified under conditions similar to example 14 by using 2-Chloro-4-[(7-ethyl-7-hydroxy-5,6-dihydrocyclopenta[b]pyridin-2-yl)amino]pyrimidine-5-carbonitrile (135.80 mg, 0.431 mmol, 1.0 eq.) and 2-methyl-3,4-dihydro-1H-isoquinolin-6-amine (70 mg, 0.431 mmol, 1 eq.) to afford the title compound (12.4 mg, 5.88%) as a brown solid. MS(ESI): mass calcd. for C₂₄H₂₄N₈O 440.21; [M+1]⁺ found, 439.15. ¹H NMR (300 MHz, DMSO-d₆) δ 9.97 (s, 1H), 9.51 (s, 1H), 8.59 (s, 1H), 8.07 (d, J=8.2 Hz, 1H), 7.80 (t, J=8.0 Hz, 1H), 7.50-7.27 (m, 3H), 6.92 (d, J=8.3 Hz, 1H), 3.94 (t, J=6.9 Hz, 2H), 3.51 (t, J=6.9 Hz, 2H), 2.68 (s, 2H), 2.58 (d, J=8.1 Hz, 2H), 2.55 (d, J=4.9 Hz, 2H), 2.33 (s, 3H), 1.98 (t, J=7.6 Hz, 2H).

### Example 73: Preparation of 2-((2,8-dimethyl-1,2,3,4-tetrahydroisoquinolin-6-yl)amino)-4-((6-(2-oxopyrrolidin-1-yl)pyridin-2-yl)amino)pyrimidine-5-carbonitrile

2-((2,8-dimethyl-1,2,3,4-tetrahydroisoquinolin-6-yl)amino)-4-((6-(2-oxopyrrolidin-1-yl)pyridin-2-yl)amino)pyrimidine-5-carbonitrile was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (44.64 mg, 0.142 mmol, 1 eq.) and 2,8-dimethyl-3,4-dihydro-1H-isoquinolin-6-amine (25 mg, 0.142 mmol, 1 eq.) to afford the title compound (3.3 mg, 5.10%). MS(ESI): mass calcd. for C₂₅H₂₆N₈O 454.22; [M+1]⁺ found, 455.20. ¹H NMR (400 MHz, DMSO-d6) δ 9.92 (s, 1H), 9.47 (s, 1H), 8.58 (s, 1H), 8.06 (d, J=8.3 Hz, 1H), 7.77 (t, J=7.9 Hz, 1H), 7.55-7.34 (m, 1H), 7.21 (s, 2H), 4.07-4.02 (m, 1H), 3.92 (s, 2H), 2.71-2.61 (m, 2H), 2.60-2.52 (m, 3H), 2.34 (d, J=21.8 Hz, 5H), 2.05 (s, 3H), 1.96 (s, 2H).

### Example 74: Preparation of 2-((2-methyl-1,2,3,4-tetrahydroisoquinolin-7-yl)amino)-4-((6-(2-oxopyrrolidin-1-yl)pyridin-2-yl)amino)pyrimidine-5-carbonitrile

2-((2-methyl-1,2,3,4-tetrahydroisoquinolin-7-yl)amino)-4-((6-(2-oxopyrrolidin-1-yl)pyridin-2-yl)amino)pyrimidine-5-carbonitrile was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (50 mg, 0.159 mmol, 1 eq.) and 2-methyl-3,4-dihydro-1H-isoquinolin-7-amine (25.77 mg, 0.159 mmol, 1 eq.) to afford the title compound (5.7 mg, 7.96%) as a brown solid. MS (ESI) [M+1]⁺ found, 441.10. ¹H NMR (400 MHz, Methanol-d₄) δ 8.47 (s, 1H), 8.04 (d, J=8.1 Hz, 1H), 7.88-7.34 (m, 4H), 7.20 (d, J=8.4 Hz, 1H), 4.21 (s, 2H), 4.09 (t, J=7.1 Hz, 2H), 3.46 (t, J=6.2 Hz, 2H), 3.14 (t, J=6.3 Hz, 2H), 2.96 (s, 3H), 2.65 (t, J=8.0 Hz, 2H), 2.13 (p, J=7.7 Hz, 2H).

### Example 75: Preparation of 2-((2,5-dimethyl-1,2,3,4-tetrahydroisoquinolin-7-yl)amino)-4-((6-(2-oxopyrrolidin-1-yl)pyridin-2-yl)amino)pyrimidine-5-carbonitrile

2-((2,5-dimethyl-1,2,3,4-tetrahydroisoquinolin-7-yl)amino)-4-((6-(2-oxopyrrolidin-1-yl)pyridin-2-yl)amino)pyrimidine-5-carbonitrile was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (50 mg, 0.170 mmol, 1 eq.) and 2,5-dimethyl-3,4-dihydro-1H-isoquinolin-7-amine (30 mg, 0.170 mmol, 1 eq.) to afford the title compound (5.3 mg, 6.36%) as a yellow solid. MS (ESI) [M+1]⁺ found, 455.10. ¹H NMR (400 MHz, Methanol-d4): δ 8.44 (s, 1H), 8.03 (d, J=8.3 Hz, 1H), 7.75 (s, 2H), 7.25 (s, 2H), 4.09 (t, J=7.2 Hz, 2H), 3.65 (s, 2H), 2.93 (d, J=6.7 Hz, 2H), 2.84 (t, J=6.1 Hz, 2H), 2.69-2.52 (m, 5H), 2.21 (s, 3H), 2.12 (p, J=7.7 Hz, 2H).

### Example 76: Preparation of 2-((8-chloro-2-methyl-1,2,3,4-tetrahydroisoquinolin-6-yl)amino)-4-((6-(2-oxopyrrolidin-1-yl)pyridin-2-yl)amino)pyrimidine-5-carbonitrile

2-((8-chloro-2-methyl-1,2,3,4-tetrahydroisoquinolin-6-yl)amino)-4-((6-(2-oxopyrrolidin-1-yl)pyridin-2-yl)amino)pyrimidine-5-carbonitrile was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (80 mg, 0.254 mmol, 1 eq.) and 8-chloro-2-methyl-3,4-dihydro-1H-isoquinolin-6-amine (49.99 mg, 0.254 mmol, 1 eq.) to afford the title compound (11.3 mg, 8.40%) as a yellow solid. MS (ESI) [M+1]⁺ found, 522.45. ¹H NMR (400 MHz, DMSO-d₆) δ 10.13 (s, 1H), 9.72 (s, 1H), 8.63 (s, 1H), 8.19 (s, 1H), 8.09 (d, J=8.2 Hz, 1H), 7.81 (t, J=8.0 Hz, 1H), 7.62 (s, 1H), 7.34 (s, 2H), 3.91 (d, J=7.3 Hz, 2H), 3.39 (s, 2H), 2.70 (s, 2H), 2.55 (q, J=6.5, 4.9 Hz, 4H), 2.37 (s, 3H), 2.00-1.91 (m, 2H).

### Example 77: Preparation of 2-((8-methyl-1,2,3,4-tetrahydroisoquinolin-6-yl)amino)-4-((6-(2-oxopyrrolidin-1-yl)pyridin-2-yl)amino)pyrimidine-5-carbonitrile

### Step 1: Preparation of tert-butyl 6-((5-cyano-4-((6-(2-oxopyrrolidin-1-yl)pyridin-2-yl)amino)pyrimidin-2-yl)amino)-8-methyl-3,4-dihydroisoquinoline-2(1H)-carboxylate

Tert-butyl 6-((5-cyano-4-((6-(2-oxopyrrolidin-1-yl)pyridin-2-yl)amino)pyrimidin-2-yl)amino)-8-methyl-3,4-dihydroisoquinoline-2(1H)-carboxylate was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (100 mg, 0.318 mmol, 1 eq.) and tert-butyl 6-amino-8-methyl-3,4-dihydro-1H-isoquinoline-2-carboxylate (83.36 mg, 0.318 mmol, 1 eq.) to afford the title compound (30 mg, 17.46%) as a yellow solid. MS(ESI): mass calcd. for C₂₉H₃₂N₈O₃ 540.26; [M+1]⁺ found, 541.30.

### Step 2: 2-((8-methyl-1,2,3,4-tetrahydroisoquinolin-6-yl)amino)-4-((6-(2-oxopyrrolidin-1-yl)pyridin-2-yl)amino)pyrimidine-5-carbonitrile

A mixture of tert-butyl 6-[(5-cyano-4-([6-(2-oxopyrrolidin-1-yl)pyridin-2-yl]aminopyrimidin-2-yl)amino]-8-methyl-3,4-dihydro-1H-isoquinoline-2-carboxylate (25 mg, 0.046 mmol, 1 eq.) and TFA (1 mL) in DCM (1 mL) was stirred at rt for 30 min. The resulting mixture was concentrated under reduced pressure. The crude product was purified by chiral prep-HPLC under Method a conditions to give 2-[(8-methyl-1,2,3,4-tetrahydroisoquinolin-6-yl)amino]-4-([6-(2-oxopyrrolidin-1-yl)pyridin-2-yl]aminopyrimidine-5-carbonitrile, trifluoroacetic acid (21.3 mg, 81.65%). MS(ESI): mass calcd. for C₂₄H₂₄N₈O 440.21; [M+1]⁺ found, 441.35. ¹H NMR (400 MHz, DMSO-d6) δ 10.06 (s, 11H), 9.48 (s, 1H), 9.13-8.92 (m, 2H), 8.62 (s, 1H), 8.08 (d, J=8.2 Hz, 1H), 7.81 (t, J=8.0 Hz, 1H), 7.50 (s, 11H), 7.35 (s, 2H), 4.13 (s, 2H), 3.96 (t, J=7.1 Hz, 2H), 3.24 (s, 2H), 2.85 (s, 2H), 2.57 (t, J=8.0 Hz, 2H), 2.11 (s, 3H), 1.99 (p, J=7.7 Hz, 2H).

### Example 78: Preparation of 2-((4-(4-hydroxy-1-methylpiperidin-4-yl)phenyl)amino)-4-((6-(2-oxopyrrolidin-1-yl)pyridin-2-yl)amino)pyrimidine-5-carbonitrile

2-((4-(4-hydroxy-1-methylpiperidin-4-yl)phenyl)amino)-4-((6-(2-oxopyrrolidin-1-yl)pyridin-2-yl)amino)pyrimidine-5-carbonitrile was prepared and purified under conditions similar to example 14 by using 2-chloro-4-[(7-ethyl-7-hydroxy-5,6-dihydrocyclopenta[b]pyridin-2-yl)amino]pyrimidine-5-carbonitrile (80 mg, 0.254 mmol, 1 eq.) and 4-(4-aminophenyl)-1-methylpiperidin-4-ol (52.44 mg, 0.254 mmol, 1 eq.) to afford the title compound (5.1 mg, 5.13%) as a light brown solid. MS(ESI): mass calcd. for C₂₆H₂₈N₈O₂ 484.23; [M+1]⁺ found, 485.20. ¹H NMR (400 MHz, DMSO-d4) δ 10.05 (s, 1H), 9.67 (s, 1H), 8.60 (s, 1H), 8.26 (s, 11H), 8.07 (d, J=8.1 Hz, 1H), 7.80 (t, J=8.0 Hz, 11H), 7.57 (d, J=8.1 Hz, 2H), 7.34 (s, 3H), 4.80 (s, 11H), 3.97 (t, J=7.1 Hz, 2H), 2.67 (s, 2H), 2.57 (t, J=8.0 Hz, 2H), 2.35-2.20 (m, 21H), 1.96 (d, J=14.8 Hz, 3H), 1.96 (d, J=14.8 Hz, 4H), 1.59 (d, J=13.0 Hz, 2H).

### Example A

The Wee1 pCDK1 and PLK1 activities were determined using known assays. The data is shown in table 2.

TABLE 2

| Ex. | Wee1 pCDK1 | PLK1 |
|---|---|---|
| 1 | B | A |
| 2 | A | A |
| 3 | D | ND |
| 4 | C | A |
| 5 | B | C |
| 6 | D | ND |
| 7 | B | A |
| 8 | B | A |
| 9 | B | E |
| 10 | B | E |
| 11 | B | ND |
| 12 | A | E |
| 13 | A | E |
| 14 | E | A |
| 15 | C | A |
| 16 | B | A |
| 17 | A | A |
| 18 | C | A |
| 19 | C | A |
| 20 | A | B |
| 21 | A | A |
| 22 | C | B |
| 23 | D | A |
| 24 | A | A |
| 25 | D | E |
| 26 | A | E |
| 27 | A | E |
| 28 | C | E |
| 29 | C | B |
| 30 | C | E |
| 31 | E | B |
| 32 | B | A |
| 33 | E | A |
| 34 | B | A |
| 35 | B | A |
| 36 | C | E |
| 37 | E | E |
| 38 | C | C |
| 39 | B | A |
| 40 | D | E |
| 41 | B | C |
| 42 | C | B |
| 43 | C | C |
| 44 | B | C |
| 45 | A | A |
| 46 | B | C |
| 47 | A | C |
| 48 | C | E |
| 49 | A | A |
| 50 | D | B |
| 51 | B | C |
| 52 | C | A |
| 53 | B | A |
| 54 | C | A |
| 55 | E | A |
| 56 | B | B |
| 57 | C | E |
| 58 | ND | ND |
| 59 | ND | ND |
| 60 | D | D |
| 61 | C | E |
| 62 | A | C |
| 63 | D | E |
| 64 | C | E |
| 65 | B | E |
| 66 | C | E |
| 67 | D | E |
| 68 | B | E |
| 69 | C | E |
| 70 | C | A |
| 71 | D | C |
| 72 | A | E |
| 73 | A | E |
| 74 | B | E |
| 75 | A | B |
| 76 | D | E |
| 77 | D | ND |
| 78 | E | E |

Wee1 pCDK1
A: IC₅₀ ≤ 100 nM;
B: 100 nM < IC₅₀ ≤ 250 nM
C: 250 nM < IC₅₀ ≤ 500 nM
D: 500 nM < IC₅₀ ≤ 1 μM
E: IC₅₀ > 1 μM
ND: not determined
PLK1
A: IC₅₀ ≤ 100 nM;
B: 100 nM < IC₅₀ ≤ 250 nM
C: 250 nM < IC₅₀ ≤ 500 nM
D: 500 nM < IC₅₀ ≤ 1 μM
E: IC₅₀ > 1 μM
ND: not determined

### Example C: Pharmaceutical Compositions

### Example C1: Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example C2: Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example C3: Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

The examples and embodiments described herein are for illustrative purposes only and in some embodiments, various modifications or changes are to be included within the purview of disclosure and scope of the appended claims.

1. A compound of Formula (I), or a pharmaceutically acceptable salt, solvate, or stereoisomer thereof: 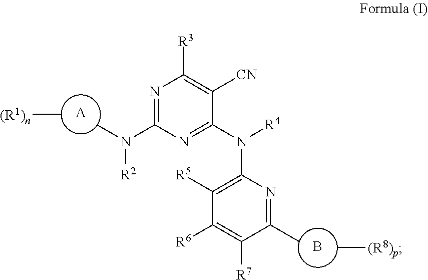
wherein:
Ring A is a monocyclic or a bicyclic ring;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1a};
or two R¹ on the same atom form an oxo;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1b};
or two R^{1a} on the same atom form an oxo;
each R^{1b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R^{1b} on the same atom form an oxo;
n is 0, 1, 2, 3, 4, 5, or 6;
R² is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R³ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁷ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring B is a cycloalkyl, heterocycloalkyl, aryl, or heteroaryl;
each R⁸ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R⁸ on the same carbon form an oxo;
p is 0, 1, 2, 3, 4, 5, or 6;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom form an oxo.

2. (canceled)

3. (canceled)

4. (canceled)

5. (canceled)

6. (canceled)

7. (canceled)

8. (canceled)

9. (canceled)

10. (canceled)

11. (canceled)

12. The compound of claim 1, or a pharmaceutically acceptable salt, solvate, or stereoisomer thereof, wherein compound is of Formula (Ia): 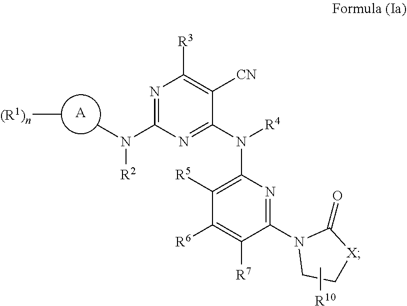
wherein:
X is —O—, —S—, or —C(R⁹)₂—;
each R⁹ is independently hydrogen deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R¹⁰ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R.

13. (canceled)

14. The compound of claim 12, or a pharmaceutically acceptable salt, solvate, or stereoisomer thereof, wherein X is —C(R⁹)₂— and each R⁹ is hydrogen.

15. (canceled)

16. (canceled)

17. The compound of claim 12, or a pharmaceutically acceptable salt, solvate, or stereoisomer thereof, wherein R¹⁰ is hydrogen.

18. (canceled)

19. The compound of claim 1, or a pharmaceutically acceptable salt, solvate, or stereoisomer thereof, wherein R² is hydrogen and R⁴ is hydrogen.

20. (canceled)

21. The compound of claim 1, or a pharmaceutically acceptable salt, solvate, or stereoisomer thereof, wherein R³ is hydrogen.

22. (canceled)

23. (canceled)

24. (canceled)

25. The compound of claim 1, or a pharmaceutically acceptable salt, solvate, or stereoisomer thereof, wherein R⁵ is hydrogen; R⁶ is hydrogen; and R⁷ is hydrogen.

26. (canceled)

27. (canceled)

28. (canceled)

29. (canceled)

30. (canceled)

31. (canceled)

32. (canceled)

33. The compound of claim 1, or a pharmaceutically acceptable salt, solvate, or stereoisomer thereof, wherein Ring A is a phenyl.

34. The compound of claim 1, or a pharmaceutically acceptable salt, solvate, or stereoisomer thereof, wherein Ring A is a bicyclic heteroaryl.

35. (canceled)

36. The compound of claim 1, or a pharmaceutically acceptable salt, solvate, or stereoisomer thereof, wherein each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{1a}.

37. (canceled)

38. (canceled)

39. The compound of claim 1, or a pharmaceutically acceptable salt, solvate, or stereoisomer thereof, wherein each R¹ is independently halogen, C₁-C₆alkyl, C₁-C₆heteroalkyl, or heterocycloalkyl; wherein the alkyl, and heterocycloalkyl is optionally substituted with one or more R^{1a}.

40. The compound of claim 1, or a pharmaceutically acceptable salt, solvate, or stereoisomer thereof, wherein each R¹ is independently heterocycloalkyl optionally substituted with one or more R^{1a}.

41. (canceled)

42. (canceled)

43. (canceled)

44. (canceled)

45. (canceled)

46. (canceled)

47. The compound of claim 1, or a pharmaceutically acceptable salt, solvate, or stereoisomer thereof, wherein each R^{1a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, is heterocycloalkyl is optionally substituted with one or more R^{1b}; or two R^{1a} on the same atom form an oxo.

48. (canceled)

49. (canceled)

50. (canceled)

51. The compound of claim 1, or a pharmaceutically acceptable salt, solvate, or stereoisomer thereof, wherein each R^{1a} is independently C₁-C₆alkyl, C₁-C₆haloalkyl, or C₁-C₆heteroalkyl.

52. (canceled)

53. (canceled)

54. The compound of claim 1, or a pharmaceutically acceptable salt, solvate, or stereoisomer thereof, wherein each R^{1b} is independently deuterium, halogen, —OH, C₁-C₆alkyl, or C₁-C₆haloalkyl.

55. (canceled)

56. (canceled)

57. (canceled)

58. The compound of claim 1, or a pharmaceutically acceptable salt, solvate, or stereoisomer thereof, wherein n is 1 or 2.

59. (canceled)

60. A compound of Formula (II), or a pharmaceutically acceptable salt, solvate, or stereoisomer thereof: 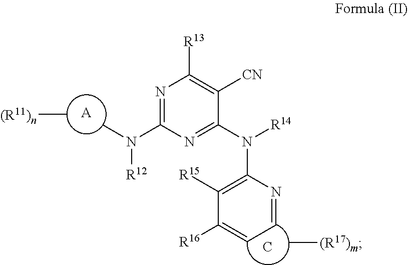
wherein:
Ring A is cycloalkyl, heterocycloalkyl, aryl, or heteroaryl;
each R¹¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{11a};
or two R¹¹ on the same atom form an oxo;
each R^{11a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{11b};
or two R^{11a} on the same atom form an oxo;
each R^{11b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R^{11b} on the same atom form an oxo;
n is 0, 1, 2, 3, 4, 5, or 6;
R¹² is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R¹³ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R¹⁴ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R¹⁵ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R¹⁶ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring C is cycloalkyl or heterocycloalkyl;
each R¹⁷ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
or two R¹⁷ on the same carbon form an oxo;
m is 0, 1, 2, 3, 4, 5, or 6;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom form an oxo;
provided that the compound is not

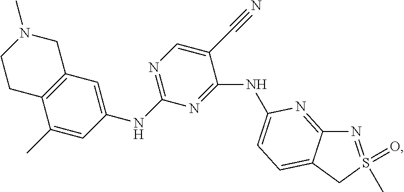 [SMILES: Cc1cc(Nc2ncc(C#N)c(Nc3ccc4c(n3)N=S(C)(=O)C4)n2)cc2c1CCN(C)C2]

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. A compound selected from the group consisting of: 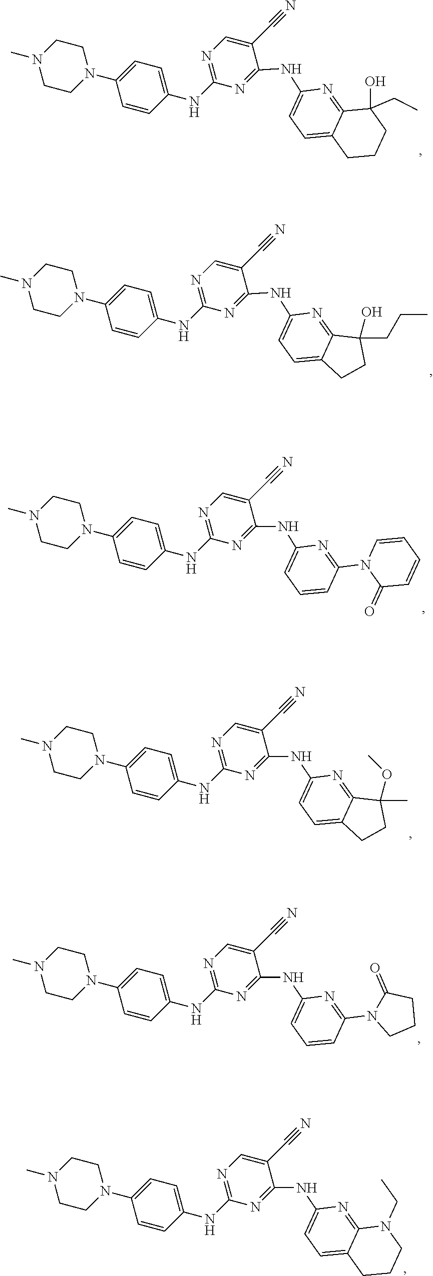 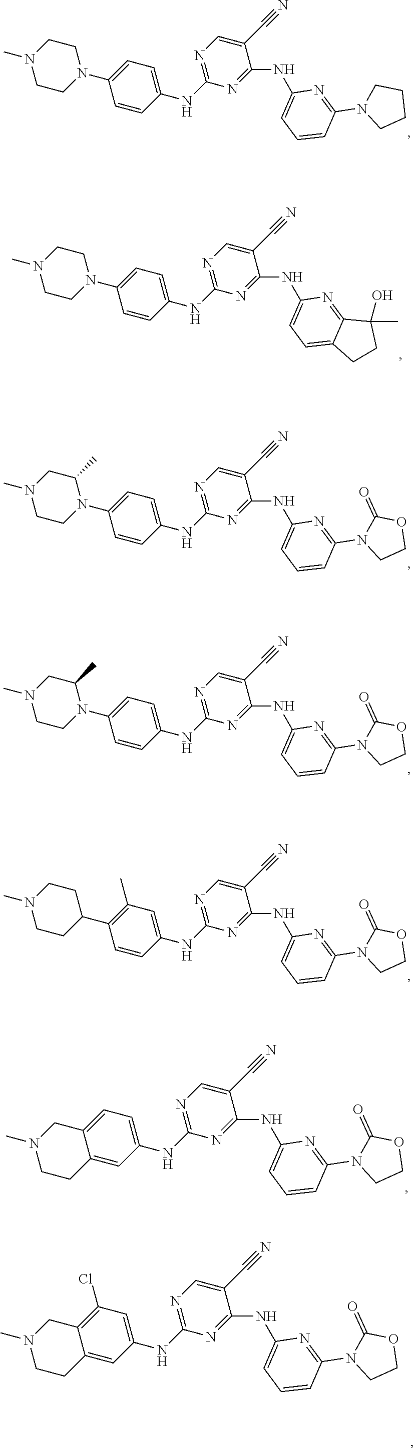 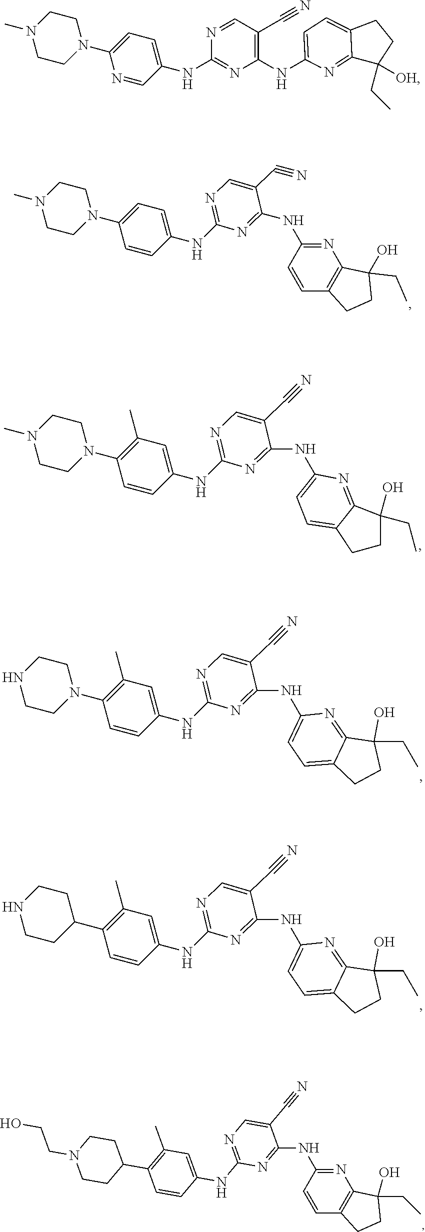 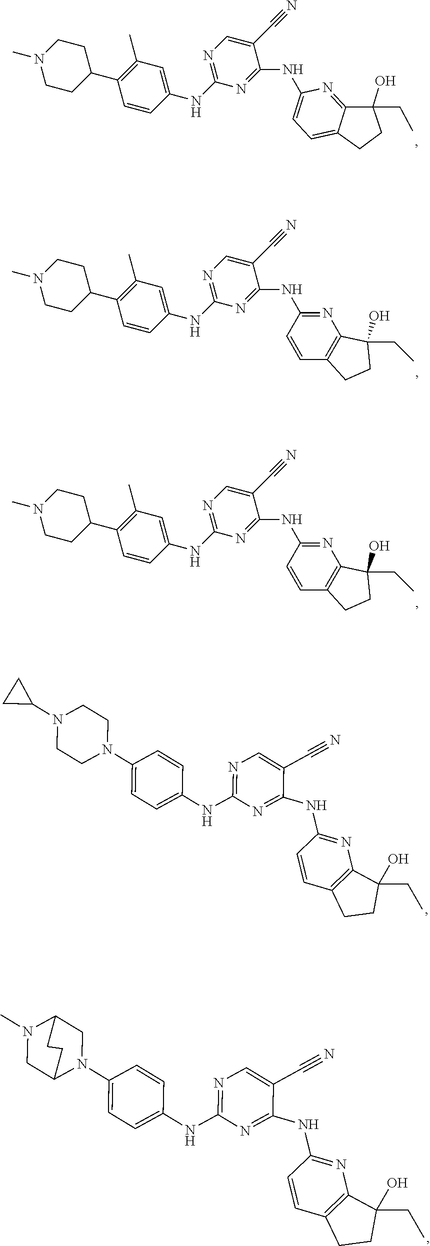 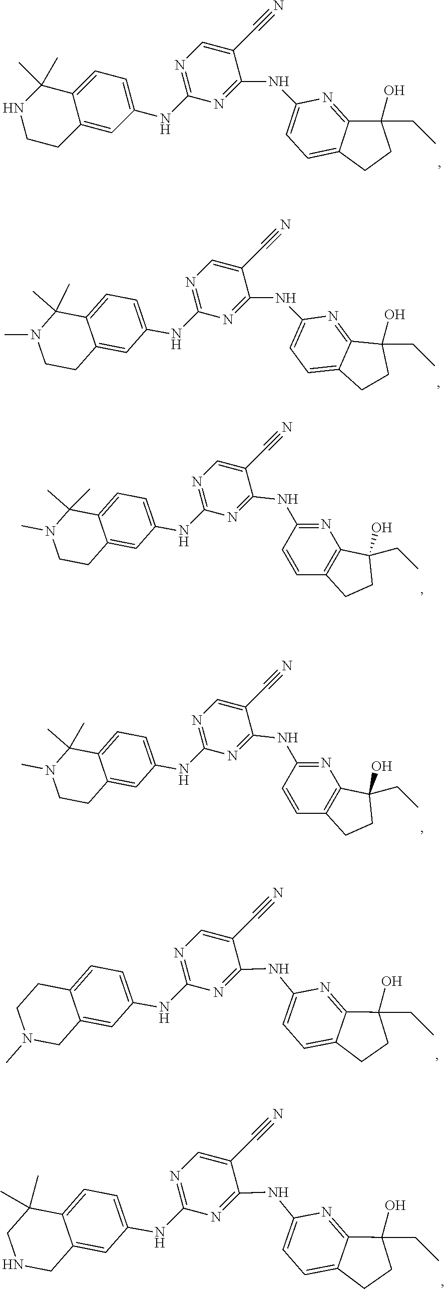 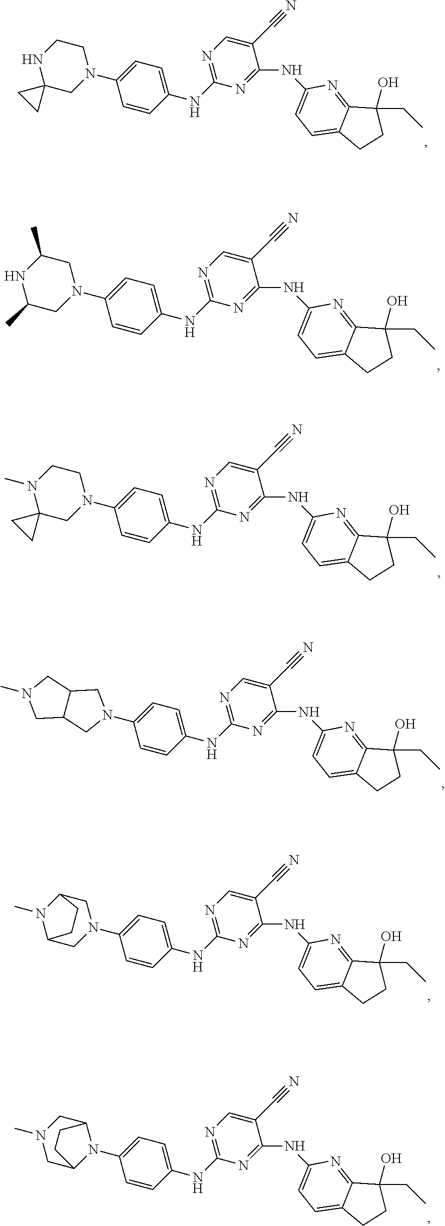 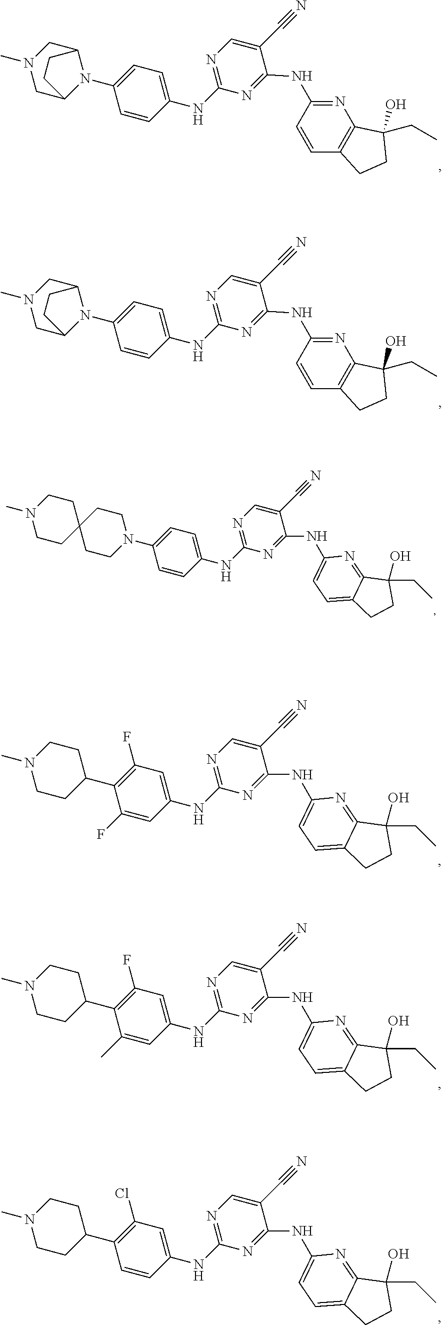 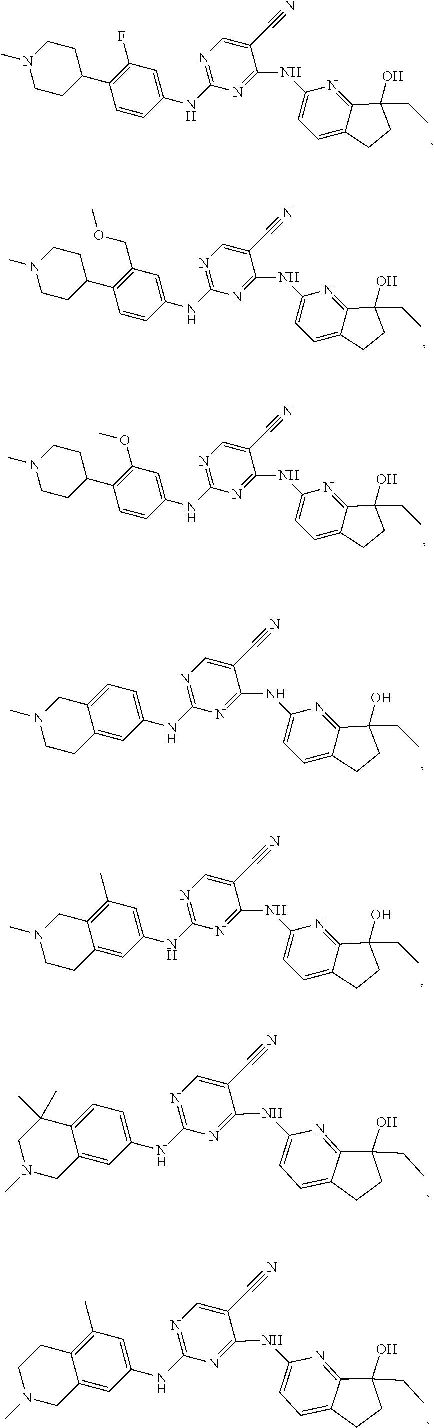 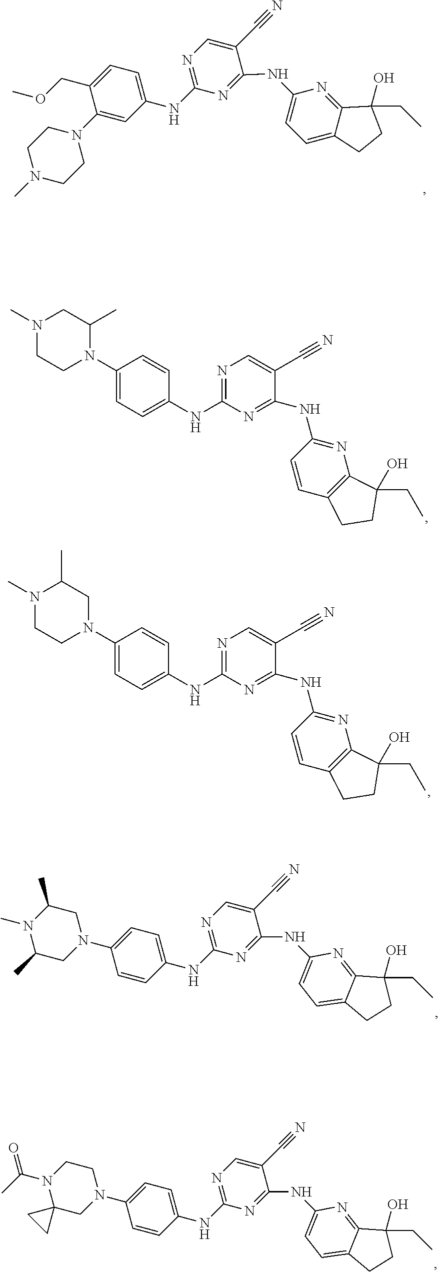 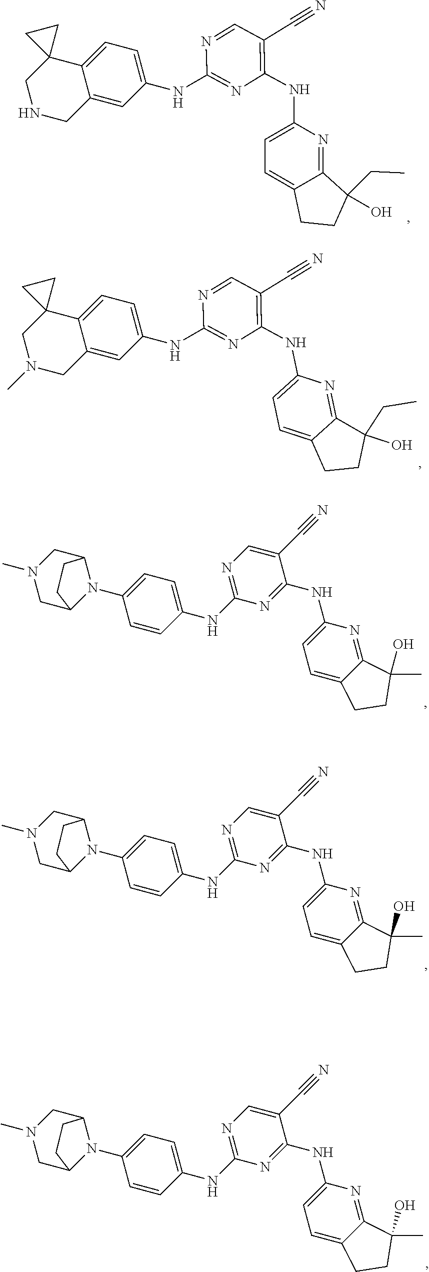 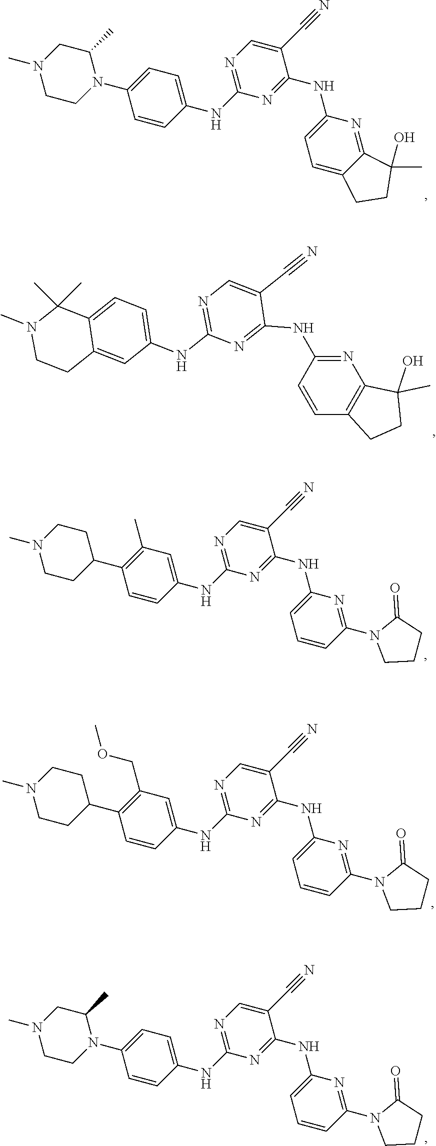 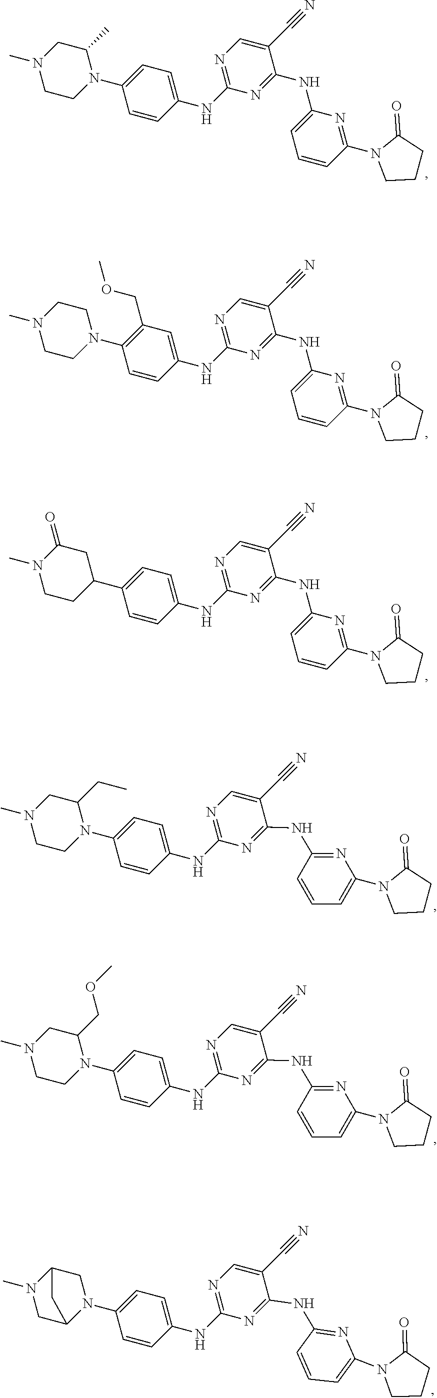 [SMILES: C[C@H]1CN(C)CCN1c1ccc(Nc2ncc(C#N)c(Nc3cccc(N4CCCC4=O)n3)n2)cc1 ; COCc1cc(Nc2ncc(C#N)c(Nc3cccc(N4CCCC4=O)n3)n2)ccc1N1CCN(C)CC1 ; CN1CCC(c2ccc(Nc3ncc(C#N)c(Nc4cccc(N5CCCC5=O)n4)n3)cc2)CC1=O ; CCC1CN(C)CCN1c1ccc(Nc2ncc(C#N)c(Nc3cccc(N4CCCC4=O)n3)n2)cc1 ; COCC1CN(C)CCN1c1ccc(Nc2ncc(C#N)c(Nc3cccc(N4CCCC4=O)n3)n2)cc1 ; CN1CC2CC1CN2c1ccc(Nc2ncc(C#N)c(Nc3cccc(N4CCCC4=O)n3)n2)cc1] 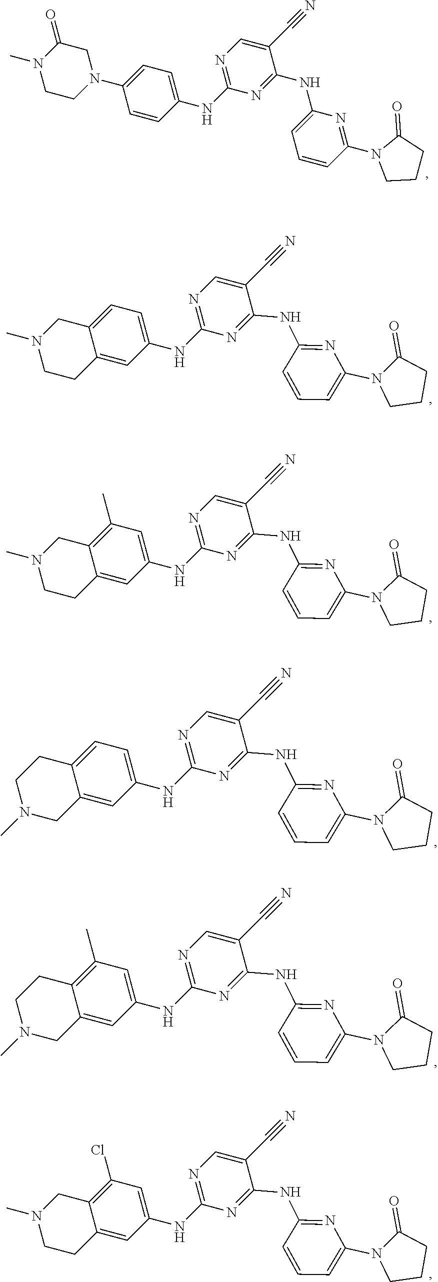 [SMILES: CN1CCN(c2ccc(Nc3ncc(C#N)c(Nc4cccc(N5CCCC5=O)n4)n3)cc2)CC1=O ; CN1CCc2cc(Nc3ncc(C#N)c(Nc4cccc(N5CCCC5=O)n4)n3)ccc2C1 ; Cc1cc(Nc2ncc(C#N)c(Nc3cccc(N4CCCC4=O)n3)n2)cc2c1CN(C)CC2 ; CN1CCc2ccc(Nc3ncc(C#N)c(Nc4cccc(N5CCCC5=O)n4)n3)cc2C1 ; Cc1cc(Nc2ncc(C#N)c(Nc3cccc(N4CCCC4=O)n3)n2)cc2c1CCN(C)C2 ; CN1CCc2cc(Nc3ncc(C#N)c(Nc4cccc(N5CCCC5=O)n4)n3)cc(Cl)c2C1] 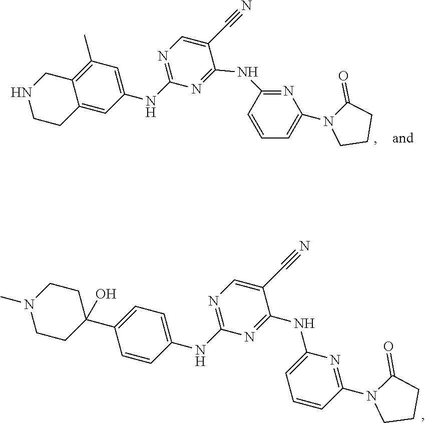 [SMILES: Cc1cc(Nc2ncc(C#N)c(Nc3cccc(N4CCCC4=O)n3)n2)cc2c1CNCC2 ; CN1CCC(O)(c2ccc(Nc3ncc(C#N)c(Nc4cccc(N5CCCC5=O)n4)n3)cc2)CC1]
or a pharmaceutically acceptable salt, solvate, tautomer, or stereoisomer thereof.

118. A pharmaceutical composition comprising a compound of claim 1, or a pharmaceutically acceptable salt, solvate, tautomer, or stereoisomer thereof, and a pharmaceutically acceptable excipient.

119. A method of treating cancer in a subject, comprising administering to the subject a compound of claim 1, or a pharmaceutically acceptable salt, solvate, tautomer, or stereoisomer thereof.

120.-136. (canceled)
